SEQUENCE LISTING (248 sequences)

SEQ ID NO: 1 - protein, 602 aa, synthetic construct
  REGION 1..602: note = NiVG protein attachment glycoprotein (602 aa)
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 2 - protein, 604 aa, synthetic construct
  REGION 1..604: note = Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 3 - protein, 603 aa, synthetic construct
  REGION 1..603: note = Hendra Virus G Protein without Met
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 4 - protein, 602 aa, synthetic construct
  REGION 1..602: note = Nipah Virus G Protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 5 - protein, 601 aa, synthetic construct
  REGION 1..601: note = Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 6 - protein, 622 aa, synthetic construct
  REGION 1..622: note = Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 7 - protein, 621 aa, synthetic construct
  REGION 1..621: note = Cedar Virus G Protein (No Met)
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 8 - protein, 632 aa, synthetic construct
  REGION 1..632: note = Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 9 - protein, 631 aa, synthetic construct
  REGION 1..631: note = Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 10 - protein, 625 aa, synthetic construct
  REGION 1..625: note = Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 11 - protein, 624 aa, synthetic construct
  REGION 1..624: note = Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 12 - protein, 597 aa, synthetic construct
  REGION 1..597: note = NiVG protein attachment glycoprotein Truncated 5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 13 - protein, 582 aa, synthetic construct
  REGION 1..582: note = NiVG protein attachment glycoprotein Truncated 20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 14 - protein, 577 aa, synthetic construct
  REGION 1..577: note = NiVG protein attachment glycoprotein Truncated 25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 500 aa, synthetic construct
  REGION 1..500: note = Nipah virus NiV-F F0 T234 truncation (aa 525-544) AND mutation on N-linked glycosylation site
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 16 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 17 - protein, 569 aa, synthetic construct
  REGION 1..569: note = NiVG protein attachment glycoprotein Truncated and mutated (E501 A, W504A, Q530A, E533A) NiV G protein (Gc 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 18 - protein, 568 aa, synthetic construct
  REGION 1..568: note = NiVG protein attachment glycoprotein Truncated and mutated (E501 A, W504A, Q530A, E533A) NiV G protein (Gc 34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 19 - protein, 524 aa, synthetic construct; identical to SEQ ID NO: 16
  REGION 1..524: note = Truncated NiV fusion glycoprotein (FcDelta22) at cytoplasmic tail (with signal sequence)

SEQ ID NO: 20 - protein, 500 aa, synthetic construct
  REGION 1..500: note = Nipah virus NiV-F F0 T234 truncation (aa 525-544)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 21 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 22 - protein, 552 aa, synthetic construct
  REGION 1..552: note = NivG protein attachment glycoprotein Without cytoplasmic tail Uniprot Q9IH62
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 23 - protein, 604 aa, synthetic construct; identical to SEQ ID NO: 2
  REGION 1..604: note = Hendra virus G protein Uniprot O89343

SEQ ID NO: 24 - protein, 603 aa, synthetic construct; identical to SEQ ID NO: 3
  REGION 1..603: note = Hendra virus G protein Uniprot O89343 Without N-terminal methionine

SEQ ID NO: 25 - protein, 555 aa, synthetic construct
  REGION 1..555: note = Hendra virus G protein Uniprot O89343 Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 26 - protein, 555 aa, synthetic construct; identical to SEQ ID NO: 25
  REGION 1..555: note = Hendra virus G protein Uniprot O89343 Without cytoplasmic tail

SEQ ID NO: 27 - protein, 602 aa, synthetic construct; identical to SEQ ID NO: 1
  REGION 1..602: note = Hendra virus G protein Uniprot O89343 Without cytoplasmic tail

SEQ ID NO: 28 - protein, 546 aa, synthetic construct
  REGION 1..546: note = Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 29 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 30 - protein, 546 aa, synthetic construct
  REGION 1..546: note = Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 31 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 32 - protein, 557 aa, synthetic construct
  REGION 1..557: note = Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 33 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 34 - protein, 545 aa, synthetic construct
  REGION 1..545: note = Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 35 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 36 - protein, 662 aa, synthetic construct
  REGION 1..662: note = Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 37 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 38 - protein, 26 aa, synthetic construct
  REGION 1..26: note = signal sequence
MVVILDKRCYCNLLILILMISECSVG

SEQ ID NO: 39 - protein, 83 aa, synthetic construct
  REGION 1..83: note = Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 40 - protein, 546 aa, synthetic construct; identical to SEQ ID NO: 30
  REGION 1..546: note = Nipah virus F Protein

SEQ ID NO: 41 - protein, 520 aa, synthetic construct; identical to SEQ ID NO: 31
  REGION 1..520: note = Nipah virus NiV-F F0 (aa 27-546)

SEQ ID NO: 42 - protein, 569 aa, synthetic construct
  REGION 1..569: note = NiVG protein attachment glycoprotein Truncated (Gc 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 43 - protein, 573 aa, synthetic construct
  REGION 1..573: note = NiVG protein attachment glycoprotein Truncated 30
MTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 44 - protein, 592 aa, synthetic construct
  REGION 1..592: note = NiVG protein attachment glycoprotein Truncated 10
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 45 - protein, 587 aa, synthetic construct
  REGION 1..587: note = NiVG protein attachment glycoprotein Truncated 15
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 46 - protein, 437 aa, synthetic construct
  REGION 1..437: note = Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 47 - protein, 21 aa, synthetic construct
  REGION 1..21: note = CD8alpha signal peptide
MALPVTALLLPLALLLHAARP

SEQ ID NO: 48 - protein, 20 aa, synthetic construct
  REGION 1..20: note = IgK signal peptide
METDTLLLWVLLLWVPGSTG

SEQ ID NO: 49 - protein, 22 aa, synthetic construct
  REGION 1..22: note = GMCSFR-alpha (CSF2RA) signal peptide
MLLLVTSLLLCELPHPAFLLIP

SEQ ID NO: 50 - protein, 45 aa, synthetic construct
  REGION 1..45: note = CD8alpha hinge domain
TTTPAPRPPTPAPTIASQPLSLRPEACRPAAGGAVHTRGLDFACD

SEQ ID NO: 51 - protein, 39 aa, synthetic construct
  REGION 1..39: note = CD28 hinge domain
IEVMYPPPYLDNEKSNGTIIHVKGKHLCPSPLFPGPSKP

SEQ ID NO: 52 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 53 - protein, 12 aa, synthetic construct
  REGION 1..12: note = IgG4 hinge domain
ESKYGPPCPPCP

SEQ ID NO: 54 - protein, 12 aa, synthetic construct
  REGION 1..12: note = IgG4 hinge domain
ESKYGPPCPSCP

SEQ ID NO: 55 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 24 aa, synthetic construct
  REGION 1..24: note = CD8alpha transmembrane domain
IYIWAPLAGTCGVLLLSLVITLYC

SEQ ID NO: 57 - protein, 27 aa, synthetic construct
  REGION 1..27: note = CD28 transmembrane domain
FWVLVVVGGVLACYSLLVTVAFIIFWV

SEQ ID NO: 58 - protein, 28 aa, synthetic construct
  REGION 1..28: note = CD28 transmembrane domain
MFWVLVVVGGVLACYSLLVTVAFIIFWV

SEQ ID NO: 59 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 60 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 61 - protein, 112 aa, synthetic construct
  REGION 1..112: note = CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 112 aa, synthetic construct
  REGION 1..112: note = CD3zeta signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 64 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 65 - protein, 6 aa, synthetic construct
  REGION 1..6: note = Anti-CD19 FMC63 scFv light chain CDR1
QDISKY

SEQ ID NO: 66 - skipped

SEQ ID NO: 67 - protein, 9 aa, synthetic construct
  REGION 1..9: note = Anti-CD19 FMC63 scFv light chain CDR3
QQGNTLPYT

SEQ ID NO: 68 - protein, 18 aa, synthetic construct
  REGION 1..18: note = Whitlow linker
GSTSGSGKPGSGEGSTKG

SEQ ID NO: 69 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 70 - protein, 8 aa, synthetic construct
  REGION 1..8: note = Anti-CD19 FMC63 scFv heavy chain CDR1
GVSLPDYG

SEQ ID NO: 71 - protein, 7 aa, synthetic construct
  REGION 1..7: note = Anti-CD19 FMC63 scFv heavy chain CDR2
IWGSETT

SEQ ID NO: 72 - protein, 14 aa, synthetic construct
  REGION 1..14: note = Anti-CD19 FMC63 scFv heavy chain CDR3
AKHYYYGGSYAMDY

SEQ ID NO: 73 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 74 - DNA, 1467 bp, synthetic construct
  source 1..1467: mol_type = other DNA
  unsure 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 75 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - DNA, 1458 bp, synthetic construct
  source 1..1458: mol_type = other DNA
  unsure 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 77 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 1383 bp, synthetic construct
  source 1..1383: mol_type = other DNA
  unsure 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 79 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1467 bp, synthetic construct
  source 1..1467: mol_type = other DNA
  unsure 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 81 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 83 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 84 - protein, 10 aa, synthetic construct
  REGION 1..10: note = Anti-CD20 Leu16 scFv light chain CDR1
RASSSVNYMD

SEQ ID NO: 85 - protein, 7 aa, synthetic construct
  REGION 1..7: note = Anti-CD20 Leu16 scFv light chain CDR2
ATSNLAS

SEQ ID NO: 86 - protein, 9 aa, synthetic construct
  REGION 1..9: note = Anti-CD20 Leu16 scFv light chain CDR3
QQWSFNPPT

SEQ ID NO: 87 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 88 - protein, 5 aa, synthetic construct
  REGION 1..5: note = Anti-CD20 Leu16 scFv heavy chain CDR1
SYNMH

SEQ ID NO: 89 - protein, 17 aa, synthetic construct
  REGION 1..17: note = Anti-CD20 Leu16 scFv heavy chain CDR2
AIYPGNGDTSYNQKFKG

SEQ ID NO: 90 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 91 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 92 - protein, 10 aa, synthetic construct
  REGION 1..10: note = Anti-CD22 m971 scFv heavy chain CDR1
GDSVSSNSAA

SEQ ID NO: 93 - protein, 9 aa, synthetic construct
  REGION 1..9: note = Anti-CD22 m971 scFv heavy chain CDR2
TYYRSKWYN

SEQ ID NO: 94 - protein, 14 aa, synthetic construct
  REGION 1..14: note = Anti-CD22 m971 scFv heavy chain CDR3
AREVTGDLEDAFDI

SEQ ID NO: 95 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 96 - protein, 6 aa, synthetic construct
  REGION 1..6: note = Anti-CD22 m971 scFv light chain CDR1
QTIWSY

SEQ ID NO: 97 - skipped

SEQ ID NO: 98 - protein, 9 aa, synthetic construct
  REGION 1..9: note = Anti-CD22 m971 scFv light chain CDR3
QQSYSIPQT

SEQ ID NO: 99 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 100 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 101 - protein, 10 aa, synthetic construct
  REGION 1..10: note = Anti-CD22 m971-L7 scFv heavy chain CDR1
GDSVSSNSVA

SEQ ID NO: 102 - protein, 9 aa, synthetic construct
  REGION 1..9: note = Anti-CD22 m971-L7 scFv heavy chain CDR2
TYYRSTWYN

SEQ ID NO: 103 - protein, 14 aa, synthetic construct
  REGION 1..14: note = Anti-CD22 m971-L7 scFv heavy chain CDR3
AREVTGDLEDAFDI

SEQ ID NO: 104 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 105 - protein, 6 aa, synthetic construct
  REGION 1..6: note = Anti-CD22 m971-L7 scFv light chain CDR1
QTIWSY

SEQ ID NO: 106 - skipped

SEQ ID NO: 107 - protein, 9 aa, synthetic construct
  REGION 1..9: note = Anti-CD22 m971-L7 scFv light chain CDR3
QQSYSIPQT

SEQ ID NO: 108 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 109 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 110 - protein, 15 aa, synthetic construct
  REGION 1..15: note = Anti-BCMA C11D5.3 scFv light chain CDR1
RASESVSVIGAHLIH

SEQ ID NO: 111 - protein, 7 aa, synthetic construct
  REGION 1..7: note = Anti-BCMA C11D5.3 scFv light chain CDR2
LASNLET

SEQ ID NO: 112 - protein, 9 aa, synthetic construct
  REGION 1..9: note = Anti-BCMA C11D5.3 scFv light chain CDR3
LQSRIFPRT

SEQ ID NO: 113 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 114 - protein, 5 aa, synthetic construct
  REGION 1..5: note = Anti-BCMA C11D5.3 scFv heavy chain CDR1
DYSIN

SEQ ID NO: 115 - protein, 17 aa, synthetic construct
  REGION 1..17: note = Anti-BCMA C11D5.3 scFv heavy chain CDR2
WINTETREPAYAYDFRG

SEQ ID NO: 116 - protein, 8 aa, synthetic construct
  REGION 1..8: note = Anti-BCMA C11D5.3 scFv heavy chain CDR3
DYSYAMDY

SEQ ID NO: 117 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 118 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 119 - protein, 15 aa, synthetic construct
  REGION 1..15: note = Anti-BCMA C12A3.2 scFv light chain CDR1
RASESVTILGSHLIY

SEQ ID NO: 120 - protein, 7 aa, synthetic construct
  REGION 1..7: note = Anti-BCMA C12A3.2 scFv light chain CDR2
LASNVQT

SEQ ID NO: 121 - protein, 9 aa, synthetic construct
  REGION 1..9: note = Anti-BCMA C12A3.2 scFv light chain CDR3
LQSRTIPRT

SEQ ID NO: 122 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 123 - protein, 5 aa, synthetic construct
  REGION 1..5: note = Anti-BCMA C12A3.2 scFv heavy chain CDR1
HYSMN

SEQ ID NO: 124 - protein, 17 aa, synthetic construct
  REGION 1..17: note = Anti-BCMA C12A3.2 scFv heavy chain CDR2
RINTESGVPIYADDFKG

SEQ ID NO: 125 - protein, 8 aa, synthetic construct
  REGION 1..8: note = Anti-BCMA C12A3.2 scFv heavy chain CDR3
DYLYSLDF

SEQ ID NO: 126 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 127 - protein, 8 aa, synthetic construct
  REGION 1..8: note = Anti-BCMA FHVH33 CDR1
GFTFSSYA

SEQ ID NO: 128 - protein, 8 aa, synthetic construct
  REGION 1..8: note = Anti-BCMA FHVH33 CDR2
ISGSGDYI

SEQ ID NO: 129 - protein, 14 aa, synthetic construct
  REGION 1..14: note = Anti-BCMA FHVH33 CDR3
AKEGTGANSSLADY

SEQ ID NO: 130 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 131 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 132 - protein, 6 aa, synthetic construct
  REGION 1..6: note = Anti-BCMA CT103A scFv light chain CDR1
QSISSY

SEQ ID NO: 133 - skipped

SEQ ID NO: 134 - protein, 8 aa, synthetic construct
  REGION 1..8: note = Anti-BCMA CT103A scFv light chain CDR3
QQKYDLLT

SEQ ID NO: 135 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 136 - protein, 10 aa, synthetic construct
  REGION 1..10: note = Anti-BCMA CT103A scFv heavy chain CDR1
GGSISSSSYY

SEQ ID NO: 137 - protein, 7 aa, synthetic construct
  REGION 1..7: note = Anti-BCMA CT103A scFv heavy chain CDR2
ISYSGST

SEQ ID NO: 138 - protein, 11 aa, synthetic construct
  REGION 1..11: note = Anti-BCMA CT103A scFv heavy chain CDR3
ARDRGDTILDV

SEQ ID NO: 139 - DNA, 1503 bp, synthetic construct
  source 1..1503: mol_type = other DNA
  unsure 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 140 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 44 aa, synthetic construct
  REGION 1..44: note = CD8 Transmembrane
ACRPAAGGAVHTRGLDFACDIYIWAPLAGTCGVLLLSLVITLYC

SEQ ID NO: 142 - protein, 25 aa, synthetic construct
  REGION 1..25: note = CD8 Hinge
TTTPAPRPPTPAPTIASQPLSLRPE

SEQ ID NO: 143 - protein, 15 aa, synthetic construct
  REGION 1..15: note = linker
GGGGSGGGGSGGGGS

SEQ ID NO: 144 - protein, 13 aa, synthetic construct
  REGION 1..13: note = Anti-CD20 Leu16 scFv heavy chain CDR3
SNYYGSSYWFFDV

SEQ ID NO: 145 - protein, 8 aa, synthetic construct
  REGION 1..8: note = CDR-H1
GFTFSGYW

SEQ ID NO: 146 - protein, 8 aa, synthetic construct
  REGION 1..8: note = CDR-H2
ISPGGGST

SEQ ID NO: 147 - protein, 14 aa, synthetic construct
  REGION 1..14: note = CDR-H3
ASSLTATHTYEYDY

SEQ ID NO: 148 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 149 - protein, 8 aa, synthetic construct
  REGION 1..8: note = CDR-H1
GYTFSNYW

SEQ ID NO: 150 - protein, 8 aa, synthetic construct
  REGION 1..8: note = CDR-H2
ILPGSGST

SEQ ID NO: 151 - protein, 13 aa, synthetic construct
  REGION 1..13: note = CDR-H3
ARRGYGYDEGFDY

SEQ ID NO: 152 - protein, 6 aa, synthetic construct
  REGION 1..6: note = CDR-L1
QDINSY

SEQ ID NO: 153 - skipped

SEQ ID NO: 154 - protein, 9 aa, synthetic construct
  REGION 1..9: note = CDR-L3
LQYDEFPPT

SEQ ID NO: 155 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 156 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 157 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 158 - protein, 8 aa, synthetic construct
  REGION 1..8: note = CDR-H1
GYTFTDYV

SEQ ID NO: 159 - protein, 8 aa, synthetic construct
  REGION 1..8: note = CDR-H2
IYPGSGSS

SEQ ID NO: 160 - protein, 10 aa, synthetic construct
  REGION 1..10: note = CDR-H3
ARPGDLGFAY

SEQ ID NO: 161 - protein, 10 aa, synthetic construct
  REGION 1..10: note = CDR-L1
QSVDYDGDSY

SEQ ID NO: 162 - skipped

SEQ ID NO: 163 - protein, 9 aa, synthetic construct
  REGION 1..9: note = CDR-L3
QQSNKDPFT

SEQ ID NO: 164 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 165 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 166 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 167 - protein, 8 aa, synthetic construct
  REGION 1..8: note = CDR-H1
GGTFSSYA

SEQ ID NO: 168 - protein, 8 aa, synthetic construct
  REGION 1..8: note = CDR-H2
INPNSGGT

SEQ ID NO: 169 - protein, 11 aa, synthetic construct
  REGION 1..11: note = CDR-H3
ARDGYSGSYSD

SEQ ID NO: 170 - protein, 12 aa, synthetic construct
  REGION 1..12: note = CDR-L1
QSVLSSSYNKNY

SEQ ID NO: 171 - skipped

SEQ ID NO: 172 - protein, 9 aa, synthetic construct
  REGION 1..9: note = CDR-L3
QQYYSTPWT

SEQ ID NO: 173 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 174 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 175 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 176 - protein, 8 aa, synthetic construct
  REGION 1..8: note = CDR-H1
GYTFTSYD

SEQ ID NO: 177 - protein, 8 aa, synthetic construct
  REGION 1..8: note = CDR-H2
IIPLSGAP

SEQ ID NO: 178 - protein, 15 aa, synthetic construct
  REGION 1..15: note = CDR-H3
ARGALYNWNDGWFDP

SEQ ID NO: 179 - protein, 6 aa, synthetic construct
  REGION 1..6: note = CDR-L1
QDIGDY

SEQ ID NO: 180 - skipped

SEQ ID NO: 181 - protein, 9 aa, synthetic construct
  REGION 1..9: note = CDR-L3
QQANSFPLT

SEQ ID NO: 182 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 183 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 184 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 185 - protein, 8 aa, synthetic construct
  REGION 1..8: note = CDR-H1
GDTFSDHA

SEQ ID NO: 186 - protein, 8 aa, synthetic construct
  REGION 1..8: note = CDR-H2
MNPKIGNT

SEQ ID NO: 187 - protein, 12 aa, synthetic construct
  REGION 1..12: note = CDR-H3
VYDSSGYDAFDI

SEQ ID NO: 188 - protein, 12 aa, synthetic construct
  REGION 1..12: note = CDR-L1
QSVLSTSYNRNF

SEQ ID NO: 189 - protein, 9 aa, synthetic construct
  REGION 1..9: note = CDR-L3
QQYYSTPYT

SEQ ID NO: 190 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 191 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 192 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 193 - protein, 8 aa, synthetic construct
  REGION 1..8: note = CDR-H1
GYSLITHW

SEQ ID NO: 194 - protein, 8 aa, synthetic construct
  REGION 1..8: note = CDR-H2
INPSDGVT

SEQ ID NO: 195 - protein, 11 aa, synthetic construct
  REGION 1..11: note = CDR-H3
AREYYGEGFDY

SEQ ID NO: 196 - protein, 6 aa, synthetic construct
  REGION 1..6: note = CDR-L1
QGISNY

SEQ ID NO: 197 - skipped

SEQ ID NO: 198 - protein, 9 aa, synthetic construct
  REGION 1..9: note = CDR-L3
QQSYSTPLT

SEQ ID NO: 199 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 200 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 201 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 202 - protein, 5 aa, synthetic construct
  REGION 1..5: note = CDR-H1
GYWMY

SEQ ID NO: 203 - protein, 17 aa, synthetic construct
  REGION 1..17: note = CDR-H2
AISPGGGSTYYPDSVKG

SEQ ID NO: 204 - protein, 12 aa, synthetic construct
  REGION 1..12: note = CDR-H3
SLTATHTYEYDY

SEQ ID NO: 205 - protein, 7 aa, synthetic construct
  REGION 1..7: note = CDR-H1
GFTFSGY

SEQ ID NO: 206 - protein, 6 aa, synthetic construct
  REGION 1..6: note = CDR-H2
SPGGGS

SEQ ID NO: 207 - protein, 5 aa, synthetic construct
  REGION 1..5: note = CDR-H1
NYWIE

SEQ ID NO: 208 - protein, 17 aa, synthetic construct
  REGION 1..17: note = CDR-H2
EILPGSGSTSYNEKFKG

SEQ ID NO: 209 - protein, 11 aa, synthetic construct
  REGION 1..11: note = CDR-H3
RGYGYDEGFDY

SEQ ID NO: 210 - protein, 11 aa, synthetic construct
  REGION 1..11: note = CDR-L1
KASQDINSYLS

SEQ ID NO: 211 - protein, 7 aa, synthetic construct
  REGION 1..7: note = CDR-L2
RANRLVD

SEQ ID NO: 212 - protein, 7 aa, synthetic construct
  REGION 1..7: note = CDR-H1
GYTFSNY

SEQ ID NO: 213 - protein, 6 aa, synthetic construct
  REGION 1..6: note = CDR-H2
LPGSGS

SEQ ID NO: 214 - protein, 5 aa, synthetic construct
  REGION 1..5: note = CDR-H1
DYVIS

SEQ ID NO: 215 - protein, 17 aa, synthetic construct
  REGION 1..17: note = CDR-H2
EIYPGSGSSYYNEKFKG

SEQ ID NO: 216 - protein, 8 aa, synthetic construct
  REGION 1..8: note = CDR-H3
PGDLGFAY

SEQ ID NO: 217 - protein, 15 aa, synthetic construct
  REGION 1..15: note = CDR-L1
KASQSVDYDGDSYMN

SEQ ID NO: 218 - protein, 7 aa, synthetic construct
  REGION 1..7: note = CDR-L2
AASNLES

SEQ ID NO: 219 - protein, 7 aa, synthetic construct
  REGION 1..7: note = CDR-H1
GYTFTDY

SEQ ID NO: 220 - protein, 6 aa, synthetic construct
  REGION 1..6: note = CDR-H2
YPGSGS

SEQ ID NO: 221 - protein, 5 aa, synthetic construct
  REGION 1..5: note = CDR-H1
SYAIS

SEQ ID NO: 222 - protein, 17 aa, synthetic construct
  REGION 1..17: note = CDR-H2
WINPNSGGTNYAQKFQG

SEQ ID NO: 223 - protein, 9 aa, synthetic construct
  REGION 1..9: note = CDR-H3
DGYSGSYSD

SEQ ID NO: 224 - protein, 17 aa, synthetic construct
  REGION 1..17: note = CDR-L1
KSSQSVLSSSYNKNYLA

SEQ ID NO: 225 - protein, 7 aa, synthetic construct
  REGION 1..7: note = CDR-L2
WASTRES

SEQ ID NO: 226 - protein, 7 aa, synthetic construct
  REGION 1..7: note = CDR-H1
GGTFSSY

SEQ ID NO: 227 - protein, 6 aa, synthetic construct
  REGION 1..6: note = CDR-H2
NPNSGG

SEQ ID NO: 228 - protein, 5 aa, synthetic construct
  REGION 1..5: note = CDR-H1
SYDIN

SEQ ID NO: 229 - protein, 17 aa, synthetic construct
  REGION 1..17: note = CDR-H2
GIIPLSGAPNYAHKFQG

SEQ ID NO: 230 - protein, 13 aa, synthetic construct
  REGION 1..13: note = CDR-H3
GALYNWNDGWFDP

SEQ ID NO: 231 - protein, 11 aa, synthetic construct
  REGION 1..11: note = CDR-L1
RASQDIGDYLA

SEQ ID NO: 232 - protein, 7 aa, synthetic construct
  REGION 1..7: note = CDR-L2
DASSLQS

SEQ ID NO: 233 - protein, 7 aa, synthetic construct
  REGION 1..7: note = CDR-H1
GYTFTSY

SEQ ID NO: 234 - protein, 6 aa, synthetic construct
  REGION 1..6: note = CDR-H2
IPLSGA

SEQ ID NO: 235 - protein, 5 aa, synthetic construct
  REGION 1..5: note = CDR-H1
DHAIN

SEQ ID NO: 236 - protein, 17 aa, synthetic construct
  REGION 1..17: note = CDR-H2
WMNPKIGNTGYAQKFQG

SEQ ID NO: 237 - protein, 10 aa, synthetic construct
  REGION 1..10: note = CDR-H3
DSSGYDAFDI

SEQ ID NO: 238 - protein, 17 aa, synthetic construct
  REGION 1..17: note = CDR-L1
KSSQSVLSTSYNRNFLA

SEQ ID NO: 239 - protein, 7 aa, synthetic construct
  REGION 1..7: note = CDR-L2
WASTRQS

SEQ ID NO: 240 - protein, 7 aa, synthetic construct
  REGION 1..7: note = CDR-H1
GDTFSDH

SEQ ID NO: 241 - protein, 6 aa, synthetic construct
  REGION 1..6: note = CDR-H2
NPKIGN

SEQ ID NO: 242 - protein, 5 aa, synthetic construct
  REGION 1..5: note = CDR-H1
THWMH

SEQ ID NO: 243 - protein, 17 aa, synthetic construct
  REGION 1..17: note = CDR-H2
MINPSDGVTYYAQTFQG

SEQ ID NO: 244 - protein, 9 aa, synthetic construct
  REGION 1..9: note = CDR-H3
EYYGEGFDY

SEQ ID NO: 245 - protein, 11 aa, synthetic construct
  REGION 1..11: note = CDR-L1
RASQGISNYLA

SEQ ID NO: 246 - protein, 7 aa, synthetic construct
  REGION 1..7: note = CDR-L2
SASNLQS

SEQ ID NO: 247 - protein, 7 aa, synthetic construct
  REGION 1..7: note = CDR-H1
GYSLITH

SEQ ID NO: 248 - protein, 6 aa, synthetic construct
  REGION 1..6: note = CDR-H2
NPSDGV

(19) United States
(12) Patent Application Publication
(10) Pub. No.: US 2024/0344083 A1
(43) Pub. Date: Oct. 17, 2024

(54) USE OF CD4-TARGETED VIRAL VECTORS

(71) Applicant: Sana Biotechnology, Inc., Seattle, WA (US)

(72) Inventors: Christie CIARLO, Seattle, WA (US); Christopher BANDORO, Brisbane, CA (US); Kutlu ELPEK, Arlington, MA (US); Hanane ENNAJDAOUI, Cambridge, MA (US); Aaron FOSTER, Bainbridge Island, WA (US); Zach FRYE, Cambridge, MA (US); Adam JOHNSON, Shoreline, WA (US); Lauren Pepper MACKENZIE, Belmont, MA (US); Albert Ruzo MATIAS, Seattle, WA (US); Jagesh Vijaykumar SHAH, Lexington, MA (US)

(73) Assignee: Sana Biotechnology, Inc., Seattle, WA (US)

(21) Appl. No.: 18/294,520

(22) PCT Filed: Aug. 3, 2022
(86) PCT No.: PCT/US2022/074484

§ 371 Date: Feb. 1, 2024

Related U.S. Application Data
(60) Provisional application No. 63/392,833, filed on Jul. 27, 2022.
(60) Provisional application No. 63/341,784, filed on May 13, 2022.
(60) Provisional application No. 63/298,213, filed on Jan. 10, 2022.
(60) Provisional application No. 63/259,717, filed on Aug. 4, 2021.

(51) Int. Cl. C12N 15/86; A61K 39/00; A61K 48/00; C07K 14/005; C07K 14/705; C07K 14/725; C07K 16/28

(52) U.S. Cl. CPC C12N 15/86; A61K 39/4611; A61K 39/4631; A61K 39/464412; A61K 48/005; C07K 14/005; C07K 14/7051; C07K 14/70578; C07K 16/2803; C07K 16/2812; C07K 2317/622; C07K 2319/02; C12N 2740/15043; C12N 2760/18222; C12N 2760/20222

(57) ABSTRACT

Provided herein are methods of transducing resting or non-activated T cells using CD4-targeted viral vectors.

### CROSS-REFERENCE TO RELATED APPLICATIONS

This application claims priority to U.S. provisional application nos. 63/259,717, filed Aug. 4, 2021, entitled “USE OF CD4-TARGETED VIRAL VECTORS,” 63/298,213, filed Jan. 10, 2022, entitled “USE OF CD4-TARGETED VIRAL VECTORS,” 63/341,784 filed May 13, 2022, entitled “USE OF CD4-TARGETED VIRAL VECTORS,” and 63/392,833, filed Jul. 27, 2022, entitled, “USE OF CD4-TARGETED VIRAL VECTORS,” the contents of each of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 186152005940SeqList.XML, created Jul. 29, 2022, which is 342,794 bytes in size. The information in the electronic format of the Sequence Listing is incorporated by reference in its entirety.

### FIELD

The present disclosure relates to methods of transducing resting or non-activated T cells using CD4-targeted viral vectors.

### BACKGROUND

Viral vectors, including lentiviral vectors, are commonly used for delivery of exogenous agents to cells. However, transduction of the viral vectors to certain target cells can be challenging. Improved viral vectors, including lentiviral vectors, for use in methods for targeting desired cells and improving delivery are needed. The provided disclosure addresses this need.

### SUMMARY

This application is based on, inter alia, the surprising finding that resting or non-activated T cells could be efficiently transduced, both in vitro and in vivo using CD4-targeted viral vectors.

Provided herein is a method of transducing T cells, the method comprising contacting a non-activated T cell with a lentiviral vector comprising a CD4 binding agent, wherein the lentiviral vector transduces the non-activated T cell. In some embodiments, the T cell is a CD4+ T cells. In some embodiments, the non-activated T cell is surface negative for one or more T cell activation markers selected from the group consisting of CD25, CD44 and CD69.

In some embodiments, the non-activated T cell has not been treated with an anti-CD3 antibody (e.g., OKT3). In some embodiments, the non-activated T cell has not been treated with an anti-CD28 antibody (e.g., CD28.2). In some embodiments, the non-activated T cell has not been treated with an anti-CD3 antibody (e.g., OKT3) or with an anti-CD28 antibody (e.g., CD28.2). In some embodiments, the non-activated T cell has not been treated with a bead coupled to an anti-CD3 antibody (e.g. OKT3) and an anti-CD28 antibody (e.g. CD28.2), optionally wherein the bead is a superparamagnetic bead. In some embodiments, the bead is a superparamagnetic bead. In some embodiments, the non-activated T cell has not been treated with a T cell activating cytokine (e.g., recombinant IL-2, IL-7, IL-15, IL-21, or combinations thereof), optionally wherein the T cell activating cytokine is a human cytokine. In some embodiments, the T cell activating cytokine is a human cytokine. In some embodiments, the non-activated T cell has not been treated with a soluble T cell costimulatory molecule (e.g. anti-CD28 antibody or soluble CD80, soluble CD86, soluble CD137L or soluble ICOS-L). In some of any provided embodiments, the lentiviral vector comprises a transgene encoding an engineered receptor that binds to or recognizes a protein or antigen expressed by or on cells associated with a disease or condition (e.g. tumor cells).

In some embodiments, the engineered receptor is an engineered T cell receptor (eTCR). In some embodiments, the engineered receptor is a chimeric antigen receptor (CAR). In some embodiments, the CAR comprises an antigen-binding domain, a transmembrane domain, and an intracellular signaling domain comprising intracellular components of a CD3zeta signaling domain and a costimulatory signaling domain. In some embodiments, the costimulatory signaling domain is a CD28 costimulatory domain. In some embodiments, the CD28 costimulatory signaling domain comprises the amino acid sequence set forth in SEQ ID NO:60. In some embodiments, the costimulatory signaling domain is a 4-1BB signaling domain. In some embodiments, the 4-1BB signaling domain comprises the amino acid sequence set forth in SEQ ID NO:59. In some embodiments, the CD3zeta signaling domain comprises the sequence set forth in SEQ ID NO:61 or SEQ ID NO:62. In some embodiments, the CD3zeta signaling domain comprises the sequence set forth in SEQ ID NO:61. In some embodiments, the CD3zeta signaling domain comprises the sequence set forth in SEQ ID NO:62. In some embodiments, the transmembrane domain comprises the sequence set forth in any one of SEQ ID NOS:56, 57, and 58. In some embodiments, the transmembrane domain comprises the sequence set forth in SEQ ID NO:56. In some embodiments, the transmembrane domain comprises the sequence set forth in SEQ ID NO:57. In some embodiments, the transmembrane domain comprises the sequence set forth in SEQ ID NO:58. In some embodiments, the CAR comprises a hinge domain. In some embodiments, the hinge domain comprises the sequence set forth in any one of SEQ ID NOS:50, 51, 52, 53, 54, 55, and 142. In some embodiments, the hinge domain comprises the sequence set forth in SEQ ID NO:51. In some embodiments, the hinge domain comprises the sequence set forth in SEQ ID NO:52. In some embodiments, the hinge domain comprises the sequence set forth in SEQ ID NO:53. In some embodiments, the hinge domain comprises the sequence set forth in SEQ ID NO:54. In some embodiments, the hinge domain comprises the sequence set forth in SEQ ID NO:55. In some embodiments, the hinge domain comprises the sequence set forth in SEQ ID NO:142.

In some embodiments, the antigen binding domain binds to an antigen selected from the group consisting of CD19, CD20, CD22, and BCMA.

In some embodiments, the antigen binding domain binds to CD19. In some embodiments, the antigen binding domain comprises a CDR-H1, a CDRH-2, and a CDR-H3 comprising the amino acid sequence set forth in SEQ ID NO: 70, 71, and 72, respectively, and a CDR-L1, a CDR-L2, and a CDR-L3 comprising the amino acid sequence set forth in SEQ ID NO: 65, 66, and 67, respectively. In some embodiments, the antigen binding domain comprises a VH region comprising the amino acid sequence set forth in SEQ ID NO:69, and a VL region comprising the amino acid sequence set forth in SEQ ID NO:64. In some embodiments, the antigen binding domain comprises the amino acid sequence set forth in SEQ ID NO:63 or 73. In some embodiments, the antigen binding domain comprises the amino acid sequence set forth in SEQ ID NO:63. In some embodiments, the antigen binding domain comprises the amino acid sequence set forth in SEQ ID NO:73. In some embodiments, the CAR comprises the amino acid sequence set forth in SEQ ID NO:75, 77, 79, or 81. In some embodiments, the CAR comprises the amino acid sequence set forth in SEQ ID NO:75. In some embodiments, the CAR comprises the amino acid sequence set forth in SEQ ID NO:77. In some embodiments, the CAR comprises the amino acid sequence set forth in SEQ ID NO:79. In some embodiments, the CAR comprises the amino acid sequence set forth in SEQ ID NO:81. In some embodiments, the CAR comprises an amino acid sequence encoded by the polynucleotide sequence set forth in SEQ ID NO:74, 76, 78, or 80. In some embodiments, the CAR comprises an amino acid sequence encoded by the polynucleotide sequence set forth in SEQ ID NO:74. In some embodiments, the CAR comprises an amino acid sequence encoded by the polynucleotide sequence set forth in SEQ ID NO:76. In some embodiments, the CAR comprises an amino acid sequence encoded by the polynucleotide sequence set forth in SEQ ID NO:78. In some embodiments, the CAR comprises an amino acid sequence encoded by the polynucleotide sequence set forth in SEQ ID NO:80.

In some embodiments, the antigen binding domain binds to CD20. In some embodiments, wherein the antigen binding domain comprises a CDR-H1, a CDRH-2, and a CDR-H3 comprising the amino acid sequence set forth in SEQ ID NO: 88, 89, and 144, respectively, and a CDR-L1, a CDR-L2, and a CDR-L3 comprising the amino acid sequence set forth in SEQ ID NO: 84, 85, and 86, respectively. a VH region comprising the amino acid sequence set forth in SEQ ID NO:87, and a VL region comprising the amino acid sequence set forth in SEQ ID NO:83. In some embodiments, wherein the antigen binding domain comprises the amino acid sequence set forth in SEQ ID NO:82.

In some embodiments, the antigen binding domain binds to CD22. In some embodiments, the antigen binding domain comprises a CDR-H1, a CDRH-2, and a CDR-H3 comprising the amino acid sequence set forth in SEQ ID NO: 92, 93, and 94, respectively, and a CDR-L1, a CDR-L2, and a CDR-L3 comprising the amino acid sequence set forth in SEQ ID NO: 96, 97, and 98, respectively. In some embodiments, the antigen binding domain comprises a CDR-H1, a CDRH-2, and a CDR-H3 comprising the amino acid sequence set forth in SEQ ID NO: 101, 102, and 103, respectively, and a CDR-L1, a CDR-L2, and a CDR-L3 comprising the amino acid sequence set forth in SEQ ID NO: 105, 106, and 107, respectively. In some embodiments, the antigen binding domain comprises a VH region comprising the amino acid sequence set forth in SEQ ID NO:91, and a VL region comprising the amino acid sequence set forth in SEQ ID NO:95. In some embodiments, the antigen binding domain comprises a VH region comprising the amino acid sequence set forth in SEQ ID NO:100, and a VL region comprising the amino acid sequence set forth in SEQ ID NO:104. In some embodiments, the antigen binding domain comprises the amino acid sequence set forth in SEQ ID NO:90 or 99. In some embodiments, the antigen binding domain comprises the amino acid sequence set forth in SEQ ID NO:90. In some embodiments, the antigen binding domain comprises the amino acid sequence set forth in SEQ ID NO:99.

In some embodiments, the antigen binding domain binds to BCMA. In some embodiments, the antigen binding domain comprises a CDR-H1, a CDRH-2, and a CDR-H3 comprising the amino acid sequence set forth in SEQ ID NO: 114, 115, and 116, respectively, and a CDR-L1, a CDR-L2, and a CDR-L3 comprising the amino acid sequence set forth in SEQ ID NO: 110, 111, and 112, respectively. In some embodiments, the antigen binding domain comprises a CDR-H1, a CDRH-2, and a CDR-H3 comprising the amino acid sequence set forth in SEQ ID NO: 123, 124, and 125, respectively, and a CDR-L1, a CDR-L2, and a CDR-L3 comprising the amino acid sequence set forth in SEQ ID NO: 119, 120, and 121, respectively. In some embodiments, the antigen binding domain comprises a CDR-H1, a CDRH-2, and a CDR-H3 comprising the amino acid sequence set forth in SEQ ID NO: 127, 128, and 129, respectively. In some embodiments, the antigen binding domain comprises a CDR-H1, a CDRH-2, and a CDR-H3 comprising the amino acid sequence set forth in SEQ ID NO: 136, 137, and 138, respectively, and a CDR-L1, a CDR-L2, and a CDR-L3 comprising the amino acid sequence set forth in SEQ ID NO:132, 133, and 134, respectively. In some embodiments, the antigen binding domain comprises a VH region comprising the amino acid sequence set forth in SEQ ID NO:113, and a VL region comprising the amino acid sequence set forth in SEQ ID NO:109. In some embodiments, the antigen binding domain comprises a VH region comprising the amino acid sequence set forth in SEQ ID NO:122, and a VL region comprising the amino acid sequence set forth in SEQ ID NO:118. In some embodiments, the antigen binding domain comprises a VH region comprising the amino acid sequence set forth in SEQ ID NO:135, and a VL region comprising the amino acid sequence set forth in SEQ ID NO:131. In some embodiments, the antigen binding domain comprises a VH region comprising the amino acid sequence set forth in SEQ ID NO:126. In some embodiments, the antigen binding domain comprises the amino acid sequence set forth in SEQ ID NO:108, 117, or 130. In some embodiments, the antigen binding domain comprises the amino acid sequence set forth in SEQ ID NO:108. In some embodiments, the antigen binding domain comprises the amino acid sequence set forth in SEQ ID NO:117. In some embodiments, the antigen binding domain comprises the amino acid sequence set forth in SEQ ID NO:130. In some embodiments, the CAR comprises the amino acid sequence set forth in SEQ ID NO:140. In some embodiments, the CAR comprises an amino acid sequence encoded by the polynucleotide sequence set forth in SEQ ID NO:139.

In some embodiments, the CAR comprises: (i) an antigen binding domain comprising the VL region set forth in SEQ ID NO:64, a linker comprising the amino acid sequence set forth in SEQ ID NO:68, and the VH region set forth in SEQ ID NO:69; and/or the scFv set forth in SEQ ID NO:63; (ii) a hinge comprising the amino acid sequence set forth in SEQ ID NO:50; (iii) a transmembrane domain comprising the amino acid sequence set forth in SEQ ID NO:56; (iv) a 4-1BB signaling domain comprises the amino acid sequence set forth in SEQ ID NO:59; and (v) a CD3zeta signaling domain comprising the amino acid sequence set forth in SEQ ID NO:61. In some embodiments, the CAR comprises the amino acid sequence set forth in SEQ ID NO:75. In some embodiments, the CAR is encoded by the nucleotide sequence set forth in SEQ ID NO:74.

In some embodiments, the non-activated T cell is a human T cell.

In some embodiments, the non-activated T cell is in a subject. In some embodiments, the non-activated T cell is in vitro. In some embodiments, the non-activated T cell is ex vivo from a subject. In some embodiments of the provided methods, prior to the contacting, the subject has not been administered a T cell activating treatment.

In some embodiments, any of the methods provided herein are carried out in vivo. In some embodiments, any of the methods provided herein are not ex vivo or are not in vitro.

In some of any embodiments of the provided methods, the subject has a disease or condition, such as a cancer. In some embodiments, the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR). In some embodiments, the engineered receptor is a chimeric antigen receptor (CAR) or an engineered T cell receptor (TCR). In some embodiments, the engineered receptor is a chimeric antigen receptor (CAR). In some embodiments, the engineered receptor is an engineered T cell receptor (TCR).

In some of any of the provided methods, the method further comprises editing the T cell to inactivate one or more of B2M, CIITA, TRAC, and TRB genes. In some embodiments, the T cell is edited to inactivate B2M, CIITA, and TRAC genes. In some of any of the provided methods, the method further comprises inserting a gene encoding CD47 at a defined locus. In some embodiments, the defined locus is selected from the group consisting of a B2M locus, a CIITA locus, a TRAC locus, a TRB locus, or a safe harbor locus. In some embodiments, the safe harbor locus is selected from the group consisting of an AAVS locus, a CCR5 locus, and a ROSA26 locus.

Also provided herein is a transduced T cell produced by the method of any of the provided methods. In some embodiment, the T cell is inactivated at both alleles of the one or more genes. Also provided herein is a composition comprising a provided transduced T cell. In some embodiments, the composition is a pharmaceutical composition.

Provided herein is a method of transducing a population of T cells, the method comprising: contacting a population of non-activated T cells with a composition comprising lentiviral vectors comprising a CD4 binding agent, wherein the population of non-activated T cells is transduced at an efficiency of at least 1%. In some embodiments, the population of non-activated T cells is transduced at an efficiency of at least 5%. In some embodiments, the population of non-activated T cells is transduced at an efficiency of at least 10%. In some embodiments, the population of non-activated T cells is transduced at an efficiency of at least 15%. In some embodiments, the population of non-activated T cells is transduced at an efficiency of at least 20%. In some embodiments, the population of non-activated T cells is transduced at an efficiency of at least 25%. In some embodiments, the population of non-activated T cells is transduced at an efficiency of at least 30%. In some embodiments, the population of non-activated T cells is transduced at an efficiency of at least 35%. In some embodiments, the population of non-activated T cells is transduced at an efficiency of at least 10%, at least 15%, at least 20%, at least 25%, at least 30%, at least 35%, at least 40%, at least 45%, at least 50%, at least 55%, at least 60%, at least 65%, at least 70%, or at least 75%.

In some embodiments, at least 75% of the T cells in the population of non-activated T cells are surface negative for one or more T cell activation markers selected from the group consisting of CD25, CD44 and CD69 (e.g. at least 80%, at least 85%, at least 90%, at least 95% of the T cells in the population are surface negative for the T cell activation marker). In some embodiments, the population of non-activated T cells comprises CD4+ T cells (e.g. at least 10%, at least 20%, at least 30%, at least 40%, at least 50%, at least 60%, at least 70%, at least 80%, at least 90% of the population of non-activated T cells are CD4+ T cells). In some embodiments, at least 75% of the CD4+ T cells are surface negative for one or more T cell activation markers selected from the group consisting of CD25, CD44 and CD69 (e.g. at least 80%, at least 85%, at least 90%, at least 95% of the CD4+ T cells in the population are surface negative for the T cell activation marker). In some embodiments, the one or more cell activation markers is CD25. In some embodiments, the one or more T cell activation markers is CD44. In some embodiments, the one or more T cell activation markers is CD69. In some embodiments, the CD4+ T cells in the population of non-activated T cells are transduced at an efficiency of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75%.

In some embodiments, the population of non-activated T cells has not been treated with an anti-CD3 antibody (e.g., OKT3). In some embodiments, the population of non-activated T cell has not been treated with an anti-CD28 antibody (e.g., CD28.2). In some embodiments, the population of non-activated T cells has not been treated with an anti-CD3 antibody (e.g., OKT3) or with an anti-CD28 antibody (e.g., CD28.2). In some embodiments, the population of non-activated T cells has not been treated with a bead coupled to an anti-CD3 antibody (e.g. OKT3) and an anti-CD28 antibody (e.g. CD28.2), optionally wherein the bead is a superparamagnetic bead. In some embodiments, the population of non-activated T cells has not been treated with a bead coupled to an anti-CD3 antibody (e.g. OKT3) and an anti-CD28 antibody (e.g. CD28.2). In some embodiments, the bead is a superparamagnetic bead. In some embodiments, the population of non-activated T cell has not been treated with a T cell activating cytokine (e.g., recombinant IL-2, IL-7, IL-15, IL-21, or combinations thereof), optionally wherein the T cell activating cytokine is a human cytokine. In some embodiments, the population of non-activated T cell has not been treated with a T cell activating cytokine (e.g., recombinant IL-2, IL-7, IL-15, IL-21, or combinations thereof). In some embodiments, the T cell activating cytokine is a human cytokine. In some embodiments, the population of non-activated T cells has not been treated with a soluble T cell costimulatory molecule (e.g. anti-CD28 antibody or soluble CD80, soluble CD86, soluble CD137L or soluble ICOS-L).

In some embodiments, the population of non-activated T cells are human cells.

In some embodiments, the population of non-activated T cells is in a subject. In some embodiments, prior to the contacting, the subject had not been administered a T cell activating treatment. In some embodiments, the population of non-activated T cells is in vitro. In some embodiments, the population of non-activated T cells is ex vivo from a subject. In some embodiments, the population of non-activated T cells comprise peripheral blood mononuclear cells (PBMCs) or a subset thereof comprising CD4+ T cells. In some embodiments, the population of non-activated cells is an enriched population of T cells selected from a biological sample from a subject, optionally wherein the T cells are selected for T cells surface positive for a T cell marker (e.g., CD3 or CD4). In some embodiments, the population of non-activated cells is an enriched population of T cells selected from a biological sample from a subject. In some embodiments, the T cells are selected for T cells surface positive for a T cell marker (e.g., CD3 or CD4). In some embodiments, the T cell marker is CD3. In some embodiments, the T cell marker is CD4. In some embodiments, the biological sample is a whole blood sample, apheresis sample or leukapheresis sample. In some embodiments, the biological sample is a whole blood sample. In some embodiments, the biological sample is an apheresis sample. In some embodiments, the biological sample is a leukapheresis sample.

In some embodiments, the subject has a disease or condition. In some embodiments, the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR). In some embodiments, the lentiviral vector comprises a transgene encoding an engineered receptor that binds to or recognizes a protein or antigen expressed by or on cells associated with the disease or condition (e.g. tumor cells). In some embodiments, the engineered receptor is a chimeric antigen receptor (CAR) or an engineered T cell receptor (TCR). In some embodiments, the engineered receptor is a chimeric antigen receptor (CAR). In some embodiments, the engineered receptor is an engineered T cell receptor (TCR).

In some of any of the provided methods, the method further comprises editing the T cell or population of T cells to inactivate one or more of B2M, CIITA, TRAC, and TRB genes. In some of any of the provided methods, the population of T cells are edited to inactivate B2M, CIITA, and TRAC genes. In some embodiments, T cells of the population of T cells is edited to inactivate B2M, CIITA, and TRB genes. In some embodiments, the method further comprises inserting a gene encoding CD47 at a defined locus. In some embodiments, the defined locus is selected from the group consisting of a B2M locus, a CIITA locus, a TRAC locus, a TRB locus, or a safe harbor locus. In some embodiments, the safe harbor locus is selected from the group consisting of an AAVS1 locus, a CCR5 locus, and a ROSA26 locus.

In some of any of the provided methods, the method further comprises expanding the population of transduced T cells. In some embodiments, the expanding comprises incubation of the transduced cells with one or more T cell activating cytokine (e.g., recombinant IL-2, IL-7, IL-15, IL-21, or combinations thereof), optionally wherein the T cell activating cytokine is a human cytokine. In some embodiments, the expanding comprises incubation of the transduced cells with one or more T cell activating cytokine (e.g., recombinant IL-2, IL-7, IL-15, IL-21, or combinations thereof). In some embodiments, the T cell activating cytokine is a human cytokine. In some of any of the provided methods, the method further comprises incubating the transduced T cells with one or more T cell activating cytokine (e.g., recombinant IL-2, IL-7, IL-15, IL-21, or combinations thereof), optionally wherein the T cell activating cytokine is a human cytokine. In some of any of the provided methods, the method further comprises incubating the transduced T cells with one or more T cell activating cytokine (e.g., recombinant IL-2, IL-7, IL-15, IL-21, or combinations thereof). In some embodiments, the T cell activating cytokine is a human cytokine.

Also provided herein is a population of transduced T cells produced by any of the provided methods. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of the cells of the population of non-activated cells are inactivated at the one or more genes. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of the non-activated CD4+ T cells in the population are transduced and are inactivated at the one or more genes. In some embodiments, at least 1% of the non-activated CD4+ T cells in the population are transduced and are inactivated at the one or more genes. In some embodiments, at least 5% of the non-activated CD4+ T cells in the population are transduced and are inactivated at the one or more genes. In some embodiments, at least 10% of the non-activated CD4+ T cells in the population are transduced and are inactivated at the one or more genes. In some embodiments, at least 11% of the non-activated CD4+ T cells in the population are transduced and are inactivated at the one or more genes. In some embodiments, at least 15% of the non-activated CD4+ T cells in the population are transduced and are inactivated at the one or more genes. In some embodiments, at least 20% of the non-activated CD4+ T cells in the population are transduced and are inactivated at the one or more genes. In some embodiments, at least 25% of the non-activated CD4+ T cells in the population are transduced and are inactivated at the one or more genes. In some embodiments, at least 30% of the non-activated CD4+ T cells in the population are transduced and are inactivated at the one or more genes. In some embodiments, at least 35% of the non-activated CD4+ T cells in the population are transduced and are inactivated at the one or more genes. In some embodiments, cells of the population are inactivated at both alleles of the one or more genes.

Also provided herein is a composition comprising the population of transduced T cells, optionally wherein the composition is a pharmaceutical composition. Also provided herein is a composition comprising the population of transduced T cells. In some embodiments, the composition is a pharmaceutical composition. Also provided herein is a pharmaceutical composition comprising the population of transduced T cells. Also provided herein is a method of treating a subject having a disease or condition, the method comprising administering to the subject any of the provided compositions comprising the population of transduced T cells. In some embodiments, the composition is not administered subcutaneously (SC). In some embodiments, the composition is not administered intramuscularly (IM). In some embodiments, the composition is administered intravenously (IV).

In some of any of the provided compositions, the composition further comprises a cyropreservant. In some embodiments, the cyropreservant is DMSO.

Provided herein is a method of in vivo transduction of T cells, the method comprising: administering to a subject a composition comprising lentiviral vectors comprising a CD4 binding agent, wherein the lentiviral vectors transduce T cells within the subject, and wherein the subject is not administered a T cell activating treatment (e.g. before, after or concurrently) with administration of the composition. Also provided herein is a method of in vivo transduction of T cells, the method comprising: administering to a subject any of the provided compositions, wherein the lentiviral vectors transduce T cells within the subject, and wherein the subject is not administered a T cell activating treatment (e.g. before, after or concurrently) with administration of the composition. In some embodiments, the subject has a disease or condition.

Also provided herein is a method of treating a subject having a disease or condition, the method comprising: administering to the subject a composition comprising lentiviral vectors comprising a CD4 binding agent, and wherein the subject is not administered a T cell activating treatment (e.g. before, after or concurrently) with administration of the composition. Also provided herein is a method of treating a subject having a disease or condition, the method comprising administering to the subject any of the provided compositions, wherein the subject is not administered a T cell activating treatment (e.g. before, after or concurrently) with administration of the composition. In some embodiments, the disease or condition is a cancer

Also provided herein is a method for expanding T cells capable of recognizing and killing tumor cells in a subject in need thereof, the method comprising: administering to the subject a composition comprising lentiviral vectors comprising a CD4 binding agent, and wherein the subject is not administered a T cell activating treatment (e.g. before, after, or concurrently) with administration of the composition. Also provided herein is a method for expanding T cells capable of recognizing and killing tumor cells in a subject in need thereof, the method comprising: administering to the subject a composition provided herein, and wherein the subject is not administered a T cell activating treatment (e.g. before, after, or concurrently) with administration of the composition. Also provided herein is a method for expanding T cells capable of recognizing and killing tumor cells in a subject in need thereof, the method comprising administering to the subject a composition provided herein. In some embodiments, the composition is not administered subcutaneously (SC). In some embodiments, the composition is not administered intramuscularly (IM). In some embodiments, the composition is administered intravenously (IV).

Also provided herein is use of a composition comprising lentiviral vectors comprising a CD4 binding agent for treating a subject having a disease or condition, optionally a cancer. Also provided herein is use of a composition provided herein for formulation of a medicament for treating a subject having a disease or condition, optionally a cancer. Also provided herein is use of a composition comprising lentiviral vectors comprising a CD4 binding agent for treating a subject having a disease or condition. Also provided herein is use of a composition provided herein for formulation of a medicament for treating a subject having a disease or condition. In some embodiments, the disease or condition is a cancer.

Also provided herein is a composition comprising lentiviral vectors comprising a CD4 binding agent for use in treating a subject having a disease or condition, optionally a cancer. Also provided herein is a composition provided herein for use in treating a subject having a disease or condition, optionally a cancer. Also provided herein is a composition comprising lentiviral vectors comprising a CD4 binding agent for use in treating a subject having a disease or condition. Also provided herein is a composition of any provided herein for use in treating a subject having a disease or condition. In some embodiments, the disease or condition is a cancer.

Also provided herein is use of a composition comprising lentiviral vectors comprising a CD4 binding agent for formulation of a medicament for expanding T cells capable of recognizing and killing tumor cells in a subject in need thereof. Also provided herein is use of a composition provided herein for formulation of a medicament for expanding T cells capable of recognizing and killing tumor cells in a subject in need thereof.

Provided herein is a composition comprising lentiviral vectors comprising a CD4 binding agent for use in expanding T cells capable of recognizing and killing tumor cells in a subject in need thereof. Also provided herein is a composition of provided herein for use in expanding T cells capable of recognizing and killing tumor cells in a subject in need thereof.

In some of any of the provided embodiments, the use or the composition for use provided herein is for use in a subject that is not administered or to be administered a T cell activating treatment (e.g. before, after or concurrently) with administration of the composition.

In some of any of the provided methods, uses or compositions for use provided herein, the disease or condition is a cancer. In some embodiments, the lentiviral vector comprises a transgene encoding an engineered receptor that binds to or recognizes a protein or antigen expressed by or on cells associated with the disease or condition (e.g. tumor cells). In some embodiments, the lentiviral vector comprises a transgene encoding an engineered receptor that binds to or recognizes a protein expressed on the tumor cells. In some embodiments, the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR). In some embodiments, the lentiviral vector comprises a transgene encoding an engineered receptor that binds to or recognizes a protein expressed on the tumor cells, optionally wherein the engineered receptor is a chimeric antigen receptor (CAR) or an engineered T cell receptor (TCR). In some embodiments, the engineered receptor is a chimeric antigen receptor (CAR) or an engineered T cell receptor (TCR)

In some embodiments, the T cell activating treatment comprises administration of an anti-CD3 antibody (e.g., OKT3). In some embodiments, the T cell activating treatment comprises administration of a soluble T cell costimulatory molecule (e.g., anti-CD28 antibody, or a recombinant CD80, CD86, CD137L, ICOS-L). In some embodiments, the T cell activating treatment comprises administration of a T cell activating cytokine (e.g., recombinant IL-2, IL-7, IL-15, IL-21). In some embodiments, the T cell activating cytokine is a human cytokine. In some embodiments, the T cell activating treatment comprises administration of a T cell activating cytokine (e.g., recombinant IL-2, IL-7, IL-15, IL-21), optionally wherein the T cell activating cytokine is a human cytokine. In some of any embodiments, the T cell activating treatment comprises administration of recombinant IL-7, optionally human IL-7. In some of any embodiments, the T cell activating treatment comprises administration of recombinant IL-7. In some embodiments, the T cell activating treatment comprises administration of recombinant human IL-7. In some of any embodiments, the T cell activating treatment comprises administration of a lymphodepleting therapy, optionally administration of cyclophosphamide and/or fludarabine. In some of any embodiments, the T cell activating treatment comprises administration of a lymphodepleting therapy. In some embodiments, the T cell activating treatment comprises administration of cyclophosphamide and/or fludarabine. In some embodiments, the T cell activating treatment comprises administration of cyclophosphamide or fludarabine. In some embodiments, the T cell activating treatment comprises administration of cyclophosphamide. In some embodiments, the T cell activating treatment comprises administration of fludarabine. In some embodiments, the T cell activating treatment comprises administration of cyclophosphamide and fludarabine.

In some of any of the provided embodiments, the subject is not administered a T cell activating treatment concurrently with the lentiviral vector. In some of any of the provided embodiments, the subject is not administered a T cell activating treatment within 1 month before the contacting with the lentiviral vector or before the administration of the composition comprising the lentiviral vectors. In some of any of the provided embodiments, the subject is not administered a T cell activating treatment within or at or about 1 week, 2 weeks, 3 weeks or 4 weeks, optionally at or about 1, 2, 3, 4, 5, 6 or 7 days, before the contacting with the lentiviral vector or before the administration of the composition comprising the lentiviral vectors. In some of any of the provided embodiments, the subject is not administered a T cell activating treatment at or about 1, 2, 3, 4, 5, 6 or 7 days, before the contacting with the lentiviral vector or before the administration of the composition comprising the lentiviral vectors. In some of any of the provided embodiments, the subject is not administered a T cell activating treatment within 1 month after the contacting with the lentiviral vector or after the administration of the composition comprising the lentiviral vectors. In some of any of the provided embodiments, the subject is not administered a T cell activating treatment within or at or about 1 week, 2 weeks, 3 weeks or 4 weeks, optionally at or about 1, 2, 3, 4, 5, 6 or 7 days, after the contacting with the lentiviral vector or after the administration of the composition comprising the lentiviral vectors. In some of any of the provided embodiments, the subject is not administered a T cell activating treatment at or about 1, 2, 3, 4, 5, 6 or 7 days, after the contacting with the lentiviral vector or after the administration of the composition comprising the lentiviral vectors.

In some of any of the provided embodiments, the lentiviral vector does not comprise or encode a T cell activating agent. In some of any of the provided embodiments, the lentiviral vector does not comprise or encode a membrane-bound T cell activating agent. In some of any of the provided embodiments, the lentiviral vector does not comprise or encode a T cell activating agent displayed on the surface. In some of any of the provided embodiments, the lentiviral vector does not comprise a T cell activating agent displayed on the surface, such as where the T cell activating agent is selected from the group consisting of a CD3 antibody (e.g. anti-CD3 scFv); a T cell activating cytokine (e.g. IL-2, IL-7, IL-15 or IL-21); or a T cell costimulatory molecule (e.g. anti-CD28 antibody, CD80, CD86, CD137L or ICOS-L). In some embodiments, the T cell activating agent is selected from the group consisting of a CD3 antibody (e.g. anti-CD3 scFv); a T cell activating cytokine (e.g. IL-2, IL-7, IL-15 or IL-21); and a T cell costimulatory molecule (e.g. anti-CD28 antibody, CD80, CD86, CD137L or ICOS-L). In some embodiments, the T cell activating agent is a polypeptide capable of binding CD3 and/or CD28. In some embodiments, the T cell activating agent is a polypeptide capable of binding CD3. In some embodiments, the T cell activating agent is a polypeptide capable of binding CD28. In some embodiments, the T cell activating agent is a lymphoproliferative element. In some embodiments, the T cell activating agent is a cytokine or a cytokine receptor or a signaling domain thereof that activates a STAT3 pathway, a STAT4 pathway, and/or a Jak/STAT5 pathway. In some embodiments, the T cell activating agent is a T cell survival motif. In some embodiments, the T cell survival motif is an IL-7 receptor, an IL-15 receptor, or CD28, or a functional portion thereof. In some embodiments, the T cell activating agent is a microRNA (miRNA) or a short hairpin RNA (shRNA). In some embodiments, the miRNA or the shRNA stimulates the STAT5 pathway. In some embodiments, the miRNA or the shRNA inhibits the SOCS pathway. In some embodiments, the miRNA or the shRNA stimulates the STAT5 pathway and inhibits the SOCS pathway.

In some embodiments, the lentiviral vector does not comprise or encode an inhibitory RNA molecule. In some embodiments, the inhibitory RNA molecule targets an mRNA transcribed from a gene expressed by T cells. In some embodiments, the inhibitory RNA molecule targets a gene encoding a component of a T cell receptor (TCR). In some embodiments, the gene is PD-1, CTLA4, TCRα, TCRβ, CD3δ, SOCS1, SMAD2, a miR-155 target, IFNγ, TRAIL2, and/or ABCG1.

In some embodiments, the lentiviral vector comprises or encodes an inhibitory RNA molecule. In some embodiments, the inhibitory RNA molecule targets an mRNA transcribed from a gene expressed by T cells. In some embodiments, the inhibitory RNA molecule targets a gene encoding a component of a T cell receptor (TCR). In some embodiments, the gene is PD-1, CTLA4, TCRα, TCRβ, CD3δ, SOCS1, SMAD2, a miR-155 target, IFNγ, TRAIL2, and/or ABCG1.

In some of any of the provided embodiments, the CD4 binding agent is an anti-CD4 antibody or an antigen-binding fragment. In some of any of the provided embodiments, the anti-CD4 antibody or antigen-binding fragment is mouse, rabbit, human, or humanized. In some embodiments, the antigen-binding fragment is a single chain variable fragment (scFv). In some embodiments, the antigen-binding fragment is an anti-CD4 scFv.

In some embodiments, the anti-CD4 scFv comprises a CDR-H1, a CDR-H2, and a CDR-H3 comprising the amino acid sequence set forth in SEQ ID NO: 149, 150, and 151, respectively. In some embodiments, the anti-CD4 scFv comprises a CDR-L1, a CDR-L2, and a CDR-L3 comprising the amino acid sequence set forth in SEQ ID NO: 152, 153, and 154, respectively. In some embodiments, the anti-CD4 scFv comprises a CDR-H1, a CDR-H2, and a CDR-H3 comprising the amino acid sequence set forth in SEQ ID NO: 149, 150, and 151, respectively; and a CDR-L1, a CDR-L2, and a CDR-L3 comprising the amino acid sequence set forth in SEQ ID NO: 152, 153, and 154, respectively. In some embodiments, the anti-CD4 scFv comprises a CDR-H1, a CDR-H2, and a CDR-H3 comprising the amino acid sequence set forth in SEQ ID NO: 207, 208, and 209, respectively. In some embodiments, the anti-CD4 scFv comprises a CDR-L1, a CDR-L2, and a CDR-L3 comprising the amino acid sequence set forth in SEQ ID NO: 210, 211, and 154, respectively. In some embodiments, the anti-CD4 scFv comprises a CDR-H1, a CDR-H2, and a CDR-H3 comprising the amino acid sequence set forth in SEQ ID NO: 207, 208, and 209, respectively; and a CDR-L1, a CDR-L2, and a CDR-L3 comprising the amino acid sequence set forth in SEQ ID NO: 210, 211, and 154, respectively. In some embodiments, the anti-CD4 scFv comprises a CDR-H1, a CDR-H2, and a CDR-H3 comprising the amino acid sequence set forth in SEQ ID NO: 212, 213, and 209, respectively. In some embodiments, the anti-CD4 scFv comprises a CDR-L1, a CDR-L2, and a CDR-L3 comprising the amino acid sequence set forth in SEQ ID NO: 210, 211, and 154, respectively. In some embodiments, the anti-CD4 scFv comprises a CDR-H1, a CDR-H2, and a CDR-H3 comprising the amino acid sequence set forth in SEQ ID NO: 212, 213, and 209, respectively; and a CDR-L1, a CDR-L2, and a CDR-L3 comprising the amino acid sequence set forth in SEQ ID NO: 210, 211, and 154, respectively. In some embodiments, the anti-CD4 scFv comprises a heavy chain variable region (VH) comprising the amino acid sequence set forth in SEQ ID NO:155. In some embodiments, the anti-CD4 scFv comprises a light chain variable region (VL) comprising the amino acid sequence set forth in SEQ ID NO:156. In some embodiments, the anti-CD4 scFv comprises a heavy chain variable region (VH) comprising the amino acid sequence set forth in SEQ ID NO:155; and a light chain variable region (VL) comprising the amino acid sequence set forth in SEQ ID NO:156. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:157.

In some embodiments, the anti-CD4 scFv comprises a CDR-H1, a CDR-H2, and a CDR-H3 comprising the amino acid sequence set forth in SEQ ID NO: 158, 159, and 160, respectively. In some embodiments, the anti-CD4 scFv comprises a CDR-L1, a CDR-L2, and a CDR-L3 comprising the amino acid sequence set forth in SEQ ID NO: 161, 162, and 163, respectively. In some embodiments, the anti-CD4 scFv comprises a CDR-H1, a CDR-H2, and a CDR-H3 comprising the amino acid sequence set forth in SEQ ID NO: 158, 159, and 160, respectively; and a CDR-L1, a CDR-L2, and a CDR-L3 comprising the amino acid sequence set forth in SEQ ID NO: 161, 162, and 163, respectively. In some embodiments, the anti-CD4 scFv comprises a CDR-H1, a CDR-H2, and a CDR-H3 comprising the amino acid sequence set forth in SEQ ID NO: 214, 215, and 216, respectively. In some embodiments, the anti-CD4 scFv comprises a CDR-L1, a CDR-L2, and a CDR-L3 comprising the amino acid sequence set forth in SEQ ID NO: 217, 218, and 163, respectively. In some embodiments, the anti-CD4 scFv comprises a CDR-H1, a CDR-H2, and a CDR-H3 comprising the amino acid sequence set forth in SEQ ID NO: 214, 215, and 216, respectively; and a CDR-L1, a CDR-L2, and a CDR-L3 comprising the amino acid sequence set forth in SEQ ID NO: 217, 218, and 163, respectively. In some embodiments, the anti-CD4 scFv comprises a CDR-H1, a CDR-H2, and a CDR-H3 comprising the amino acid sequence set forth in SEQ ID NO: 219, 220, and 216, respectively. In some embodiments, the anti-CD4 scFv comprises a CDR-L1, a CDR-L2, and a CDR-L3 comprising the amino acid sequence set forth in SEQ ID NO: 217, 218, and 163, respectively. In some embodiments, the anti-CD4 scFv comprises a CDR-H1, a CDR-H2, and a CDR-H3 comprising the amino acid sequence set forth in SEQ ID NO: 219, 220, and 216, respectively; and a CDR-L1, a CDR-L2, and a CDR-L3 comprising the amino acid sequence set forth in SEQ ID NO: 217, 218, and 163, respectively. In some embodiments, the anti-CD4 scFv comprises a heavy chain variable region (VH) comprising the amino acid sequence set forth in SEQ ID NO:164. In some embodiments, the anti-CD4 scFv comprises a light chain variable region (VL) comprising the amino acid sequence set forth in SEQ ID NO:165. In some embodiments, the anti-CD4 scFv comprises a heavy chain variable region (VH) comprising the amino acid sequence set forth in SEQ ID NO:164; and a light chain variable region (VL) comprising the amino acid sequence set forth in SEQ ID NO:165. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:166.

In some embodiments, the anti-CD4 scFv comprises a CDR-H1, a CDR-H2, and a CDR-H3 comprising the amino acid sequence set forth in SEQ ID NO: 167, 168, and 169, respectively. In some embodiments, the anti-CD4 scFv comprises a CDR-L1, a CDR-L2, and a CDR-L3 comprising the amino acid sequence set forth in SEQ ID NO: 170, 171, and 172, respectively. In some embodiments, the anti-CD4 scFv comprises a CDR-H1, a CDR-H2, and a CDR-H3 comprising the amino acid sequence set forth in SEQ ID NO: 167, 168, and 169, respectively; and a CDR-L1, a CDR-L2, and a CDR-L3 comprising the amino acid sequence set forth in SEQ ID NO: 170, 171, and 172, respectively. In some embodiments, the anti-CD4 scFv comprises a CDR-H1, a CDR-H2, and a CDR-H3 comprising the amino acid sequence set forth in SEQ ID NO: 221, 222, 223, respectively. In some embodiments, the anti-CD4 scFv comprises a CDR-L1, a CDR-L2, and a CDR-L3 comprising the amino acid sequence set forth in SEQ ID NO: 224, 225, and 172, respectively. In some embodiments, the anti-CD4 scFv comprises a CDR-H1, a CDR-H2, and a CDR-H3 comprising the amino acid sequence set forth in SEQ ID NO: 221, 222, 223, respectively; and a CDR-L1, a CDR-L2, and a CDR-L3 comprising the amino acid sequence set forth in SEQ ID NO: 224, 225, and 172, respectively. In some embodiments, the anti-CD4 scFv comprises a CDR-H1, a CDR-H2, and a CDR-H3 comprising the amino acid sequence set forth in SEQ ID NO: 226, 227, 223, respectively. In some embodiments, the anti-CD4 scFv comprises a CDR-L1, a CDR-L2, and a CDR-L3 comprising the amino acid sequence set forth in SEQ ID NO: 224, 225, and 172, respectively. In some embodiments, the anti-CD4 scFv comprises a CDR-H1, a CDR-H2, and a CDR-H3 comprising the amino acid sequence set forth in SEQ ID NO: 226, 227, 223, respectively; and a CDR-L1, a CDR-L2, and a CDR-L3 comprising the amino acid sequence set forth in SEQ ID NO: 224, 225, and 172, respectively. In some embodiments, the anti-CD4 scFv comprises a heavy chain variable region (VH) comprising the amino acid sequence set forth in SEQ ID NO:173. In some embodiments, the anti-CD4 scFv comprises a light chain variable region (VL) comprising the amino acid sequence set forth in SEQ ID NO:174. In some embodiments, the anti-CD4 scFv comprises a heavy chain variable region (VH) comprising the amino acid sequence set forth in SEQ ID NO:173; and a light chain variable region (VL) comprising the amino acid sequence set forth in SEQ ID NO:174. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:175.

In some embodiments, the anti-CD4 scFv comprises a CDR-H1, a CDR-H2, and a CDR-H3 comprising the amino acid sequence set forth in SEQ ID NO: 176, 177, and 178, respectively. In some embodiments, the anti-CD4 scFv comprises a CDR-L1, a CDR-L2, and a CDR-L3 comprising the amino acid sequence set forth in SEQ ID NO: 179, 180, and 181, respectively. In some embodiments, the anti-CD4 scFv comprises a CDR-H1, a CDR-H2, and a CDR-H3 comprising the amino acid sequence set forth in SEQ ID NO: 176, 177, and 178, respectively; and a CDR-L1, a CDR-L2, and a CDR-L3 comprising the amino acid sequence set forth in SEQ ID NO: 179, 180, and 181, respectively. In some embodiments, the anti-CD4 scFv comprises a CDR-H1, a CDR-H2, and a CDR-H3 comprising the amino acid sequence set forth in SEQ ID NO: 228, 229, 230, respectively. In some embodiments, the anti-CD4 scFv comprises a CDR-L1, a CDR-L2, and a CDR-L3 comprising the amino acid sequence set forth in SEQ ID NO: 231, 232, and 181, respectively. In some embodiments, the anti-CD4 scFv comprises a CDR-H1, a CDR-H2, and a CDR-H3 comprising the amino acid sequence set forth in SEQ ID NO: 228, 229, 230, respectively; and a CDR-L1, a CDR-L2, and a CDR-L3 comprising the amino acid sequence set forth in SEQ ID NO: 231, 232, and 181, respectively. In some embodiments, the anti-CD4 scFv comprises a CDR-H1, a CDR-H2, and a CDR-H3 comprising the amino acid sequence set forth in SEQ ID NO: 233, 234, 230, respectively. In some embodiments, the anti-CD4 scFv comprises a CDR-L1, a CDR-L2, and a CDR-L3 comprising the amino acid sequence set forth in SEQ ID NO: 231, 232, and 181, respectively. In some embodiments, the anti-CD4 scFv comprises a CDR-H1, a CDR-H2, and a CDR-H3 comprising the amino acid sequence set forth in SEQ ID NO: 233, 234, 230, respectively; and a CDR-L1, a CDR-L2, and a CDR-L3 comprising the amino acid sequence set forth in SEQ ID NO: 231, 232, and 181, respectively. In some embodiments, the anti-CD4 scFv comprises a heavy chain variable region (VH) comprising the amino acid sequence set forth in SEQ ID NO:182. In some embodiments, the anti-CD4 scFv comprises a light chain variable region (VL) comprising the amino acid sequence set forth in SEQ ID NO:183. In some embodiments, the anti-CD4 scFv comprises a heavy chain variable region (VH) comprising the amino acid sequence set forth in SEQ ID NO:182; and a light chain variable region (VL) comprising the amino acid sequence set forth in SEQ ID NO:183. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:184.

In some embodiments, the anti-CD4 scFv comprises a CDR-H1, a CDR-H2, and a CDR-H3 comprising the amino acid sequence set forth in SEQ ID NO: 185, 186, and 187, respectively. In some embodiments, the anti-CD4 scFv comprises a CDR-L1, a CDR-L2, and a CDR-L3 comprising the amino acid sequence set forth in SEQ ID NO: 188, 171, and 189, respectively. In some embodiments, the anti-CD4 scFv comprises a CDR-H1, a CDR-H2, and a CDR-H3 comprising the amino acid sequence set forth in SEQ ID NO: 185, 186, and 187, respectively; and a CDR-L1, a CDR-L2, and a CDR-L3 comprising the amino acid sequence set forth in SEQ ID NO: 188, 171, and 189, respectively. In some embodiments, the anti-CD4 scFv comprises a CDR-H1, a CDR-H2, and a CDR-H3 comprising the amino acid sequence set forth in SEQ ID NO: 235, 236, and 237, respectively. In some embodiments, the anti-CD4 scFv comprises a CDR-L1, a CDR-L2, and a CDR-L3 comprising the amino acid sequence set forth in SEQ ID NO: 238, 239, and 189, respectively. In some embodiments, the anti-CD4 scFv comprises a CDR-H1, a CDR-H2, and a CDR-H3 comprising the amino acid sequence set forth in SEQ ID NO: 235, 236, and 237, respectively; and a CDR-L1, a CDR-L2, and a CDR-L3 comprising the amino acid sequence set forth in SEQ ID NO: 238, 239, and 189, respectively. In some embodiments, the anti-CD4 scFv comprises a CDR-H1, a CDR-H2, and a CDR-H3 comprising the amino acid sequence set forth in SEQ ID NO: 240, 241, and 237, respectively. In some embodiments, the anti-CD4 scFv comprises a CDR-L1, a CDR-L2, and a CDR-L3 comprising the amino acid sequence set forth in SEQ ID NO: 238, 239, and 189, respectively. In some embodiments, the anti-CD4 scFv comprises a CDR-H1, a CDR-H2, and a CDR-H3 comprising the amino acid sequence set forth in SEQ ID NO: 240, 241, and 237, respectively; and a CDR-L1, a CDR-L2, and a CDR-L3 comprising the amino acid sequence set forth in SEQ ID NO: 238, 239, and 189, respectively. In some embodiments, the anti-CD4 scFv comprises a heavy chain variable region (VH) comprising the amino acid sequence set forth in SEQ ID NO:190. In some embodiments, the anti-CD4 scFv comprises a light chain variable region (VL) comprising the amino acid sequence set forth in SEQ ID NO:191. In some embodiments, the anti-CD4 scFv comprises a heavy chain variable region (VH) comprising the amino acid sequence set forth in SEQ ID NO:190; and a light chain variable region (VL) comprising the amino acid sequence set forth in SEQ ID NO:191. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:192.

In some embodiments, the anti-CD4 scFv comprises a CDR-H1, a CDR-H2, and a CDR-H3 comprising the amino acid sequence set forth in SEQ ID NO: 193, 194, and 195, respectively. In some embodiments, the anti-CD4 scFv comprises a CDR-L1, a CDR-L2, and a CDR-L3 comprising the amino acid sequence set forth in SEQ ID NO: 196, 197, and 198, respectively. In some embodiments, the anti-CD4 scFv comprises a CDR-H1, a CDR-H2, and a CDR-H3 comprising the amino acid sequence set forth in SEQ ID NO: 193, 194, and 195, respectively; and a CDR-L1, a CDR-L2, and a CDR-L3 comprising the amino acid sequence set forth in SEQ ID NO: 196, 197, and 198, respectively. In some embodiments, the anti-CD4 scFv comprises a CDR-H1, a CDR-H2, and a CDR-H3 comprising the amino acid sequence set forth in SEQ ID NO: 242, 243, and 244, respectively. In some embodiments, the anti-CD4 scFv comprises a CDR-L1, a CDR-L2, and a CDR-L3 comprising the amino acid sequence set forth in SEQ ID NO: 245, 246, and 198, respectively. In some embodiments, the anti-CD4 scFv comprises a CDR-H1, a CDR-H2, and a CDR-H3 comprising the amino acid sequence set forth in SEQ ID NO: 242, 243, and 244, respectively; and a CDR-L1, a CDR-L2, and a CDR-L3 comprising the amino acid sequence set forth in SEQ ID NO: 245, 246, and 198, respectively. In some embodiments, the anti-CD4 scFv comprises a CDR-H1, a CDR-H2, and a CDR-H3 comprising the amino acid sequence set forth in SEQ ID NO: 247, 248, and 244, respectively. In some embodiments, the anti-CD4 scFv comprises a CDR-L1, a CDR-L2, and a CDR-L3 comprising the amino acid sequence set forth in SEQ ID NO: 245, 246, and 198, respectively. In some embodiments, the anti-CD4 scFv comprises a CDR-H1, a CDR-H2, and a CDR-H3 comprising the amino acid sequence set forth in SEQ ID NO: 247, 248, and 244, respectively; and a CDR-L1, a CDR-L2, and a CDR-L3 comprising the amino acid sequence set forth in SEQ ID NO: 245, 246, and 198, respectively. In some embodiments, the anti-CD4 scFv comprises a heavy chain variable region (VH) comprising the amino acid sequence set forth in SEQ ID NO:199. In some embodiments, the anti-CD4 scFv comprises a light chain variable region (VL) comprising the amino acid sequence set forth in SEQ ID NO:200. In some embodiments, the anti-CD4 scFv comprises a heavy chain variable region (VH) comprising the amino acid sequence set forth in SEQ ID NO:199; and a light chain variable region (VL) comprising the amino acid sequence set forth in SEQ ID NO:200. In some embodiments, the VH and VL are joined by a linker. In some embodiments, the linker comprises the amino acid sequence set forth in SEQ ID NO:143. In some embodiments, the anti-CD4 scFv comprises the amino acid sequence set forth in SEQ ID NO:201.

In some embodiments, the anti-CD4 antibody or antigen-binding fragment is a single domain antibody. In some embodiments, the anti-CD4 antibody or antigen-binding fragment is a camelid (e.g. llama, alpaca, camel) anti-CD4 antibody or antigen-binding fragment (e.g. VHH). In some embodiments, the anti-CD4 antibody or antigen-binding fragment is an anti-CD4 VHH. In some embodiments, the anti-CD4 VHH comprises a CDR-H1, a CDR-H2, and a CDR-H3 comprising the amino acid sequence set forth in SEQ ID NO: 145, 146, and 147, respectively. In some embodiments, the anti-CD4 VHH comprises a CDR-H1, a CDR-H2, and a CDR-H3 comprising the amino acid sequence set forth in SEQ ID NO: 202, 203, and 204, respectively. In some embodiments, the anti-CD4 VHH comprises a CDR-H1, a CDR-H2, and a CDR-H3 comprising the amino acid sequence set forth in SEQ ID NO: 205, 206, and 204, respectively. In some embodiments, the anti-CD4 VHH comprises the amino acid sequence set forth in SEQ ID NO:148.

In some of any of the provided embodiments, the CD4 binding agent is exposed on the surface of the lentiviral vector. In some embodiments, the CD4 binding agent is fused to a transmembrane domain incorporated in the viral envelope.

In some embodiments, the lentiviral vector is pseudotyped with a viral fusion protein. In some embodiments, the viral fusion protein is a VSV-G protein or a functional variant thereof. In some embodiments, the virial fusion protein is a Cocal virus G protein or a functional variant thereof. In some embodiments, the viral fusion protein is an Alphavirus fusion protein (e.g. Sindbis virus) or a functional variant thereof. In some embodiments, the viral fusion protein is a Paramyxoviridae fusion protein (e.g., a Morbillivirus or a Henipavirus) or a functional variant thereof. In some embodiments, the viral fusion protein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the viral fusion protein is a Henipavirus fusion protein (e.g., Nipah virus, Hendra virus, Cedar virus, Kumasi virus, Mójiāng virus) or a functional variant thereof.

In some of any of the provided embodiments, the viral fusion protein comprises one or modifications to reduce binding to its native receptor.

In some of any of the provided embodiments, the viral fusion protein is fused to the CD4 binding agent. In some embodiments, the viral fusion protein is or comprises a canine distemper virus protein. In some embodiments, the viral fusion protein is a canine distemper virus protein or a functional variant thereof. In some embodiments, the viral fusion protein comprises a canine distemper virus F protein or a biologically active portion thereof. In some embodiments, the CD4 binding agent is fused to the canine distemper virus F protein or the biologically active portion thereof. In some embodiments, the viral fusion protein comprises a canine distemper virus F protein or a biologically active portion thereof, wherein the CD4 binding agent is fused to the canine distemper virus F protein or the biologically active portion thereof. In some embodiments, the CD4 binding protein is fused directly or via a peptide linker.

In some of any of the provided embodiments, the viral fusion protein is fused to the CD4 binding agent. In some embodiments, the viral fusion protein is or comprises a Paramyxovirus (e.g., measles virus or Nipah virus) fusion protein (e.g., a Paramyxovirus G protein). In some embodiments, the viral fusion protein is a Nipah virus fusion protein or a functional variant thereof. In some embodiments, the viral fusion protein comprises a Nipah virus F glycoprotein (NiV-F) or a biologically active portion thereof and a Nipah virus G glycoprotein (NiV-G) or a biologically active portion thereof. In some embodiments, the CD4 binding agent is fused to the NiV-G or the biologically active portion thereof. In some embodiments, the viral fusion protein comprises a Nipah virus F glycoprotein (NiV-F) or a biologically active portion thereof and a Nipah virus G glycoprotein (NiV-G) or a biologically active portion thereof, and wherein the CD4 binding agent is fused to the NiV-G or the biologically active portion thereof. In some embodiments, the CD4 binding agent is fused to the C-terminus of the Nipah virus G glycoprotein or the biologically active portion thereof. In some embodiments, the CD4 binding protein is fused directly or via a peptide linker.

In some embodiments, the NiV-G protein or the biologically active portion thereof is a wild-type NiV-G protein or a functionally active variant or biologically active portion thereof.

In some embodiments, the NiV-G protein or the biologically active portion is truncated and lacks up to 40 contiguous amino acid residues at or near the N-terminus of the wild-type NiV-G protein (SEQ ID NO:1,SEQ ID NO:4 or SEQ ID NO:5). In some embodiments, the NiV-G protein or the biologically active portion has a 5 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:12, or a sequence of amino acids that exhibits at least at or about 80%, 85%, 90% or 95% sequence identity to the sequence set forth in SEQ ID NO:12. In some embodiments, the NiV-G protein or the biologically active portion thereof has the amino acid sequence set forth in SEQ ID NO:12. In some embodiments, the NiV-G protein or the biologically active portion has a 5 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:12, or a sequence of amino acids that exhibits at least at or about 80%, 85%, 90% or 95% sequence identity to the sequence set forth in SEQ ID NO:12. In some embodiments, the NiV-G protein or the biologically active portion has a 10 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion has a 10 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44. In some embodiments, the NiV-G protein or the biologically active portion thereof has the amino acid sequence set forth in SEQ ID NO:44. In some embodiments, the NiV-G protein or the biologically active portion has a 15 amino acid truncation at or near the N-terminus of the wild-type NiV-G protein (SEQ ID NO:9, SEQ ID NO:4 or SEQ ID NO:5), optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion thereof has the amino acid sequence set forth in SEQ ID NO:45. In some embodiments, the NiV-G protein or the biologically active portion has a 15 amino acid truncation at or near the N-terminus of the wild-type NiV-G protein (SEQ ID NO:9, SEQ ID NO:4 or SEQ ID NO:5). In some embodiments, the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45. In some embodiments, the NiV-G protein or the biologically active portion has a 20 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:13, or a sequence of amino acids that exhibits at least at or about 80%, 85%, 90% or 95% sequence identity to the sequence set forth in SEQ ID NO:13. In some embodiments, the NiV-G protein or the biologically active portion has a 20 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:13, or a sequence of amino acids that exhibits at least at or about 80%, 85%, 90% or 95% sequence identity to the sequence set forth in SEQ ID NO:13. In some embodiments, the NiV-G protein or the biologically active portion thereof has the amino acid sequence set forth in SEQ ID NO:13. In some embodiments, the NiV-G protein or the biologically active portion has a 25 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:14, or a sequence of amino acids that exhibits at least at or about 80%, 85%, 90% or 95% sequence identity to the sequence set forth in SEQ ID NO: 14. In some embodiments, the NiV-G protein or the biologically active portion has a 25 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:14, or a sequence of amino acids that exhibits at least at or about 80%, 85%, 90% or 95% sequence identity to the sequence set forth in SEQ ID NO: 14. In some embodiments, the NiV-G protein or the biologically active portion thereof has the amino acid sequence set forth in SEQ ID NO:14. In some embodiments, the NiV-G protein or the biologically active portion has a 30 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43. In some embodiments, the NiV-G protein or the biologically active portion has a 30 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43. In some embodiments, the NiV-G protein or the biologically active portion thereof has the amino acid sequence set forth in SEQ ID NO:43. In some embodiments, the NiV-G protein or the biologically active portion has a 34 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42. In some embodiments, the NiV-G protein or the biologically active portion thereof has the amino acid sequence set forth in SEQ ID NO:42. In some embodiments, the NiV-G protein or the biologically active portion has a 34 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42. In some embodiments, the NiV-G protein or the biologically active portion has a 34 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42. In some embodiments, the NiV-G protein or the biologically active portion has a 34 amino acid truncation at or near the N-terminus of the wild-type NiV-G protein (SEQ ID NO:1, SEQ ID NO:4 or SEQ ID NO:5). In some embodiments, the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

In some embodiments, the NiV-G-protein or the biologically active portion thereof is a mutant NiV-G protein. In some of any of the provided embodiments, the NiV-G-protein or the biologically active portion thereof is a mutant NiV-G protein that exhibits reduced binding to Ephrin B2 or Ephrin B3. In some of any of the provided embodiments, the mutant NiV-G protein or the biologically active portion comprises: one or more amino acid substitutions corresponding to amino acid substitutions selected from the group consisting of E501A, W504A, Q530A and E533A with reference to numbering set forth in SEQ ID NO:4. In some embodiments, the mutant NiV-G protein or the biologically active portion has the amino acid sequence set forth in SEQ ID NO: 17 or a sequence of amino acids that exhibits at least at or about 80%, 85%, 90% or 95% sequence identity to the sequence set forth in SEQ ID NO: 17. In some embodiments, the mutant NiV-G protein or the biologically active portion has the amino acid sequence set forth in SEQ ID NO: 17. In some embodiments, the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18. In some embodiments, the NiV-G protein or the biologically active portion has the amino acid sequence set forth in SEQ ID NO: 18.

In some of any of the provided embodiments, the NiV-F protein or the biologically active portion thereof is a wild-type NiV-F protein or is a functionally active variant or a biologically active portion thereof. In some of any of the provided embodiments, the NiV-F protein or the biologically active portion thereof has a 20 amino acid truncation at or near the C-terminus of the wild-type NiV-F protein (SEQ ID NO: 41 or SEQ ID NO:40 without signal sequence), optionally wherein the NiV-F protein or the biologically active portion thereof has the sequence set forth in SEQ ID NO: 20 or a sequence of amino acids that exhibits at least at or about 80%, 85%, 90% or 95% sequence identity to the sequence set forth in SEQ ID NO: 20. In some of any of the provided embodiments, the NiV-F protein or the biologically active portion thereof has a 20 amino acid truncation at or near the C-terminus of the wild-type NiV-F protein (SEQ ID NO: 41). In some embodiments, the NiV-F protein or the biologically active portion thereof has the sequence set forth in SEQ ID NO: 20 or a sequence of amino acids that exhibits at least at or about 80%, 85%, 90% or 95% sequence identity to the sequence set forth in SEQ ID NO: 20. In some embodiments, the NiV-F protein or the biologically active portion thereof has the sequence set forth in SEQ ID NO: 20. In some of any of the provided embodiments, the NiV-F protein or the biologically active portion thereof comprises: i) a 20 amino acid truncation at or near the C-terminus of the wild-type NiV-F protein (SEQ ID NO: 41); and ii) a point mutation on an N-linked glycosylation site, optionally wherein the NiV-F protein or the biologically active portion thereof has the sequence set forth in SEQ ID NO: 15, or a sequence of amino acids that exhibits at least at or about 80%, 85%, 90% or 95% sequence identity to the sequence set forth in SEQ ID NO: 15. In some of any of the provided embodiments, the NiV-F protein or the biologically active portion thereof comprises: i) a 20 amino acid truncation at or near the C-terminus of the wild-type NiV-F protein (SEQ ID NO: 41 or SEQ ID NO:40 without signal sequence); and ii) a point mutation on an N-linked glycosylation site. In some embodiments, the NiV-F protein or the biologically active portion thereof has the sequence set forth in SEQ ID NO: 15, or a sequence of amino acids that exhibits at least at or about 80%, 85%, 90% or 95% sequence identity to the sequence set forth in SEQ ID NO: 15. In some embodiments, the NiV-F protein or the biologically active portion thereof has the sequence set forth in SEQ ID NO: 15. In some of any of the provided embodiments, the NiV-F protein or the biologically active portion thereof has a 22 amino acid truncation at or near the C-terminus of the wild-type NiV-F protein (SEQ ID NO: 4 or SEQ ID NO:40 without signal sequence1), optionally wherein the NiV-F protein or the biologically active portion thereof has the sequence set forth in SEQ ID NO: 16 or 21 or a sequence of amino acids that exhibits at least at or about 80%, 85%, 90% or 95% sequence identity to the sequence set forth in SEQ ID NO: 16 or 21. In some of any of the provided embodiments, the NiV-F protein or the biologically active portion thereof has a 22 amino acid truncation at or near the C-terminus of the wild-type NiV-F protein (SEQ ID NO: 41). In some embodiments, the NiV-F protein or the biologically active portion thereof has the sequence set forth in SEQ ID NO: 16 or 21 or a sequence of amino acids that exhibits at least at or about 80%, 85%, 90% or 95% sequence identity to the sequence set forth in SEQ ID NO: 16 or 21. In some embodiments, the NiV-F protein or the biologically active portion thereof has the sequence set forth in SEQ ID NO: 16 or a sequence of amino acids that exhibits at least at or about 80%, 85%, 90% or 95% sequence identity to the sequence set forth in SEQ ID NO: 16. In some embodiments, the NiV-F protein or the biologically active portion thereof has the sequence set forth in SEQ ID NO: 16. In some embodiments, the NiV-F protein or the biologically active portion thereof has the sequence set forth in SEQ ID NO: 21 or a sequence of amino acids that exhibits at least at or about 80%, 85%, 90% or 95% sequence identity to the sequence set forth in SEQ ID NO: 21. In some embodiments, the NiV-F protein or the biologically active portion thereof has the sequence set forth in SEQ ID NO: 21.

In some embodiments, the NiV-G protein or the biologically active portion comprises the amino acid sequence set forth in SEQ ID NO: 17, and the NiV-F protein or the biologically active portion thereof comprises the sequence set forth in SEQ ID NO: 21. In some embodiments, the NiV-G protein or the biologically active portion consists of the amino acid sequence set forth in SEQ ID NO: 17, and the NiV-F protein or the biologically active portion thereof consists of the sequence set forth in SEQ ID NO: 21.

In some of any of the provided embodiments, the lentiviral vector comprises a transgene. In some embodiments, the transgene comprises a nucleic acid sequence encoding an RNA sequence capable of RNA interference (e.g. pre-miRNA, siRNA, or shRNA). In some embodiments, the transgene is selected from the group consisting of a therapeutic gene, a reporter gene, a gene encoding an enzyme, a gene encoding a pro-drug enzyme, a gene encoding an apoptosis inducer, a gene encoding a fluorescent protein, a gene encoding a pro-drug-activating enzyme, a gene encoding an apoptotic protein, a gene encoding an apoptotic enzyme, a gene encoding a suicide protein, a gene encoding a cytokine, a gene encoding an anti-immunosuppressive protein, a gene encoding an epigenetic modulator, a gene encoding a T cell receptor (TCR), a gene encoding a chimeric antigen receptor (CAR), a gene encoding a protein that modifies the cell surface of transduced cells, a gene encoding a protein that modifies the expression of the endogenous TCR, and a gene encoding a switch receptor that converts pro-tumor into anti-tumor signals. In some embodiments, the transgene encodes an engineered receptor that binds to or recognizes a protein or antigen expressed by cells or a lesion (e.g. tumor) associated with a disease or condition, optionally wherein the engineered receptor is a chimeric antigen receptor (CAR) or an engineered T cell receptor (TCR). In some embodiments, the transgene encodes an engineered receptor that binds to or recognizes a protein or antigen expressed by cells or a lesion (e.g. tumor) associated with a disease or condition. In some embodiments, the engineered receptor is a chimeric antigen receptor (CAR) or an engineered T cell receptor (TCR).

In some embodiments, the transgene encodes a chimeric antigen receptor (CAR). In some embodiments, the transgene encodes an engineered T cell receptor (TCR).

In some embodiments, the contacting is carried out by ex vivo administration of the lentiviral vector to a subject using a closed fluid circuit. In some embodiments, the administering is carried out by ex vivo administration of the lentiviral vector to a subject using a closed fluid circuit. In some embodiments, the ex vivo administration comprises (a) obtaining whole blood from a subject; (b) collecting the fraction of blood containing leukocyte components comprising T cells (e.g. CD4+ T cells); (c) contacting the leukocyte components comprising T cells (e.g. CD4+ T cells) with a composition comprising the lentiviral vector; and (d) reinfusing the contacted leukocyte components comprising T cells (e.g. CD4+ T cells) into the subject, wherein steps (a)-(d) are performed in-line in a closed fluid circuit. In some embodiments, the contacting in step (c) is for no more than 24 hours, no more than 18 hours, no more than 12 hours, or no more than 6 hours.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### BRIEF DESCRIPTION OF THE DRAWINGS

FIG. 1 depicts an exemplary system for ex vivo dosing.

FIG. 2A shows tumor burden at Day 21 in CD19+ tumor bearing mice treated with 2.5E6, 5E6, or 1E7 integrating units (IU) of CD4-targeted CD19 CAR fusosomes, as assessed by bioluminescence imaging.

FIG. 2B shows the percentage of CD4+ T cells that express CAR at Day 15 in CD19+ tumor bearing mice treated with 2.5E6, 5E6, or 1E7 integrating units (IU) of CD4-targeted CD19 CAR fusosomes, as assessed by flow cytometry.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 June 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3. Table 1, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-H1 | H31--H35B | H26--H32 . . . | H26--H35B | H30--H35B |
| (Kabat | | 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given VHH amino acid sequence, it is understood that such a CDR has a sequence of the corresponding CDR (e.g., CDR-H3) within the VHH, as defined by any of the aforementioned schemes. In some embodiments, specific CDR sequences are specified. Exemplary CDR sequences of provided antibodies are described using various numbering schemes (see e.g. Table 1), although it is understood that a provided antibody can include CDRs as described according to any of the other aforementioned numbering schemes or other numbering schemes known to a skilled artisan.

As used herein, “fusosome” refers to a particle containing a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell. In some embodiments, the fusosome is derived from a vector, such as a viral vector (e.g., a lentiviral vector).

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a viral vector, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

### II. METHODS

Provided herein are methods of transducing a T cell or a population thereof, comprising contacting a non-activated T cell or a population thereof with a lentiviral vector comprising a CD4 binding agent, wherein the lentiviral vector transduces the non-activated T cell(s). In some embodiments, the population of non-activated T cells is transduced at an efficiency of at least 1%.

Also provided herein are methods of in vivo transduction of T cells comprising administering to a subject a composition comprising a lentiviral vector comprising a CD4 binding agent, wherein the lentiviral vector transduces T cells within the subject. Also provided herein are methods of treating a subject having a disease or condition, the method comprising administering to the subject a composition comprising a lentiviral vector comprising a CD4 binding agent. Also provided herein are methods for expanding T cells capable of recognizing and killing tumor cells in a subject in need thereof, the method comprising administering to the subject a composition comprising a lentiviral vector comprising a CD4 binding agent. In some embodiments, the subject is not administered a T cell activating treatment (e.g., before, after, or concurrently) with administration of the composition.

In some aspects, resting or non-activated T cells are contacted with a viral vector (e.g., a retroviral vector or lentiviral vector) that includes a CD4 binding agent. The contacting may be performed in vitro (e.g., with T cells derived from a healthy donor or a donor in need of cellular therapy) or in vivo by administration of the viral vector to a subject.

In some embodiments, the resting or non-activated T cells are not treated with one or more T cell stimulatory molecules (e.g., an anti CD-3 antibody), one or more T cell costimulatory molecules, and/or one or more T cell activating cytokines. In some embodiments, the resting or non-activated T cells are not treated with any of one or more T cell stimulatory molecules (e.g., an anti CD-3 antibody), one or more T cell costimulatory molecules, and/or one or more T cell activating cytokines.

In additional aspects, the application includes methods of administration to a subject, including any of those as described in Sections VI and VIII. In some embodiments, the methods include administration to a subject of a viral vector that includes an anti-CD4 binding agent, wherein the subject is not administered or has not been administered a T cell activating treatment. In some embodiments, the T cell activating treatment includes one or more T cell stimulatory molecules (e.g., an anti CD-3 antibody), one or more T cell costimulatory molecules, and/or one or more T cell activating cytokines. In some embodiments, the subject is not administered or has not been administered any of one or more T cell stimulatory molecules (e.g., an anti CD-3 antibody), one or more T cell costimulatory molecules, and/or one or more T cell activating cytokines. In some embodiments, the T cell activating treatment is lymphodepletion. In some embodiments, the subject is not administered or has not been administered a lymphodepleting therapy. In certain embodiments, the subject is not administered or has not been administered the T cell activating treatment within 1 month before or after administration of the viral vector. In some embodiments, the subject is not administered or has not been administered the T cell activating treatment within 1 month before administration of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector. In some embodiments, the subject is not administered the T cell activating treatment within 1 month after administration of the viral vector, such as within or at or about 4 weeks, 3 weeks, 2 weeks or 1 weeks, such as at or about 1 day, 2 days, 3 days, 4 days, 5 days, 6 days or 7 days after administration of the viral vector.

In some aspects, the viral vector does not include or encode a T cell activating agent. In some embodiments, the viral vector does not include or encode a membrane-bound T cell activating agent. In some embodiments, the viral vector does not include or encode a T cell activating agent that is displayed on the surface. In some embodiments, the T cell activating agent is an anti-CD3 antibody (e.g. an anti-CD3 scFv), a T cell activating cytokine (e.g. IL-2, IL-7, IL-15 or IL-21), or a T cell costimulatory molecule (e.g. anti-CD28 antibody, CD80, CD86, CD137L or ICOS-L. In some embodiments, the T cell activating agent is a polypeptide capable of binding CD3, a polypeptide capable of binding to CD28, or both. In some aspects, the viral vector does not include one or more T cell stimulatory molecules (e.g., an anti CD3 antibody), one or more T cell costimulatory molecules, and/or one or more T cell activating cytokines.

The use of anti-CD3 antibodies is well-known for activation of T cells. The anti-CD3 antibodies can be of any species, e.g., mouse, rabbit, human, humanized, or camelid. Exemplary antibodies include OKT3, CRIS-7, I2C the anti-CD3 antibody included in DYNABEADS Human T-Activator CD3/CD28 (Thermo Fisher), and the anti-CD3 domains of approved and clinically studied molecules such as blinatumomab, catumaxomab, fotetuzumab, teclistamab, ertumaxomab, epcoritamab, talquetamab, odronextamab, cibistamab, obrindatamab, tidutamab, duvortuxizumab, solitomab, eluvixtamab, pavurutamab, tepoditamab, vibecotamab, plamotamab, glofitamab, etevritamab, and tarlatamab.

In some embodiments, the one or more T cell costimulatory molecules include CD28 ligands (e.g., CD80 and CD86); antibodies that bind to CD28 such as CD28.2, the anti-CD28 antibody included in DYNABEADS Human T-Activator CD3/CD28 (Thermo Fisher) and anti-CD28 domains disclosed in US2020/0199234, US2020/0223925, US2020/0181260, US2020/0239576, US2020/0199233, US2019/0389951, US2020/0299388, US2020/0399369, and US2020/0140552; CD137 ligand (CD137L); anti-CD137 antibodies such as urelumab and utomilumab; ICOS ligand (ICOS-L); and anti-ICOS antibodies such as feladilimab, vopratelimab, and the anti-ICOS domain of izuralimab.

In some embodiments, the one or more T cell activating cytokines include IL-2, IL-7, IL-15, IL-21, interferons (e.g., interferon-gamma), and functional variants and modified versions thereof.

In some aspects, the viral vector does not include or encode a T cell activating agent. In some embodiments, the viral vector does not include or encode a membrane-bound T cell activating agent. In some embodiments, the viral vector does not include or encode a T cell activating agent that is displayed on the surface. In some embodiments, the T cell activating agent is a lymphoproliferative element. In some embodiments, the lymphoproliferative element is a cytokine or a cytokine receptor or a signaling domain thereof that activates a STAT3 pathway, a STAT4 pathway, and/or a Jak/STAT5 pathway. In some embodiments, the lymphoproliferative element is a T cell survival motif, such as an IL-7 receptor, an IL-15 receptor, or CD28, or a functional portion thereof. In some embodiments, the lymphoproliferative element is a micro RNA (miRNA) or a short hairpin RNA (shRNA) that stimulates the STAT5 pathway, inhibits the SOCS pathway, or both.

In some embodiments, the vector does not include or encode an inhibitory RNA molecule. In some embodiments, the inhibitory RNA molecule targets an mRNA transcribed from a gene expressed by T cells, a gene encoding a component of a T cell receptor (TCR), or both. In some embodiments, the gene is PD-1, CTLA4, TCRα, TCRβ, CD3δ, SOCS1, SMAD2, a miR-155 target, IFNγ, TRAIL2, and/or ABCG1.

In some embodiments, the vector includes or encodes an inhibitory RNA molecule. In some embodiments, the inhibitory RNA molecule targets an mRNA transcribed from a gene expressed by T cells, a gene encoding a component of a T cell receptor (TCR), or both. In some embodiments, the gene is PD-1, CTLA4, TCRα, TCRβ, CD3δ, SOCS1, SMAD2, a miR-155 target, IFNγ, TRAIL2, and/or ABCG1.

In some embodiments, the methods further include administering a lymphodepleting therapy to a subject. In some embodiments, the T cell activating treatment comprises administration of a lymphodepleting therapy to a subject. Lymphodepletion may be induced by various treatments that destroy lymphocytes and T cells in the subject. For example, the lymphodepletion may include myeloablative chemotherapies, such as fludarabine, cyclophosphamide, bendamustine, and combinations thereof. Lymphodepletion may also be induced by irradiation (e.g., full-body irradiation) of the subject. In some embodiments, a lymphodepleting therapy comprises cyclophosphamide and/or fludarabine. In some embodiments, the methods further comprise administering cyclophosphamide and/or fludarabine.

### III. Viral Vectors

Provided herein are viral vectors, such as for transducing T cells. In some embodiments, a viral vector that binds a cell surface receptor for delivery of an exogenous agent (e.g., a transgene) through membrane fusion is provided as a “fusosome.” Thus, in some cases, a fusosome refers to a viral vector disclosed herein.

In some embodiments, the viral vector disclosed herein is a retroviral vector (e.g., a lentiviral vector). In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740).

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used. In some embodiments, the virus particles are derived from lentivirus. In some embodiments, the lentiviral vector particle is Human Immunodeficiency Virus-1 (HIV-1).

In some embodiments, the viral vector such as retrovirus or lentiviral vector, comprises one or more of gag polyprotein, polymerase (e.g., pol), integrase (e.g., a functional or non-functional variant), protease, and a fusogen. In some embodiments, the vector further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the retroviral nucleic acid comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the non-retroviral nucleic acid further comprises one or more insulator element. In some embodiments, the recognition sites are situated between the poly A tail sequence and the WPRE.

### 1. Transfer Vectors

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. In some aspects, vector particles will typically include various viral components and sometimes also host cell components in addition to nucleic acid(s). In some embodiments, a vector comprises e.g., a virus or viral particle capable of transferring a nucleic acid into a cell, or to the transferred nucleic acid (e.g., as naked mRNA). In some embodiments, viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the vectors described herein at least part of one or more protein coding regions that contribute to or are essential for replication may be absent compared to the corresponding wild-type virus. In some embodiments, the viral vector is replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are calledU3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-1 and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence. The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked b the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

### 2 Packaging Vectors

Large scale vector particle production is often useful to achieve a desired concentration of vector particles. Particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a producer cell, and are introduced into the cell via transfection, transduction or infection. A retroviral, e.g., lentiviral, transfer vector can be introduced into a producer cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 1OT1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

In some embodiments, the retroviral vector systems described herein comprise viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. In some embodiments, by separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, a viral vector particle which comprises a sequence that is devoid of or lacking viral RNA may be the result of removing or eliminating the viral RNA from the sequence. In one embodiment this may be achieved by using an endogenous packaging signal binding site on gag. In some embodiments, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles are used to deliver therapeutic RNA, in which case functional integrase and/or reverse transcriptase is not required. In some embodiments, the vector particles could also be used to deliver a therapeutic gene of interest, in which case pol is typically included.

In some embodiments, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

In some embodiments, an alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1.

### IV. Fusogens

In some embodiments, the viral vector is provided as a fusosome. In some embodiments, the viral vector comprises one or more fusogens. In some embodiments, the fusogen facilitates the fusion of the viral vector to a membrane. In some embodiments, the membrane is a plasma cell membrane.

In some embodiments, the viral vector comprising the fusogen (also called a “fusosome” herein) integrates into the membrane into a lipid bilayer of a target cell. In some embodiments, one or more of the fusogens described herein may be included in the viral vector.

### A. Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen results in mixing between lipids in the viral vector and lipids in the target cell. In some embodiments, the fusogen results in formation of one or more pores between the interior of the viral vector and the cytosol of the target cell.

### 1. Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 3), any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

### 2 Viral Proteins

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G), Cocal virus G protein), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of a Morbillivirus (e.g., measles virus (MeV), canine distemper virus, Cetacean morbillivirus, Peste-des-petits-ruminants virus, Phocine distemper virus, Rinderpest virus), Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., El protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and 3 sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

a. G Proteins

In some embodiments the G protein is a Paramyxovirus (e.g., Morbillivirus or Henipavirus) G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. A non-limited list of exemplary G proteins is shown in Table 2.

The attachment G proteins are type II transmembrane glycoproteins containing an N-terminal cytoplasmic tail (e.g. corresponding to amino acids 1-49 of SEQ ID NO:1), a transmembrane domain (e.g. corresponding to amino acids 50-70 of SEQ ID NO:1, and an extracellular domain containing an extracellular stalk (e.g. corresponding to amino acids 71-187 of SEQ ID NO:1), and a globular head (corresponding to amino acids 188-602 of SEQ ID NO:1). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

TABLE 2
Exemplary Henipavirus G Proteins

| | | | SEQ ID NO |
|---|---|---|---|
| | | SEQ | (without |
| Viral G | | ID | N-terminal |
| Protein | Sequence | NO | methionine) |
| Hendra Virus G | MMADSKLVSLNNNLSGKIKDQGKVIKNYYGTMDI | 2 | 3 |
| Protein | KKINDGLLDSKILGAFNTVIALLGSIIIIVMNIM | | |
| | IIQNYTRTTDNQALIKESLQSVQQQIKALTDKIG | | |
| | TEIGPKVSLIDTSSTITIPANIGLLGSKISQSTS | | |
| | SINENVNDKCKFTLPPLKIHECNISCPNPLPFRE | | |
| | YRPISQGVSDLVGLPNQICLQKTTSTILKPRLIS | | |
| | YTLPINTREGVCITDPLLAVDNGFFAYSHLEKIG | | |
| | SCTRGIAKQRIIGVGEVLDRGDKVPSMFMTNVWT | | |
| | PPNPSTIHHCSSTYHEDFYYTLCAVSHVGDPILN | | |
| | STSWTESLSLIRLAVRPKSDSGDYNQKYIAITKV | | |
| | ERGKYDKVMPYGPSGIKQGDTLYFPAVGFLPRTE | | |
| | FQYNDSNCPIIHCKYSKAENCRLSMGVNSKSHYI | | |
| | LRSGLLKYNLSLGGDIILQFIEIADNRLTIGSPS | | |
| | KIYNSLGQPVFYQASYSWDTMIKLGDVDTVDPLR | | |
| | VQWRNNSVISRPGQSQCPRFNVCPEVCWEGTYND | | |
| | AFLIDRLNWVSAGVYLNSNQTAENPVFAVFKDNE | | |
| | ILYQVPLAEDDTNAQKTITDCFLLENVIWCISLV | | |
| | EIYDTGDSVIRPKLFAVKIPAQCSES | | |
| Nipah Virus G | MPAENKKVRFENTTSDKGKIPSKVIKSYYGTMDI | 4 | 5 |
| Protein | KKINEGLLDSKILSAFNTVIALLGSIVIIVMNIM | | |
| | IIQNYTRSTDNQAVIKDALQGIQQQIKGLADKIG | | |
| | TEIGPKVSLIDTSSTITIPANIGLLGSKISQSTA | | |
| | SINENVNEKCKFTLPPLKIHECNISCPNPLPFRE | | |
| | YRPQTEGVSNLVGLPNNICLQKTSNQILKPKLIS | | |
| | YTLPVVGQSGTCITDPLLAMDEGYFAYSHLERIG | | |
| | SCSRGVSKQRIIGVGEVLDRGDEVPSLFMTNVWT | | |
| | PPNPNTVYHCSAVYNNEFYYVLCAVSTVGDPILN | | |
| | STYWSGSLMMTRLAVKPKSNGGGYNQHQLALRSI | | |
| | EKGRYDKVMPYGPSGIKQGDTLYFPAVGFLVRTE | | |
| | FKYNDSNCPITKCQYSKPENCRLSMGIRPNSHYI | | |
| | LRSGLLKYNLSDGENPKVVFIEISDQRLSIGSPS | | |
| | KIYDSLGQPVFYQASFSWDTMIKFGDVLTVNPLV | | |
| | VNWRNNTVISRPGQSQCPRFNTCPEICWEGVYND | | |
| | AFLIDRINWISAGVFLDSNQTAENPVFTVFKDNE | | |
| | ILYRAQLASEDTNAQKTITNCFLLKNKIWCISLV | | |
| | EIYDTGDNVIRPKLFAVKIPEQCT | | |
| Cedar Virus G | MLSQLQKNYLDNSNQQGDKMNNPDKKLSVNFNPL | 6 | 7 |
| Protein | ELDKGQKDLNKSYYVKNKNYNVSNLLNESLHDIK | | |
| | FCIYCIFSLLIIITIINIITISIVITRLKVHEEN | | |
| | NGMESPNLQSIQDSLSSLTNMINTEITPRIGILV | | |
| | TATSVTLSSSINYVGTKTNQLVNELKDYITKSCG | | |
| | FKVPELKLHECNISCADPKISKSAMYSTNAYAEL | | |
| | AGPPKIFCKSVSKDPDFRLKQIDYVIPVQQDRSI | | |
| | CMNNPLLDISDGFFTYIHYEGINSCKKSDSFKVL | | |
| | LSHGEIVDRGDYRPSLYLLSSHYHPYSMQVINCV | | |
| | PVTCNQSSFVFCHISNNTKTLDNSDYSSDEYYIT | | |
| | YFNGIDRPKTKKIPINNMTADNRYIHFTFSGGGG | | |
| | VCLGEEFIIPVTTVINTDVFTHDYCESFNCSVQT | | |
| | GKSLKEICSESLRSPTNSSRYNLNGIMIISQNNM | | |
| | TDFKIQLNGITYNKLSFGSPGRLSKTLGQVLYYQ | | |
| | SSMSWDTYLKAGFVEKWKPFTPNWMNNTVISRPN | | |
| | QGNCPRYHKCPEICYGGTYNDIAPLDLGKDMYVS | | |
| | VILDSDQLAENPEITVFNSTTILYKERVSKDELN | | |
| | TRSTTTSCFLFLDEPWCISVLETNRFNGKSIRPE | | |
| | IYSYKIPKYC | | |
| Bat | MPQKTVEFINMNSPLERGVSTLSDKKTLNQSKIT | 8 | 9 |
| Paramyxovirus | KQGYFGLGSHSERNWKKQKNQNDHYMTVSTMILE | | |
| G Protein, | ILVVLGIMFNLIVLTMVYYQNDNINQRMAELTSN | | |
| Eid_hel/ | ITVLNLNLNQLTNKIQREIIPRITLIDTATTITI | | |
| GH-M74a/ | PSAITYILATLTTRISELLPSINQKCEFKTPTLV | | |
| GHA/2009 | LNDCRINCTPPLNPSDGVKMSSLATNLVAHGPSP | | |
| | CRNFSSVPTIYYYRIPGLYNRTALDERCILNPRL | | |
| | TISSTKFAYVHSEYDKNCTRGFKYYELMTFGEIL | | |
| | EGPEKEPRMFSRSFYSPTNAVNYHSCTPIVTVNE | | |
| | GYFLCLECTSSDPLYKANLSNSTFHLVILRHNKD | | |
| | EKIVSMPSFNLSTDQEYVQIIPAEGGGTAESGNL | | |
| | YFPCIGRLLHKRVTHPLCKKSNCSRTDDESCLKS | | |
| | YYNQGSPQHQVVNCLIRIRNAQRDNPTWDVITVD | | |
| | LTNTYPGSRSRIFGSFSKPMLYQSSVSWHTLLQV | | |
| | AEITDLDKYQLDWLDTPYISRPGGSECPFGNYCP | | |
| | TVCWEGTYNDVYSLTPNNDLFVTVYLKSEQVAEN | | |
| | PYFAIFSRDQILKEFPLDAWISSARTTTISCFMF | | |
| | NNEIWCIAALEITRLNDDIIRPIYYSFWLPTDCR | | |
| | TPYPHTGKMTRVPLRSTYNY | | |
| Mojiang virus, | MATNRDNTITSAEVSQEDKVKKYYGVETAEKVAD | 10 | 11 |
| Tongguan 1 G | SISGNKVFILMNTLLILTGAIITITLNITNLTAA | | |
| Protein | KSQQNMLKIIQDDVNAKLEMFVNLDQLVKGEIKP | | |
| | KVSLINTAVSVSIPGQISNLQTKFLQKYVYLEES | | |
| | ITKQCTCNPLSGIFPTSGPTYPPTDKPDDDTTDD | | |
| | DKVDTTIKPIEYPKPDGCNRTGDHFTMEPGANFY | | |
| | TVPNLGPASSNSDECYTNPSFSIGSSIYMFSQEI | | |
| | RKTDCTAGEILSIQIVLGRIVDKGQQGPQASPLL | | |
| | VWAVPNPKIINSCAVAAGDEMGWVLCSVTLTAAS | | |
| | GEPIPHMFDGFWLYKLEPDTEVVSYRITGYAYLL | | |
| | DKQYDSVFIGKGGGIQKGNDLYFQMYGLSRNRQS | | |
| | FKALCEHGSCLGTGGGGYQVLCDRAVMSFGSEES | | |
| | LITNAYLKVNDLASGKPVIIGQTFPPSDSYKGSN | | |
| | GRMYTIGDKYGLYLAPSSWNRYLRFGITPDISVR | | |
| | STTWLKSQDPIMKILSTCTNTDRDMCPEICNTRG | | |
| | YQDIFPLSEDSEYYTYIGITPNNGGTKNFVAVRD | | |
| | SDGHIASIDILQNYYSITSATISCFMYKDEIWCI | | |
| | AITEGKKQKDNPQRIYAHSYKIRQMCYNMKSATV | | |
| | TVGNAKNITIRRY | | |

In some embodiments, the G protein has a sequence set forth in any of SEQ ID NOs: 1-11I or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs:1, 2, 3, 4, 5, 6, 7, 8, 9, 10 or 11. In some embodiments, the G protein has a sequence set forth in SEQ ID NO:1I or is a functionally active variant or biologically active portion thereof that has a sequence that is at least at or about 80%, at least at or about 90%, at least at or about 95%, or at least at or about 99% identical to SEQ ID NO: 1. In some embodiments, the G protein has a sequence set forth in SEQ ID NO:4 or is a functionally active variant or biologically active portion thereof that has a sequence that is at least at or about 80%, at least at or about 90%, at least at or about 95%, or at least at or about 99% identical to SEQ ID NO:4. In some embodiments, the G protein has a sequence set forth in SEQ ID NO:5 or is a functionally active variant or biologically active portion thereof that has a sequence that is at least at or about 80%, at least at or about 90%, at least at or about 95%, or at least at or about 99% identical to SEQ ID NO:5.

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 and retains fusogenic activity in conjunction with a Henipavirus F protein (e.g., NiV—F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 and retains fusogenic activity in conjunction with a Henipavirus F protein (e.g., NiV—F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:1, SEQ ID NO:4 or SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1, SEQ ID NO:4 or SEQ ID NO:5. In some embodiments, the G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1. In some embodiments, the G protein is a NiV-G protein that has the sequence set forth in SEQ ID NO:1. In some embodiments, the G protein is a NiV-G protein that has the sequence set forth in SEQ ID NO:4, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:4. In some embodiments, the G protein is a NiV-G protein that has the sequence set forth in SEQ ID NO:4. In some embodiments, the G protein is a NiV-G protein that has the sequence set forth in SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:5. In some embodiments, the G protein is a NiV-G protein that has the sequence set forth in SEQ ID NO:5.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:1, SEQ ID NO:4 or SEQ ID NO:5), up to 6 contiguous amino acid residues at or near the N-terminus of the wild-type NiV-G protein (SEQ ID NO:1, SEQ ID NO:4 or SEQ ID NO:5), up to 7 contiguous amino acid residues at or near the N-terminus of the wild-type NiV-G protein (SEQ ID NO:1, SEQ ID NO:4 or SEQ ID NO:5), up to 8 contiguous amino acid residues at or near the N-terminus of the wild-type NiV-G protein (SEQ ID NO:1, SEQ ID NO:4 or SEQ ID NO:5), up to 9 contiguous amino acid residues at or near the N-terminus of the wild-type NiV-G protein (SEQ ID NO:1, SEQ ID NO:4 or SEQ ID NO:5) up to 10 contiguous amino acid residues at or near the N-terminus of the wild-type NiV-G protein (SEQ ID NO:1, SEQ ID NO:4 or SEQ ID NO:5), up to 11 contiguous amino acid residues at or near the N-terminus of the wild-type NiV-G protein (SEQ ID NO:1, SEQ ID NO:4 or SEQ ID NO:5), up to 12 contiguous amino acid residues at or near the N-terminus of the wild-type NiV-G protein SEQ ID NO:1, SEQ ID NO:4, or SEQ ID NO:5), up to 13 contiguous amino acid residues at or near the N-terminus of the wild-type NiV-G protein (SEQ ID NO:1, SEQ ID NO:4 or SEQ ID NO:5), up to 14 contiguous amino acid residues at or near the N-terminus of the wild-type NiV-G protein (SEQ ID NO:1, SEQ ID NO:4 or SEQ ID NO:5), up to 15 contiguous amino acid residues at or near the N-terminus of the wild-type NiV-G protein (SEQ ID NO:1, SEQ ID NO:4 or SEQ ID NO:5) up to 16 contiguous amino acid residues at or near the N-terminus of the wild-type NiV-G protein (SEQ ID NO:1, SEQ ID NO:4 or SEQ ID NO:5), up to 17 contiguous amino acid residues at or near the N-terminus of the wild-type NiV-G protein (SEQ ID NO:1, SEQ ID NO:4 or SEQ ID NO:5), up to 18 contiguous amino acid residues at or near the N-terminus of the wild-type NiV-G protein (SEQ ID NO:1, SEQ ID NO:4 or SEQ ID NO:5), up to 19 contiguous amino acid residues at or near the N-terminus of the wild-type NiV-G protein (SEQ ID NO:1, SEQ ID NO:4 or SEQ ID NO:5), up to 20 contiguous amino acid residues at or near the N-terminus of the wild-type NiV-G protein (SEQ ID NO:1, SEQ ID NO:4 or SEQ ID NO:5), up to 21 contiguous amino acid residues at or near the N-terminus of the wild-type NiV-G protein SEQ ID NO:1, SEQ ID NO:4 or SEQ ID NO:5), up to 22 contiguous amino acid residues at or near the N-terminus of the wild-type NiV-G protein (SEQ ID NO:1, SEQ ID NO:4 or SEQ ID NO:5), up to 23 contiguous amino acid residues at or near the N-terminus of the wild-type NiV-G protein (SEQ ID NO:1, SEQ ID NO:4 or SEQ ID NO:5), up to 24 contiguous amino acid residues at or near the N-terminus of the wild-type NiV-G protein (SEQ ID NO:1, SEQ ID NO:4 or SEQ ID NO:5), up to 25 contiguous amino acid residues at or near the N-terminus of the wild-type NiV-G protein (SEQ ID NO:1, SEQ ID NO:4 or SEQ ID NO:5), up to 26 contiguous amino acid residues at or near the N-terminus of the wild-type NiV-G protein (SEQ ID NO:1, SEQ ID NO:4 or SEQ ID NO:5), up to 27 contiguous amino acid residues at or near the N-terminus of the wild-type NiV-G protein (SEQ ID NO:1, SEQ ID NO:4 or SEQ ID NO:5), up to 28 contiguous amino acid residues at or near the N-terminus of the wild-type NiV-G protein (SEQ ID NO:1, SEQ ID NO:4 or SEQ ID NO:5), up to 29 contiguous amino acid residues at or near the N-terminus of the wild-type NiV-G protein (SEQ ID NO:1, SEQ ID NO:4 or SEQ ID NO:5), up to 30 contiguous amino acid residues at or near the N-terminus of the wild-type NiV-G protein (SEQ ID NO:1, SEQ ID NO:4 or SEQ ID NO:5), up to 31 contiguous amino acid residues at or near the N-terminus of the wild-type NiV-G protein (SEQ ID NO:1, SEQ ID NO:4 or SEQ ID NO:5), up to 32 contiguous amino acid residues at or near the N-terminus of the wild-type NiV-G protein (SEQ ID NO:1, SEQ ID NO:4 or SEQ ID NO:5), up to 33 contiguous amino acid residues at or near the N-terminus of the wild-type NiV-G protein (SEQ ID NO:1, SEQ ID NO:4 or SEQ ID NO:5), up to 34 contiguous amino acid residues at or near the N-terminus of the wild-type NiV-G protein (SEQ ID NO:1, SEQ ID NO:4 or SEQ ID NO:5), up to 35 contiguous amino acid residues at or near the N-terminus of the wild-type NiV-G protein (SEQ ID NO:1, SEQ ID NO:4 or SEQ ID NO:5), up to 36 contiguous amino acid residues at or near the N-terminus of the wild-type NiV-G protein (SEQ ID NO:1, SEQ ID NO:4 or SEQ ID NO:5), up to 37 contiguous amino acid residues at or near the N-terminus of the wild-type NiV-G protein (SEQ ID NO:1, SEQ ID NO:4 or SEQ ID NO:5) up to 38 contiguous amino acid residues at or near the N-terminus of the wild-type NiV-G protein (SEQ ID NO:1, SEQ ID NO:4 or SEQ ID NO:5), up to 39 contiguous amino acid residues at or near the N-terminus of the wild-type NiV-G protein (SEQ ID NO:1, SEQ ID NO:4 or SEQ ID NO:5), up to 40 contiguous amino acid residues at or near the N-terminus of the wild-type NiV-G protein (SEQ ID NO:1, SEQ ID NO:4 or SEQ ID NO:5), up to 41 contiguous amino acid residues at or near the N-terminus of the wild-type NiV-G protein (SEQ ID NO:1, SEQ ID NO:4 or SEQ ID NO:5), up to 42 contiguous amino acid residues at or near the N-terminus of the wild-type NiV-G protein (SEQ ID NO:1, SEQ ID NO:4 or SEQ ID NO:5), up to 43 contiguous amino acid residues at or near the N-terminus of the wild-type NiV-G protein (SEQ ID NO:1, SEQ ID NO:4 or SEQ ID NO:5), up to 44 contiguous amino acid residues at or near the N-terminus of the wild-type NiV-G protein (SEQ ID NO:1, SEQ ID NO:4 or SEQ ID NO:5), or up to 45 contiguous amino acid residues at or near the N-terminus of the wild-type NiV-G protein (SEQ ID NO:1, SEQ ID NO:4 or SEQ ID NO:5).

In some embodiments, the mutant NiV-G protein is truncated and lacks 5 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:12. In some embodiments, the mutant NiV-G protein is truncated and lacks 10 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:44. In some embodiments, the mutant NiV-G protein is truncated and lacks 15 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:45. In some embodiments, the mutant NiV-G protein is truncated and lacks 20 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:13. In some embodiments, the mutant NiV-G protein is truncated and lacks 25 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:14. In some embodiments, the mutant NiV-G protein is truncated and lacks 30 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:43. In some embodiments, the mutant NiV-G protein is truncated and lacks 34 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:42.

In some embodiments, the NiV-G protein is a biologically active portion that does not contain a cytoplasmic domain. In some embodiments, the NiV-G protein without the cytoplasmic domain is encoded by SEQ ID NO:22.

In some embodiments, the mutant NiV-G protein comprises a sequence set forth in any of SEQ ID NOS: 12-14, 17, 18 and 22, or 42-45 or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 12-14, 17, 18 and 22 or 42-45.

In some embodiments, the mutant NiV-G protein has a 5 amino acid truncation at or near the N-terminus of the wild-type NiV-G protein (SEQ ID NO:1, SEQ ID NO:4 or SEQ ID NO:5), such as set forth in SEQ ID NO:12 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12 or such as set forth in SEQ ID NO:17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In some embodiments, the mutant NiV-G protein has a 10 amino acid truncation at or near the N-terminus of the wild-type NiV-G protein (SEQ ID NO:1, SEQ ID NO:4 or SEQ ID NO:5), such as set forth in SEQ ID NO:44 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44. In some embodiments, the mutant NiV-G protein has a 20 amino acid truncation at or near the N-terminus of the wild-type NiV-G protein (SEQ ID NO:1, SEQ ID NO:4 or SEQ ID NO:5), such as set forth in SEQ ID NO:13 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13. In some embodiments, the mutant NiV-G protein has a 25 amino acid truncation at or near the N-terminus of the wild-type NiV-G protein (SEQ ID NO:1, SEQ ID NO:4 or SEQ ID NO:5), such as set forth in SEQ ID NO:14 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some embodiments, the mutant NiV-G protein has a 33 amino acid truncation at or near the N-terminus of the wild-type NiV-G protein (SEQ ID NO:1, SEQ ID NO:4 or SEQ ID NO:5), such as set forth in SEQ ID NO:17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In some embodiments, the mutant NiV-G protein has a 34 amino acid truncation at or near the N-terminus of the wild-type NiV-G protein (SEQ ID NO:1, SEQ ID NO:4 or SEQ ID NO:5), such as set forth in SEQ ID NO:18 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In some embodiments, the mutant NiV-G protein has a 48 amino acid truncation at or near the N-terminus of the wild-type NiV-G protein (SEQ ID NO:1, SEQ ID NO:4 or SEQ ID NO:5), such as set forth in SEQ ID NO:22 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the mutant NiV-G protein has a 15 amino acid truncation at or near the N-terminus of the wild-type NiV-G protein (SEQ ID NO:1, SEQ ID NO:4, or SEQ ID NO:5), such as set forth in SEQ ID NO:45 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some embodiments, the mutant NiV-G protein has a 20 amino acid truncation at or near the N-terminus of the wild-type NiV-G protein (SEQ ID NO:1, SEQ ID NO:4, or SEQ ID NO:5), such as set forth in SEQ ID NO:13 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

In some embodiments, the mutant NiV-G protein has a 25 amino acid truncation at or near the N-terminus of the wild-type NiV-G protein (SEQ ID NO:1, SEQ ID NO:4, or SEQ ID NO:5), such as set forth in SEQ ID NO:14 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

In some embodiments, the mutant NiV-G protein has a 30 amino acid truncation at or near the N-terminus of the wild-type NiV-G protein (SEQ ID NO:1, SEQ ID NO:4, or SEQ ID NO:5), such as set forth in SEQ ID NO:43 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:43.

In some embodiments, the mutant NiV-G protein has a 34 amino acid truncation at or near the N-terminus of the wild-type NiV-G protein (SEQ ID NO:1, SEQ ID NO:4, or SEQ ID NO:5), such as set forth in SEQ ID NO:42 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:42.

In some embodiments, the mutant NiV-G protein has a 48 amino acid truncation at or near the N-terminus of the wild-type NiV-G protein (SEQ ID NO:1, SEQ ID NO:4, or SEQ ID NO:5), such as set forth in SEQ ID NO:22 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the G protein is a mutant HeV-G protein that is a biologically active portion of a wild-type HeV-G. In some embodiments, the biologically active portion is an N-terminally truncated fragment.

In some embodiments, the G protein is a wild-type HeV-G protein that has the sequence set forth in SEQ ID NO:23 or 24, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23 or 24.

In some embodiments, the G protein is a mutant HeV-G protein that is a biologically active portion of a wild-type HeV-G (SEQ ID NO:23 or SEQ ID NO:24). In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:23 or 24), up to 6 contiguous amino acid residues at or near the N-terminus of the wild-type HeV-G protein (SEQ ID NO:23 or 24), up to 7 contiguous amino acid residues at or near the N-terminus of the wild-type HeV-G protein (SEQ ID NO:23 or 24) or up to 8 contiguous amino acid residues at or near the N-terminus of the wild-type HeV-G protein (SEQ ID NO:23 or 24), up to 9 contiguous amino acid residues at or near the N-terminus of the wild-type HeV-G protein (SEQ ID NO:23 or 24), up to 10 contiguous amino acid residues at or near the N-terminus of the wild-type HeV-G protein (SEQ ID NO:23 or 24), up to 11 contiguous amino acid residues at or near the N-terminus of the wild-type HeV-G protein (SEQ ID NO:23 or 24), up to 12 contiguous amino acid residues at or near the N-terminus of the wild-type HeV-G protein (SEQ ID NO:23 or 24), up to 13 contiguous amino acid residues at or near the N-terminus of the wild-type HeV-G protein (SEQ ID NO:23 or 24), up to 14 contiguous amino acid residues at or near the N-terminus of the wild-type HeV-G protein (SEQ ID NO:23 or 24), up to 15 contiguous amino acid residues at or near the N-terminus of the wild-type HeV-G protein (SEQ ID NO:23 or 24), up to 16 contiguous amino acid residues at or near the N-terminus of the wild-type HeV-G protein (SEQ ID NO:23 or 24), up to 17 contiguous amino acid residues at or near the N-terminus of the wild-type HeV-G protein (SEQ ID NO:23 or 24), up to 18 contiguous amino acid residues at or near the N-terminus of the wild-type HeV-G protein (SEQ ID NO:23 or 24), up to 19 contiguous amino acid residues at or near the N-terminus of the wild-type HeV-G protein (SEQ ID NO:23 or 24), up to 20 contiguous amino acid residues at or near the N-terminus of the wild-type HeV-G protein (SEQ ID NO:23 or 24), up to 21 contiguous amino acid residues at or near the N-terminus of the wild-type HeV-G protein (SEQ ID NO:23 or 24), up to 22 contiguous amino acid residues at or near the N-terminus of the wild-type HeV-G protein (SEQ ID NO:23 or 24), up to 23 contiguous amino acid residues at or near the N-terminus of the wild-type HeV-G protein SEQ ID NO:23 or 24), up to 24 contiguous amino acid residues at or near the N-terminus of the wild-type HeV-G protein (SEQ ID NO:23 or 24), up to 25 contiguous amino acid residues at or near the N-terminus of the wild-type HeV-G protein (SEQ ID NO:23 or 24), up to 26 contiguous amino acid residues at or near the N-terminus of the wild-type HeV-G protein (SEQ ID NO:23 or 24), up to 27 contiguous amino acid residues at or near the N-terminus of the wild-type HeV-G protein (SEQ ID NO:23 or 24), up to 28 contiguous amino acid residues at or near the N-terminus of the wild-type HeV-G protein (SEQ ID NO:23 or 24), up to 29 contiguous amino acid residues at or near the N-terminus of the wild-type HeV-G protein (SEQ ID NO:23 or 24), up to 30 contiguous amino acid residues at or near the N-terminus of the wild-type HeV-G protein (SEQ ID NO:23 or 24), up to 31 contiguous amino acid residues at or near the N-terminus of the wild-type HeV-G protein (SEQ ID NO:23 or 24), up to 32 contiguous amino acid residues at or near the N-terminus of the wild-type HeV-G protein (SEQ ID NO:23 or 24), up to 33 contiguous amino acid residues at or near the N-terminus of the wild-type HeV-G protein (SEQ ID NO:23 or 24), up to 34 contiguous amino acid residues at or near the N-terminus of the wild-type HeV-G protein (SEQ ID NO:23 or 24), up to 35 contiguous amino acid residues at or near the N-terminus of the wild-type HeV-G protein (SEQ ID NO:23 or 24), up to 36 contiguous amino acid residues at or near the N-terminus of the wild-type HeV-G protein (SEQ ID NO:23 or 24), up to 37 contiguous amino acid residues at or near the N-terminus of the wild-type HeV-G protein (SEQ ID NO:23 or 24), up to 38 contiguous amino acid residues at or near the N-terminus of the wild-type HeV-G protein (SEQ ID NO:23 or 24), up to 39 contiguous amino acid residues at or near the N-terminus of the wild-type HeV-G protein (SEQ ID NO:23 or 24), up to 40 contiguous amino acid residues at or near the N-terminus of the wild-type HeV-G protein (SEQ ID NO:23 or 24), up to 41 contiguous amino acid residues at or near the N-terminus of the wild-type HeV-G protein (SEQ ID NO:23 or 24), up to 42 contiguous amino acid residues at or near the N-terminus of the wild-type HeV-G protein (SEQ ID NO:23 or 24), up to 43 contiguous amino acid residues at or near the N-terminus of the wild-type HeV-G protein (SEQ ID NO:23 or 24), up to 44 contiguous amino acid residues at or near the N-terminus of the wild-type HeV-G protein (SEQ ID NO:23 or 24), or up to 45 contiguous amino acid residues at or near the N-terminus of the wild-type HeV-G protein (SEQ ID NO:23 or 24).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:23 or 24), such as set forth in SEQ ID NO:25 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:25. In some embodiments, the mutant HeV-G protein lacks the N-terminal cytoplasmic domain of the wild-type HeV-G protein (SEQ ID NO:23 or 24), such as set forth in SEQ ID NO:26 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26.

In some embodiments, the G protein or the functionally active variant or biologically active portion thereof binds to Ephrin B2 or Ephrin B3. In some aspects, the G protein has the sequence of amino acids set forth in any one of SEQ ID NO:24, SEQ ID NO:23, SEQ ID NO:4, SEQ ID NO:6, SEQ ID NO:5, SEQ ID NO:8 or SEQ ID NO:10, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:24, SEQ ID NO:23, SEQ ID NO:4, SEQ ID NO:6, SEQ ID NO:5, SEQ ID NO:8 or SEQ ID NO:10, or a functionally active variant or biologically active portion thereof, and retains binding to Ephrin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, and retains binding to Ephrin B2 or B3. Reference to retaining binding to Ephrin B2 or B3 includes binding that is at least or at least about 5% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 10% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 15% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 20% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 25% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion, 30% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 35% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 40% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 45% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 50% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 55% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 60% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 65% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 70% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10 or a functionally active variant or biologically active portion thereof, such as at least or at least about 75% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, such as at least or at least about 80% of the level or degree of binding of the corresponding wild-type G protein, such as set forth in SEQ ID NO:27, SEQ ID NO:23, SEQ ID NO:4, NO:4,SEQ ID NO:6, SEQ ID NO:5, SEQ ID NO:8 or SEQ ID NO:10, or a functionally active variant or biologically active portion thereof, such as at least or at least about 85% of the level or degree of binding of the corresponding wild-type G protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof, such as at least or at least about 90% of the level or degree of binding of the corresponding wild-type G protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof, or such as at least or at least about 95% of the level or degree of binding of the corresponding wild-type protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:4, SEQ ID NO:5 or SEQ ID NO:27, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4, SEQ ID NO:5 or SEQ ID NO:27 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:4, SEQ ID NO:5 or SEQ ID NO:27, 10% of the level or degree of binding of the corresponding wild-type NiV-G, such as set forth in SEQ ID NO:4, SEQ ID NO:5 or SEQ ID NO:27, 15% of the level or degree of binding of the corresponding wild-type NiV-G, such as set forth in SEQ ID NO:4, SEQ ID NO:5 or SEQ ID NO:27, 20% of the level or degree of binding of the corresponding wild-type NiV-G, such as set forth in SEQ ID NO:4, SEQ ID NO:5 or SEQ ID NO:27, 25% of the level or degree of binding of the corresponding wild-type NiV-G, such as set forth in SEQ ID NO:4, SEQ ID NO:5 or SEQ ID NO:27, 30% of the level or degree of binding of the corresponding wild-type NiV-G, such as set forth in SEQ ID NO:4, SEQ ID NO:5 or SEQ ID NO:27, 35% of the level or degree of binding of the corresponding wild-type NiV-G, such as set forth in SEQ ID NO:4, SEQ ID NO:5 or SEQ ID NO:27, 40% of the level or degree of binding of the corresponding wild-type NiV-G, such as set forth in SEQ ID NO:4, SEQ ID NO:5 or SEQ ID NO:27, 45% of the level or degree of binding of the corresponding wild-type NiV-G, such as set forth in SEQ ID NO:4, SEQ ID NO:5 or SEQ ID NO:27 50% of the level or degree of binding of the corresponding wild-type NiV-G, such as set forth in SEQ ID NO:4, SEQ ID NO:5 or SEQ ID NO:27, 55% of the level or degree of binding of the corresponding wild-type NiV-G, such as set forth in SEQ ID NO:4, SEQ ID NO:5 or SEQ ID NO:27, 60% of the level or degree of binding of the corresponding wild-type NiV-G, such as set forth in SEQ ID NO:4, SEQ ID NO:5 or SEQ ID NO:27, 65% of the level or degree of binding of the corresponding wild-type NiV-G, such as set forth in SEQ ID NO:4, SEQ ID NO:5 or SEQ ID NO:27, 70% of the level or degree of binding of the corresponding wild-type NiV-G, such as set forth in SEQ ID NO:4, SEQ ID NO:5 or SEQ ID NO:27, such as at least or at least about 75% of the level or degree of binding of the corresponding wild-type NiV-G, such as set forth in SEQ ID NO:4, SEQ ID NO:5 or SEQ ID NO:27, such as at least or at least about 80% of the level or degree of binding of the corresponding wild-type NIV-G, such as set forth in SEQ ID NO:4, SEQ ID NO:5 or SEQ ID NO:27, such as at least or at least about 85% of the level or degree of binding of the corresponding wild-type NiV-G, such as set forth in SEQ ID NO:4, SEQ ID NO:5 or SEQ ID NO:27, such as at least or at least about 90% of the level or degree of binding of the corresponding wild-type NiV-G, such as set forth in SEQ ID NO:4, SEQ ID NO:5 or SEQ ID NO:27, or such as at least or at least about 95% of the level or degree of binding of the corresponding wild-type NiV-G, such as set forth in SEQ ID NO:4, SEQ ID NO:5 or SEQ ID NO:27.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:23 or 24, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23 or 24 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO:23 or 24, 10% of the level or degree of binding of the corresponding wild-type HeV-G, such as set forth in SEQ ID NO:23 or 24, 15% of the level or degree of binding of the corresponding wild-type HeV-G, such as set forth in SEQ ID NO:23 or 24, 20% of the level or degree of binding of the corresponding wild-type HeV-G, such as set forth in SEQ ID NO:23 or 24, 25% of the level or degree of binding of the corresponding wild-type HeV-G, such as set forth in SEQ ID NO:23 or 24, 30% of the level or degree of binding of the corresponding wild-type HeV-G, such as set forth in SEQ ID NO:23 or 24, 35% of the level or degree of binding of the corresponding wild-type HeV-G, such as set forth in SEQ ID NO:23 or 24, 40% of the level or degree of binding of the corresponding wild-type HeV-G, such as set forth in SEQ ID NO:23 or 24, 45% of the level or degree of binding of the corresponding wild-type HeV-G, such as set forth in SEQ ID NO:23 or 24, 50% of the level or degree of binding of the corresponding wild-type HeV-G, such as set forth in SEQ ID NO:23 or 24, 55% of the level or degree of binding of the corresponding wild-type HeV-G, such as set forth in SEQ ID NO:23 or 24, 60% of the level or degree of binding of the corresponding wild-type HeV-G, such as set forth in SEQ ID NO:23 or 24, 65% of the level or degree of binding of the corresponding wild-type HeV-G, such as set forth in SEQ ID NO:23 or 24, 70% of the level or degree of binding of the corresponding wild-type HeV-G, such as set forth in SEQ ID NO:23 or 24, such as at least or at least about 75% of the level or degree of binding of the corresponding wild-type HeV-G, such as set forth in SEQ ID NO:23 or 24, such as at least or at least about 80% of the level or degree of binding of the corresponding wild-type NIV-G, such as set forth in SEQ ID NO:23 or 24, such as at least or at least about 85% of the level or degree of binding of the corresponding wild-type HeV-G, such as set forth in SEQ ID NO:23 or 24, such as at least or at least about 90% of the level or degree of binding of the corresponding wild-type HeV-G, such as set forth in SEQ ID NO:23 or 24, or such as at least or at least about 95% of the level or degree of binding of the corresponding wild-type HeV-G, such as set forth in SEQ ID NO:23 or 24.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:4.

In some embodiments, the G protein is a mutant G protein. In some embodiments, the G protein is a mutant G protein containing one or more amino acid substitutions selected from the group consisting of E501A, W504A, Q530A and E533A with reference to numbering set forth in SEQ ID NO:4. In some embodiments, the G protein is a mutant G protein that contains one or more amino acid substitutions elected from the group consisting of E501A, W504A, Q530A and E533A with reference to SEQ ID NO:4 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO:4), 6 contiguous amino acid residues at or near the N-terminus of the wild-type NiV-G protein (SEQ ID NO:4), 7 contiguous amino acid residues at or near the N-terminus of the wild-type NiV-G protein (SEQ ID NO:4), 8 contiguous amino acid residues at or near the N-terminus of the wild-type NiV-G protein (SEQ ID NO:4), 9 contiguous amino acid residues at or near the N-terminus of the wild-type NiV-G protein (SEQ ID NO:4), up to 10 contiguous amino acid residues at or near the N-terminus of the wild-type NiV-G protein (SEQ ID NO:4), 11 contiguous amino acid residues at or near the N-terminus of the wild-type NiV-G protein (SEQ ID NO:4), 12 contiguous amino acid residues at or near the N-terminus of the wild-type NiV-G protein (SEQ ID NO:4), 13 contiguous amino acid residues at or near the N-terminus of the wild-type NiV-G protein (SEQ ID NO:4), 14 contiguous amino acid residues at or near the N-terminus of the wild-type NiV-G protein (SEQ ID NO:4), up to 15 contiguous amino acid residues at or near the N-terminus of the wild-type NiV-G protein (SEQ ID NO:4), 16 contiguous amino acid residues at or near the N-terminus of the wild-type NiV-G protein (SEQ ID NO:4), 17 contiguous amino acid residues at or near the N-terminus of the wild-type NiV-G protein (SEQ ID NO:4), 18 contiguous amino acid residues at or near the N-terminus of the wild-type NiV-G protein (SEQ ID NO:4), 19 contiguous amino acid residues at or near the N-terminus of the wild-type NiV-G protein (SEQ ID NO:4), up to 20 contiguous amino acid residues at or near the N-terminus of the wild-type NiV-G protein (SEQ ID NO:4), 21 contiguous amino acid residues at or near the N-terminus of the wild-type NiV-G protein (SEQ ID NO:4) 22 contiguous amino acid residues at or near the N-terminus of the wild-type NiV-G protein (SEQ ID NO:4), 23 contiguous amino acid residues at or near the N-terminus of the wild-type NiV-G protein (SEQ ID NO:4), 24 contiguous amino acid residues at or near the N-terminus of the wild-type NiV-G protein (SEQ ID NO:4), up to 25 contiguous amino acid residues at or near the N-terminus of the wild-type NiV-G protein (SEQ ID NO:4), 26 contiguous amino acid residues at or near the N-terminus of the wild-type NiV-G protein (SEQ ID NO:4), 27 contiguous amino acid residues at or near the N-terminus of the wild-type NiV-G protein (SEQ ID NO:4), 28 contiguous amino acid residues at or near the N-terminus of the wild-type NiV-G protein (SEQ ID NO:4), 29 contiguous amino acid residues at or near the N-terminus of the wild-type NiV-G protein (SEQ ID NO:4), up to 30 contiguous amino acid residues at or near the N-terminus of the wild-type NiV-G protein (SEQ ID NO: 4), up to 31 contiguous amino acid residues at or near the N-terminus of the wild-type NiV-G protein (SEQ ID NO:4), 32 contiguous amino acid residues at or near the N-terminus of the wild-type NiV-G protein (SEQ ID NO:4), 33 contiguous amino acid residues at or near the N-terminus of the wild-type NiV-G protein (SEQ ID NO:4) 34 contiguous amino acid residues at or near the N-terminus of the wild-type NiV-G protein (SEQ ID NO:4), 35 contiguous amino acid residues at or near the N-terminus of the wild-type NiV-G protein (SEQ ID NO:4) up to 36 contiguous amino acid residues at or near the N-terminus of the wild-type NiV-G protein (SEQ ID NO:4), up to 37 contiguous amino acid residues at or near the N-terminus of the wild-type NiV-G protein (SEQ ID NO:4), up to 38 contiguous amino acid residues at or near the N-terminus of the wild-type NiV-G protein (SEQ ID NO: 4), up to 39 contiguous amino acid residues at or near the N-terminus of the wild-type NiV-G protein (SEQ ID NO:4), or up to 40 contiguous amino acid residues at or near the N-terminus of the wild-type NiV-G protein (SEQ ID NO: 4).

In some embodiments, the mutant NiV-G protein has the amino acid sequence set forth in SEQ ID NO:17 or 18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17 or 18. In particular embodiments, the G protein has the sequence of amino acids set forth in SEQ ID NO: 17 or 18. In some embodiments, the mutant NiV-G protein has the amino acid sequence set forth in SEQ ID NO:17 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In particular embodiments, the G protein has the sequence of amino acids set forth in SEQ ID NO 17. In some embodiments, the mutant NiV-G protein has the amino acid sequence set forth in SEQ ID NO:18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In particular embodiments, the G protein has the sequence of amino acids set forth in SEQ ID NO 18.

In some embodiments, the G protein is a mutant G protein containing one or more amino acid substitutions selected from the group consisting of E501A, W504A, Q530A and E533A with reference to numbering set forth in SEQ ID NO:4. In some embodiments, the G protein is a mutant G protein that contains one or more amino acid substitutions elected from the group consisting of E501A, W504A, Q530A and E533A with reference to SEQ ID NO:4 and is a biologically active portion thereof containing an N-terminal truncation.

b. F Proteins

In some embodiments, the vector-surface targeting moiety comprises a protein with a hydrophobic fusion peptide domain. In some embodiments, the vector-surface targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

Table 3 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:28). Following cleavage of the signal peptide, the mature F₀ (e.g. SEQ ID NO:29) is transported to the cell surface, then endocytosed and cleaved by cathepsin L into the mature fusogenic subunits F1 and F2. In some embodiments, the signal peptide comprises the amino acid sequence set forth in SEQ ID NO: 38. In some embodiments, the F₀ comprises the amino acid sequence of SEQ ID NO:41. In some embodiments, the F1 subunit comprises the sequence amino acid sequence set forth in SEQ ID NO:46. In some embodiments, the F2 subunit comprises the sequence amino acid sequence set forth in SEQ ID NO:39. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 3
F proteins

| | | | SEQ ID |
|---|---|---|---|
| | | | (without |
| Full Gene | | SEQ | signal |
| Name | Sequence | ID | sequence) |
| Hendra virus | MATQEVRLKCLLCGIIVLVLSLEGLGILHYEKLSKIGLV | 28 | 29 |
| F Protein | KGITRKYKIKSNPLTKDIVIKMIPNVSNVSKCTGTVMEN | | |
| | YKSRLTGILSPIKGAIELYNNNTHDLVGDVKLAGVVMAG | | |
| | IAIGIATAAQITAGVALYEAMKNADNINKLKSSIESTNE | | |
| | AVVKLQETAEKTVYVLTALQDYINTNLVPTIDQISCKQT | | |
| | ELALDLALSKYLSDLLFVFGPNLQDPVSNSMTIQAISQA | | |
| | FGGNYETLLRTLGYATEDFDDLLESDSIAGQIVYVDLSS | | |
| | YYIIVRVYFPILTEIQQAYVQELLPVSFNNDNSEWISIV | | |
| | PNFVLIRNTLISNIEVKYCLITKKSVICNQDYATPMTAS | | |
| | VRECLTGSTDKCPRELVVSSHVPRFALSGGVLFANCISV | | |
| | TCQCQTTGRAISQSGEQTLLMIDNTTCTTVVLGNIIISL | | |
| | GKYLGSINYNSESIAVGPPVYTDKVDISSQISSMNQSLQ | | |
| | QSKDYIKEAQKILDTVNPSLISMLSMIILYVLSIAALCI | | |
| | GLITFISFVIVEKKRGNYSRLDDRQVRPVSNGDLYYIGT | | |
| Nipah virus | MVVILDKRCYCNLLILILMISECSVGILHYEKLSKIGLV | 30 | 31 |
| F Protein | KGVTRKYKIKSNPLTKDIVIKMIPNVSNMSQCTGSVMEN | | |
| | YKTRLNGILTPIKGALEIYKNNTHDLVGDVRLAGVIMAG | | |
| | VAIGIATAAQITAGVALYEAMKNADNINKLKSSIESTNE | | |
| | AVVKLQETAEKTVYVLTALQDYINTNLVPTIDKISCKQT | | |
| | ELSLDLALSKYLSDLLFVFGPNLQDPVSNSMTIQAISQA | | |
| | FGGNYETLLRTLGYATEDFDDLLESDSITGQIIYVDLSS | | |
| | YYIIVRVYFPILTEIQQAYIQELLPVSFNNDNSEWISIV | | |
| | PNFILVRNTLISNIEIGFCLITKRSVICNQDYATPMTNN | | |
| | MRECLTGSTEKCPRELVVSSHVPRFALSNGVLFANCISV | | |
| | TCQCQTTGRAISQSGEQTLLMIDNTTCPTAVLGNVIISL | | |
| | GKYLGSVNYNSEGIAIGPPVFTDKVDISSQISSMNQSLQ | | |
| | QSKDYIKEAQRLLDTVNPSLISMLSMIILYVLSIASLCI | | |
| | GLITFISFIIVEKKRNTYSRLEDRRVRPTSSGDLYYIGT | | |
| Cedar Virus | MSNKRTTVLIIISYTLFYLNNAAIVGFDFDKLNKIGVVQ | 32 | 33 |
| F Protein | GRVLNYKIKGDPMTKDLVLKFIPNIVNITECVREPLSRY | | |
| | NETVRRLLLPIHNMLGLYLNNTNAKMTGLMIAGVIMGGI | | |
| | AIGIATAAQITAGFALYEAKKNTENIQKLTDSIMKTQDS | | |
| | IDKLTDSVGTSILILNKLQTYINNQLVPNLELLSCRQNK | | |
| | IEFDLMLTKYLVDLMTVIGPNINNPVNKDMTIQSLSLLF | | |
| | DGNYDIMMSELGYTPQDFLDLIESKSITGQIIYVDMENL | | |
| | YVVIRTYLPTLIEVPDAQIYEFNKITMSSNGGEYLSTIP | | |
| | NFILIRGNYMSNIDVATCYMTKASVICNQDYSLPMSQNL | | |
| | RSCYQGETEYCPVEAVIASHSPRFALTNGVIFANCINTI | | |
| | CRCQDNGKTITQNINQFVSMIDNSTCNDVMVDKFTIKVG | | |
| | KYMGRKDINNINIQIGPQIIIDKVDLSNEINKMNQSLKD | | |
| | SIFYLREAKRILDSVNISLISPSVQLFLIIISVLSFIIL | | |
| | LIIIVYLYCKSKHSYKYNKFIDDPDYYNDYKRERINGKA | | |
| | SKSNNIYYVGD | | |
| Mojiang | MALNKNMFSSLFLGYLLVYATTVQSSIHYDSLSKVGVIK | 34 | 35 |
| virus, | GLTYNYKIKGSPSTKLMVVKLIPNIDSVKNCTQKQYDEY | | |
| Tongguan 1 | KNLVRKALEPVKMAIDTMLNNVKSGNNKYRFAGAIMAGV | | |
| F Protein | ALGVATAATVTAGIALHRSNENAQAIANMKSAIQNTNEA | | |
| | VKQLQLANKQTLAVIDTIRGEINNNIIPVINQLSCDTIG | | |
| | LSVGIRLTQYYSEIITAFGPALQNPVNTRITIQAISSVF | | |
| | NGNFDELLKIMGYTSGDLYEILHSELIRGNIIDVDVDAG | | |
| | YIALEIEFPNLTLVPNAVVQELMPISYNIDGDEWVTLVP | | |
| | RFVLTRTTLLSNIDTSRCTITDSSVICDNDYALPMSHEL | | |
| | IGCLQGDTSKCAREKVVSSYVPKFALSDGLVYANCLNTI | | |
| | CRCMDTDTPISQSLGATVSLLDNKRCSVYQVGDVLISVG | | |
| | SYLGDGEYNADNVELGPPIVIDKIDIGNQLAGINQTLQE | | |
| | AEDYIEKSEEFLKGVNPSIITLGSMVVLYIFMILIAIVS | | |
| | VIALVLSIKLTVKGNVVRQQFTYTQHVPSMENINYVSH | | |
| Bat | MKKKTDNPTISKRGHNHSRGIKSRALLRETDNYSNGLIV | 36 | 37 |
| Paramyxo- | ENLVRNCHHPSKNNLNYTKTQKRDSTIPYRVEERKGHYP | | |
| virus | KIKHLIDKSYKHIKRGKRRNGHNGNIITIILLLILILKT | | |
| Eid_hel/ | QMSEGAIHYETLSKIGLIKGITREYKVKGTPSSKDIVIK | | |
| GH-M74a/ | LIPNVTGLNKCTNISMENYKEQLDKILIPINNIIELYAN | | |
| GHA/2009 F | STKSAPGNARFAGVIIAGVALGVAAAAQITAGIALHEAR | | |
| protein | QNAERINLLKDSISATNNAVAELQEATGGIVNVITGMQD | | |
| | YINTNLVPQIDKLQCSQIKTALDISLSQYYSEILTVFGP | | |
| | NLQNPVTTSMSIQAISQSFGGNIDLLLNLLGYTANDLLD | | |
| | LLESKSITGQITYINLEHYFMVIRVYYPIMTTISNAYVQ | | |
| | ELIKISFNVDGSEWVSLVPSYILIRNSYLSNIDISECLI | | |
| | TKNSVICRHDFAMPMSYTLKECLTGDTEKCPREAVVTSY | | |
| | VPRFAISGGVIYANCLSTTCQCYQTGKVIAQDGSQTLMM | | |
| | IDNQTCSIVRIEEILISTGKYLGSQEYNTMHVSVGNPVF | | |
| | TDKLDITSQISNINQSIEQSKFYLDKSKAILDKINLNLI | | |
| | GSVPISILFIIAILSLILSIITFVIVMIIVRRYNKYTPL | | |
| | INSDPSSRRSTIQDVYIIPNPGEHSIRSAARSIDRDRD | | |

In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37. In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

In particular embodiments, the F protein or the functionally active variant or biologically active portion thereof retains fusogenic activity in conjunction with a Henipavirus G protein, such as a G protein set forth in Section IV.A.2 (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:30).

In particular embodiments, the F protein has the sequence of amino acids set forth in SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 28-37. In some embodiments, the mutant F protein is truncated and lacks up to 20 contiguous amino acids, such as up to 19, 18, 17, 16, 15, 14, 13, 12, 11, 10, 9, 8, 7, 6, 5, 4, 3, 2, or 1 contiguous amino acids at the C-terminus of the wild-type F protein. In some embodiments, the mutant F protein comprises the sequence set forth in SEQ ID NO:15. In some embodiments, the mutant F protein comprises the sequence set forth in SEQ ID NO:20. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO:20. The encoding nucleic acid can encode a signal peptide sequence that has the sequence MVVILDKRCY CNLLILILMI SECSVG (SEQ ID NO:38). In some examples, the F protein is cleaved into an F1 subunit comprising the sequence set forth in SEQ ID NO:46 and an F2 subunit comprising the sequence set forth in SEQ ID NO:39.

In some embodiments, the F protein is a NiV-F protein that is encoded by a sequence of nucleotides encoding the sequence set forth in SEQ ID NO:30, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:30. In some embodiments, the F protein is a NiV-F protein that is encoded by a sequence of nucleotides encoding the sequence set forth in SEQ ID NO:30. In some embodiments, the NiV-F-protein has the sequence of set forth in 30, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to 30. In some embodiments, the NiV-F-protein has the sequence of set forth in 30. In In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO:46, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein or the functionally active variant or the biologically active portion thereof includes an F1 subunit that has the sequence set forth in SEQ ID NO:46, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:40). In some embodiments, the mutant NiV-F protein comprises an amino acid sequence set forth in SEQ ID NO:20. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20. In some embodiments, the mutant F protein contains an F1 protein that has the sequence set forth in SEQ ID NO:46. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein is a mutant NiV-F protein that is a biologically active portion thereof that comprises a 20 amino acid truncation at or near the C-terminus of the wild-type NiV-F protein (SEQ ID NO:40); and a point mutation on an N-linked glycosylation site. In some embodiments, the mutant NiV-F protein comprises an amino acid sequence set forth in SEQ ID NO:15. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15.

In some embodiments, the F protein is a mutant NiV-F protein that is a biologically active portion thereof that comprises a 25 amino acid truncation at or near the C-terminus of the wild-type NiV-F protein (SEQ ID NO:40). In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein is encoded by a nucleotide sequence that encodes the sequence set forth in SEQ ID NO:20. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20.

In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein comprises the amino acid sequence set forth in SEQ ID NO:21, or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21. In some embodiments, the NiV-F protein is encoded by a nucleotide sequence that encodes the sequence set forth in SEQ ID NO:21. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21.

### B. CD4 Binding Agents

In some embodiments, a viral vector described herein is re-targeted by virtue of a binding agent (e.g., a CD4 binding agent). For example, in some cases, a viral vector comprises a fusogen to facilitate the fusion of the viral vector to the membrane, and the fusogen is modified to comprise a CD4 binding agent to re-target the viral vector. In some cases, the fusogen comprises a Nipah virus F glycoprotein (NiV-F) or a biologically active portion thereof and a Nipah virus G glycoprotein (NiV-G) or a biologically active portion thereof. In some embodiments, the CD4 binding agent is fused to the NiV-G protein. Thus, in some cases, the viral vector is re-targeted by virtue of comprising a re-targeted fusogen comprising a NiV-G fused to a CD4 binding agent.

The viral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963,U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or targeting protein (e.g. the hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, a VHH domain can be conjugated to fusogens to redirect fusion activity towards cells that display the VHH binding target. In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s.

In some embodiments, the CD4 binding agent is a peptide.

In some embodiments, the CD4 binding agent is an antibody, such as a single-chain variable fragment (scFv).

In some embodiments, the CD4 binding agent comprises a CDR-H1, a CDR-H2, and a CDR-H3 comprising the amino acid sequence set forth in SEQ ID NO: 149, 150, and 151, respectively. In some embodiments, the CD4 binding agent comprises a CDR-L1, a CDR-L2, and a CDR-L3 comprising the amino acid sequence set forth in SEQ ID NO: 152, 153, and 154, respectively. In some embodiments, the CD4 binding agent comprises a CDR-H1, a CDR-H2, and a CDR-H3 comprising the amino acid sequence set forth in SEQ ID NO: 149, 150, and 151, respectively; and a CDR-L1, a CDR-L2, and a CDR-L3 comprising the amino acid sequence set forth in SEQ ID NO: 152, 153, and 154, respectively. In some embodiments, the CD4 binding agent comprises a CDR-H1, a CDR-H2, and a CDR-H3 comprising the amino acid sequence set forth in SEQ ID NO: 207, 208, and 209, respectively. In some embodiments, the CD4 binding agent comprises a CDR-L1, a CDR-L2, and a CDR-L3 comprising the amino acid sequence set forth in SEQ ID NO: 210, 211, and 154, respectively. In some embodiments, the CD4 binding agent comprises a CDR-H1, a CDR-H2, and a CDR-H3 comprising the amino acid sequence set forth in SEQ ID NO: 207, 208, and 209, respectively; and a CDR-L1, a CDR-L2, and a CDR-L3 comprising the amino acid sequence set forth in SEQ ID NO: 210, 211, and 154, respectively. In some embodiments, the CD4 binding agent comprises a CDR-H1, a CDR-H2, and a CDR-H3 comprising the amino acid sequence set forth in SEQ ID NO: 212, 213, and 209, respectively. In some embodiments, the CD4 binding agent comprises a CDR-L1, a CDR-L2, and a CDR-L3 comprising the amino acid sequence set forth in SEQ ID NO: 210, 211, and 154, respectively. In some embodiments, the CD4 binding agent comprises a CDR-H1, a CDR-H2, and a CDR-H3 comprising the amino acid sequence set forth in SEQ ID NO: 212, 213, and 209, respectively; and a CDR-L1, a CDR-L2, and a CDR-L3 comprising the amino acid sequence set forth in SEQ ID NO: 210, 211, and 154, respectively. In some embodiments, the CD4 binding agent comprises a heavy chain variable region (VH) comprising the amino acid sequence set forth in SEQ ID NO:155. In some embodiments, the CD4 binding agent comprises a light chain variable region (VL) comprising the amino acid sequence set forth in SEQ ID NO:156. In some embodiments, the CD4 binding agent comprises a heavy chain variable region (VH) comprising the amino acid sequence set forth in SEQ ID NO:155; and a light chain variable region (VL) comprising the amino acid sequence set forth in SEQ ID NO:156. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:157.

In some embodiments, the CD4 binding agent comprises a CDR-H1, a CDR-H2, and a CDR-H3 comprising the amino acid sequence set forth in SEQ ID NO: 158, 159, and 160, respectively. In some embodiments, the CD4 binding agent comprises a CDR-L1, a CDR-L2, and a CDR-L3 comprising the amino acid sequence set forth in SEQ ID NO: 161, 162, and 163, respectively. In some embodiments, the CD4 binding agent comprises a CDR-H1, a CDR-H2, and a CDR-H3 comprising the amino acid sequence set forth in SEQ ID NO: 158, 159, and 160, respectively; and a CDR-L1, a CDR-L2, and a CDR-L3 comprising the amino acid sequence set forth in SEQ ID NO: 161, 162, and 163, respectively. In some embodiments, the CD4 binding agent comprises a CDR-H1, a CDR-H2, and a CDR-H3 comprising the amino acid sequence set forth in SEQ ID NO: 214, 215, and 216, respectively. In some embodiments, the CD4 binding agent comprises a CDR-L1, a CDR-L2, and a CDR-L3 comprising the amino acid sequence set forth in SEQ ID NO: 217, 218, and 163, respectively. In some embodiments, the CD4 binding agent comprises a CDR-H1, a CDR-H2, and a CDR-H3 comprising the amino acid sequence set forth in SEQ ID NO: 214, 215, and 216, respectively; and a CDR-L1, a CDR-L2, and a CDR-L3 comprising the amino acid sequence set forth in SEQ ID NO: 217, 218, and 163, respectively. In some embodiments, the CD4 binding agent comprises a CDR-H1, a CDR-H2, and a CDR-H3 comprising the amino acid sequence set forth in SEQ ID NO: 219, 220, and 216, respectively. In some embodiments, the CD4 binding agent comprises a CDR-L1, a CDR-L2, and a CDR-L3 comprising the amino acid sequence set forth in SEQ ID NO: 217, 218, and 163, respectively. In some embodiments, the CD4 binding agent comprises a CDR-H1, a CDR-H2, and a CDR-H3 comprising the amino acid sequence set forth in SEQ ID NO: 219, 220, and 216, respectively; and a CDR-L1, a CDR-L2, and a CDR-L3 comprising the amino acid sequence set forth in SEQ ID NO: 217, 218, and 163, respectively. In some embodiments, the CD4 binding agent comprises a heavy chain variable region (VH) comprising the amino acid sequence set forth in SEQ ID NO:164. In some embodiments, the CD4 binding agent comprises a light chain variable region (VL) comprising the amino acid sequence set forth in SEQ ID NO:165. In some embodiments, the CD4 binding agent comprises a heavy chain variable region (VH) comprising the amino acid sequence set forth in SEQ ID NO:164; and a light chain variable region (VL) comprising the amino acid sequence set forth in SEQ ID NO:165. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:166.

In some embodiments, the CD4 binding agent comprises a CDR-H1, a CDR-H2, and a CDR-H3 comprising the amino acid sequence set forth in SEQ ID NO: 167, 168, and 169, respectively. In some embodiments, the CD4 binding agent comprises a CDR-L1, a CDR-L2, and a CDR-L3 comprising the amino acid sequence set forth in SEQ ID NO: 170, 171, and 172, respectively. In some embodiments, the CD4 binding agent comprises a CDR-H1, a CDR-H2, and a CDR-H3 comprising the amino acid sequence set forth in SEQ ID NO: 167, 168, and 169, respectively; and a CDR-L1, a CDR-L2, and a CDR-L3 comprising the amino acid sequence set forth in SEQ ID NO: 170, 171, and 172, respectively. In some embodiments, the CD4 binding agent comprises a CDR-H1, a CDR-H2, and a CDR-H3 comprising the amino acid sequence set forth in SEQ ID NO: 221, 222, 223, respectively. In some embodiments, the CD4 binding agent comprises a CDR-L1, a CDR-L2, and a CDR-L3 comprising the amino acid sequence set forth in SEQ ID NO: 224, 225, and 172, respectively. In some embodiments, the CD4 binding agent comprises a CDR-H1, a CDR-H2, and a CDR-H3 comprising the amino acid sequence set forth in SEQ ID NO: 221, 222, 223, respectively; and a CDR-L1, a CDR-L2, and a CDR-L3 comprising the amino acid sequence set forth in SEQ ID NO: 224, 225, and 172, respectively. In some embodiments, the CD4 binding agent comprises a CDR-H1, a CDR-H2, and a CDR-H3 comprising the amino acid sequence set forth in SEQ ID NO: 226, 227, 223, respectively. In some embodiments, the CD4 binding agent comprises a CDR-L1, a CDR-L2, and a CDR-L3 comprising the amino acid sequence set forth in SEQ ID NO: 224, 225, and 172, respectively. In some embodiments, the CD4 binding agent comprises a CDR-H1, a CDR-H2, and a CDR-H3 comprising the amino acid sequence set forth in SEQ ID NO: 226, 227, 223, respectively; and a CDR-L1, a CDR-L2, and a CDR-L3 comprising the amino acid sequence set forth in SEQ ID NO: 224, 225, and 172, respectively. In some embodiments, the CD4 binding agent comprises a heavy chain variable region (VH) comprising the amino acid sequence set forth in SEQ ID NO:173. In some embodiments, the CD4 binding agent comprises a light chain variable region (VL) comprising the amino acid sequence set forth in SEQ ID NO:174. In some embodiments, the CD4 binding agent comprises a heavy chain variable region (VH) comprising the amino acid sequence set forth in SEQ ID NO:173; and a light chain variable region (VL) comprising the amino acid sequence set forth in SEQ ID NO:174. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:175.

In some embodiments, the CD4 binding agent comprises a CDR-H1, a CDR-H2, and a CDR-H3 comprising the amino acid sequence set forth in SEQ ID NO: 176, 177, and 178, respectively. In some embodiments, the CD4 binding agent comprises a CDR-L1, a CDR-L2, and a CDR-L3 comprising the amino acid sequence set forth in SEQ ID NO: 179, 180, and 181, respectively. In some embodiments, the CD4 binding agent comprises a CDR-H1, a CDR-H2, and a CDR-H3 comprising the amino acid sequence set forth in SEQ ID NO: 176, 177, and 178, respectively; and a CDR-L1, a CDR-L2, and a CDR-L3 comprising the amino acid sequence set forth in SEQ ID NO: 179, 180, and 181, respectively. In some embodiments, the CD4 binding agent comprises a CDR-H1, a CDR-H2, and a CDR-H3 comprising the amino acid sequence set forth in SEQ ID NO: 228, 229, 230, respectively. In some embodiments, the CD4 binding agent comprises a CDR-L1, a CDR-L2, and a CDR-L3 comprising the amino acid sequence set forth in SEQ ID NO: 231, 232, and 181, respectively. In some embodiments, the CD4 binding agent comprises a CDR-H1, a CDR-H2, and a CDR-H3 comprising the amino acid sequence set forth in SEQ ID NO: 228, 229, 230, respectively; and a CDR-L1, a CDR-L2, and a CDR-L3 comprising the amino acid sequence set forth in SEQ ID NO: 231, 232, and 181, respectively. In some embodiments, the CD4 binding agent comprises a CDR-H1, a CDR-H2, and a CDR-H3 comprising the amino acid sequence set forth in SEQ ID NO: 233, 234, 230, respectively. In some embodiments, the CD4 binding agent comprises a CDR-L1, a CDR-L2, and a CDR-L3 comprising the amino acid sequence set forth in SEQ ID NO: 231, 232, and 181, respectively. In some embodiments, the CD4 binding agent comprises a CDR-H1, a CDR-H2, and a CDR-H3 comprising the amino acid sequence set forth in SEQ ID NO: 233, 234, 230, respectively; and a CDR-L1, a CDR-L2, and a CDR-L3 comprising the amino acid sequence set forth in SEQ ID NO: 231, 232, and 181, respectively. In some embodiments, the CD4 binding agent comprises a heavy chain variable region (VH) comprising the amino acid sequence set forth in SEQ ID NO:182. In some embodiments, the CD4 binding agent comprises a light chain variable region (VL) comprising the amino acid sequence set forth in SEQ ID NO:183. In some embodiments, the CD4 binding agent comprises a heavy chain variable region (VH) comprising the amino acid sequence set forth in SEQ ID NO:182; and a light chain variable region (VL) comprising the amino acid sequence set forth in SEQ ID NO:183. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:184.

In some embodiments, the CD4 binding agent comprises a CDR-H1, a CDR-H2, and a CDR-H3 comprising the amino acid sequence set forth in SEQ ID NO: 185, 186, and 187, respectively. In some embodiments, the CD4 binding agent comprises a CDR-L1, a CDR-L2, and a CDR-L3 comprising the amino acid sequence set forth in SEQ ID NO: 188, 171, and 189, respectively. In some embodiments, the CD4 binding agent comprises a CDR-H1, a CDR-H2, and a CDR-H3 comprising the amino acid sequence set forth in SEQ ID NO: 185, 186, and 187, respectively; and a CDR-L1, a CDR-L2, and a CDR-L3 comprising the amino acid sequence set forth in SEQ ID NO: 188, 171, and 189, respectively. In some embodiments, the CD4 binding agent comprises a CDR-H1, a CDR-H2, and a CDR-H3 comprising the amino acid sequence set forth in SEQ ID NO: 235, 236, and 237, respectively. In some embodiments, the CD4 binding agent comprises a CDR-L1, a CDR-L2, and a CDR-L3 comprising the amino acid sequence set forth in SEQ ID NO: 238, 239, and 189, respectively. In some embodiments, the CD4 binding agent comprises a CDR-H1, a CDR-H2, and a CDR-H3 comprising the amino acid sequence set forth in SEQ ID NO: 235, 236, and 237, respectively; and a CDR-L1, a CDR-L2, and a CDR-L3 comprising the amino acid sequence set forth in SEQ ID NO: 238, 239, and 189, respectively. In some embodiments, the CD4 binding agent comprises a CDR-H1, a CDR-H2, and a CDR-H3 comprising the amino acid sequence set forth in SEQ ID NO: 240, 241, and 237, respectively. In some embodiments, the CD4 binding agent comprises a CDR-L1, a CDR-L2, and a CDR-L3 comprising the amino acid sequence set forth in SEQ ID NO: 238, 239, and 189, respectively. In some embodiments, the CD4 binding agent comprises a CDR-H1, a CDR-H2, and a CDR-H3 comprising the amino acid sequence set forth in SEQ ID NO: 240, 241, and 237, respectively; and a CDR-L1, a CDR-L2, and a CDR-L3 comprising the amino acid sequence set forth in SEQ ID NO: 238, 239, and 189, respectively. In some embodiments, the CD4 binding agent comprises a heavy chain variable region (VH) comprising the amino acid sequence set forth in SEQ ID NO:190. In some embodiments, the CD4 binding agent comprises a light chain variable region (VL) comprising the amino acid sequence set forth in SEQ ID NO:191. In some embodiments, the CD4 binding agent comprises a heavy chain variable region (VH) comprising the amino acid sequence set forth in SEQ ID NO:190; and a light chain variable region (VL) comprising the amino acid sequence set forth in SEQ ID NO:191. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:192.

In some embodiments, the CD4 binding agent comprises a CDR-H1, a CDR-H2, and a CDR-H3 comprising the amino acid sequence set forth in SEQ ID NO: 193, 194, and 195, respectively. In some embodiments, the CD4 binding agent comprises a CDR-L1, a CDR-L2, and a CDR-L3 comprising the amino acid sequence set forth in SEQ ID NO: 196, 197, and 198, respectively. In some embodiments, the CD4 binding agent comprises a CDR-H1, a CDR-H2, and a CDR-H3 comprising the amino acid sequence set forth in SEQ ID NO: 193, 194, and 195, respectively; and a CDR-L1, a CDR-L2, and a CDR-L3 comprising the amino acid sequence set forth in SEQ ID NO: 196, 197, and 198, respectively. In some embodiments, the CD4 binding agent comprises a CDR-H1, a CDR-H2, and a CDR-H3 comprising the amino acid sequence set forth in SEQ ID NO: 242, 243, and 244, respectively. In some embodiments, the CD4 binding agent comprises a CDR-L1, a CDR-L2, and a CDR-L3 comprising the amino acid sequence set forth in SEQ ID NO: 245, 246, and 198, respectively. In some embodiments, the CD4 binding agent comprises a CDR-H1, a CDR-H2, and a CDR-H3 comprising the amino acid sequence set forth in SEQ ID NO: 242, 243, and 244, respectively; and a CDR-L1, a CDR-L2, and a CDR-L3 comprising the amino acid sequence set forth in SEQ ID NO: 245, 246, and 198, respectively. In some embodiments, the CD4 binding agent comprises a CDR-H1, a CDR-H2, and a CDR-H3 comprising the amino acid sequence set forth in SEQ ID NO: 247, 248, and 244, respectively. In some embodiments, the CD4 binding agent comprises a CDR-L1, a CDR-L2, and a CDR-L3 comprising the amino acid sequence set forth in SEQ ID NO: 245, 246, and 198, respectively. In some embodiments, the CD4 binding agent comprises a CDR-H1, a CDR-H2, and a CDR-H3 comprising the amino acid sequence set forth in SEQ ID NO: 247, 248, and 244, respectively; and a CDR-L1, a CDR-L2, and a CDR-L3 comprising the amino acid sequence set forth in SEQ ID NO: 245, 246, and 198, respectively. In some embodiments, the CD4 binding agent comprises a heavy chain variable region (VH) comprising the amino acid sequence set forth in SEQ ID NO:199. In some embodiments, the CD4 binding agent comprises a light chain variable region (VL) comprising the amino acid sequence set forth in SEQ ID NO:200. In some embodiments, the CD4 binding agent comprises a heavy chain variable region (VH) comprising the amino acid sequence set forth in SEQ ID NO:199; and a light chain variable region (VL) comprising the amino acid sequence set forth in SEQ ID NO:200. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:201.

In some embodiments, the CD4 binding agent is an antibody, such as a single domain antibody. In some embodiments, the antibody can be human or humanized. In some embodiments, the CD4 binding agent is a VHH. In some embodiments, the CD4 binding agent comprises a CDR-H1, a CDR-H2, and a CDR-H3 comprising the amino acid sequence set forth in SEQ ID NO: 145, 146, and 147, respectively. In some embodiments, the CD4 binding agent comprises a CDR-H1, a CDR-H2, and a CDR-H3 comprising the amino acid sequence set forth in SEQ ID NO: 202, 203, and 204, respectively. In some embodiments, the CD4 binding agent comprises a CDR-H1, a CDR-H2, and a CDR-H3 comprising the amino acid sequence set forth in SEQ ID NO: 205, 206, and 204, respectively. In some embodiments, the CD4 binding agent comprises the amino acid sequence set forth in SEQ ID NO:148.

In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the viral vector. In some embodiments, the CD4 binding agent is the only membrane bound non-viral sequence of the viral vector. In some embodiments, the viral vector does not contain a molecule that engages or stimulates T cells other than the CD4 binding agent.

In some embodiments, viral vectors may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

### V. Engineered Receptor Payloads

In some embodiments, a viral vector disclosed herein encodes an engineered receptor. In some embodiments, the cells for use in or administered in connection with the provided methods contain or are engineered to contain an engineered receptor, e.g., an engineered antigen receptor, such as a chimeric antigen receptor (CAR). Also provided are populations of such cells, compositions containing such cells and/or enriched for such cells, such as in which cells of a certain type such as T cells or CD4+ cells are enriched or selected. Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, gene transfer is accomplished without first stimulating the cells, such as by combining it with a stimulus that induces a response such as proliferation, survival, and/or activation, e.g., as measured by expression of a cytokine or activation marker, followed by introduction of the nucleic acids, e.g., by transduction, into the stimulated cells, and optionally incubation or expansion in culture to numbers sufficient for clinical applications.

The viral vectors may express recombinant receptors, such as antigen receptors including chimeric antigen receptors (CARs), and other antigen-binding receptors such as transgenic T cell receptors (TCRs). Also among the receptors are other chimeric receptors.

### A. Chimeric Antigen Receptors (CARs)

In some embodiments of the provided methods and uses, chimeric receptors, such as a chimeric antigen receptors, contain one or more domains that combine an antigen- or ligand-binding domain (e.g. antibody or antibody fragment) that provides specificity for a desired antigen (e.g., tumor antigen) with intracellular signaling domains. In some embodiments, the intracellular signaling domain is a stimulating or an activating intracellular domain portion, such as a T cell stimulating or activating domain according a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in W0200014257, WO2013126726, WO2012/129514, WO2014031687, WO2013/166321, WO2013/071154, WO2013/123061, U.S. patent app. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent app. No. EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in WO/2014055668. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., (2013) Nature Reviews Clinical Oncology, 10, 267-276; Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The recombinant receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the antigen binding domain of the CAR molecule comprises an antibody, an antibody fragment, an scFv, a Fv, a Fab, a (Fab′)₂, a single domain antibody (SdAb), a VH or VL domain, or a camelid VHH domain.

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD20 antibody; CD21 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen targeted by the receptor includes antigens associated with a B cell malignancy, such as any of a number of known B cell marker. In some embodiments, the antigen targeted by the receptor is CD20, CD19, CD22, ROR1, CD45, CD47, CD21, CD5, CD33, Igkappa, Iglambda, CD79a, CD79b or CD30.

In some embodiments, the CAR binds to CD19. In some embodiments, the CAR binds to CD22. In some embodiments, the CAR binds to CD19 and CD22. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antigen targeted by the antigen-binding domain is CD19. In some aspects, the antigen-binding domain of the recombinant receptor, e.g., CAR, and the antigen-binding domain binds, such as specifically binds or specifically recognizes, a CD19, such as a human CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the antigen is CD19. In some embodiments, the scFv contains a VH and a VL derived from an antibody or an antibody fragment specific to CD 19. In some embodiments, the antibody or antibody fragment that binds CD 19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Naim-1 and −16 cells expressing CD19 of human origin (Fing, N. R., et al. (1987). Leucocyte typing III. 302).

In some embodiments, the antibody portion of the recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, WO2014031687, U.S. Pat. No. 8,822,647 or published app. No. US 2014/0271635. In some embodiments, the constant region or portion is of a human IgG, such as IgG4 or IgG1.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an IT AM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcRIγ, CD16, OX40/CD134, CD3δ, CD3α, CD3γ, CD3δ, TCRα, TCRβ, TCRδ, CD32, CD64, CD64, CD45, CD5, CD9, CD22, CD37, CD80, CD86, CD40, CD40L/CD154, VEGFR2, FAS, and FGFR2B, or functional variant thereof. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD 137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or IT AMs. Examples of IT AM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD 16. For example, in some aspects, the CAR or other chimeric receptor is a chimeric molecule of CD3-zeta (CD3-z) or Fc receptor and a portion of one of CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB. In some aspects, the T cell costimulatory molecule is 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, a CD19 specific CAR includes an anti-CD19 single-chain antibody fragment (scFv), a transmembrane domain such as one derived from human CD8α, a 4-1BB (CD137) co-stimulatory signaling domain, and a CD3δ (signaling domain. In some embodiments, a CD22 specific CAR includes an anti-CD22 scFv, a transmembrane domain such as one derived from human CD8α, a 4-1BB (CD137) co-stimulatory signaling domain, and a CD3δ (signaling domain. In some embodiments, a CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3(signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

Also provided herein are cells comprising a chimeric antigen receptor (CAR). In some embodiments, a cell described herein comprises a polynucleotide encoding a chimeric antigen receptor (CAR) comprising an antigen binding domain. In some embodiments, a cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, epidermal growth factor receptors (EGFR) (including ErbB1/EGFR, ErbB2/HER2, ErbB3/HER3, and ErbB4/HER4), fibroblast growth factor receptors (FGFR) (including FGF1, FGF2, FGF3, FGF4, FGF5, FGF6, FGF7, FGF18, and FGF21), vascular endothelial growth factor receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs, T-cell alpha chains, T-cell β chains, T-cell γ chains, T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1BB), CD163, F4/80, IL-4Ra, Sca-1, CTLA-4, GITR, GARP, LAP, granzyme B, LFA-1, transferrin receptor, NKp46, perforin, CD4+, Th1, Th2, Th17, Th40, Th22, Th9, Tfh, canonical Treg. FoxP3+, Trl, Th3, Treg17, TREG; CDCP, NT5E, EpCAM, CEA, gpA33, mucins, TAG-72, carbonic anhydrase IX, PSMA, folate binding protein, gangliosides (e.g., CD2, CD3, GM2), Lewis-γ², VEGF, VEGFR 1/2/3, aVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, ANTXR1, folate receptor alpha (FRa), ERBB2 (Her2/neu), EphA2, IL-13Ra2, epidermal growth factor receptor (EGFR), mesothelin, TSHR, CD19, CD123, CD22, CD30, CD171, CS-1, CLL-1, CD33, EGFRvIII, GD2, GD3, BCMA, MUC16 (CA125), LiCAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-II receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gp100, bcr-abl, tyrosinase, Fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCRI, ADRB3, PANX3, GPR20, LY6K, OR51E2, TARP, WTi, NY-ESO-1, LAGE-la, MAGE-A1, legumain, HPV E6, E7, ETV6-AML, sperm protein 17, XAGE1, Tie 2, MAD-CT-1, MAD-CT-2, major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant. hTERT, sarcoma translocation breakpoints, ML-IAP, ERG (TMPRSS2 ETS fusion gene), NA17, PAX3, androgen receptor, cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIRI, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3E); CD3G (CD3γ); CD4; CD8; CD28; CD45; CD80 (B7-1); CD86 (B7-2); CD247 (CD3δ); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture syndrome, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3δ); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Examples of exemplary components of a CAR are described in Table 4. In provided aspects, the sequences of each component in a CAR can include any combination listed in Table 4.

TABLE 4
CAR components and Exemplary Sequences

| Component | Sequence |
|---|---|
| Extracellular binding domain | |
| Anti-CD19 scFv (FMC63) | DIQMTQTTSSLSASLGDRVTISCRASQDISKYLNWYQQKPD |
| SEQ ID NO: 63 | GTVKLLIYHTSRLHSGVPSRFSGSGSGTDYSLTISNLEQEDIA |
| | TYFCQQGNTLPYTFGGGTKLEITGSTSGSGKPGSGEGSTKG |
| | EVKLQESGPGLVAPSQSLSVTCTVSGVSLPDYGVSWIRQPP |
| | RKGLEWLGVIWGSETTYYNSALKSRLTIIKDNSKSQVFLKM |
| | NSLQTDDTAIYYCAKHYYYGGSYAMDYWGQGTSVTVSS |
| Anti-CD19 scFv (FMC63) | DIQMTQTTSSLSASLGDRVTISCRASQDISKYLNWYQQKPD |
| SEQ ID NO: 73 | GTVKLLIYHTSRLHSGVPSRFSGSGSGTDYSLTISNLEQEDIA |
| | TYFCQQGNTLPYTFGGGTKLEITGGGGSGGGGSGGGGSEV |
| | KLQESGPGLVAPSQSLSVTCTVSGVSLPDYGVSWIRQPPRK |
| | GLEWLGVIWGSETTYYNSALKSRLTIIKDNSKSQVFLKMNS |
| | LQTDDTAIYYCAKHYYYGGSYAMDYWGQGTSVTVSS |
| Spacer (e.g. hinge) | |
| IgG4 Hinge | ESKYGPPCPPCP |
| SEQ ID NO: 53 | |
| CD8 Hinge | TTTPAPRPPTPAPTIASQPLSLRPE |
| SEQ ID NO: 142 | |
| CD28 | IEVMYPPPYLDNEKSNGTIIHVKGKHLCPSPLFPGPSKP |
| SEQ ID NO: 51 | |
| Transmembrane | |
| CD8 | ACRPAAGGAVHTRGLDFACDIYIWAPLAGTCGVLLLSLVIT |
| SEQ ID NO: 141 | LYC |
| CD28 | FWVLVVVGGVLACYSLLVTVAFIIFWV |
| SEQ ID NO: 57 | |
| CD28 | FWVLVVVGGVLACYSLLVTVAFIIFWV |
| SEQ ID NO: 58 | |
| Costimulatory domain | |
| CD28 | RSKRSRLLHSDYMNMTPRRPGPTRKHYQPYAPPRDFAAYR |
| SEQ ID NO: 60 | S |
| 4-1BB | KRGRKKLLYIFKQPFMRPVQTTQEEDGCSCRFPEEEEGGCE |
| SEQ ID NO: 59 | L |
| Primary Signaling Domain | |
| CD3zeta | RVKFSRSADAPAYQQGQNQLYNELNLGRREEYDVLDKRR |
| SEQ ID NO: 61 | GRDPEMGGKPRRKNPQEGLYNELQKDKMAEAYSEIGMKG |
| | ERRRGKGHDGLYQGLSTATKDTYDALHMQALPPR |
| CD3zeta | RVKFSRSADAPAYKQGQNQLYNELNLGRREEYDVLDKRR |
| SEQ ID NO: 62 | GRDPEMGGKPRRKNPQEGLYNELQKDKMAEAYSEIGMKG |
| | ERRRGKGHDGLYQGLSTATKDTYDALHMQALPPR |

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD 137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some aspects, the spacer contains only a hinge region of an IgG, such as only a hinge of IgG4 or IgG1In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

### B. T Cell Receptors (TCRs)

In some embodiments, engineered cells, such as T cells, used in connection with the provided methods, uses, articles of manufacture or compositions are cells that express a T cell receptor (TCR) or antigen-binding portion thereof that recognizes a peptide epitope or T cell epitope of a target polypeptide, such as an antigen of a tumor, viral or autoimmune protein.

In some embodiments, a “T cell receptor” or “TCR” is a molecule that contains a variable a and b chains (also known as TCRalpha and TCRbeta, respectively) or a variable g and d chains (also known as TCRalpha and TCRbeta, respectively), or antigen-binding portions thereof, and which is capable of specifically binding to a peptide bound to an MHC molecule. In some embodiments, the TCR is in the ab form. Typically, TCRs that exist in alpha-beta and gamma-delta forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the ab form or gd form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable b chain of a TCR, sufficient to form a binding site for binding to a specific MHC-peptide complex. Generally, the variable chains of a TCR contain complementarity determining regions involved in recognition of the peptide, MHC and/or MHC-peptide complex.

### C. Multi-Targeting

In some embodiments, the cells used in connection with the provided methods, uses, articles of manufacture and compositions include cells employing multi-targeting strategies, such as expression of two or more genetically engineered receptors on the cell, each recognizing the same of a different antigen and typically each including a different intracellular signaling component. Such multi-targeting strategies are described, for example, in WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR) which is capable of inducing an activating or stimulatory signal to the cell, generally upon specific binding to the antigen recognized by the first receptor, e.g., the first antigen. In some embodiments, the cell further includes a second genetically engineered antigen receptor (e.g., CAR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-KB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-IBB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

Domain which upon successful signaling of the CAR induces expression of a cytokine gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and/or CAR-T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

### D. Chimeric Auto-Antibody Receptor (CAAR)

In some embodiments, the recombinant receptor is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g. an intracellular signaling domain or region of a CD3-zeta) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsgl) and Dsg3.

In some embodiments, the encoded nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et al., Blood. 2014 Apr. 24; 123(17):e68-78, herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the encoded nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the viral vector in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### E. Additional Descriptions of CARs

In certain embodiments, the cell may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 5 below.

TABLE 5
Exemplary sequences of signal peptides

| SEQ ID | | |
|---|---|---|
| NO: | Sequence | Description |
| 47 | MALPVTALLLPLALLLHAARP | CD8α signal peptide |
| 48 | METDTLLLWVLLLWVPGSTG | IgK signal peptide |
| 49 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The VH and the V_{L} may be connected in either order, i.e., V_{H}-linker-V_{L} or V_{L}-linker-V_{H}. Non-limiting examples of linkers include Whitlow linker, (G₄S)n (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 6 below.

TABLE 6
Exemplary sequences of hinge domains

| SEQ ID | | |
|---|---|---|
| NO: | Sequence | Description |
| 50 | TTTPAPRPPTPAPTIASQPLSLRPEACRPAAGGAVH | CD8α hinge domain |
| | TRGLDFACD | |
| 51 | IEVMYPPPYLDNEKSNGTIIHVKGKHLCPSPLFPGP | CD28 hinge domain |
| | SKP | |
| 52 | AAAIEVMYPPPYLDNEKSNGTIIHVKGKHLCPSPLF | CD28 hinge domain |
| | PGPSKP | |
| 53 | ESKYGPPCPPCP | IgG4 hinge domain |
| 54 | ESKYGPPCPSCP | IgG4 hinge domain |
| 55 | ESKYGPPCPPCPAPEFLGGPSVFLFPPKPKDTLMISR | IgG4 hinge-CH2-CH3 |
| | TPEVTCVVVDVSQEDPEVQFNWYVDGVEVHNAK | domain |
| | TKPREEQFNSTYRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD80, 4-1BB/CD137, CD28, CD34, CD4, FcRIγ, CD16, OX40/CD134, CD3δ, CD3α, CD3γ, CD3δ, TCRα, TCRβ, TCRδ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 7 provides the amino acid sequences of a few exemplary transmembrane domains.

TABLE 7
Exemplary sequences of transmembrane domains

| SEQ ID | | |
|---|---|---|
| NO: | Sequence | Description |
| 56 | IYIWAPLAGTCGVLLLSLVITLYC | CD8α transmembrane domain |
| 57 | FWVLVVVGGVLACYSLLVTVAFIIFWV | CD28 transmembrane domain |
| 58 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3δ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3δ domain, an ITAM, a CD28 domain, 4-1BB domain, or a functional variant thereof. Table 8 provides the amino acid sequences of a few exemplary intracellular costimulatory and/or signaling domains. In certain embodiments, as in the case of tisagenlecleucel as described below, the CD3δ (signaling domain of SEQ ID NO:99 may have a mutation, e.g., a glutamine (Q) to lysine (K) mutation, at amino acid position 14 (see SEQ ID NO:62).

TABLE 8
Exemplary sequences of intracellular costimulatory and/or signaling domains

| SEQ ID | | |
|---|---|---|
| NO: | Sequence | Description |
| 59 | KRGRKKLLYIFKQPFMRPVQTTQEEDGCSCR | 4-1BB costimulatory domain |
| | FPEEEEGGCEL | |
| 60 | RSKRSRLLHSDYMNMTPRRPGPTRKHYQPY | CD28 costimulatory domain |
| | APPRDFAAYRS | |
| 61 | RVKFSRSADAPAYQQGQNQLYNELNLGRRE | CD3ζ signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 62 | RVKFSRSADAPAYKQGQNQLYNELNLGRRE | CD3ζ signaling domain (with Q |
| | EYDVLDKRRGRDPEMGGKPRRKNPQEGLYN | to K mutation at position 14) |
| | ELQKDKMAEAYSEIGMKGERRRGKGHDGLY | |
| | QGLSTATKDTYDALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 9 below. In some embodiments, the CD19-specific scFv comprises or consists of an amino acid sequence set forth in SEQ ID NO:63, 64, or 69, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:63, 64, or 69. In some embodiments, the CD19-specific scFv may comprise one or more CDRs having amino acid sequences set forth in SEQ ID NOs: 65-67 and 70-72. In some embodiments, the CD19-specific scFv may comprise a light chain with one or more CDRs having amino acid sequences set forth in SEQ ID NOs: 65-67. In some embodiments, the CD19-specific scFv may comprise a heavy chain with one or more CDRs having amino acid sequences set forth in SEQ ID NOs: 70-72. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the VH and the V_{L} portions of the scFv is a Whitlow linker having an amino acid sequence set forth in SEQ ID NO:68. In some embodiments, the Whitlow linker may be replaced by a different linker, for example, a 3xG₄S linker having an amino acid sequence set forth in SEQ ID NO:143, which gives rise to a different FMC63-derived scFv having an amino acid sequence set forth in SEQ ID NO:73. In certain of these embodiments, the CD19-specific scFv comprises or consists of an amino acid sequence set forth in SEQ ID NO:73 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:73.

TABLE 9
Exemplary sequences of anti-CD19 scFv and components

| SEQ ID NO: | Amino Acid Sequence | Description |
|---|---|---|
| 63 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 164 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 65 | QDISKY | Anti-CD19 FMC63 scFv |
| | | light chain CDR1 |
| 66 | HTS | Anti-CD19 FMC63 scFv |
| | | light chain CDR2 |
| 67 | QQGNTLPYT | Anti-CD19 FMC63 scFv |
| | | light chain CDR3 |
| 68 | GSTSGSGKPGSGEGSTKG | Whitlow linker |
| 69 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 70 | GVSLPDYG | Anti-CD19 FMC63 scFv |
| | | heavy chain CDR1 |
| 71 | IWGSETT | Anti-CD19 FMC63 scFv |
| | | heavy chain CDR2 |
| 72 | AKHYYYGGSYAMDY | Anti-CD19 FMC63 scFv |
| | | heavy chain CDR3 |
| 73 | DIQMTQTTSSLSASLGDRVTISCRASQDI | Anti-CD19 FMC63 scFv |
| | SKYLNWYQQKPDGTVKLLIYHTSRLHS | entire sequence, with 3xG₄S |
| | GVPSRFSGSGSGTDYSLTISNLEQEDIAT | linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 143 | GGGGSGGGGSGGGGS | 3xG₄S linker |

In some embodiments, the extracellular binding domain of the CD 19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (( ) signaling domain. CD3(associates with T cell receptors (TCRs) to produce a signal and contains immunoreceptor tyrosine-based activation motifs (ITAMs). The CD3δ (signaling domain refers to amino acid residues from the cytoplasmic domain of the zeta chain that are sufficient to functionally transmit an initial signal necessary for T cell activation. In some embodiments, the CD3δ (signaling domain is human. In some embodiments, the CD3δ (signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:63 or SEQ ID NO:73, the CD8α hinge domain of SEQ ID NO:50, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:63 or SEQ ID NO:73, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:63 or SEQ ID NO:73, the CD28 hinge domain of SEQ ID NO:51, the CD28 transmembrane domain of SEQ ID NO:57, the CD28 costimulatory domain of SEQ ID NO:60, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:74 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:74 (see Table 10). The encoded CD19 CAR has a corresponding amino acid sequence set forth in SEQ ID NO:75 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:75, with the following components: CD8α signal peptide, FMC63 scFv (V_{L}—Whitlow linker-V_{H}), CD8α hinge domain, CD8α transmembrane domain, 4-1BB costimulatory domain, and CD3δ (signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3xG₄S linker-V_{H}), CD8α hinge domain, CD8α transmembrane domain, 4-1BB costimulatory domain, and CD3(signaling domain. The nucleotide and amino acid sequence of the CD19 CAR in tisagenlecleucel are provided in Table 10, with annotations of the sequences provided in Table 11.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}—Whitlow linker-V_{H}), IgG4 hinge domain, CD28 transmembrane domain, 4-1BB costimulatory domain, and CD3δ (signaling domain. The nucleotide and amino acid sequence of the CD19 CAR in lisocabtagene maraleucel are provided in Table 10, with annotations of the sequences provided in Table 12.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}—Whitlow linker-V_{H}), CD28 hinge domain, CD28 transmembrane domain, CD28 costimulatory domain, and CD3δ (signaling domain. The nucleotide and amino acid sequence of the CD19 CAR in axicabtagene ciloleucel are provided in Table 10, with annotations of the sequences provided in Table 13.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-a signal peptide, FMC63 scFv, CD28 hinge domain, CD28 transmembrane domain, CD28 costimulatory domain, and CD3(signaling domain.

In some embodiments, the polycistronic vector comprises an expression cassette that contains a nucleotide sequence encoding a CD19 CAR as set forth in SEQ ID NO: 76, 78, or 80, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 76, 78, or 80. The encoded CD19 CAR has a corresponding amino acid sequence set forth in SEQ ID NO: 77, 79, or 81, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 77, 79, or 81, respectively.

TABLE 10
Exemplary sequences of CD19 CARs

| SEQ ID NO: | Sequence | Description |
|---|---|---|
| 74 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattegcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaceggegcccaccatcgcgtcgcagc | |
| | ccctgtccctgcgcccagaggcgtgccggccagcggggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctogc | |
| 75 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 76 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagectgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaceggegcccaccategegtegcagcccctgtccctgc | |
| | gcccagaggcgtgccggccagcggggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 77 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 78 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcageggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagegaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 79 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 80 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 81 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 11
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3 × G₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 12
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 13
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 76, 78, or 80, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 76, 78, or 80. The encoded CD19 CAR has a corresponding amino acid sequence set forth in SEQ ID NO: 77, 79, or 81, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 77, 79, or 81, respectively.

### CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48.

In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3xG₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 14 below. In some embodiments, the CD20-specific scFv comprises or consists of an amino acid sequence set forth in SEQ ID NO:82, 83, or 87, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:82, 83, or 87. In some embodiments, the CD20-specific scFv may comprise one or more CDRs having amino acid sequences set forth in SEQ ID NOs: 84-86, 88, 89, and 144. In some embodiments, the CD20-specific scFv may comprise a light chain with one or more CDRs having amino acid sequences set forth in SEQ ID NOs: 84-86. In some embodiments, the CD20-specific scFv may comprise a heavy chain with one or more CDRs having amino acid sequences set forth in SEQ ID NOs: 88, 89, and 144. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 14
Exemplary sequences of anti-CD20 scFv and components

| SEQ ID NO: | Amino Acid Sequence | Description |
|---|---|---|
| 82 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 83 | DIVLTQSPAILSASPGEKVTMTCRASSS | Anti-CD20 Leu16 scFv |
| | VNYMDWYQKKPGSSPKPWIYATSNLA | light chain variable region |
| | SGVPARFSGSGSGTSYSLTISRVEAEDA | |
| | ATYYCQQWSFNPPTFGGGTKLEIK | |
| 84 | RASSSVNYMD | Anti-CD20 Leu16 scFv |
| | | light chain CDR1 |
| 85 | ATSNLAS | Anti-CD20 Leu16 scFv |
| | | light chain CDR2 |
| 86 | QQWSFNPPT | Anti-CD20 Leu16 scFv |
| | | light chain CDR3 |
| 87 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 88 | SYNMH | Anti-CD20 Leu16 scFv |
| | | heavy chain CDR1 |
| 89 | AIYPGNGDTSYNQKFKG | Anti-CD20 Leu16 scFv |
| | | heavy chain CDR2 |
| 144 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the CD20 CAR comprises a CD3 zeta (0 signaling domain, for example, a human CD3δ (signaling domain. In some embodiments, the CD3δ (signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the CD8α hinge domain of SEQ ID NO:50, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the CD28 hinge domain of SEQ ID NO:51, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the IgG4 hinge domain of SEQ ID NO:53 or SEQ ID NO:54, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the CD8α hinge domain of SEQ ID NO:50, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the CD28 hinge domain of SEQ ID NO:51, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:82, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3xG₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:90, 91, or 95, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:90, 91, or 95. In some embodiments, the CD22-specific scFv may comprise one or more CDRs having amino acid sequences set forth in SEQ ID NOs: 92-94 and 96-98. In some embodiments, the CD22-specific scFv may comprise a heavy chain with one or more CDRs having amino acid sequences set forth in SEQ ID NOs: 92-94. In some embodiments, the CD22-specific scFv may comprise a light chain with one or more CDRs having amino acid sequences set forth in SEQ ID NOs: 96-98. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the VH and the V_{L} of m971-L7 connected by a 3xG₄S linker. In other embodiments, the Whitlow linker may be used instead. In some embodiments, the amino acid sequences of the entire m971-L7-derived scFv (also referred to as m971-L7 scFv) and its different portions are provided in Table 15 below. In some embodiments, the CD22-specific scFv comprises or consists of an amino acid sequence set forth in SEQ ID NO:99, 100, or 104, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:99, 100, or 104. In some embodiments, the CD22-specific scFv may comprise one or more CDRs having amino acid sequences set forth in SEQ ID NOs: 101-103 and 105-107. In some embodiments, the CD22-specific scFv may comprise a heavy chain with one or more CDRs having amino acid sequences set forth in SEQ ID NOs: 101-103. In some embodiments, the CD22-specific scFv may comprise a light chain with one or more CDRs having amino acid sequences set forth in SEQ ID NOs: 105-107. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 15
Exemplary sequences of anti-CD22 scFv and components

| SEQ ID NO: | Amino Acid Sequence | Description |
|---|---|---|
| 90 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 91 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 92 | GDSVSSNSAA | Anti-CD22 m971 scFv |
| | | heavy chain CDR1 |
| 93 | TYYRSKWYN | Anti-CD22 m971 scFv |
| | | heavy chain CDR2 |
| 94 | AREVTGDLEDAFDI | Anti-CD22 m971 scFv |
| | | heavy chain CDR3 |
| 95 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 96 | QTIWSY | Anti-CD22 m971 scFv |
| | | light chain CDR1 |
| 97 | AAS | Anti-CD22 m971 scFv |
| | | light chain CDR2 |
| 98 | QQSYSIPQT | Anti-CD22 m971 scFv |
| | | light chain CDR3 |
| 99 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 100 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 101 | GDSVSSNSVA | Anti-CD22 m971-L7 scFv |
| | | heavy chain CDR1 |
| 102 | TYYRSTWYN | Anti-CD22 m971-L7 scFv |
| | | heavy chain CDR2 |
| 103 | AREVTGDLEDAFDI | Anti-CD22 m971-L7 scFv |
| | | heavy chain CDR3 |
| 104 | DIQMIQSPSSLSASVGDRVTITCRASQTI | Anti-CD22 m971-L7 scFv |
| | WSYLNWYRQRPGEAPNLLIYAASSLQS | light chain variable region |
| | GVPSRFSGRGSGTDFTLTISSLQAEDFAT | |
| | YYCQQSYSIPQTFGQGTKLEIK | |
| 105 | QTIWSY | Anti-CD22 m971-L7 scFv |
| | | light chain CDR1 |
| 106 | AAS | Anti-CD22 m971-L7 scFv |
| | | light chain CDR2 |
| 107 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the CD22 CAR comprises a CD3 zeta (δ) signaling domain, for example, a human CD3δ (signaling domain. In some embodiments, the CD3δ (signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the CD8α hinge domain of SEQ ID NO:50, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the CD28 hinge domain of SEQ ID NO:51, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the IgG4 hinge domain of SEQ ID NO:53 or SEQ ID NO:54, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the CD8α hinge domain of SEQ ID NO:50, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the CD28 hinge domain of SEQ ID NO:51, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:90 or SEQ ID NO:99, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 16 below. In some embodiments, the BCMA-specific extracellular binding domain comprises or consists of an amino acid sequence set forth in SEQ ID NO:108, 109, or 113, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:108, 109, or 113. In some embodiments, the BCMA-specific extracellular binding domain may comprise one or more CDRs having amino acid sequences set forth in SEQ ID NOs: 110-112 and 114-116. In some embodiments, the BCMA-specific extracellular binding domain may comprise a light chain with one or more CDRs having amino acid sequences set forth in SEQ ID NOs: 110-112. In some embodiments, the BCMA-specific extracellular binding domain may comprise a heavy chain with one or more CDRs having amino acid sequences set forth in SEQ ID NOs: 114-116. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 16 below. In some embodiments, the BCMA-specific extracellular binding domain comprises or consists of an amino acid sequence set forth in SEQ ID NO:117, 118, or 122, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, 118, or 122. In some embodiments, the BCMA-specific extracellular binding domain may comprise one or more CDRs having amino acid sequences set forth in SEQ ID NOs: 119-121 and 123-125. In some embodiments, the BCMA-specific extracellular binding domain may comprise a light chain with one or more CDRs having amino acid sequences set forth in SEQ ID NOs: 119-121. In some embodiments, the BCMA-specific extracellular binding domain may comprise a heavy chain with one or more CDRs having amino acid sequences set forth in SEQ ID NOs: 123-135. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 16 below. In some embodiments, the BCMA-specific extracellular binding domain comprises or consists of an amino acid sequence set forth in SEQ ID NO:126 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:126. In some embodiments, the BCMA-specific extracellular binding domain may comprise one or more CDRs having amino acid sequences set forth in SEQ ID NOs: 127-129. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 16 below. In some embodiments, the BCMA-specific extracellular binding domain comprises or consists of an amino acid sequence set forth in SEQ ID NO:130, 131, or 135, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 130, 131, or 135. In some embodiments, the BCMA-specific extracellular binding domain may comprise one or more CDRs having amino acid sequences set forth in SEQ ID NOs: 132-134 and 136-138. In some embodiments, the BCMA-specific extracellular binding domain may comprise a light chain with one or more CDRs having amino acid sequences set forth in SEQ ID NOs: 132-134. In some embodiments, the BCMA-specific extracellular binding domain may comprise a heavy chain with one or more CDRs having amino acid sequences set forth in SEQ ID NOs: 136-138. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 16
Exemplary sequences of anti-BCMA binder and components

| SEQ ID NO: | Amino Acid Sequence | Description |
|---|---|---|
| 108 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 109 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 110 | RASESVSVIGAHLIH | Anti-BCMA C11D5.3 |
| | | scFv light chain CDR1 |
| 111 | LASNLET | Anti-BCMA C11D5.3 |
| | | scFv light chain CDR2 |
| 112 | LQSRIFPRT | Anti-BCMA C11D5.3 |
| | | scFv light chain CDR3 |
| 113 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 114 | DYSIN | Anti-BCMA C11D5.3 |
| | | scFv heavy chain CDR1 |
| 115 | WINTETREPAYAYDFRG | Anti-BCMA C11D5.3 |
| | | scFv heavy chain CDR2 |
| 116 | DYSYAMDY | Anti-BCMA C11D5.3 |
| | | scFv heavy chain CDR3 |
| 117 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 118 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 119 | RASESVTILGSHLIY | Anti-BCMA C12A3.2 |
| | | scFv light chain CDR1 |
| 120 | LASNVQT | Anti-BCMA C12A3.2 |
| | | scFv light chain CDR2 |
| 121 | LQSRTIPRT | Anti-BCMA C12A3.2 |
| | | scFv light chain CDR3 |
| 122 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 123 | HYSMN | Anti-BCMA C12A3.2 |
| | | scFv heavy chain CDR1 |
| 124 | RINTESGVPIYADDFKG | Anti-BCMA C12A3.2 |
| | | scFv heavy chain CDR2 |
| 125 | DYLYSLDF | Anti-BCMA C12A3.2 |
| | | scFv heavy chain CDR3 |
| 126 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 127 | GFTFSSYA | Anti-BCMA FHVH33 |
| | | CDR1 |
| 128 | ISGSGDYI | Anti-BCMA FHVH33 |
| | | CDR2 |
| 129 | AKEGTGANSSLADY | Anti-BCMA FHVH33 |
| | | CDR3 |
| 130 | DIQMTQSPSSLSASVGDRVTITCRASQSI | Anti-BCMA CT103A |
| | SSYLNWYQQKPGKAPKLLIYAASSLQS | scFv entire sequence, with |
| | GVPSRFSGSGSGTDFTLTISSLQPEDFAT | Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 131 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 132 | QSISSY | Anti-BCMA CT103A |
| | | scFv light chain CDR1 |
| 133 | AAS | Anti-BCMA CT103A |
| | | scFv light chain CDR2 |
| 134 | QQKYDLLT | Anti-BCMA CT103A |
| | | scFv light chain CDR3 |
| 135 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 136 | GGSISSSSYY | Anti-BCMA CT103A |
| | | scFv heavy chain CDR1 |
| 137 | ISYSGST | Anti-BCMA CT103A |
| | | scFv heavy chain CDR2 |
| 138 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the BCMA CAR comprises a CD3 zeta (δ) signaling domain, for example, a human CD3δ (signaling domain. In some embodiments, the CD3δ (signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:50, the CD8α transmembrane domain of SEQ ID NO:56, the 4-1BB costimulatory domain of SEQ ID NO:59, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8(a signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:50, the CD8α transmembrane domain of SEQ ID NO:56, the CD28 costimulatory domain of SEQ ID NO:60, the CD3δ (signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:139 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:139 (see Table 17). The encoded BCMA CAR has a corresponding amino acid sequence set forth in SEQ ID NO:140 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:140, with the following components: CD8α signal peptide, CT103A scFv (V_{L}—Whitlow linker-V_{H}), CD8α hinge domain, CD8α transmembrane domain, 4-1BB costimulatory domain, and CD3δ (signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3δ (signaling domain.

TABLE 17
Exemplary sequences of BCMA CARs

| SEQ ID NO: | Sequence | Description |
|---|---|---|
| 139 | atggccttaccagtgaccgccttgctcctgccgctggccttgctgctcca | Exemplary BCMA |
| | cgccgccaggccggacatccagatgacccagtctccatectccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaategccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 140 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### VI. Manufacture and Administration of Engineered T Cells

In some embodiments, resting or non-activated T cells are engineered in vitro by contacting with a viral vector comprising a CD4 binding agent, such as by any of the methods described in Section II. In some aspects of the exemplary process for generating or manufacturing engineered cells, CD4+ cells are selected from human peripheral blood mononuclear cells (PBMCs), for example, that are obtained by leukapheresis, generating an enriched CD4+ cell composition. In some aspects, such cells can be cryopreserved. In some aspects, the CD4+ composition can be thawed and subject to steps for transduction and expansion.

In some aspects of the exemplary process for generating or manufacturing engineered cells, CD4+ cells are not stimulated, for example, in the presence of paramagnetic polystyrene-coated beads coupled to anti-CD3 and anti-CD28 antibodies. In some aspects, the stimulation is not carried out in media containing human recombinant IL-2, human recombinant IL-15, or N-Acetyl Cysteine (NAC). In some aspects, the cell culture media for does not include human recombinant IL-7. In some aspects, the CD4+ cells are not stimulated in the presence of any of anti-CD3 and/or anti-CD28 antibodies, IL-2, IL-15, N-acetyl-cysteine, or IL-7.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD4+ and CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokinesecretion profile, and/or degree of differentiation. With reference to the subject to be treated, the cells may be allogeneic and/or autologous. In some embodiments, the methods include isolating cells from the subject, preparing, processing, culturing, and/or engineering them, and re-introducing them into the same subject, before or after cryopreservation.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, at least a portion of the selection step includes incubation of cells with a selection reagent, e.g., to select for CD4+ T cells. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In particular embodiments, a biological sample, e.g., a sample of PBMCs or other white blood cells, are subjected to selection of CD4+ or CD8+ T cells, where both the negative and positive fractions are retained, such that the selected cells comprise CD4+ and CD8+ T cells. In particular embodiments, a biological sample, e.g., a sample of PBMCs or other white blood cells, are subjected to selection of CD8+ T cells, where both the negative and positive fractions are retained, and CD4+ T cells are selected from the negative fraction. In particular embodiments, a biological sample, e.g., a sample of PBMCs or other white blood cells, are subjected to selection of CD4+ T cells, where both the negative and positive fractions are retained, and CD4+ T cells are selected from the positive fraction.

In some embodiments, T cells are separated from a PBMC sample by negative selection of markers expressed on non-T cells, such as B cells, monocytes, or other white blood cells, such as CD14. In some embodiments, the separated T cells comprise CD4+ and CD8+ T cells. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD4+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long term survival, expansion, and/or engraftment following administration of compositions containing CD4+T cells or CD4+ and CD8+ T cells, which in some aspects is particularly robust in such sub-populations. See Blaeschke et al., Cancer Immunol. Immunother. (2018) 67(7):2155-57 and Zhang et al., Experimental Hematol. and Oncol. (2020) 9:34. In some embodiments, combining T_{CM}-enriched CD4+ T cells and CD4+ T cells further enhances efficacy.

In some embodiments, CD8+ cells are also further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long term survival, expansion, and/or engraftment following administration of compositions containing CD4+ and CD8+ T cells, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8+ T cells and CD4+ T cells further enhances efficacy.

In embodiments, memory T cells are present in both CD62L+ and CD62L-subsets of CD8+ peripheral blood lymphocytes, such as in a composition of CD4+ and CD8+ T cells. PBMC can be enriched for or depleted of CD62L-CD8+ and/or CD62L+CD8+ fractions, such as using anti-CD8 and anti-CD62L antibodies.

In certain embodiments, the one or more compositions is or includes a composition of CD4+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of CD4+ T cells contains less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free of or substantially free of CD8+ T cells. In some embodiments, the composition of enriched T cells consists essentially of CD4+ T cells.

In some embodiments, the methods for generating the engineered cells, e.g., for cell therapy in accord with any of provided methods, uses, articles of manufacture or compositions, include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. Thus, in some embodiments, a composition of CAR-positive T cells that has been engineered by transduction or transfection with a recombinant polynucleotide encoding the CAR, is cultivated under conditions that promote proliferation and/or expansion.

In one aspect, the T cells are engineered for reduced expression or lack of expression of MHC class I and/or MHC class II human leukocyte antigens, and have reduced expression or lack of expression of a T-cell receptor (TCR) complex. The primary T cells can be engineered overexpress CD47 and a chimeric antigen receptor (CAR) in addition to reduced expression or lack of expression of MHC class I and/or MHC class II human leukocyte antigens, and have reduced expression or lack expression of a T-cell receptor (TCR) complex. In some instances, the CAR is a CD19-specific CAR. In other instances, the CAR is a CD22-specific CAR. In some instances, the CAR is a bispecific CAR. In certain instances, the CAR is a CD19/CD22 bispecific CAR. Any of the cells can express a bispecific CAR that binds to CD19 and CD22.

In some embodiments, the T cells overexpress CD47 and a chimeric antigen receptor (CAR), and include a genomic modification of the B2M gene. In some embodiments, the T cells are engineered to overexpress CD47 and include a genomic modification of the CIITA gene. In some embodiments, the T cells are engineered to overexpress CD47 and a CAR, and include a genomic modification of the TRAC gene. In some embodiments, hypoimmune T cells and primary T cells overexpress CD47 and a CAR, and include a genomic modification of the TRB gene. In some embodiments, hypoimmune T cells and primary T cells overexpress CD47 and a CAR, and include one or more genomic modifications selected from the group consisting of the B2M, CIITA, TRAC, and TRB genes. In some embodiments, hypoimmune T cells and primary T cells overexpress CD47 and a CAR, and include genomic modifications of the B2M, CIITA, TRAC, and TRB genes. In some embodiments, the cells are B2M^{-/-}, CIITA^{-/-}, TRAC^{-/-}, CD47tg cells that also express chimeric antigen receptors.

In some embodiments, the cells are B2M^{-/-}, CIITA^{-/-}, TRB^{-/-}, CD47tg cells that also express chimeric antigen receptors. In some embodiments, the cells are B2M, CIITA, TRAC, TRB, CD47tg cells that also express chimeric antigen receptors. In many embodiments, the cells are B2M^{indel/indel}, CIITA^{indel/indel}, TRAC^{indel/indel}, CD47tg cells that also express chimeric antigen receptors. In many embodiments, the cells are B₂M^{indel/indel}, CIITA^{indel/indel}, TRB^{indel/indel}, CD47tg cells that also express chimeric antigen receptors. In many embodiments, the cells are B2M^{indel/indel}, CIITA^{indel/indel}, TRAC^{indel/indel}, TRB^{indel/indel}, CD47tg cells that also express chimeric antigen receptors. In some embodiments, the modified cells described are pluripotent stem cells, induced pluripotent stem cells, cells differentiated from such pluripotent stem cells and induced pluripotent stem cells, or primary T cells. Non-limiting examples of primary T cells include CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells.

In some embodiments, a CD47 transgene is inserted into a pre-selected locus of the cell. In some embodiments, a transgene encoding a CAR is inserted into a pre-selected locus of the cell. In many embodiments, a CD47 transgene and a transgene encoding a CAR are inserted into a pre-selected locus of the cell. The pre-selected locus can be a safe harbor locus. Non-limiting examples of a safe harbor locus includes the AAVS1 locus, the CCR5 locus, and the ROSA26 locus. In some embodiments, the pre-selected locus is selected from the group consisting of the B2M locus, the CIITA locus, the TRAC locus, and the TRB locus. In some embodiments, the pre-selected locus is the B2M locus. In some embodiments, the pre-selected locus is the CIITA locus. In some embodiments, the pre-selected locus is the TRAC locus. In some embodiments, the pre-selected locus is the TRB locus.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor locus. In many instances, a transgene encoding a CAR is inserted into a safe harbor locus. In some instances, a CD47 transgene is inserted into a B2M locus. In some instances, a transgene encoding a CAR is inserted into a B2M locus. In certain instances, a CD47 transgene is inserted into a CIITA locus. In certain instances, a transgene encoding a CAR is inserted into a CIITA locus. In particular instances, a CD47 transgene is inserted into a TRAC locus. In particular instances, a transgene encoding a CAR is inserted into a TRAC locus. In many other instances, a CD47 transgene is inserted into a TRB locus. In many other instances, a transgene encoding a CAR is inserted into a TRB locus. In some embodiments, a CD47 transgene and a transgene encoding a CAR are inserted into a safe harbor locus (e.g., the AAVS1 locus, the CCR5 locus, or the ROSA26 locus).

In many embodiments, a CD47 transgene and a transgene encoding a CAR are inserted into a safe harbor locus. In many embodiments, a CD47 transgene and a transgene encoding a CAR are controlled by a single promoter and are inserted into a safe harbor locus. In many embodiments, a CD47 transgene and a transgene encoding a CAR are controlled by their own promoters and are inserted into a safe harbor locus. In many embodiments, a CD47 transgene and a transgene encoding a CAR are inserted into a TRAC locus. In many embodiments, a CD47 transgene and a transgene encoding a CAR are controlled by a single promoter and are inserted into a TRAC locus. In many embodiments, a CD47 transgene and a transgene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2M locus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1 alpha promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1 alpha promoter and a transgene encoding a CAR is controlled by an EF1 alpha promoter. In other embodiments, expression of both a CD47 transgene and a transgene encoding a CAR is controlled by a single EF1 alpha promoter.

The present technology contemplates altering target polynucleotide sequences in any manner which is available to the skilled artisan utilizing a rare cutting nuclease or CRISPR/Cas system of the present technology. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

Methods and edited cells are also disclosed in WO2016/183041 and U.S. provisional patent application Ser. No. 63/133,171, each of which is incorporated by reference herein in its entirety.

As is described in further detail herein, provided herein are methods for treating a patient with a disorder through administration of hypoimmunogenic cells, particularly hypoimmunogenic T cells. As will be appreciated, for all the multiple embodiments described herein related to the timing and/or combinations of therapies, the administration of the cells is accomplished by a method or route which results in at least partial localization of the introduced cells at a desired site. The cells can be infused, implanted, or transplanted directly to the desired site, or alternatively be administered by any appropriate route which results in delivery to a desired location in the subject where at least a portion of the implanted cells or components of the cells remain viable. In some embodiments, the cells are not provided by subcutaneous (SC) or intramuscular (IM) administration to a subject. In some embodiments, the cells are provided by intravenous (IV) administration to a subject.

The engineered T cells described herein may be used in methods for treating a patient with a disorder that includes administration of a population of cells to a subject, e.g., a human patient, including any of those as described in Sections II and VIII.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### VII. Pharmaceutical Compositions and Methods of Manufacture

The present disclosure also provides, in some aspects, a pharmaceutical composition comprising a viral vector or T cell composition described herein and pharmaceutically acceptable carrier. The pharmaceutical compositions can include any of the described viral vectors.

In some embodiments, composition meets a pharmaceutical or good manufacturing practices (GMP) standard. In some embodiments, the composition is made according to good manufacturing practices (GMP). In some embodiments, the composition has a pathogen level below a predetermined reference value, e.g., is substantially free of pathogens. In some embodiments, the composition has a contaminant level below a predetermined reference value, e.g., is substantially free of contaminants. In some embodiments, the composition has low immunogenicity.

In some embodiments, provided herein are the use of pharmaceutical compositions of the invention or salts thereof to practice the methods of the invention. Such a pharmaceutical composition may consist of at least one compound or conjugate of the invention or a salt thereof in a form suitable for administration to a subject, or the pharmaceutical composition may comprise at least one compound or conjugate of the invention or a salt thereof, and one or more pharmaceutically acceptable carriers, one or more additional ingredients, or some combination of these. In some embodiments, the compound or conjugate of the invention may be present in the pharmaceutical composition in the form of a physiologically acceptable salt, such as in combination with a physiologically acceptable cation or anion, as is well known in the art.

In some embodiments, the relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. In some embodiments, the composition may comprise between 0.1% and 100% (w/w) active ingredient.

In some embodiments, pharmaceutical compositions that are useful in the methods of the invention may be suitably developed for intravenous, intratumoral oral, rectal, vaginal, parenteral, topical, pulmonary, intranasal, buccal, ophthalmic, or another route of administration. In some embodiments, a composition useful within the methods of the invention may be directly administered to the skin, vagina or any other tissue of a mammal. In some embodiments, formulations include liposomal preparations, resealed erythrocytes containing the active ingredient, and immunologically based formulations. In some embodiments, the route(s) of administration will be readily apparent to the skilled artisan and will depend upon any number of factors including the type and severity of the disease being treated, the type and age of the veterinary or human subject being treated, and the like.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, although the descriptions of pharmaceutical compositions provided herein are principally directed to pharmaceutical compositions that are suitable for ethical administration to humans, it will be understood by the skilled artisan that such compositions are generally suitable for administration to animals of all sorts. In some embodiments, modification of pharmaceutical compositions suitable for administration to humans in order to render the compositions suitable for administration to various animals is well understood, and the ordinarily skilled veterinary pharmacologist may design and perform such modification with merely ordinary, if any, experimentation. In some embodiments, subjects to which administration of the pharmaceutical compositions of the invention is contemplated include humans and other primates, mammals including commercially relevant mammals such as cattle, pigs, horses, sheep, cats, and dogs.

In some of any embodiments, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. In some embodiments, pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

In some embodiments, the carrier may be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity may be maintained, for example, by the use of a coating such as lecithin, by the maintenance of the required particle size in the case of dispersion and by the use of surfactants. In some embodiments, prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. In some embodiments, prolonged absorption of the injectable compositions may be brought about by including in the composition an agent that delays absorption, for example, aluminum monostearate or gelatin. In one embodiment, the pharmaceutically acceptable carrier is not DMSO alone.

In some embodiments, formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents.

In some embodiments, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

In some embodiments, the composition of the invention may comprise a preservative from about 0.005% to 2.0% by total weight of the composition. In some embodiments, the preservative is used to prevent spoilage in the case of exposure to contaminants in the environment. In some embodiments, examples of preservatives useful in accordance with the invention included but are not limited to those selected from the group consisting of benzyl alcohol, sorbic acid, parabens, imidurea and combinations thereof. In some embodiments, a particularly preferred preservative is a combination of about 0.5% to 2.0% benzyl alcohol and 0.05% to 0.5% sorbic acid.

In some embodiments, the composition preferably includes an anti-oxidant and a chelating agent that inhibits the degradation of the compound. In some embodiments, antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. In some embodiments, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. In some embodiments, the chelating agent is useful for chelating metal ions in the composition that may be detrimental to the shelf life of the formulation. In some embodiments, other suitable and equivalent antioxidants and chelating agents may be substituted therefore as would be known to those skilled in the art.

In some embodiments, liquid suspensions may be prepared using conventional methods to achieve suspension of the active ingredient in an aqueous or oily vehicle. In some embodiments, aqueous vehicles include, for example, water, and isotonic saline. In some embodiments, oily vehicles include, for example, almond oil, oily esters, ethyl alcohol, vegetable oils such as arachis, olive, sesame, or coconut oil, fractionated vegetable oils, and mineral oils such as liquid paraffin. In some embodiments, liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. In some embodiments, oily suspensions may further comprise a thickening agent. In some embodiments, suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. In some embodiments, dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In some embodiments, liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. In some embodiments, liquid solutions of the pharmaceutical composition of the invention may comprise each of the components described with regard to liquid suspensions, it being understood that suspending agents will not necessarily aid dissolution of the active ingredient in the solvent. In some embodiments, aqueous solvents include, for example, water, and isotonic saline. In some embodiments, oily solvents include, for example, almond oil, oily esters, ethyl alcohol, vegetable oils such as arachis, olive, sesame, or coconut oil, fractionated vegetable oils, and mineral oils such as liquid paraffin.

In some embodiments, powdered and granular formulations of a pharmaceutical preparation of the invention may be prepared using known methods. In some embodiments, formulations may be administered directly to a subject, used, for example, to form tablets, to fill capsules, or to prepare an aqueous or oily suspension or solution by addition of an aqueous or oily vehicle thereto. In some of any embodiments, formulations may further comprise one or more of dispersing or wetting agent, a suspending agent, and a preservative. Additional excipients, such as fillers and sweetening, flavoring, or coloring agents, may also be included in these formulations.

In some embodiments, a pharmaceutical composition of the invention may also be prepared, packaged, or sold in the form of oil-in-water emulsion or a water-in-oil emulsion. In some embodiments, the oily phase may be a vegetable oil such as olive or arachis oil, a mineral oil such as liquid paraffin, or a combination of these. In some embodiments, compositions further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. In some embodiments, emulsions may also contain additional ingredients including, for example, sweetening or flavoring agents.

### VIII. Methods of Delivery and Treatment

In some embodiments, the viral vector provided herein is capable of delivering (e.g., delivers) an exogenous agent to a target cell. Among provided methods herein are methods that comprise delivering an agent to a target cell, such as by any of the methods described in Section II. In some embodiments, the exogenous agent is an agent that is entirely heterologous or not produced or normally expressed by the target cell. In some embodiments, delivery of the exogenous agent to the target cell can provide a therapeutic effect to treat a disease or condition in the subject. The therapeutic effect may be by targeting, modulating or altering an antigen or protein present or expressed by the target cell that is associated with or involved in a disease or condition. The therapeutic effect may be by providing an exogenous agent in which the exogenous agent is a protein (or a nucleic acid encoding the protein, e.g., an mRNA encoding the protein) which is absent, mutant, or at a lower level than wild-type in the target cell. In some embodiments, the target cell is from a subject having a genetic disease, e.g., a monogenic disease, e.g., a monogenic intracellular protein disease.

The viral vectors described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In some embodiments, the disease or condition may be one that is treated by delivery of the exogenous agent contained in the administered viral vector to a target cell in the subject.

This disclosure also provides, in certain aspects, a method of administering a viral vector to a subject (e.g., a human subject), a target tissue, or a cell, comprising administering to the subject, or contacting the target tissue or the cell with a composition comprising a plurality of viral vectors described herein, a viral vector composition described herein, or a pharmaceutical composition described herein, thereby administering the viral vector composition to the subject.

This disclosure also provides, in certain aspects, a method of delivering an exogenous agent, for instance a therapeutic agent (e.g., a polypeptide, a nucleic acid, a metabolite, an organelle, or a subcellular structure), to a subject, a target tissue, or a cell, comprising administering to the subject, or contacting the target tissue or the cell with, a plurality of viral vectors described herein, a viral vector composition comprising a plurality of viral vectors described herein, or a pharmaceutical composition described herein, wherein the composition is administered in an amount and/or time such that the therapeutic agent is delivered.

This disclosure also provides, in certain aspects, a method of delivering a function to a subject, a target tissue, or a cell, comprising administering to the subject, or contacting the target tissue or the cell with, a plurality of viral vectors described herein, a viral vector composition comprising a plurality of viral vectors described herein, a viral vector composition described herein, or a pharmaceutical composition described herein, wherein the viral vector composition is administered in an amount and/or time such that the function is delivered via delivery by the viral vector composition of an exogenous agent (e.g., therapeutic agent) to the target tissue or the cell.

In some embodiments, the target cell or tissue is any such listed in any of WO 2020/102499, WO 2020/102485, WO 2019/222403, WO 2020/014209, and WO 2020/102503, each of which is hereby incorporated by reference in its entirety. In some embodiments, the target cell is a T cell. In some embodiments, the target cell is any of a CD4+ T cell, a CD8+ T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+ Foxp3+ regulatory T cell, or a CD4+FoxP3−type 1 regulatory T (Trl) cell), a helper T cell (e.g., a CD4+helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell. In some embodiments, the target cell is a CD4+ T cell. In some embodiments, the target cell is a non-CD4+ T cell and is in a composition comprising a CD4+ T cell.

### A. Delivery

In some embodiments, the viral vector delivers the exogenous agent to at least 40%, 45%, 50%, 55%, 60%, 65%, 70%, 75%, 80%, 85%, 90%, 95%, 96%, 97%, 98%, or 99% of the number of cells in the target cell population (e.g., CD4+ T cells). In some embodiments, the viral vector delivers at least 40%, 45%, 50%, 55%, 60%, 65%, 70%, 75%, 80%, 85%, 90%, 95%, 96%, 97%, 98%, or 99% of the exogenous agent to the target cell population (e.g., CD4+ T cells).

In some embodiments, the viral vector delivers at least 40%, 45%, 50%, 55%, 60%, 65%, 70%, 75%, 80%, 85%, 90%, 95%, 96%, 97%, 98%, or 99% more of the exogenous agent to the target cell population (e.g., CD4+ T cells) compared to a non-target cell population. In some embodiments, the viral vector delivers more exogenous agent to the target cell population based on the viral vector comprising a fusogen or re-target fusogen that facilitates binding to the target cell population, but not the non-target cell population. The viral vector can comprise any of the exemplary fusogens and re-targeted fusogens described herein. In some embodiments, when the plurality of viral vectors are contacted with a cell population comprising target cells (e.g., CD4+ T cells) and non-target cells, the exogenous agent is present in at least 10-fold more target cells than non-target cells. In some embodiments, when the plurality of viral vectors are contacted with a cell population comprising target cells (e.g., CD4+ T cells) and non-target cells, the exogenous agent is present at least 2-fold, 5-fold, 10-fold, 20-fold, or 50-fold higher in target cells than non-target cells and/or the exogenous agent is present at least 2-fold, 5-fold, 10-fold, 20-fold, or 50-fold higher in target cells than non-target cells. In some embodiments, the viral vectors of the plurality fuse at a higher rate with a target cell than with a non-target cell by at least 50%.

In some embodiments, the viral vector is capable of delivering (e.g., delivers) a nucleic acid to a target cell, e.g., to stably modify the genome of the target cell, e.g., for gene therapy. Similarly, in some embodiments, a method herein comprises delivering a nucleic acid to a target cell.

In some embodiments, a method herein comprises causing ligand presentation on the surface of a target cell by presenting cell surface ligands on the viral vector. In some embodiments, the viral vector is capable of causing cell death of the target cell. In some embodiments, the viral vector is from a NK source cell.

In some embodiments, a viral vector or target cell is capable of phagocytosis (e.g., of a pathogen). Similarly, in some embodiments, a method herein comprises causing phagocytosis.

In some embodiments, the viral vector comprises (e.g., is capable of delivering to the target cell) a membrane protein or a nucleic acid encoding the membrane protein. In

In some embodiments, the viral vector, e.g., fusosome, fuses at a higher rate with a target cell (e.g., a CD4+ T cells) than with a non-target cell based on the viral vector comprising a fusogen or re-target fusogen that facilitates binding to the target cell, but not the non-target cell. The viral vector can comprise any of the exemplary fusogens and re-targeted fusogens described herein. In some embodiments, the viral vector, e.g., fusosome, fuses at a higher rate with a target cell than with a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold. In some embodiments, the viral vector, e.g., fusosome, fuses at a higher rate with a target cell than with other viral vectors, e.g., by at least 10%, 20%, 30%, 40%, 50%, 60%, 70%, 80%, or 90%. In some embodiments, the viral vector, e.g., fusosome, fuses with target cells at a rate such that an exogenous agent or nucleic acid encoding an exogenous agent in the viral vector is delivered to at least 10%, 20%, 30%, 40%, 50%, 60%, 70%, 80%, or 90%, of target cells after 24, 48, or 72 hours. In embodiments, the amount of targeted fusion is about 30%-70%, 35%-65%, 40%-60%, 45%-55%, or 45%-50%. In embodiments, the amount of targeted fusion is about 20%-40%, 25%-35%, or 30%-35%.

In some embodiments, the fusogen is present at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, at least 10%, 20%, 30%, 40%, 50%, 60%, 70%, 80%, 90%, 95%, 96%, 97%, 98%, or 99% of the fusogen comprised by the viral vector is disposed in the cell membrane. In embodiments, the viral vector e also comprises fusogen internally, e.g., in the cytoplasm or an organelle. In some embodiments, the fusogen comprises (or is identified as comprising) about 0.1%, 0.2%, 0.3%, 0.4%, 0.5%, 0.6%, 0.7%, 0.8%, 0.9%, 1%, 5%, 10%, 11%, 12%, 13%, 14%, 15%, 20%, or more, or about 1-30%, 5-20%, 10-15%, 12-15%, 13-14%, or 13.6% of the total protein in a viral vector, e.g., as determined by a mass spectrometry assay. In embodiments, the fusogen comprises (or is identified as comprising) about 13.6% of the total protein in the viral vector. In some embodiments, the fusogen is (or is identified as being) more or less abundant than one or more additional proteins of interest. In an embodiment, the fusogen has (or is identified as having) a ratio to EGFP of about 140, 145, 150, 151, 152, 153, 154, 155, 156, 157 (e.g., 156.9), 158, 159, 160, 165, or 170. In another embodiment, the fusogen has (or is identified as having) a ratio to CD63 of about 2700, 2800, 2900, 2910 (e.g., 2912), 2920, 2930, 2940, 2950, 2960, 2970, 2980, 2990, or 3000, or about 1000-5000, 2000-4000, 2500-3500, 2900-2930, 2910-2915, or 2912.0, e.g., by a mass spectrometry assay. In an embodiment, the fusogen has (or is identified as having) a ratio to ARRDC1 of about 600, 610, 620, 630, 640, 650, 660 (e.g., 664.9), 670, 680, 690, or 700. In another embodiment, the fusogen has (or is identified as having) a ratio to GAPDH of about 50, 55, 60, 65, 70 (e.g., 69), 75, 80, or 85, or about 1-30%, 5-20%, 10-15%, 12-15%, 13-14%, or 13.6%. In another embodiment, the fusogen has (or is identified as having) a ratio to CNX of about 500, 510, 520, 530, 540, 550, 560 (e.g., 558.4), 570, 580, 590, or 600, or about 300-800, 400-700, 500-600, 520-590, 530-580, 540-570, 550-560, or 558.4, e.g., by a mass spectrometry assay.

### B. Systems for Delivery

Provided herein are methods of administering a lentiviral vector comprising a CD4 binding agent to a subject. In some embodiments the method comprises a) obtaining whole blood from the subject; b) collecting the fraction of blood containing leukocyte components including CD4+ T cells; c) contacting the leukocyte components including CD4+ T cells with a composition comprising the lentiviral vector to create a transfection mixture; and d) reinfusing the contacted leukocyte components including CD4+ T cells and/or the transfection mixture to the subject, thereby administering the lipid particle and/or payload gene to the subject. In some embodiments, the T cells (e.g. CD4+ T cells) are not activated during the method.

The method according to the present disclosure is capable of delivering a lentiviral particle to an ex vivo system. The method may include the use of a combination of various apheresis machine hardware components, a software control module, and a sensor module to measure citrate or other solute levels in-line to ensure the maximum accuracy and safety of treatment prescriptions, and the use of replacement fluids designed to fully exploit the design of the system according to the present methods. It is understood that components described for one system according to the present invention can be implemented within other systems according to the present invention as well.

In some embodiments, the method for administration of the lentiviral vector to the subject comprises the use of a blood processing set for obtaining the whole blood from the subject, a separation chamber for collecting the fraction of blood containing leukocyte components including CD4+ T cells, a contacting container for the contacting the CD4+ T cells with the composition comprising the lentiviral vector, and a further fluid circuit for reinfusion of CD4+ T cells to the patient. In some embodiments, the method further comprises any of i) a washing component for concentrating T cells, and ii) a sensor and/or module for monitoring cell density and/or concentration. In some embodiments, the methods allow processing of blood directly from the patient, transduction with the lentiviral vector, and reinfusion directly to the patient without any steps of selection for T cells or for CD4+ T cells. Further the methods also can be carried out without cryopreserving or freezing any cells before or between any one or more of the steps, such that there is no step of formulating cells with a cryoprotectant, e.g. DMSO. In some embodiments, the provided methods also do not include a lymphodepletion regimen. In some embodiments, the method including steps (a)-(d) can be carried out for a time of no more than 24 hours, such as between 2 hours and 12 hours, for example 3 hours to 6 hours.

In some embodiments, the method is performed in-line. In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile.

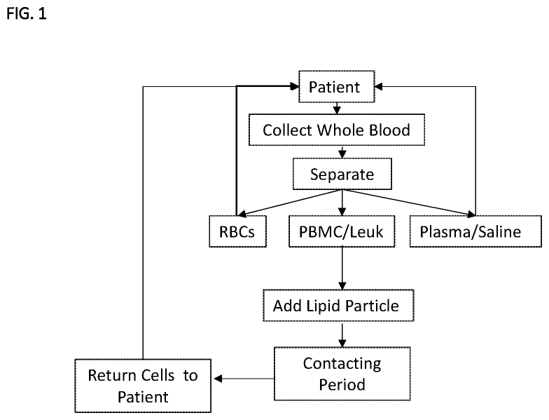

Also provided herein are systems for administration of a lentiviral vector comprising a CD4 binding agent to a subject, including any of those described in U.S. Patent Application No. 63/298,196, herein incorporated by reference in its entirety. An exemplary system for administration is shown in FIG. 1.

### C. Treatment and Uses

In some embodiments, the viral vectors provided herein, or pharmaceutical compositions thereof as described herein can be administered to a subject, e.g. a mammal, e.g. a human. In some embodiments, the administration delivers the viral vectors to a target cell (e.g., CD4+ T cells) in the subject. In such embodiments, the subject may be at risk of, may have a symptom of, or may be diagnosed with or identified as having, a particular disease or condition. In some embodiments, the methods thereby treat the disease or condition or disorder in the subject. In one embodiment, the subject has cancer. In one embodiment, the subject has an infectious disease. In some embodiments, the viral vector, e.g. retroviral particles other viral vectors or fusosomes thereof, contains nucleic acid sequences encoding an exogenous agent for treating the disease or condition in the subject. For example, the exogenous agent is one that targets or is specific for a protein of a neoplastic cells and the viral vector, e.g. retroviral particles other viral vectors or fusosomes thereof, is administered to a subject for treating a tumor or cancer in the subject. In another example, the exogenous agent is an inflammatory mediator or immune molecule, such as a cytokine, and the viral vector, e.g. retroviral particles other viral vectors or fusosomes thereof, is administered to a subject for treating any condition in which it is desired to modulate (e.g. increase) the immune response, such as a cancer or infectious disease. In some embodiments, the viral vector, e.g. retroviral particles other viral vectors or fusosomes thereof, is administered in an effective amount or dose to effect treatment of the disease, condition or disorder.

Provided herein are uses of any of the provided viral vectors, e.g. retroviral particles other viral vectors or fusosomes thereof, in such methods and treatments, and in the preparation of a medicament in order to carry out such therapeutic methods. In some embodiments, the methods are carried out by administering the viral vector, e.g. retroviral particles other viral vectors or fusosomes thereof, or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the viral vectors may be administered alone or formulated as a pharmaceutical composition. In some embodiments, the viral vectors or pharmaceutical compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the viral vectors may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

In some embodiments, the composition may be administered to a subject as frequently as several times daily, or it may be administered less frequently, such as once a day, once a week, once every two weeks, once a month, or even less frequently, such as once every several months or even once a year or less. In some embodiments, the amount of a composition may be administered, in non-limiting examples, every day, every other day, every 2 days, every 3 days, every 4 days, or every 5 days. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the compositions provided herein containing a provided viral vector such as any of the viral vectors or virus-based particles described herein, can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10 to about 10¹⁵ GC units, inclusive. In some embodiments, the dosage of administration of a viral vector or virus-like particle is from about 105 to about 10¹ GC units, inclusive. In some embodiments, the dosage of administration of a viral vector or virus-like particle is from about 106 to about 109 GC units, inclusive. In some embodiments, the dosage of administration of a viral vector or virus-like particle is from about 10′ to about 10¹² GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10¹² infectious units. In some embodiments, the dosage of administration of a viral vector or virus-like particle is from about 10⁹ to about 10¹² infectious units, inclusive. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive. In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10⁹ to about 10¹² pfu, inclusive. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some aspects, the dosage of administration of a vehicle within the pharmaceutical compositions provided herein varies depending on a subject's body weight. For example, a composition may be formulated as GC/kg, infectious units/kg, pfu/kg, etc. In some aspects, the dosage at which a therapeutic effect is obtained is from at or about 10⁸ GC/kg to at or about 10¹⁴ GC/kg of the subject's body weight, inclusive. In some aspects, the dosage at which a therapeutic effect is obtained is at or about 10⁸ GC/kg of the subject's body weight (GC/kg). In some aspects, the dosage is from at or about 10⁸ infectious units/kg to at or about 10¹⁴ infectious units/kg of the subject's body weight, inclusive.

In some of any embodiments, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors

In some of any embodiments, the present disclosure is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intratumoral intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some of any embodiments, the viral vector composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the viral vector compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of viral vectors are from the same subject that is administered a viral vector composition. In other embodiments, they are different. In some embodiments, the source of viral vectors and recipient tissue may be autologous (from the same subject) or heterologous (from different subjects). In some embodiments, the donor tissue for viral vector compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the viral vector composition described herein may be administered to a subject having a cancer, an autoimmune disease, an infectious disease, a metabolic disease, a neurodegenerative disease, or a genetic disease (e.g., enzyme deficiency).

### IX. EXEMPLARY EMBODIMENTS

Among the provided embodiments are:
1. A method of transducing T cells, the method comprising:
contacting a non-activated T cell with a lentiviral vector comprising a CD4 binding agent, wherein the lentiviral vector transduces the non-activated T cell.
2. The method of embodiment 1, wherein the T cell is a CD4+ T cells.
3. The method of embodiment 1 or embodiment 2, wherein the non-activated T cell is surface negative for one or more T cell activation markers selected from the group consisting of CD25, CD44 and CD69.
4. The method of any of embodiments 1-3, wherein the non-activated T cell has not been treated with an anti-CD3 antibody (e.g., OKT3).
5. The method of any of embodiments 1-4, wherein the non-activated T cell has not been treated with an anti-CD28 antibody (e.g., CD28.2).
6. The method of any of embodiments 1-5, wherein the non-activated T cell has not been treated with a bead coupled to an anti-CD3 antibody (e.g. OKT3) and an anti-CD28 antibody (e.g. CD28.2), optionally wherein the bead is a superparamagnetic bead.
7. The method of any of embodiments 1-6, wherein the non-activated T cell has not been treated with a T cell activating cytokine (e.g., recombinant IL-2, IL-7, IL-15, IL-21, or combinations thereof), optionally wherein the T cell activating cytokine is a human cytokine.
8. The method of any of embodiments 1-7, wherein the non-activated T cell has not been treated with a soluble T cell costimulatory molecule (e.g. anti-CD28 antibody or soluble CD80, soluble CD86, soluble CD137L or soluble ICOS-L).
9. The method of any of embodiments 1-8, wherein the lentiviral vector comprises a transgene encoding an engineered receptor that binds to or recognizes a protein or antigen expressed by or on cells associated with a disease or condition (e.g. tumor cells).
10. The method of embodiment 9, wherein the engineered receptor is a chimeric antigen receptor (CAR).
11. The method of embodiment 9 or embodiment 10, wherein the CAR comprises an antigen-binding domain, a transmembrane domain, and an intracellular signaling domain comprising intracellular components of a CD3zeta signaling domain and a costimulatory signaling domain.
12. The method of embodiment 11, wherein the costimulatory signaling domain is a 4-1BB signaling domain.
13. The method of embodiment 9, wherein the engineered T cell receptor (TCR).
14. The method of any of embodiments 1-13, wherein the non-activated T cell is a human T cell.
15. The method of any of embodiments 1-14, wherein the non-activated T cell is in a subject.
16. The method of any of embodiments 1-14, wherein the non-activated T cell is in vitro.
17. The method of any of embodiments 1-14, wherein the non-activated T cell is ex vivo from a subject.
18. The method of embodiment 15 or embodiment 17, wherein, prior to the contacting, the subject had not been administered a T cell activating treatment.
19. The method of embodiment 15, 17 or 18 wherein the subject has a disease or condition.
20. A transduced T cell produced by the method of any of embodiments 1-14, 16-19 and 56-119.
21. A composition comprising the transduced T cell of embodiment 20, optionally wherein the composition is a pharmaceutical composition.
22. A method of transducing a population of T cells, the method comprising:
contacting a population of non-activated T cells with a composition comprising lentiviral vectors comprising a CD4 binding agent, wherein the population of non-activated T cells is transduced at an efficiency of at least 1%.
23. The method of embodiment 22, wherein the population of non-activated T cells is transduced at an efficiency of at least 5%.
24. The method of embodiment 22 or embodiment 23, wherein the population of non-activated T cells is transduced at an efficiency of at least 10%, at least 15%, at least 20%, at least 25%, at least 30%, at least 35%, at least 40%, at least 45%, at least 50%, at least 55%, at least 60%, at least 65%, at least 70%, or at least 75%.
25. The method of any of embodiments 22-24, wherein at least 75% of the T cells in the population of non-activated T cells are surface negative for one or more T cell activation markers selected from the group consisting of CD25, CD44 and CD69 (e.g. at least 80%, at least 85%, at least 90%, at least 95% of the T cells in the population are surface negative for the T cell activation marker).
26. The method of any of embodiments 22-25, wherein the population of non-activated T cells comprises CD4+ T cells (e.g. at least 10%, at least 20%, at least 30%, at least 40%, at least 50%, at least 60%, at least 70%, at least 80%, at least 90% of the population of non-activated T cells are CD4+ T cells).
27. The method of embodiment 26, wherein at least 75% of the CD4+ T cells are surface negative for one or more T cell activation markers selected from the group consisting of CD25, CD44 and CD69 (e.g. at least 80%, at least 85%, at least 90%, at least 95% of the CD4+ T cells in the population are surface negative for the T cell activation marker).
28. The method of embodiment 26 or embodiment 27, wherein the CD4+ T cells in the population of non-activated T cells are transduced at an efficiency of at least 1%, at least 5%, at least 10%, at least 15%, at least 20%, at least 25%, at least 30%, at least 35%, or at least 40%.
29. The method of any of embodiments 22-28, wherein the population of non-activated T cells has not been treated with an anti-CD3 antibody (e.g., OKT3).
30. The method of any of embodiments 22-29, wherein the population of non-activated T cell has not been treated with an anti-CD28 antibody (e.g., CD28.2).
31. The method of any of embodiments 22-30, wherein the population of non-activated T cells has not been treated with a bead coupled to an anti-CD3 antibody (e.g. OKT3) and an anti-CD28 antibody (e.g. CD28.2), optionally wherein the bead is a superparamagnetic bead.
32. The method of any of embodiments 22-31, wherein the population of non-activated T cell has not been treated with a T cell activating cytokine (e.g., recombinant IL-2, IL-7, IL-15, IL-21, or combinations thereof), optionally wherein the T cell activating cytokine is a human cytokine.
33. The method of any of embodiments 22-32, wherein the population of non-activated T cells has not been treated with a soluble T cell costimulatory molecule (e.g. anti-CD28 antibody or soluble CD80, soluble CD86, soluble CD137L or soluble ICOS-L).
34. The method of any of embodiments 22-33, wherein the population of non-activated T cells are human cells.
35. The method of any of embodiments 22-34, wherein the population of non-activated T cells is in a subject.
36. The method of embodiment 35, wherein, prior to the contacting, the subject had not been administered a T cell activating treatment.
37. The method of any of embodiments 22-34, wherein the population of non-activated T cells is in vitro.
38. The method of any of embodiments 22-34, wherein the population of non-activated T cells is ex vivo from a subject.
39. The method of any of embodiments 22-34, 37 and 38, wherein the population of non-activated T cells comprise peripheral blood mononuclear cells (PBMCs) or a subset thereof comprising CD4+ T cells.
40. The method of any of embodiments 22-34 and 37-39, wherein the population of non-activated cells is an enriched population of T cells selected from a biological sample from a subject, optionally wherein the T cells are selected for T cells surface positive for a T cell marker (e.g., CD3 or CD4).
41. The method of embodiment 40, wherein the biological sample is a whole blood sample, apheresis sample or leukapheresis sample.
42. The method of embodiment 35, 36 and 38-41, wherein the subject has a disease or condition.
43. The method of any of embodiments 22-34 and 37-42, further comprising expanding the population of transduced T cells.
44. The method of embodiment 43, wherein the expanding comprises incubation of the transduced cells with one or more T cell activating cytokine (e.g., recombinant IL-2, IL-7, IL-15, IL-21, or combinations thereof), optionally wherein the T cell activating cytokine is a human cytokine.
45. The method of any of embodiments 22-34 and 37-43, further comprising incubating the transduced T cells with one or more T cell activating cytokine (e.g., recombinant IL-2, IL-7, IL-15, IL-21, or combinations thereof), optionally wherein the T cell activating cytokine is a human cytokine.
46. A population of transduced T cells produced by the method of any of embodiments 22-34, 37-45 and 56-119.
47. A composition comprising a population of transduced T cells produced by the method of any of embodiments 22-34, 37-45 and 56-119, optionally wherein the composition is a pharmaceutical composition.
48. The composition of embodiment 21 or embodiment 47 further comprising a cyropreservant, optionally wherein the cyropreservant is DMSO.
49. A method of in vivo transduction of T cells, the method comprising:
administering to a subject a composition comprising lentiviral vectors comprising a CD4 binding agent, wherein the lentiviral vectors transduce T cells within the subject, and wherein the subject is not administered a T cell activating treatment (e.g. before, after or concurrently) with administration of the composition.
50. The method of embodiment 49, wherein the subject has a disease or condition.
51. A method of treating a subject having a disease or condition, the method comprising: administering to the subject a composition comprising lentiviral vectors comprising a CD4 binding agent, and wherein the subject is not administered a T cell activating treatment (e.g. before, after or concurrently) with administration of the composition.
52. The method of any of embodiments 19, 42 and 51, wherein the disease or condition is a cancer.
53. The method of any of embodiment 19, 42, 51 and 52, wherein the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR).
54. A method for expanding T cells capable of recognizing and killing tumor cells in a subject in need thereof, the method comprising:
administering to the subject a composition comprising lentiviral vectors comprising a CD4 binding agent, and wherein the subject is not administered a T cell activating treatment (e.g. before, after, or concurrently) with administration of the composition.
55. The method of embodiment 54, wherein the lentiviral vector comprises a transgene encoding an engineered receptor that binds to or recognizes a protein expressed on the tumor cells, optionally wherein the engineered receptor is a chimeric antigen receptor (CAR) or an engineered T cell receptor (TCR).
56. The method of any of embodiments 18, 36, 49-55, 10⁸-112 and 129-131, wherein the T cell activating treatment comprises administration of an anti-CD3 antibody (e.g., OKT3).
57. The method of any of embodiments 18, 36, 49-56, 10⁸-112 and 129-131, wherein the T cell activating treatment comprises administration of a soluble T cell costimulatory molecule (e.g., anti-CD28 antibody, or a recombinant CD80, CD86, CD137L, ICOS-L).
58. The method of any of embodiments 18, 36, 49-57, 10⁸-112 and 129-131 wherein the T cell activating treatment comprises administration of a T cell activating cytokine (e.g., recombinant IL-2, IL-7, IL-15, IL-21), optionally wherein the T cell activating cytokine is a human cytokine.
59. The method of any of embodiments 18, 36, 49-58, 10⁸-112 and 129-131, wherein the T cell activating treatment comprises administration of recombinant IL-7, optionally human IL-7.
60. The method of any of embodiments 18, 36, 49-59 and 10⁸-112 and 129-131, wherein the T cell activating treatment comprises administration of a lymphodepleting therapy, optionally administration of cyclophosphamide and/or fludarabine.
61. The method of any of embodiments 1-60, wherein the CD4 binding agent is an anti-CD4 antibody or an antigen-binding fragment.
62. The method of embodiment 61, wherein the anti-CD4 antibody or antigen-binding fragment is mouse, rabbit, human, or humanized.
63. The method of embodiment 61 or embodiment 62, wherein the antigen-binding fragment is a single chain variable fragment (scFv).
64. The method of embodiment 61 or embodiment 62, wherein the anti-CD4 antibody or antigen-binding fragment is a single domain antibody.
65. The method of embodiment 61 or embodiment 64, wherein the anti-CD4 antibody or antigen-binding fragment is a camelid (e.g. llama, alpaca, camel) (e.g. VHH).
66. The method of any of embodiments 1-65, wherein the CD4 binding agent is an anti-CD4 VHH.
67. The method of any of embodiments 1-66, wherein the CD4 binding agent is exposed on the surface of the lentiviral vector.
68. The method of any of embodiments 1-67, wherein the CD4 binding agent is fused to a transmembrane domain incorporated in the viral envelope.
69. The method of any of embodiments 1-68, wherein the lentiviral vector is pseudotyped with a viral fusion protein.
70. The method of embodiment 69, wherein the viral fusion protein is a VSV-G protein or a functional variant thereof.
71. The method of embodiment 69, wherein the virial fusion protein is a Cocal virus G protein or a functional variant thereof.
72. The method of embodiment 69, wherein the viral fusion protein is an Alphavirus fusion protein (e.g. Sindbis virus) or a functional variant thereof.
73. The method of embodiment 69, wherein the viral fusion protein is a Paramyxoviridae fusion protein (e.g., a Morbillivirus or a Henipavirus) or a functional variant thereof.
74. The method of embodiment 69 or embodiment 73, wherein the viral fusion protein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.
75. The method of embodiment 69 or embodiment 63, wherein the viral fusion protein is a Henipavirus fusion protein (e.g., Nipah virus, Hendra virus, Cedar virus, Kumasi virus, Mojiang virus) or a functional variant thereof.
76. The method of any of embodiments 69-75, wherein the viral fusion protein comprises one or modifications to reduce binding to its native receptor.
77. The method of any of embodiments 69-76, wherein the viral fusion protein is fused to the CD4 binding agent.
78. The method any of embodiments 69, 73 and 75-77, wherein the viral fusion protein comprises a Nipah virus F glycoprotein (NiV-F) or a biologically active portion thereof and a Nipah virus G glycoprotein (NiV-G) or a biologically active portion thereof, and wherein the CD4 binding agent is fused to the NiV-G or the biologically active portion thereof.
79. The method of embodiment 78, wherein the CD4 binding agent is fused to the C-terminus of the Nipah virus G glycoprotein or the biologically active portion thereof.
80. The method of any of embodiments 77-79, wherein the CD4 binding protein is fused directly or via a peptide linker.
81. The method of any of embodiments 78-80, wherein the NiV-G or the biologically active portion thereof is a wild-type NiV-G protein or a functionally active variant or biologically active portion thereof.
82. The method of any of embodiments 78-81, wherein the NiV-G protein or the biologically active portion is truncated and lacks up to 40 contiguous amino acid residues at or near the N-terminus of the wild-type NiV-G protein (SEQ ID NO:19,SEQ ID NO:4 or SEQ ID NO:5).
83. The method of any of embodiments 78-82, wherein the NiV-G protein or the biologically active portion has a 5 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:12, or a sequence of amino acids that exhibits at least at or about 80%, 85%, 90% or 95% sequence identity to the sequence set forth in SEQ ID NO:12.
84. The method of any of embodiments 78-82, wherein the NiV-G protein or the biologically active portion has a 10 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44.
85. The method of any of embodiments 78-82, wherein the NiV-G protein or the biologically active portion has a 15 amino acid truncation at or near the N-terminus of the wild-type NiV-G protein (SEQ ID NO:9, SEQ ID NO:4 or SEQ ID NO:5), optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45.
86. The method of any of embodiments 78-82, wherein the NiV-G protein or the biologically active portion has a 20 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:13, or a sequence of amino acids that exhibits at least at or about 80%, 85%, 90% or 95% sequence identity to the sequence set forth in SEQ ID NO:13.
87. The method of any of embodiments 78-82, wherein the NiV-G protein or the biologically active portion has a 25 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:14, or a sequence of amino acids that exhibits at least at or about 80%, 85%, 90% or 95% sequence identity to the sequence set forth in SEQ ID NO: 14.
88. The method of any of embodiments 78-82, wherein the NiV-G protein or the biologically active portion has a 30 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43.
89. The method of any of embodiments 78-82, wherein the NiV-G protein or the biologically active portion has a 34 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.
90. The method of any of embodiments 78-82, wherein the NiV-G protein or the biologically active portion has a 34 amino acid truncation at or near the N-terminus of the wild-type NiV-G protein (SEQ ID NO:1, SEQ ID NO:4 or SEQ ID NO:5), optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.
91. The method any of embodiments 78-90, wherein the NiV-G-protein or the biologically active portion thereof is a mutant NiV-G protein that exhibits reduced binding to Ephrin B2 or Ephrin B3.
92. The method of embodiment 91, wherein the mutant NiV-G protein or the biologically active portion comprises:
one or more amino acid substitutions corresponding to amino acid substitutions selected from the group consisting of E501A, W504A, Q530A and E533A with reference to numbering set forth in SEQ ID NO:4.
93. The method of embodiment 91 or embodiment 92, wherein the mutant NiV-G protein or the biologically active portion has the amino acid sequence set forth in SEQ ID NO: 17 or a sequence of amino acids that exhibits at least at or about 80%, 85%, 90% or 95% sequence identity to the sequence set forth in SEQ ID NO: 17.
94. The method of embodiment 91 or embodiment 92, wherein the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18.
95. The method of any of embodiments 78-94, wherein the NiV-F protein or the biologically active portion thereof is a wild-type NiV-F protein or is a functionally active variant or a biologically active portion thereof.
96. The method of any of embodiments 78-95, wherein the NiV-F protein or the biologically active portion thereof has a 20 amino acid truncation at or near the C-terminus of the wild-type NiV-F protein (SEQ ID NO: 41), optionally wherein the NiV-F protein or the biologically active portion thereof has the sequence set forth in SEQ ID NO: 20 or a sequence of amino acids that exhibits at least at or about 80%, 85%, 90% or 95% sequence identity to the sequence set forth in SEQ ID NO: 20.
97. The method of any of embodiments 78-96, wherein the NiV-F protein or the biologically active portion thereof comprises:
i) a 20 amino acid truncation at or near the C-terminus of the wild-type NiV-F protein (SEQ ID NO: 41); and
ii) a point mutation on an N-linked glycosylation site, optionally wherein the NiV-F protein or the biologically active portion thereof has the sequence set forth in SEQ ID NO: 15, or a sequence of amino acids that exhibits at least at or about 80%, 85%, 90% or 95% sequence identity to the sequence set forth in SEQ ID NO: 15.
98. The method of any of embodiments 78-95, wherein the NiV-F protein or the biologically active portion thereof has a 22 amino acid truncation at or near the C-terminus of the wild-type NiV-F protein (SEQ ID NO: 41), optionally wherein the NiV-F protein or the biologically active portion thereof has the sequence set forth in SEQ ID NO: 16 or 21 or a sequence of amino acids that exhibits at least at or about 80%, 85%, 90% or 95% sequence identity to the sequence set forth in SEQ ID NO: 16 or 21.
99. The method of any of embodiments 1-98, wherein the lentiviral vector comprises a transgene.
100. The method of embodiment 99, wherein the transgene comprises a nucleic acid sequence encoding an RNA sequence capable of RNA interference (e.g. pre-miRNA, siRNA, or shRNA).
101. The method of embodiment 99, wherein the transgene is selected from the group consisting of a therapeutic gene, a reporter gene, a gene encoding an enzyme, a gene encoding a pro-drug enzyme, a gene encoding an apoptosis inducer, a gene encoding a fluorescent protein, a gene encoding a pro-drug-activating enzyme, a gene encoding an apoptotic protein, a gene encoding an apoptotic enzyme, a gene encoding a suicide protein, a gene encoding a cytokine, a gene encoding an anti-immunosuppressive protein, a gene encoding an epigenetic modulator, a gene encoding a T cell receptor (TCR), a gene encoding a chimeric antigen receptor (CAR), a gene encoding a protein that modifies the cell surface of transduced cells, a gene encoding a protein that modifies the expression of the endogenous TCR, and a gene encoding a switch receptor that converts pro-tumor into anti-tumor signals.
102. The method of embodiment 99, wherein the transgene encodes an engineered receptor that binds to or recognizes a protein or antigen expressed by cells or a lesion (e.g. tumor) associated with a disease or condition, optionally wherein the engineered receptor is a chimeric antigen receptor (CAR) or an engineered T cell receptor (TCR).
103. The method of embodiment 53, embodiment 55, embodiment 99 or embodiment 102, wherein the transgene encodes a chimeric antigen receptor (CAR).
104. The method of embodiment 103, wherein the CAR comprises an antigen-binding domain, a transmembrane domain, and an intracellular signaling domain comprising intracellular components of CD3zeta signaling domain and a costimulatory signaling domain.
105. The method of embodiment 104, wherein the costimulatory signaling domain is a 4-1BB signaling domain.
106. The method of any of embodiments 53, 55, 99 and 102, wherein the transgene encodes an engineered T cell receptor (TCR).
107. The method of any of embodiments 1-106, wherein the lentiviral vector does not comprise a T cell activating agent displayed on the surface, optionally wherein the T cell activating agent is selected from the group consisting of a CD3 antibody (e.g. anti-CD3 scFv); a T cell activating cytokine (e.g. IL-2, IL-7, IL-15 or IL-21); or a T cell costimulatory molecule (e.g. anti-CD28 antibody, CD80, CD86, CD137L or ICOS-L).
108. The method of any of embodiments 1-106, wherein the lentiviral vector does not comprise or encode a T cell activating agent, optionally wherein the T cell activating agent is a lymphoproliferative agent.
109. The method of embodiment 108, wherein the T cell activating agent is:
a polypeptide capable of binding CD3 and/or CD28;
a CD3 antibody (e.g. anti-CD3 scFv); a T cell activating cytokine (e.g. IL-2, IL-7, IL-15 or IL-21); or a T cell costimulatory molecule (e.g. anti-CD28 antibody, CD80, CD86, CD137L or ICOS-L);
a cytokine or a cytokine receptor or a signaling domain thereof that activates a STAT3 pathway, a STAT4 pathway, and/or a Jak/STAT5 pathway;
a T cell survival motif, optionally an IL-7 receptor, an IL-15 receptor, or CD28, or a functional portion thereof; and/or
a microRNA (miRNA) or short hairpin RNA (shRNA), wherein the miRNA or the shRNA stimulates the STAT5 pathway and/or inhibits the SOCS pathway.
110. The method of any of embodiments 1-106, wherein the lentiviral vector does not comprise or encode a T cell activating agent that is membrane bound and/or displayed on the surface, optionally wherein the T cell activating agent is a lymphoproliferative agent.
111. The method of any of embodiments 18, 36 and 49-110, wherein the subject is not administered a T cell activating treatment concurrently with the lentiviral vector.
112. The method of any of embodiments 18, 36 and 49-111, wherein the subject is not administered a T cell activating treatment within 1 month before the contacting with the lentiviral vector or before the administration of the composition comprising the lentiviral vectors.
113. The method of any of embodiments 18, 36, 49-112, wherein the subject is not administered a T cell activating treatment within or at or about 1 week, 2 weeks, 3 weeks or 4 weeks, optionally at or about 1, 2, 3, 4, 5, 6 or 7 days, before the contacting with the lentiviral vector or before the administration of the composition comprising the lentiviral vectors.
114. The method of any of embodiments 18, 36 and 49-113, wherein the subject is not administered a T cell activating treatment within 1 month after the contacting with the lentiviral vector or after the administration of the composition comprising the lentiviral vectors.
115. The method of any of embodiments 18, 36, 49-114, wherein the subject is not administered a T cell activating treatment within or at or about 1 week, 2 weeks, 3 weeks or 4 weeks, optionally at or about 1, 2, 3, 4, 5, 6 or 7 days, after the contacting with the lentiviral vector or after the administration of the composition comprising the lentiviral vectors.
116. The method of any one of embodiments 1-45, further comprising editing the T cell or population of T cells to inactivate one or more of B2M, CIITA, TRAC, and TRB genes.
117. The method of embodiment 113, wherein the T cell or population of T cells is edited to inactivate B2M, CIITA, and TRAC genes.
118. The method of embodiment 116, wherein the T cell of population of T cells is edited to inactivate B2M, CIITA, and TRB genes.
119. The method of any one of embodiments 116-118, further comprising inserting a gene encoding CD47 at a defined locus.
120. The method of embodiment 119, wherein the defined locus is selected from the group consisting of a B2M locus, a CIITA locus, a TRAC locus, a TRB locus, or a safe harbor locus.
121. The method of embodiment 120, wherein the safe harbor locus is selected from the group consisting of an AAVS1 locus, a CCR5 locus, and a ROSA26 locus.
122. The method of any of embodiments 116-121, wherein the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR).
123. A transduced T cell produced by the method of any of embodiments 116-122.
124. The transduced T cell of embodiment 123, wherein the T cell is inactivated at both alleles of the one or more genes.
125. A composition comprising the transduced T cell of embodiment 123 or embodiment 124, optionally wherein the composition is a pharmaceutical composition.
126. A population of transduced T cells produced by the method of any of embodiments 116-122.
127. The population of transduced T cells of embodiment 126, wherein at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of the cells of the population of non-activated cells are inactivated at the one or more genes.
128. The population of transduced T cells of embodiment 126 or embodiment 127, wherein at least 1%, at least 5%, at least 10%, at least 15%, at least 20%, at least 25%, at least 30%, at least 35%, or at least 40% of the non-activated CD4+ T cells in the population are transduced and are inactivated at the one or more genes.
129. The population of transduced T cells of any of embodiments 126-128, wherein cells of the population are inactivated at both alleles of the one or more genes.
130. A composition comprising the population of transduced T cells of any of embodiments 126-129, optionally wherein the composition is a pharmaceutical composition.
131. The composition of embodiment 125 or embodiment 130, further comprising a cyropreservant, optionally wherein the cyropreservant is DMSO.
132. A method of treating a subject having a disease or condition, the method comprising: administering to the subject a composition of any of embodiments 21, 47, 48, 125, 130 and 131, wherein the subject is not administered a T cell activating treatment (e.g. before, after or concurrently) with administration of the composition.
133. The method of embodiment 132, wherein the disease or condition is a cancer.
134. A method for expanding T cells capable of recognizing and killing tumor cells in a subject in need thereof, the method comprising:
administering to the subject a composition of any of embodiments 21, 47, 48, 125, 130 and 131, and wherein the subject is not administered a T cell activating treatment (e.g. before, after, or concurrently) with administration of the composition.
135. Use of a composition comprising lentiviral vectors comprising a CD4 binding agent for treating a subject having a disease or condition, optionally a cancer.
136. Use of a composition of any of embodiments 21, 47, 48, 125, 130 and 131 for formulation of a medicament for treating a subject having a disease or condition, optionally a cancer.
137. A composition comprising lentiviral vectors comprising a CD4 binding agent for use in treating a subject having a disease or condition, optionally a cancer.
138. A composition of any of embodiments 21, 47, 48, 125, 130 and 131 for use in treating a subject having a disease or condition, optionally a cancer.
139. Use of a composition comprising lentiviral vectors comprising a CD4 binding agent for formulation of a medicament for expanding T cells capable of recognizing and killing tumor cells in a subject in need thereof.
140. Use of a composition of any of embodiments 21, 47, 48, 125, 130 and 131 for formulation of a medicament for expanding T cells capable of recognizing and killing tumor cells in a subject in need thereof.
141. A composition comprising lentiviral vectors comprising a CD4 binding agent for use in expanding T cells capable of recognizing and killing tumor cells in a subject in need thereof.
142. A composition of any of embodiments 21, 47, 48, 125, 130 and 131 for use in expanding T cells capable of recognizing and killing tumor cells in a subject in need thereof.
143. The use or the composition of any of embodiments 135-142 that is for use in a subject that is not administered or to be administered a T cell activating treatment (e.g. before, after or concurrently) with administration of the composition.
144. The method of any of embodiments 11-19, 104, 105, 107-115, and 117-122, wherein the costimulatory signaling domain is a CD28 costimulatory domain, optionally wherein the CD28 costimulatory signaling domain comprises the amino acid sequence set forth in SEQ ID NO:60.
145. The method of any of embodiments 12-19, 105, 107-115, 117-122, and 144, wherein the 4-1BB signaling domain comprises the amino acid sequence set forth in SEQ ID NO:59.
146. The method of any of embodiments 11-19, 104, 105, 107-115, 117-122, 144, and 145, wherein the CD3zeta signaling domain comprises the sequence set forth in SEQ ID NO:61 or SEQ ID NO:62.
147. The method of any of embodiments 11-19, 104, 105, 107-115, 117-122, and 144-146, wherein the transmembrane domain comprises the sequence set forth in any one of SEQ ID NOS:56, 57, and 58.
148. The method of any of embodiments 10-19, 103-105, 107-115, 117-122, and 144-147, wherein the CAR comprises a hinge domain, optionally wherein the hinge domain comprises the sequence set forth in any one of SEQ ID NOS:50, 51, 52, 53, 54, 55, and 142.
149. The method of any of embodiments 11-19, 104, 105, 107-115, 117-122, and 144-148, wherein the antigen binding domain binds to an antigen selected from the group consisting of CD19, CD20, CD22, and BCMA.
150. The method of any of embodiments 11-19, 104, 105, 107-115, 117-122, and 144-149, wherein the antigen binding domain binds to CD19.
151. The method of any of embodiments 11-19, 104, 105, 107-115, 117-122, and 144-150, wherein the antigen binding domain comprises:
(a) a CDR-H1, a CDRH-2, and a CDR-H3 comprising the amino acid sequence set forth in SEQ ID NO: 70, 71, and 72, respectively, and a CDR-L1, a CDR-L2, and a CDR-L3 comprising the amino acid sequence set forth in SEQ ID NO: 65, 66, and 67, respectively;
(b) a VH region comprising the amino acid sequence set forth in SEQ ID NO:69, and a VL region comprising the amino acid sequence set forth in SEQ ID NO:64; and/or
(c) the amino acid sequence set forth in SEQ ID NO:63 or 73.
152. The method of any of embodiments 11-19, 104, 105, 107-115, 117-122, and 144-151, wherein the CAR comprises the amino acid sequence set forth in SEQ ID NO:75, 77, 79, or 81 and/or an amino acid sequence encoded by the polynucleotide sequence set forth in SEQ ID NO:74, 76, 78, or 80.
153. The method of any of embodiments 11-19, 104, 105, 107-115, 117-122, and 144-149, wherein the antigen binding domain binds to CD20.
154. The method of any of embodiments 11-19, 104, 105, 107-115, 117-122, 144-149 and 153, wherein the antigen binding domain comprises:
(a) a CDR-H1, a CDRH-2, and a CDR-H3 comprising the amino acid sequence set forth in SEQ ID NO: 88, 89, and 144, respectively, and a CDR-L1, a CDR-L2, and a CDR-L3 comprising the amino acid sequence set forth in SEQ ID NO: 84, 85, and 86, respectively;
(b) a VH region comprising the amino acid sequence set forth in SEQ ID NO:87, and a VL region comprising the amino acid sequence set forth in SEQ ID NO:83; and/or
(c) the amino acid sequence set forth in SEQ ID NO:82.
155. The method of any of embodiments 11-19, 104, 105, 107-115, 117-122, and 144-149, wherein the antigen binding domain binds to CD22.
156. The method of any of embodiments 11-19, 104, 105, 107-115, 117-122, and 144-149 and 155, wherein the antigen binding domain comprises:
(a) a CDR-H1, a CDRH-2, and a CDR-H3 comprising the amino acid sequence set forth in SEQ ID NO: 92, 93, and 94, respectively, and a CDR-L1, a CDR-L2, and a CDR-L3 comprising the amino acid sequence set forth in SEQ ID NO: 96, 97, and 98, respectively; or
a CDR-H1, a CDRH-2, and a CDR-H3 comprising the amino acid sequence set forth in SEQ ID NO: 101, 102, and 103, respectively, and a CDR-L1, a CDR-L2, and a CDR-L3 comprising the amino acid sequence set forth in SEQ ID NO: 105, 106, and 107, respectively; and/or
(b) a VH region comprising the amino acid sequence set forth in SEQ ID NO:129, and a VL region comprising the amino acid sequence set forth in SEQ ID NO:95; or a VH region comprising the amino acid sequence set forth in SEQ ID NO:100, and a VL region comprising the amino acid sequence set forth in SEQ ID NO:104; and/or
(c) the amino acid sequence set forth in SEQ ID NO:90 or 99.
157. The method of any of embodiments 11-19, 104, 105, 107-115, 117-122, and 144-149, wherein the antigen binding domain binds to BCMA.
158. The method of any of embodiments 11-19, 104, 105, 107-115, 117-122, 144-149 and 157, wherein the antigen binding domain comprises:
(a) a CDR-H1, a CDRH-2, and a CDR-H3 comprising the amino acid sequence set forth in SEQ ID NO: 114, 115, and 116, respectively, and a CDR-L1, a CDR-L2, and a CDR-L3 comprising the amino acid sequence set forth in SEQ ID NO: 110, 111, and 112, respectively;
a CDR-H1, a CDRH-2, and a CDR-H3 comprising the amino acid sequence set forth in SEQ ID NO: 123, 124, and 125, respectively, and a CDR-L1, a CDR-L2, and a CDR-L3 comprising the amino acid sequence set forth in SEQ ID NO: 119, 120, and 121, respectively;
a CDR-H1, a CDRH-2, and a CDR-H3 comprising the amino acid sequence set forth in SEQ ID NO: 127, 128, and 129, respectively; or
a CDR-H1, a CDRH-2, and a CDR-H3 comprising the amino acid sequence set forth in SEQ ID NO: 136, 137, and 138, respectively, and a CDR-L1, a CDR-L2, and a CDR-L3 comprising the amino acid sequence set forth in SEQ ID NO:132, 133, and 134, respectively; and/or
(b) a VH region comprising the amino acid sequence set forth in SEQ ID NO:113, and a VL region comprising the amino acid sequence set forth in SEQ ID NO:109;
a VH region comprising the amino acid sequence set forth in SEQ ID NO:122, and a VL region comprising the amino acid sequence set forth in SEQ ID NO:118;
a VH region comprising the amino acid sequence set forth in SEQ ID NO:135, and a VL region comprising the amino acid sequence set forth in SEQ ID NO:131; or
a VH region comprising the amino acid sequence set forth in SEQ ID NO:126; and/or
(c) the amino acid sequence set forth in SEQ ID NO:108, 117, or 130.
159. The method of any of embodiments 11-19, 104, 105, 107-115, 117-122, 144-149, 157, and 158, wherein the CAR comprises the amino acid sequence set forth in SEQ ID NO:140 and/or an amino acid sequence encoded by the polynucleotide sequence set forth in SEQ ID NO:139.
160. The method of any of embodiments 11-19, 104, 105, 107-115, 117-122, and 144-152, wherein the CAR comprises:
(a) an antigen binding domain comprising the VL region set forth in SEQ ID NO:64, a linker comprising the amino acid sequence set forth in SEQ ID NO:68, and the VH region set forth in SEQ ID NO:69; and/or the scFv set forth in SEQ ID NO:63;
(b) a hinge comprising the amino acid sequence set forth in SEQ ID NO:50;
(c) a transmembrane domain comprising the amino acid sequence set forth in SEQ ID NO:56;
(d) a 4-1BB signaling domain comprises the amino acid sequence set forth in SEQ ID NO:59; and/or
(e) a CD3zeta signaling domain comprising the amino acid sequence set forth in SEQ ID NO:61.
161. The method of any of embodiments 11-19, 104, 105, 107-115, 117-122, 144-152, and 160, wherein the CAR comprises the amino acid sequence set forth in SEQ ID NO:75 and/or is encoded by the nucleotide sequence set forth in SEQ ID NO:74.
162. The method, use, or composition of any of embodiments 78-105, 107-122, and 144-161, wherein the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21.
163. The method, use, or composition of any of embodiments 78-105, 107-122, and 144-162, wherein the Niv-G protein comprises the amino acid sequence set forth in SEQ ID NO: 17, and the Niv-F protein comprises the amino acid sequence set forth in SEQ ID NO:21.
164. The method of any one of embodiments 1-19, 22-45,49-122, 132-134, and 144-163, wherein the contacting or the administering is carried out by ex vivo administration of the lentiviral vector to a subject using a closed fluid circuit.
165. The method of embodiment 164, wherein the ex vivo administration comprises:
(a) obtaining whole blood from a subject;
(b) collecting the fraction of blood containing leukocyte components comprising T cells (e.g. CD4+ T cells);
(c) contacting the leukocyte components comprising T cells (e.g. CD4+ T cells) with a composition comprising the lentiviral vector; and
(d) reinfusing the contacted leukocyte components comprising T cells (e.g. CD4+ T cells) into the subject, wherein steps (a)-(d) are performed in-line in a closed fluid circuit.
166. The method of embodiment 165, wherein the contacting in step (c) is for no more than 24 hours, no more than 18 hours, no more than 12 hours, or no more than 6 hours.

### X. EXAMPLES

The following examples are included for illustrative purposes only and are not intended to limit the scope of the invention.

### Example 1: Transduction of Resting T Helper Cells Using a CD4 Targeted Fusosome to Generate CAR T Cells

This Example describes the assessment of the transduction efficiency of CD4 retargeted Nipah fusogens and VSV-G.

Anti-CD4 single chain antibody fragments were cloned in-frame into the G-protein of the Nipah virus envelope. Fusosomes (lentiviral vector; LVV) with transfer plasmids expressing green fluorescent protein (GFP) were generated and tested for infectious titers against the CD4+ SupT1 cell line CD4 targeted fusosomes efficiently transduced CD4+ SupT1 cells (SupT1 titers>1E6).

CD4 fusosomes were compared to VSV-G pseudotyped virus for efficiency and specificity in transducing PBMCs using the GFP transgene. Specificity was also evaluated by the ability of the CD4-NiV-G pseudotyped LVV to transduce non-CD4 expressing cells. To confirm specificity, activated PBMCs were exposed to a CD4 LVV-GFP or VSV-G pseudotyped LVV-GFP. VSV-G enabled transduction of both CD4+ and CD8+ T cells, whereas CD4 fusosomes only transduced CD4+ T cells.

Subsequently, a CD19-specific CAR encoding 4-1BB and the CD3zeta endo-domains (CD19 CAR) was generated to examine CD4+ CAR T transduction efficiency and functionality. PBMCs were thawed and activated with anti-CD3/anti-CD28 beads and exposed to GFP-expressing CD4 fusosomes, and specificity of targeting CD4+ T cells was measured by flow cytometry.

Subsequently, CD19 CAR fusosomes targeting CD4 were used to test transduction efficiency against activated (CD3/CD28 or IL-7 treated) or resting CD4+ T cells, and to measure T cell function against CD19+ and CD19 CRISPR/Cas9-knockout lymphoma cells (Nalm-6) (e.g., tumor co-culture and rechallenge assays and cytokine production) in vitro. Vector copy number (VCN) was determined by a multiplex ddPCR assay and reported as copies per diploid genome (c/dg). CD4-targeted CD19 CAR fusosomes could efficiently transduce both activated (34%±1.5% CD4+ CAR+; 0.54±0.18 c/dg), and resting CD4-selected T cells, albeit at a lower expression and integration level (20%±0.5% CD4+CAR+; 0.28±0.14 c/dg). Resting CD4-transduced CAR T cells demonstrated specific cytotoxicity and cytokine production (GM-CSF, IFN-7, TNF-α, IL-2, IL-6, and IL-10) against CD19+Nalm-6 cells, but did not recognize CD19 knockout tumor cells. In long-term co-culture assays (9-day) with repetitive stimulation with fresh tumor cells (3×), CD4+CD19 CAR T cells transduced without prior activation continued to show potent tumor cell killing.

CD4-specific fusosomes were observed to efficiently deliver an integrating CAR payload to resting and activated CD4+ T cells. Modified CD4+CAR T cells demonstrated potent anti-tumor activity against CD19+ tumor cells. Without wishing to be bound by theory, these data are consistent with a finding that targeting the CD4 co-receptor through in vivo delivery using a novel pseudotyped LVV can produce functional CAR T cells.

### Example 2: CD4-Targeted Fusosomes Reduce CD19+ Tumor Burden Al r/Ro

CD4-targeted CD19 CAR fusosomes (lentiviral vector) were generated substantially as described in Example 1 and assessed for their ability to reduce tumor burden in vivo. The fusosomes were pseudotyped with Nipah virus fusogen retargeted with a CD4 VHH. NSG mice were injected with 1E6 Nalm6-Luc leukemia B cells via intravenous (IV) injection, followed three days later by an IV injection of 1E7 human peripheral blood mononuclear cells (hPBMC). A day after hPBMC injection, 2.5E6, 5E6, or 1E7 integrating units (IU) of CD4-targeted CD19 CAR fusosomes were injected into separate groups of mice. Nalm6 tumor progression was tracked via bioluminescent imaging (BLI) weekly throughout the duration of the study. The CD19 CAR contained an anti-scFv directed against CD19 and an intracellular signaling domain containing intracellular components of 4-1BB and CD3-zeta.

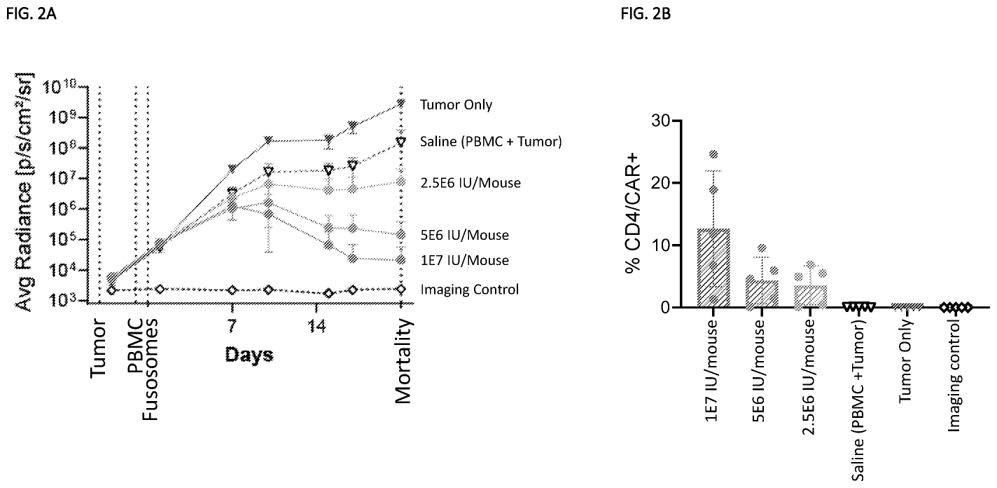

As shown in FIG. 2A, CD4-targeted CD19 CAR fusosomes resulted in control of Nalm6 tumor growth at Day 21. Additionally, peripheral blood of the mice was collected by mandibular puncture at study Day 15 to assess CAR positivity in CD4+ T-cells by flow cytometry. As shown in FIG. 2B, expression of the CAR in CD4+ T cells was dose-dependent. These data indicate that in vivo delivery of a CD19 CAR transgene payload with CD4-targeted fusosomes in CD19+ tumor bearing mice demonstrates robust production of CAR T cells and CD19+ tumor eradication.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | Description |
|---|---|---|
| 1 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 2 | MMADSKLVSLNNNLSGKIKDQGKVIKNYYGTMDIKKINDGLLDSKI | Hendra Virus G |
| | LGAFNTVIALLGSIIIIVMNIMIIQNYTRTTDNQALIKESLQSVQQQIKA | Protein |
| | LTDKIGTEIGPKVSLIDTSSTITIPANIGLLGSKISQSTSSINENVNDKCK | |
| | FTLPPLKIHECNISCPNPLPFREYRPISQGVSDLVGLPNQICLQKTTSTI | |
| | LKPRLISYTLPINTREGVCITDPLLAVDNGFFAYSHLEKIGSCTRGIAK | |
| | QRIIGVGEVLDRGDKVPSMFMTNVWTPPNPSTIHHCSSTYHEDFYYT | |
| | LCAVSHVGDPILNSTSWTESLSLIRLAVRPKSDSGDYNQKYIAITKVE | |
| | RGKYDKVMPYGPSGIKQGDTLYFPAVGFLPRTEFQYNDSNCPIIHCK | |
| | YSKAENCRLSMGVNSKSHYILRSGLLKYNLSLGGDIILQFIEIADNRL | |
| | TIGSPSKIYNSLGQPVFYQASYSWDTMIKLGDVDTVDPLRVQWRNNS | |
| | VISRPGQSQCPRFNVCPEVCWEGTYNDAFLIDRLNWVSAGVYLNSN | |
| | QTAENPVFAVFKDNEILYQVPLAEDDTNAQKTITDCFLLENVIWCISL | |
| | VEIYDTGDSVIRPKLFAVKIPAQCSES | |
| 3 | MADSKLVSLNNNLSGKIKDQGKVIKNYYGTMDIKKINDGLLDSKILG | Hendra Virus G |
| | AFNTVIALLGSIIIIVMNIMIIQNYTRTTDNQALIKESLQSVQQQIKALT | Protein without |
| | DKIGTEIGPKVSLIDTSSTITIPANIGLLGSKISQSTSSINENVNDKCKFT | Met |
| | LPPLKIHECNISCPNPLPFREYRPISQGVSDLVGLPNQICLQKTTSTILK | |
| | PRLISYTLPINTREGVCITDPLLAVDNGFFAYSHLEKIGSCTRGIAKQRI | |
| | IGVGEVLDRGDKVPSMFMTNVWTPPNPSTIHHCSSTYHEDFYYTLCA | |
| | VSHVGDPILNSTSWTESLSLIRLAVRPKSDSGDYNQKYIAITKVERGK | |
| | YDKVMPYGPSGIKQGDTLYFPAVGFLPRTEFQYNDSNCPIIHCKYSK | |
| | AENCRLSMGVNSKSHYILRSGLLKYNLSLGGDIILQFIEIADNRLTIGS | |
| | PSKIYNSLGQPVFYQASYSWDTMIKLGDVDTVDPLRVQWRNNSVIS | |
| | RPGQSQCPRFNVCPEVCWEGTYNDAFLIDRLNWVSAGVYLNSNQTA | |
| | ENPVFAVFKDNEILYQVPLAEDDTNAQKTITDCFLLENVIWCISLVEI | |
| | YDTGDSVIRPKLFAVKIPAQCSES | |
| 4 | MPAENKKVRFENTTSDKGKIPSKVIKSYYGTMDIKKINEGLLDSKILS | Nipah Virus G |
| | AFNTVIALLGSIVIIVMNIMIIQNYTRSTDNQAVIKDALQGIQQQIKGL | Protein |
| | ADKIGTEIGPKVSLIDTSSTITIPANIGLLGSKISQSTASINENVNEKCKF | |
| | TLPPLKIHECNISCPNPLPFREYRPQTEGVSNLVGLPNNICLQKTSNQI | |
| | LKPKLISYTLPVVGQSGTCITDPLLAMDEGYFAYSHLERIGSCSRGVS | |
| | KQRIIGVGEVLDRGDEVPSLFMTNVWTPPNPNTVYHCSAVYNNEFY | |
| | YVLCAVSTVGDPILNSTYWSGSLMMTRLAVKPKSNGGGYNQHQLA | |
| | LRSIEKGRYDKVMPYGPSGIKQGDTLYFPAVGFLVRTEFKYNDSNCP | |
| | ITKCQYSKPENCRLSMGIRPNSHYILRSGLLKYNLSDGENPKVVFIEIS | |
| | DQRLSIGSPSKIYDSLGQPVFYQASFSWDTMIKFGDVLTVNPLVVNW | |
| | RNNTVISRPGQSQCPRFNTCPEICWEGVYNDAFLIDRINWISAGVFLD | |
| | SNQTAENPVFTVFKDNEILYRAQLASEDTNAQKTITNCFLLKNKIWCI | |
| | SLVEIYDTGDNVIRPKLFAVKIPEQCT | |
| 5 | PAENKKVRFENTTSDKGKIPSKVIKSYYGTMDIKKINEGLLDSKILSA | Nipah Virus G |
| | FNTVIALLGSIVIIVMNIMIIQNYTRSTDNQAVIKDALQGIQQQIKGLA | Protein (No |
| | DKIGTEIGPKVSLIDTSSTITIPANIGLLGSKISQSTASINENVNEKCKFT | Met) |
| | LPPLKIHECNISCPNPLPFREYRPQTEGVSNLVGLPNNICLQKTSNQIL | |
| | KPKLISYTLPVVGQSGTCITDPLLAMDEGYFAYSHLERIGSCSRGVSK | |
| | QRIIGVGEVLDRGDEVPSLFMTNVWTPPNPNTVYHCSAVYNNEFYY | |
| | VLCAVSTVGDPILNSTYWSGSLMMTRLAVKPKSNGGGYNQHQLAL | |
| | RSIEKGRYDKVMPYGPSGIKQGDTLYFPAVGFLVRTEFKYNDSNCPI | |
| | TKCQYSKPENCRLSMGIRPNSHYILRSGLLKYNLSDGENPKVVFIEIS | |
| | DQRLSIGSPSKIYDSLGQPVFYQASFSWDTMIKFGDVLTVNPLVVNW | |
| | RNNTVISRPGQSQCPRFNTCPEICWEGVYNDAFLIDRINWISAGVFLD | |
| | SNQTAENPVFTVFKDNEILYRAQLASEDTNAQKTITNCFLLKNKIWCI | |
| | SLVEIYDTGDNVIRPKLFAVKIPEQCT | |
| 6 | MLSQLQKNYLDNSNQQGDKMNNPDKKLSVNFNPLELDKGQKDLNK | Cedar Virus G |
| | SYYVKNKNYNVSNLLNESLHDIKFCIYCIFSLLIIITIINIITISIVITRLKV | Protein |
| | HEENNGMESPNLQSIQDSLSSLTNMINTEITPRIGILVTATSVTLSSSIN | |
| | YVGTKTNQLVNELKDYITKSCGFKVPELKLHECNISCADPKISKSAM | |
| | YSTNAYAELAGPPKIFCKSVSKDPDFRLKQIDYVIPVQQDRSICMNNP | |
| | LLDISDGFFTYIHYEGINSCKKSDSFKVLLSHGEIVDRGDYRPSLYLLS | |
| | SHYHPYSMQVINCVPVTCNQSSFVFCHISNNTKTLDNSDYSSDEYYIT | |
| | YFNGIDRPKTKKIPINNMTADNRYIHFTFSGGGGVCLGEEFIIPVTTVI | |
| | NTDVFTHDYCESFNCSVQTGKSLKEICSESLRSPTNSSRYNLNGIMIIS | |
| | QNNMTDFKIQLNGITYNKLSFGSPGRLSKTLGQVLYYQSSMSWDTY | |
| | LKAGFVEKWKPFTPNWMNNTVISRPNQGNCPRYHKCPEICYGGTYN | |
| | DIAPLDLGKDMYVSVILDSDQLAENPEITVENSTTILYKERVSKDELN | |
| | TRSTTTSCFLFLDEPWCISVLETNRFNGKSIRPEIYSYKIPKYC | |
| 7 | LSQLQKNYLDNSNQQGDKMNNPDKKLSVNFNPLELDKGQKDLNKS | Cedar Virus G |
| | YYVKNKNYNVSNLLNESLHDIKFCIYCIFSLLIIITIINIITISIVITRLKVH | Protein (No |
| | EENNGMESPNLQSIQDSLSSLTNMINTEITPRIGILVTATSVTLSSSINY | Met) |
| | VGTKTNQLVNELKDYITKSCGFKVPELKLHECNISCADPKISKSAMY | |
| | STNAYAELAGPPKIFCKSVSKDPDFRLKQIDYVIPVQQDRSICMNNPL | |
| | LDISDGFFTYIHYEGINSCKKSDSFKVLLSHGEIVDRGDYRPSLYLLSS | |
| | HYHPYSMQVINCVPVTCNQSSFVFCHISNNTKTLDNSDYSSDEYYIT | |
| | YFNGIDRPKTKKIPINNMTADNRYIHFTFSGGGGVCLGEEFIIPVTTVI | |
| | NTDVFTHDYCESFNCSVQTGKSLKEICSESLRSPTNSSRYNLNGIMIIS | |
| | QNNMTDFKIQLNGITYNKLSFGSPGRLSKTLGQVLYYQSSMSWDTY | |
| | LKAGFVEKWKPFTPNWMNNTVISRPNQGNCPRYHKCPEICYGGTYN | |
| | DIAPLDLGKDMYVSVILDSDQLAENPEITVFNSTTILYKERVSKDELN | |
| | TRSTTTSCFLFLDEPWCISVLETNRFNGKSIRPEIYSYKIPKYC | |
| 8 | MPQKTVEFINMNSPLERGVSTLSDKKTLNQSKITKQGYFGLGSHSER | Bat |
| | NWKKQKNQNDHYMTVSTMILEILVVLGIMENLIVLTMVYYQNDNIN | Paramyxovirus |
| | QRMAELTSNITVLNLNLNQLTNKIQREIIPRITLIDTATTITIPSAITYILA | G Protein |
| | TLTTRISELLPSINQKCEFKTPTLVLNDCRINCTPPLNPSDGVKMSSLA | |
| | TNLVAHGPSPCRNFSSVPTIYYYRIPGLYNRTALDERCILNPRLTISST | |
| | KFAYVHSEYDKNCTRGFKYYELMTFGEILEGPEKEPRMFSRSFYSPT | |
| | NAVNYHSCTPIVTVNEGYFLCLECTSSDPLYKANLSNSTFHLVILRHN | |
| | KDEKIVSMPSFNLSTDQEYVQIIPAEGGGTAESGNLYFPCIGRLLHKR | |
| | VTHPLCKKSNCSRTDDESCLKSYYNQGSPQHQVVNCLIRIRNAQRDN | |
| | PTWDVITVDLTNTYPGSRSRIFGSFSKPMLYQSSVSWHTLLQVAEITD | |
| | LDKYQLDWLDTPYISRPGGSECPFGNYCPTVCWEGTYNDVYSLTPN | |
| | NDLFVTVYLKSEQVAENPYFAIFSRDQILKEFPLDAWISSARTTTISCF | |
| | MFNNEIWCIAALEITRLNDDIIRPIYYSFWLPTDCRTPYPHTGKMTRV | |
| | PLRSTYNY | |
| 9 | PQKTVEFINMNSPLERGVSTLSDKKTLNQSKITKQGYFGLGSHSERN | Bat |
| | WKKQKNQNDHYMTVSTMILEILVVLGIMFNLIVLTMVYYQNDNINQ | Paramyxovirus |
| | RMAELTSNITVLNLNLNQLTNKIQREIIPRITLIDTATTITIPSAITYILAT | G Protein (No |
| | LTTRISELLPSINQKCEFKTPTLVLNDCRINCTPPLNPSDGVKMSSLAT | Met) |
| | NLVAHGPSPCRNFSSVPTIYYYRIPGLYNRTALDERCILNPRLTISSTK | |
| | FAYVHSEYDKNCTRGFKYYELMTFGEILEGPEKEPRMFSRSFYSPTN | |
| | AVNYHSCTPIVTVNEGYFLCLECTSSDPLYKANLSNSTFHLVILRHNK | |
| | DEKIVSMPSFNLSTDQEYVQIIPAEGGGTAESGNLYFPCIGRLLHKRV | |
| | THPLCKKSNCSRTDDESCLKSYYNQGSPQHQVVNCLIRIRNAQRDNP | |
| | TWDVITVDLTNTYPGSRSRIFGSFSKPMLYQSSVSWHTLLQVAEITDL | |
| | DKYQLDWLDTPYISRPGGSECPFGNYCPTVCWEGTYNDVYSLTPNN | |
| | DLFVTVYLKSEQVAENPYFAIFSRDQILKEFPLDAWISSARTTTISCFM | |
| | FNNEIWCIAALEITRLNDDIIRPIYYSFWLPTDCRTPYPHTGKMTRVPL | |
| | RSTYNY | |
| 10 | MATNRDNTITSAEVSQEDKVKKYYGVETAEKVADSISGNKVFILMN | Mojiang virus, |
| | TLLILTGAIITITLNITNLTAAKSQQNMLKIIQDDVNAKLEMFVNLDQL | Tongguan 1 G |
| | VKGEIKPKVSLINTAVSVSIPGQISNLQTKFLQKYVYLEESITKQCTCN | Protein |
| | PLSGIFPTSGPTYPPTDKPDDDTTDDDKVDTTIKPIEYPKPDGCNRTG | |
| | DHFTMEPGANFYTVPNLGPASSNSDECYTNPSFSIGSSIYMFSQEIRKT | |
| | DCTAGEILSIQIVLGRIVDKGQQGPQASPLLVWAVPNPKIINSCAVAA | |
| | GDEMGWVLCSVTLTAASGEPIPHMFDGFWLYKLEPDTEVVSYRITG | |
| | YAYLLDKQYDSVFIGKGGGIQKGNDLYFQMYGLSRNRQSFKALCEH | |
| | GSCLGTGGGGYQVLCDRAVMSFGSEESLITNAYLKVNDLASGKPVII | |
| | GQTFPPSDSYKGSNGRMYTIGDKYGLYLAPSSWNRYLRFGITPDISV | |
| | RSTTWLKSQDPIMKILSTCTNTDRDMCPEICNTRGYQDIFPLSEDSEY | |
| | YTYIGITPNNGGTKNFVAVRDSDGHIASIDILQNYYSITSATISCFMYK | |
| | DEIWCIAITEGKKQKDNPQRIYAHSYKIRQMCYNMKSATVTVGNAK | |
| | NITIRRY | |
| 11 | ATNRDNTITSAEVSQEDKVKKYYGVETAEKVADSISGNKVFILMNTL | Mojiang virus, |
| | LILTGAIITITLNITNLTAAKSQQNMLKIIQDDVNAKLEMFVNLDQLV | Tongguan 1 G |
| | KGEIKPKVSLINTAVSVSIPGQISNLQTKFLQKYVYLEESITKQCTCNP | (No Met) |
| | LSGIFPTSGPTYPPTDKPDDDTTDDDKVDTTIKPIEYPKPDGCNRTGD | |
| | HFTMEPGANFYTVPNLGPASSNSDECYTNPSFSIGSSIYMFSQEIRKTD | |
| | CTAGEILSIQIVLGRIVDKGQQGPQASPLLVWAVPNPKIINSCAVAAG | |
| | DEMGWVLCSVTLTAASGEPIPHMFDGFWLYKLEPDTEVVSYRITGY | |
| | AYLLDKQYDSVFIGKGGGIQKGNDLYFQMYGLSRNRQSFKALCEHG | |
| | SCLGTGGGGYQVLCDRAVMSFGSEESLITNAYLKVNDLASGKPVIIG | |
| | QTFPPSDSYKGSNGRMYTIGDKYGLYLAPSSWNRYLRFGITPDISVRS | |
| | TTWLKSQDPIMKILSTCTNTDRDMCPEICNTRGYQDIFPLSEDSEYYT | |
| | YIGITPNNGGTKNFVAVRDSDGHIASIDILQNYYSITSATISCFMYKDE | |
| | IWCIAITEGKKQKDNPQRIYAHSYKIRQMCYNMKSATVTVGNAKNIT | |
| | IRRY | |
| 12 | MKVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ5 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVELDSN QTAENPVFTV | |
| | FKDNEILYRA QLASEDTNAQ KTITNCFLLK NKIWCISLVE | |
| | IYDTGDNVIR PKLFAVKIPE QCT | |
| 13 | MSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ20 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRENTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QCT | |
| 14 | MSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ25 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRENTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QCT | |
| 15 | ILHY EKLSKIGLVK GVTRKYKIKS NPLTKDIVIK MIPNVSNMSQ | Nipah virus |
| | CTGSVMENYK TRLNGILTPI KGALEIYKNQ THDLVGDVRL | NIV-F F0 T234 |
| | AGVIMAGVAI GIATAAQITA GVALYEAMKN ADNINKLKSS | truncation (aa |
| | IESTNEAVVK LQETAEKTVY VLTALQDYIN TNLVPTIDKI | 525-544) AND |
| | SCKQTELSLD LALSKYLSDL LFVFGPNLQD PVSNSMTIQA | mutation on N- |
| | ISQAFGGNYE TLLRTLGYAT EDFDDLLESD SITGQIIYVD | linked |
| | LSSYYIIVRV YFPILTEIQQ AYIQELLPVS FNNDNSEWIS | glycosylation |
| | IVPNFILVRN TLISNIEIGF CLITKRSVIC NQDYATPMTN | site |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK | |
| | KRNTGT | |
| 16 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | (with signal |
| | TLLRTLGYAT EDFDDLLESD SITGQIIYVD LSSYYIIVRV | sequence) |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 17 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated and |
| | PKLISYTLPV VGQSGTCITD PLLAMDEGYF AYSHLERIGS | mutated |
| | CSRGVSKQRI IGVGEVLDRG DEVPSLFMTN VWTPPNPNTV | (E501 A, |
| | YHCSAVYNNE FYYVLCAVST VGDPILNSTY WSGSLMMTRL | W504A, Q530A, |
| | AVKPKSNGGG YNQHQLALRS IEKGRYDKVM PYGPSGIKQG | E533A) NiV G |
| | DTLYFPAVGF LVRTEFKYND SNCPITKCQY SKPENCRLSM | protein (Gc A |
| | GIRPNSHYIL RSGLLKYNLS DGENPKVVFI EISDQRLSIG | 34) |
| | SPSKIYDSLG QPVFYQASFS WDTMIKFGDV LTVNPLVVNW | |
| | RNNTVISRPG QSQCPRENTC PAICAEGVYN DAFLIDRINW | |
| | ISAGVFLDSN ATAANPVFTV FKDNEILYRA QLASEDTNAQ | |
| | KTITNCFLLK NKIWCISLVE IYDTGDNVIR PKLFAVKIPE QCT | |
| 18 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated and |
| | PKLISYTLPV VGQSGTCITD PLLAMDEGYF AYSHLERIGS | mutated |
| | CSRGVSKQRI IGVGEVLDRG DEVPSLFMTN VWTPPNPNTV | (E501 A, |
| | YHCSAVYNNE FYYVLCAVST VGDPILNSTY WSGSLMMTRL | W504A, Q530A, |
| | AVKPKSNGGG YNQHQLALRS IEKGRYDKVM PYGPSGIKQG | E533A) NiV G |
| | DTLYFPAVGF LVRTEFKYND SNCPITKCQY SKPENCRLSM | protein (Gc A |
| | GIRPNSHYIL RSGLLKYNLS DGENPKVVFI EISDQRLSIG | 34) Without N- |
| | SPSKIYDSLG QPVFYQASFS WDTMIKFGDV LTVNPLVVNW | terminal |
| | RNNTVISRPG QSQCPRENTC PAICAEGVYN DAFLIDRINW | methionine |
| | ISAGVFLDSN ATAANPVFTV FKDNEILYRA QLASEDTNAQ | |
| | KTITNCFLLK NKIWCISLVE IYDTGDNVIR PKLFAVKIPE QCT | |
| 19 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | (with signal |
| | TLLRTLGYAT EDFDDLLESD SITGQIIYVD LSSYYIIVRV | sequence) |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 20 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NIV-F F0 T234 |
| | AGVIMAGVAI GIATAAQITA GVALYEAMKN ADNINKLKSS | truncation (aa |
| | IESTNEAVVK LQETAEKTVY VLTALQDYIN TNLVPTIDKI | 525-544) |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK | |
| | KRNTGT | |
| 21 | ILHY EKLSKIGLVK GVTRKYKIKS NPLTKDIVIK MIPNVSNMSQ | Truncated |
| | CTGSVMENYK TRLNGILTPI KGALEIYKNN THDLVGDVRL | mature NiV |
| | AGVIMAGVAI GIATAAQITA GVALYEAMKN ADNINKLKSS | fusion |
| | IESTNEAVVK LQETAEKTVY VLTALQDYIN TNLVPTIDKI | glycoprotein |
| | SCKQTELSLD LALSKYLSDL LFVFGPNLQD PVSNSMTIQA | (FcDelta22) at |
| | ISQAFGGNYE TLLRTLGYAT EDFDDLLESD SITGQIIYVD | cytoplasmic tail |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK | |
| | KRNT | |
| 22 | FNTVIALLGS IVIIVMNIMI IQNYTRSTDN QAVIKDALQG | NivG protein |
| | IQQQIKGLAD KIGTEIGPKV SLIDTSSTIT IPANIGLLGS KISQSTASIN | attachment |
| | ENVNEKCKFT LPPLKIHECN ISCPNPLPFR EYRPQTEGVS | glycoprotein |
| | NLVGLPNNIC LQKTSNQILK PKLISYTLPV VGQSGTCITD | Without |
| | PLLAMDEGYF AYSHLERIGS CSRGVSKQRI IGVGEVLDRG | cytoplasmic tail |
| | DEVPSLFMTN VWTPPNPNTV YHCSAVYNNE FYYVLCAVST | Uniprot |
| | VGDPILNSTY WSGSLMMTRL AVKPKSNGGG YNQHQLALRS | Q9IH62 |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 23 | MMADSKLVSL NNNLSGKIKD QGKVIKNYYG TMDIKKINDG | Hendra virus G |
| | LLDSKILGAF | protein Uniprot |
| | NTVIALLGSI IIIVMNIMII QNYTRTTDNQ ALIKESLQSV | O89343 |
| | QQQIKALTDK IGTEIGPKVS LIDTSSTITI PANIGLLGSK ISQSTSSINE | |
| | NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISLVEI | |
| | YDTGDSVIRP KLFAVKIPAQ CSES | |
| 24 | MADSKLVSL NNNLSGKIKD QGKVIKNYYG TMDIKKINDG | Hendra virus G |
| | LLDSKILGAF | protein Uniprot |
| | NTVIALLGSI IIIVMNIMII QNYTRTTDNQ ALIKESLQSV | O89343 |
| | QQQIKALTDK IGTEIGPKVS LIDTSSTITI PANIGLLGSK ISQSTSSINE | Without N- |
| | NVNDKCKFTL | terminal |
| | PPLKIHECNI SCPNPLPFRE YRPISQGVSD LVGLPNQICL | methionine |
| | QKTTSTILKP RLISYTLPIN TREGVCITDP LLAVDNGFFA | |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISLVEI | |
| | YDTGDSVIRP KLFAVKIPAQ CSES | |
| 25 | FNTVIALLGSI IIIVMNIMII QNYTRTTDNQ ALIKESLQSV | Hendra virus G |
| | QQQIKALTDK | protein Uniprot |
| | IGTEIGPKVS LIDTSSTITI PANIGLLGSK ISQSTSSINE NVNDKCKFTL | O89343 |
| | PPLKIHECNI SCPNPLPFRE YRPISQGVSD LVGLPNQICL | Without |
| | QKTTSTILKP | cytoplasmic tail |
| | RLISYTLPIN TREGVCITDP LLAVDNGFFA YSHLEKIGSC | |
| | TRGIAKQRII | |
| | GVGEVLDRGD KVPSMFMTNV WTPPNPSTIH HCSSTYHEDF | |
| | YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 26 | FNTVIALLGSI IIIVMNIMII QNYTRTTDNQ ALIKESLQSV | Hendra virus G |
| | QQQIKALTDK | protein Uniprot |
| | IGTEIGPKVS LIDTSSTITI PANIGLLGSK ISQSTSSINE NVNDKCKFTL | O89343 |
| | PPLKIHECNI SCPNPLPFRE YRPISQGVSD LVGLPNQICL | Without |
| | QKTTSTILKP | cytoplasmic tail |
| | RLISYTLPIN TREGVCITDP LLAVDNGFFA YSHLEKIGSC | |
| | TRGIAKQRII | |
| | GVGEVLDRGD KVPSMFMTNV WTPPNPSTIH HCSSTYHEDF | |
| | YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 27 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 28 | MATQEVRLKCLLCGIIVLVLSLEGLGILHYEKLSKIGLVKGITRKYKI | Hendra virus F |
| | KSNPLTKDIVIKMIPNVSNVSKCTGTVMENYKSRLTGILSPIKGAIELY | Protein |
| | NNNTHDLVGDVKLAGVVMAGIAIGIATAAQITAGVALYEAMKNAD | |
| | NINKLKSSIESTNEAVVKLQETAEKTVYVLTALQDYINTNLVPTIDQIS | |
| | CKQTELALDLALSKYLSDLLFVFGPNLQDPVSNSMTIQAISQAFGGN | |
| | YETLLRTLGYATEDFDDLLESDSIAGQIVYVDLSSYYIIVRVYFPILTEI | |
| | QQAYVQELLPVSFNNDNSEWISIVPNFVLIRNTLISNIEVKYCLITKKS | |
| | VICNQDYATPMTASVRECLTGSTDKCPRELVVSSHVPRFALSGGVLF | |
| | ANCISVTCQCQTTGRAISQSGEQTLLMIDNTTCTTVVLGNIIISLGKYL | |
| | GSINYNSESIAVGPPVYTDKVDISSQISSMNQSLQQSKDYIKEAQKILD | |
| | TVNPSLISMLSMIILYVLSIAALCIGLITFISFVIVEKKRGNYSRLDDRQ | |
| | VRPVSNGDLYYIGT | |
| 29 | ILHYEKLSKIGLVKGITRKYKIKSNPLTKDIVIKMIPNVSNVSKCTGTV | Hendra virus F |
| | MENYKSRLTGILSPIKGAIELYNNNTHDLVGDVKLAGVVMAGIAIGI | Protein, |
| | ATAAQITAGVALYEAMKNADNINKLKSSIESTNEAVVKLQETAEKT | Without signal |
| | VYVLTALQDYINTNLVPTIDQISCKQTELALDLALSKYLSDLLFVFGP | sequence |
| | NLQDPVSNSMTIQAISQAFGGNYETLLRTLGYATEDFDDLLESDSIAG | |
| | QIVYVDLSSYYIIVRVYFPILTEIQQAYVQELLPVSFNNDNSEWISIVP | |
| | NFVLIRNTLISNIEVKYCLITKKSVICNQDYATPMTASVRECLTGSTD | |
| | KCPRELVVSSHVPRFALSGGVLFANCISVTCQCQTTGRAISQSGEQTL | |
| | LMIDNTTCTTVVLGNIIISLGKYLGSINYNSESIAVGPPVYTDKVDISS | |
| | QISSMNQSLQQSKDYIKEAQKILDTVNPSLISMLSMIILYVLSIAALCIG | |
| | LITFISFVIVEKKRGNYSRLDDRQVRPVSNGDLYYIGT | |
| 30 | MVVILDKRCYCNLLILILMISECSVGILHYEKLSKIGLVKGVTRKYKI | Nipah virus F |
| | KSNPLTKDIVIKMIPNVSNMSQCTGSVMENYKTRLNGILTPIKGALEI | Protein |
| | YKNNTHDLVGDVRLAGVIMAGVAIGIATAAQITAGVALYEAMKNA | |
| | DNINKLKSSIESTNEAVVKLQETAEKTVYVLTALQDYINTNLVPTIDK | |
| | ISCKQTELSLDLALSKYLSDLLFVFGPNLQDPVSNSMTIQAISQAFGG | |
| | NYETLLRTLGYATEDFDDLLESDSITGQIIYVDLSSYYIIVRVYFPILTE | |
| | IQQAYIQELLPVSFNNDNSEWISIVPNFILVRNTLISNIEIGFCLITKRSVI | |
| | CNQDYATPMTNNMRECLTGSTEKCPRELVVSSHVPRFALSNGVLFA | |
| | NCISVTCQCQTTGRAISQSGEQTLLMIDNTTCPTAVLGNVIISLGKYL | |
| | GSVNYNSEGIAIGPPVFTDKVDISSQISSMNQSLQQSKDYIKEAQRLL | |
| | DTVNPSLISMLSMIILYVLSIASLCIGLITFISFIIVEKKRNTYSRLEDRR | |
| | VRPTSSGDLYYIGT | |
| 31 | ILHYEKLSKIGLVKGVTRKYKIKSNPLTKDIVIKMIPNVSNMSQCTGS | Nipah virus F |
| | VMENYKTRLNGILTPIKGALEIYKNNTHDLVGDVRLAGVIMAGVAIG | Protein, without |
| | IATAAQITAGVALYEAMKNADNINKLKSSIESTNEAVVKLQETAEKT | signal sequence |
| | VYVLTALQDYINTNLVPTIDKISCKQTELSLDLALSKYLSDLLFVFGP | |
| | NLQDPVSNSMTIQAISQAFGGNYETLLRTLGYATEDFDDLLESDSITG | |
| | QIIYVDLSSYYIIVRVYFPILTEIQQAYIQELLPVSFNNDNSEWISIVPNF | |
| | ILVRNTLISNIEIGFCLITKRSVICNQDYATPMTNNMRECLTGSTEKCP | |
| | RELVVSSHVPRFALSNGVLFANCISVTCQCQTTGRAISQSGEQTLLMI | |
| | DNTTCPTAVLGNVIISLGKYLGSVNYNSEGIAIGPPVFTDKVDISSQIS | |
| | SMNQSLQQSKDYIKEAQRLLDTVNPSLISMLSMIILYVLSIASLCIGLIT | |
| | FISFIIVEKKRNTYSRLEDRRVRPTSSGDLYYIGT | |
| 32 | MSNKRTTVLIIISYTLFYLNNAAIVGFDFDKLNKIGVVQGRVLNYKIK | Cedar Virus F |
| | GDPMTKDLVLKFIPNIVNITECVREPLSRYNETVRRLLLPIHNMLGLY | Protein |
| | LNNTNAKMTGLMIAGVIMGGIAIGIATAAQITAGFALYEAKKNTENI | |
| | QKLTDSIMKTQDSIDKLTDSVGTSILILNKLQTYINNQLVPNLELLSCR | |
| | QNKIEFDLMLTKYLVDLMTVIGPNINNPVNKDMTIQSLSLLFDGNYD | |
| | IMMSELGYTPQDFLDLIESKSITGQIIYVDMENLYVVIRTYLPTLIEVP | |
| | DAQIYEFNKITMSSNGGEYLSTIPNFILIRGNYMSNIDVATCYMTKAS | |
| | VICNQDYSLPMSQNLRSCYQGETEYCPVEAVIASHSPRFALTNGVIFA | |
| | NCINTICRCQDNGKTITQNINQFVSMIDNSTCNDVMVDKFTIKVGKY | |
| | MGRKDINNINIQIGPQIIIDKVDLSNEINKMNQSLKDSIFYLREAKRILD | |
| | SVNISLISPSVQLFLIIISVLSFIILLIIIVYLYCKSKHSYKYNKFIDDPDY | |
| | YNDYKRERINGKASKSNNIYYVGD | |
| 33 | TVLIIISYTLFYLNNAAIVGFDFDKLNKIGVVQGRVLNYKIKGDPMTK | Cedar Virus F |
| | DLVLKFIPNIVNITECVREPLSRYNETVRRLLLPIHNMLGLYLNNTNA | Protein, without |
| | KMTGLMIAGVIMGGIAIGIATAAQITAGFALYEAKKNTENIQKLTDSI | signal sequence |
| | MKTQDSIDKLTDSVGTSILILNKLQTYINNQLVPNLELLSCRQNKIEFD | |
| | LMLTKYLVDLMTVIGPNINNPVNKDMTIQSLSLLFDGNYDIMMSELG | |
| | YTPQDFLDLIESKSITGQIIYVDMENLYVVIRTYLPTLIEVPDAQIYEFN | |
| | KITMSSNGGEYLSTIPNFILIRGNYMSNIDVATCYMTKASVICNQDYS | |
| | LPMSQNLRSCYQGETEYCPVEAVIASHSPRFALTNGVIFANCINTICR | |
| | CQDNGKTITQNINQFVSMIDNSTCNDVMVDKFTIKVGKYMGRKDIN | |
| | NINIQIGPQIIIDKVDLSNEINKMNQSLKDSIFYLREAKRILDSVNISLIS | |
| | PSVQLFLIIISVLSFIILLIIIVYLYCKSKHSYKYNKFIDDPDYYNDYKRE | |
| | RINGKASKSNNIYYVGD | |
| 34 | MALNKNMFSSLFLGYLLVYATTVQSSIHYDSLSKVGVIKGLTYNYKI | Mojiang virus, |
| | KGSPSTKLMVVKLIPNIDSVKNCTQKQYDEYKNLVRKALEPVKMAI | Tongguan 1 F |
| | DTMLNNVKSGNNKYRFAGAIMAGVALGVATAATVTAGIALHRSNE | Protein |
| | NAQAIANMKSAIQNTNEAVKQLQLANKQTLAVIDTIRGEINNNIIPVI | |
| | NQLSCDTIGLSVGIRLTQYYSEIITAFGPALQNPVNTRITIQAISSVENG | |
| | NFDELLKIMGYTSGDLYEILHSELIRGNIIDVDVDAGYIALEIEFPNLT | |
| | LVPNAVVQELMPISYNIDGDEWVTLVPRFVLTRTTLLSNIDTSRCTIT | |
| | DSSVICDNDYALPMSHELIGCLQGDTSKCAREKVVSSYVPKFALSDG | |
| | LVYANCLNTICRCMDTDTPISQSLGATVSLLDNKRCSVYQVGDVLIS | |
| | VGSYLGDGEYNADNVELGPPIVIDKIDIGNQLAGINQTLQEAEDYIEK | |
| | SEEFLKGVNPSIITLGSMVVLYIFMILIAIVSVIALVLSIKLTVKGNVVR | |
| | QQFTYTQHVPSMENINYVSH | |
| 35 | IHYDSLSKVGVIKGLTYNYKIKGSPSTKLMVVKLIPNIDSVKNCTQKQ | Mojiang virus, |
| | YDEYKNLVRKALEPVKMAIDTMLNNVKSGNNKYRFAGAIMAGVAL | Tongguan 1 F |
| | GVATAATVTAGIALHRSNENAQAIANMKSAIQNTNEAVKQLQLANK | Protein, without |
| | QTLAVIDTIRGEINNNIIPVINQLSCDTIGLSVGIRLTQYYSEIITAFGPA | signal sequence |
| | LQNPVNTRITIQAISSVENGNFDELLKIMGYTSGDLYEILHSELIRGNII | |
| | DVDVDAGYIALEIEFPNLTLVPNAVVQELMPISYNIDGDEWVTLVPR | |
| | FVLTRTTLLSNIDTSRCTITDSSVICDNDYALPMSHELIGCLQGDTSKC | |
| | AREKVVSSYVPKFALSDGLVYANCLNTICRCMDTDTPISQSLGATVS | |
| | LLDNKRCSVYQVGDVLISVGSYLGDGEYNADNVELGPPIVIDKIDIG | |
| | NQLAGINQTLQEAEDYIEKSEEFLKGVNPSIITLGSMVVLYIFMILIAIV | |
| | SVIALVLSIKLTVKGNVVRQQFTYTQHVPSMENINYVSH | |
| 36 | MKKKTDNPTISKRGHNHSRGIKSRALLRETDNYSNGLIVENLVRNCH | Bat |
| | HPSKNNLNYTKTQKRDSTIPYRVEERKGHYPKIKHLIDKSYKHIKRG | Paramyxovirus |
| | KRRNGHNGNIITIILLLILILKTQMSEGAIHYETLSKIGLIKGITREYKV | F Protein |
| | KGTPSSKDIVIKLIPNVTGLNKCTNISMENYKEQLDKILIPINNIIELYA | |
| | NSTKSAPGNARFAGVIIAGVALGVAAAAQITAGIALHEARQNAERIN | |
| | LLKDSISATNNAVAELQEATGGIVNVITGMQDYINTNLVPQIDKLQCS | |
| | QIKTALDISLSQYYSEILTVFGPNLQNPVTTSMSIQAISQSFGGNIDLLL | |
| | NLLGYTANDLLDLLESKSITGQITYINLEHYFMVIRVYYPIMTTISNAY | |
| | VQELIKISFNVDGSEWVSLVPSYILIRNSYLSNIDISECLITKNSVICRH | |
| | DFAMPMSYTLKECLTGDTEKCPREAVVTSYVPRFAISGGVIYANCLS | |
| | TTCQCYQTGKVIAQDGSQTLMMIDNQTCSIVRIEEILISTGKYLGSQE | |
| | YNTMHVSVGNPVFTDKLDITSQISNINQSIEQSKFYLDKSKAILDKINL | |
| | NLIGSVPISILFIIAILSLILSIITFVIVMIIVRRYNKYTPLINSDPSSRRSTI | |
| | QDVYIIPNPGEHSIRSAARSIDRDRD | |
| 37 | SRALLRETDNYSNGLIVENLVRNCHHPSKNNLNYTKTQKRDSTIPYR | Bat |
| | VEERKGHYPKIKHLIDKSYKHIKRGKRRNGHNGNIITIILLLILILKTQ | Paramyxovirus |
| | MSEGAIHYETLSKIGLIKGITREYKVKGTPSSKDIVIKLIPNVTGLNKC | F Protein, |
| | TNISMENYKEQLDKILIPINNIIELYANSTKSAPGNARFAGVIIAGVAL | without signal |
| | GVAAAAQITAGIALHEARQNAERINLLKDSISATNNAVAELQEATGG | sequence |
| | IVNVITGMQDYINTNLVPQIDKLQCSQIKTALDISLSQYYSEILTVFGP | |
| | NLQNPVTTSMSIQAISQSFGGNIDLLLNLLGYTANDLLDLLESKSITG | |
| | QITYINLEHYFMVIRVYYPIMTTISNAYVQELIKISFNVDGSEWVSLVP | |
| | SYILIRNSYLSNIDISECLITKNSVICRHDFAMPMSYTLKECLTGDTEK | |
| | CPREAVVTSYVPRFAISGGVIYANCLSTTCQCYQTGKVIAQDGSQTL | |
| | MMIDNQTCSIVRIEEILISTGKYLGSQEYNTMHVSVGNPVFTDKLDIT | |
| | SQISNINQSIEQSKFYLDKSKAILDKINLNLIGSVPISILFIIAILSLILSIIT | |
| | FVIVMIIVRRYNKYTPLINSDPSSRRSTIQDVYIIPNPGEHSIRSAARSID | |
| | RDRD | |
| 38 | MVVILDKRCY CNLLILILMI SECSVG | signal sequence |
| 39 | ILHYEKLSKIGLVKGVTRKYKIKSNPLTKDIVIKMIPNVSNMSQCTGS | Nipah virus |
| | VMENYKTRLNGILTPIKGALEIYKNNTHDLVGDVR | NiV-F F2 (aa |
| | | 27-109) |
| 40 | MVVILDKRCYCNLLILILMISECSVGILHYEKLSKIGLVKGVTRKYKI | Nipah virus F |
| | KSNPLTKDIVIKMIPNVSNMSQCTGSVMENYKTRLNGILTPIKGALEI | Protein |
| | YKNNTHDLVGDVRLAGVIMAGVAIGIATAAQITAGVALYEAMKNA | |
| | DNINKLKSSIESTNEAVVKLQETAEKTVYVLTALQDYINTNLVPTIDK | |
| | ISCKQTELSLDLALSKYLSDLLFVFGPNLQDPVSNSMTIQAISQAFGG | |
| | NYETLLRTLGYATEDFDDLLESDSITGQIIYVDLSSYYIIVRVYFPILTE | |
| | IQQAYIQELLPVSFNNDNSEWISIVPNFILVRNTLISNIEIGFCLITKRSVI | |
| | CNQDYATPMTNNMRECLTGSTEKCPRELVVSSHVPRFALSNGVLFA | |
| | NCISVTCQCQTTGRAISQSGEQTLLMIDNTTCPTAVLGNVIISLGKYL | |
| | GSVNYNSEGIAIGPPVFTDKVDISSQISSMNQSLQQSKDYIKEAQRLL | |
| | DTVNPSLISMLSMIILYVLSIASLCIGLITFISFIIVEKKRNTYSRLEDRR | |
| | VRPTSSGDLYYIGT | |
| 41 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 (aa |
| | AGVIMAGVAI GIATAAQITA GVALYEAMKN ADNINKLKSS | 27-546) |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK | |
| | KRNTYSRLED RRVRPTSSGD LYYIGT | |
| 42 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated (Gc |
| | PKLISYTLPV VGQSGTCITD PLLAMDEGYF AYSHLERIGS | Δ34) |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 43 | MTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRENTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QCT | |
| 44 | MGNTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ10 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRENTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 45 | MGKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ15 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRENTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 46 | LAGVIMAGVAIGIATAAQITAGVALYEAMKNADNINKLKSSIESTNE | Nipah virus |
| | AVVKLQETAEKTVYVLTALQDYINTNLVPTIDKISCKQTELSLDLALS | NIV F F1 (aa |
| | KYLSDLLFVFGPNLQDPVSNSMTIQAISQAFGGNYETLLRTLGYATE | 110-546) |
| | DFDDLLESDSITGQIIYVDLSSYYIIVRVYFPILTEIQQAYIQELLPVSFN | |
| | NDNSEWISIVPNFILVRNTLISNIEIGFCLITKRSVICNQDYATPMTNN | |
| | MRECLTGSTEKCPRELVVSSHVPRFALSNGVLFANCISVTCQCQTTG | |
| | RAISQSGEQTLLMIDNTTCPTAVLGNVIISLGKYLGSVNYNSEGIAIGP | |
| | PVFTDKVDISSQISSMNQSLQQSKDYIKEAQRLLDTVNPSLISMLSMII | |
| | LYVLSIASLCIGLITFISFIIVEKKRNTYSRLEDRRVRPTSSGDLYYIGT | |
| 47 | MALPVTALLLPLALLLHAARP | CD8α signal |
| | | peptide |
| 48 | METDTLLLWVLLLWVPGSTG | IgK signal |
| | | peptide |
| 49 | MLLLVTSLLLCELPHPAFLLIP | GMCSFR-α |
| | | (CSF2RA) |
| | | signal peptide |
| 50 | TTTPAPRPPTPAPTIASQPLSLRPEACRPAAGGAVHTRGLDFACD | CD8α hinge |
| | | domain |
| 51 | IEVMYPPPYLDNEKSNGTIIHVKGKHLCPSPLFPGPSKP | CD28 hinge |
| | | domain |
| 52 | AAAIEVMYPPPYLDNEKSNGTIIHVKGKHLCPSPLFPGPSKP | CD28 hinge |
| | | domain |
| 53 | ESKYGPPCPPCP | IgG4 hinge |
| | | domain |
| 54 | ESKYGPPCPSCP | IgG4 hinge |
| | | domain |
| 55 | ESKYGPPCPPCPAPEFLGGPSVFLFPPKPKDTLMISRTPEVTCVVVDVS | IgG4 hinge- |
| | QEDPEVQFNWYVDGVEVHNAKTKPREEQFNSTYRVVSVLTVLHQD | CH2-CH3 |
| | WLNGKEYKCKVSNKGLPSSIEKTISKAKGQPREPQVYTLPPSQEEMT | domain |
| | KNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| 56 | IYIWAPLAGTCGVLLLSLVITLYC | CD8α |
| | | transmembrane |
| | | domain |
| 57 | FWVLVVVGGVLACYSLLVTVAFIIFWV | CD28 |
| | | transmembrane |
| | | domain |
| 58 | MFWVLVVVGGVLACYSLLVTVAFIIFWV | CD28 |
| | | transmembrane |
| | | domain |
| 59 | KRGRKKLLYIFKQPFMRPVQTTQEEDGCSCRFPEEEEGGCEL | 4-1BB |
| | | costimulatory |
| | | domain |
| 60 | RSKRSRLLHSDYMNMTPRRPGPTRKHYQPYAPPRDFAAYRS | CD28 |
| | | costimulatory |
| | | domain |
| 61 | RVKFSRSADAPAYQQGQNQLYNELNLGRREEYDVLDKRRGRDPEM | CD3ζ signaling |
| | GGKPRRKNPQEGLYNELQKDKMAEAYSEIGMKGERRRGKGHDGLY | domain |
| | QGLSTATKDTYDALHMQALPPR | |
| 62 | RVKFSRSADAPAYKQGQNQLYNELNLGRREEYDVLDKRRGRDPEM | CD3ζ signaling |
| | GGKPRRKNPQEGLYNELQKDKMAEAYSEIGMKGERRRGKGHDGLY | domain (with Q |
| | QGLSTATKDTYDALHMQALPPR | to K mutation at |
| | | position 14) |
| 63 | DIQMTQTTSSLSASLGDRVTISCRASQDISKYLNWYQQKPDGTVKLLI | Anti-CD19 |
| | YHTSRLHSGVPSRFSGSGSGTDYSLTISNLEQEDIATYFCQQGNTLPY | FMC63 scFv |
| | TFGGGTKLEITGSTSGSGKPGSGEGSTKGEVKLQESGPGLVAPSQSLS | entire sequence, |
| | VTCTVSGVSLPDYGVSWIRQPPRKGLEWLGVIWGSETTYYNSALKS | with Whitlow |
| | RLTIIKDNSKSQVFLKMNSLQTDDTAIYYCAKHYYYGGSYAMDYW | linker |
| | GQGTSVTVSS | |
| 64 | DIQMTQTTSSLSASLGDRVTISCRASQDISKYLNWYQQKPDGTVKLLI | Anti-CD19 |
| | YHTSRLHSGVPSRFSGSGSGTDYSLTISNLEQEDIATYFCQQGNTLPY | FMC63 scFv |
| | TFGGGTKLEIT | light chain |
| | | variable region |
| 65 | QDISKY | Anti-CD19 |
| | | FMC63 scFv |
| | | light chain |
| | | CDR1 |
| 66 | HTS | Anti-CD19 |
| | | FMC63 scFv |
| | | light chain |
| | | CDR2 |
| 67 | QQGNTLPYT | Anti-CD19 |
| | | FMC63 scFv |
| | | light chain |
| | | CDR3 |
| 68 | GSTSGSGKPGSGEGSTKG | Whitlow linker |
| 69 | EVKLQESGPGLVAPSQSLSVTCTVSGVSLPDYGVSWIRQPPRKGLEW | Anti-CD19 |
| | LGVIWGSETTYYNSALKSRLTIIKDNSKSQVFLKMNSLQTDDTAIYY | FMC63 scFv |
| | CAKHYYYGGSYAMDYWGQGTSVTVSS | heavy chain |
| | | variable region |
| 70 | GVSLPDYG | Anti-CD19 |
| | | FMC63 scFv |
| | | heavy chain |
| | | CDR1 |
| 71 | IWGSETT | Anti-CD19 |
| | | FMC63 scFv |
| | | heavy chain |
| | | CDR2 |
| 72 | AKHYYYGGSYAMDY | Anti-CD19 |
| | | FMC63 scFv |
| | | heavy chain |
| | | CDR3 |
| 73 | DIQMTQTTSSLSASLGDRVTISCRASQDISKYLNWYQQKPDGTVKLLI | Anti-CD19 |
| | YHTSRLHSGVPSRFSGSGSGTDYSLTISNLEQEDIATYFCQQGNTLPY | FMC63 scFv |
| | TFGGGTKLEITGGGGSGGGGSGGGGSEVKLQESGPGLVAPSQSLSVT | entire sequence, |
| | CTVSGVSLPDYGVSWIRQPPRKGLEWLGVIWGSETTYYNSALKSRLT | with 3xG4S |
| | IIKDNSKSQVFLKMNSLQTDDTAIYYCAKHYYYGGSYAMDYWGQG | linker |
| | TSVTVSS | |
| 74 | atggccttaccagtgaccgccttgctcctgccgctggccttgctgctccacgccgccaggccggacatcc | Exemplary |
| | agatgacacagactacatcctccctgtctgcctctctgggagacagagtcaccatcagttgcagggcaag | CD19 CAR |
| | tcaggacattagtaaatatttaaattggtatcagcagaaaccagatggaactgttaaactcctgatctac | nucleotide |
| | catacatcaagattacactcaggagtcccatcaaggttcagtggcagtgggtctggaacagattattctc | sequence |
| | tcaccattagcaacctggagcaagaagatattgccacttacttttgccaacagggtaatacgcttccgta | |
| | cacgttcggaggggggaccaagctggagatcacaggctccacctctggatccggcaagcccggatctggc | |
| | gagggatccaccaagggcgaggtgaaactgcaggagtcaggacctggcctggtggcgccctcacagagcc | |
| | tgtccgtcacatgcactgtctcaggggtctcattacccgactatggtgtaagctggattcgccagcctcc | |
| | acgaaagggtctggagtggctgggagtaatatggggtagtgaaaccacatactataattcagctctcaaa | |
| | tccagactgaccatcatcaaggacaactccaagagccaagttttcttaaaaatgaacagtctgcaaactg | |
| | atgacacagccatttactactgtgccaaacattattactacggtggtagctatgctatggactactgggg | |
| | ccaaggaacctcagtcaccgtctcctcaaccacgacgccagcgccgcgaccaccaacaccggcgcccacc | |
| | atcgcgtcgcagcccctgtccctgcgcccagaggcgtgccggccagcggggggggcgcagtgcacacgag | |
| | ggggctggacttcgcctgtgatatctacatctgggcgcccttggccgggacttgtggggtccttctcctg | |
| | tcactggttatcaccctttactgcaaacggggcagaaagaaactcctgtatatattcaaacaaccattta | |
| | tgagaccagtacaaactactcaagaggaagatggctgtagctgccgatttccagaagaagaagaaggagg | |
| | atgtgaactgagagtgaagttcagcaggagcgcagacgcccccgcgtaccagcagggccagaaccagctc | |
| | tataacgagctcaatctaggacgaagagaggagtacgatgttttggacaagagacgtggccgggaccctg | |
| | agatggggggaaagccgagaaggaagaaccctcaggaaggcctgtacaatgaactgcagaaagataagat | |
| | ggcggaggcctacagtgagattgggatgaaaggcgagcgccggaggggcaaggggcacgatggcctttac | |
| | cagggtctcagtacagccaccaaggacacctacgacgcccttcacatgcaggccctgccccctcgc | |
| 75 | MALPVTALLLPLALLLHAARPDIQMTQTTSSLSASLGDRVTISCRASQ | Exemplary |
| | DISKYLNWYQQKPDGTVKLLIYHTSRLHSGVPSRFSGSGSGTDYSLTI | CD19 CAR |
| | SNLEQEDIATYFCQQGNTLPYTFGGGTKLEITGSTSGSGKPGSGEGST | amino acid |
| | KGEVKLQESGPGLVAPSQSLSVTCTVSGVSLPDYGVSWIRQPPRKGL | sequence |
| | EWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQ | |
| | PLSLRPEACRPAAGGAVHTRGLDFACDIYIWAPLAGTCGVLLLSLVIT | |
| | LYCKRGRKKLLYIFKQPFMRPVQTTQEEDGCSCRFPEEEEGGCELRV | |
| | KFSRSADAPAYQQGQNQLYNELNLGRREEYDVLDKRRGRDPEMGG | |
| | KPRRKNPQEGLYNELQKDKMAEAYSEIGMKGERRRGKGHDGLYQG | |
| | LSTATKDTYDALHMQALPPR | |
| 76 | atggccttaccagtgaccgccttgctcctgccgctggccttgctgctccacgccgccaggccggacatcc | Tisagenlecleuce |
| | agatgacacagactacatcctccctgtctgcctctctgggagacagagtcaccatcagttgcagggcaag | 1 CD19 CAR |
| | tcaggacattagtaaatatttaaattggtatcagcagaaaccagatggaactgttaaactcctgatcta | nucleotide |
| | ccatacatcaagattacactcaggagtcccatcaaggttcagtggcagtgggtctggaacagattattct | sequence |
| | ctcaccattagcaacctggagcaagaagatattgccacttacttttgccaacagggtaatacgcttccgt | |
| | acacgttcggaggggggaccaagctggagatcacaggtggcggtggctcgggcggtggtgggtcgggtgg | |
| | cggcggatctgaggtgaaactgcaggagtcaggacctggcctggtggcgccctcacagagcctgtccgtc | |
| | acatgcactgtctcaggggtctcattacccgactatggtgtaagctggattcgccagcctccacgaaag | |
| | ggtctggagtggctgggagtaatatggggtagtgaaaccacatactataattcagctctcaaatccaga | |
| | ctgaccatcatcaaggacaactccaagagccaagttttcttaaaaatgaacagtctgcaaactgatgaca | |
| | cagccatttactactgtgccaaacattattactacggtggtagctatgctatggactactggggccaagg | |
| | aacctcagtcaccgtctcctcaaccacgacgccagcgccgcgaccaccaacaccggcgcccaccatcgcg | |
| | tcgcagcccctgtccctgcgcccagaggcgtgccggccagcggggggggcgcagtgcacacgagggggct | |
| | ggacttcgcctgtgatatctacatctgggcgcccttggccgggacttgtggggtccttctcctgtcactg | |
| | gttatcaccctttactgcaaacggggcagaaagaaactcctgtatatattcaaacaaccatttatgagac | |
| | cagtacaaactactcaagaggaagatggctgtagctgccgatttccagaagaagaagaaggaggatgtga | |
| | actgagagtgaagttcagcaggagcgcagacgcccccgcgtacaagcagggccagaaccagctctataac | |
| | gagctcaatctaggacgaagagaggagtacgatgttttggacaagagacgtggccgggaccctgagatgg | |
| | ggggaaagccgagaaggaagaaccctcaggaaggcctgtacaatgaactgcagaaagataagatggcgga | |
| | ggcctacagtgagattgggatgaaaggcgagcgccggaggggcaaggggcacgatggcctttaccagggt | |
| | ctcagtacagccaccaaggacacctacgacgcccttcacatgcaggccctgccccctcgc | |
| 77 | MALPVTALLLPLALLLHAARPDIQMTQTTSSLSASLGDRVTISCRASQ | Tisagenlecleuce |
| | DISKYLNWYQQKPDGTVKLLIYHTSRLHSGVPSRFSGSGSGTDYSLTI | 1 CD19 CAR |
| | SNLEQEDIATYFCQQGNTLPYTFGGGTKLEITGGGGSGGGGSGGGGS | amino acid |
| | EVKLQESGPGLVAPSQSLSVTCTVSGVSLPDYGVSWIRQPPRKGLEW | sequence |
| | LGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLS | |
| | LRPEACRPAAGGAVHTRGLDFACDIYIWAPLAGTCGVLLLSLVITLY | |
| | CKRGRKKLLYIFKQPFMRPVQTTQEEDGCSCRFPEEEEGGCELRVKF | |
| | SRSADAPAYKQGQNQLYNELNLGRREEYDVLDKRRGRDPEMGGKP | |
| | RRKNPQEGLYNELQKDKMAEAYSEIGMKGERRRGKGHDGLYQGLS | |
| | TATKDTYDALHMQALPPR | |
| 78 | atgctgctgctggtgaccagcctgctgctgtgcgagctgccccaccccgcctttctgctgatccccgaca | Lisocabtagene |
| | tccagatgacccagaccacctccagcctgagcgccagcctgggcgaccgggtgaccatcagctgccgggc | maraleucel |
| | cagccaggacatcagcaagtacctgaactggtatcagcagaagcccgacggcaccgtcaagctgctgatc | CD19 CAR |
| | taccacaccagccggctgcacagcggcgtgcccagccggtttagcggcagcggctccggcaccgactaca | nucleotide |
| | gcctgaccatctccaacctggaacaggaagatatcgccacctacttttgccagcagggcaacacactgcc | sequence |
| | ctacacctttggcggcggaacaaagctggaaatcaccggcagcacctccggcagcggcaagcctggcagc | |
| | ggcgagggcagcaccaagggcgaggtgaagctgcaggaaagcggccctggcctggtggcccccagccaga | |
| | gcctgagcgtgacctgcaccgtgagcggcgtgagcctgcccgactacggcgtgagctggatccggcagcc | |
| | ccccaggaagggcctggaatggctgggcgtgatctggggcagcgagaccacctactacaacagcgccctg | |
| | aagagccggctgaccatcatcaaggacaacagcaagagccaggtgttcctgaagatgaacagcctgcaga | |
| | ccgacgacaccgccatctactactgcgccaagcactactactacggcggcagctacgccatggactactg | |
| | gggccagggcaccagcgtgaccgtgagcagcgaatctaagtacggaccgccctgccccccttgccctatg | |
| | ttctgggtgctggtggtggtcggaggcgtgctggcctgctacagcctgctggtcaccgtggccttcatca | |
| | tcttttgggtgaaacggggcagaaagaaactcctgtatatattcaaacaaccatttatgagaccagtaca | |
| | aactactcaagaggaagatggctgtagctgccgatttccagaagaagaagaaggaggatgtgaactgcgg | |
| | gtgaagttcagcagaagcgccgacgcccctgcctaccagcagggccagaatcagctgtacaacgagctga | |
| | acctgggcagaagggaagagtacgacgtcctggataagcggagaggccgggaccctgagatgggcggcaa | |
| | gcctcggcggaagaacccccaggaaggcctgtataacgaactgcagaaagacaagatggccgaggcctac | |
| | agcgagatcggcatgaagggcgagcggaggcggggcaagggccacgacggcctgtatcagggcctgtcca | |
| | ccgccaccaaggatacctacgacgccctgcacatgcaggccctgcccccaagg | |
| 79 | MLLLVTSLLLCELPHPAFLLIPDIQMTQTTSSLSASLGDRVTISCRASQ | Lisocabtagene |
| | DISKYLNWYQQKPDGTVKLLIYHTSRLHSGVPSRFSGSGSGTDYSLTI | maraleucel |
| | SNLEQEDIATYFCQQGNTLPYTFGGGTKLEITGSTSGSGKPGSGEGST | CD19 CAR |
| | KGEVKLQESGPGLVAPSQSLSVTCTVSGVSLPDYGVSWIRQPPRKGL | amino acid |
| | EWLGVIWGSETTYYNSALKSRLTIIKDNSKSQVFLKMNSLQTDDTAI | sequence |
| | YYCAKHYYYGGSYAMDYWGQGTSVTVSSESKYGPPCPPCPMFWVL | |
| | VVVGGVLACYSLLVTVAFIIFWVKRGRKKLLYIFKQPFMRPVQTTQE | |
| | EDGCSCRFPEEEEGGCELRVKFSRSADAPAYQQGQNQLYNELNLGR | |
| | REEYDVLDKRRGRDPEMGGKPRRKNPQEGLYNELQKDKMAEAYSE | |
| | IGMKGERRRGKGHDGLYQGLSTATKDTYDALHMQALPPR | |
| 80 | atgcttctcctggtgacaagccttctgctctgtgagttaccacacccagcattcctcctgatcccagaca | Axicabtagene |
| | tccagatgacacagactacatcctccctgtctgcctctctgggagacagagtcaccatcagttgcagggc | ciloleucel CD19 |
| | aagtcaggacattagtaaatatttaaattggtatcagcagaaaccagatggaactgttaaactcctgatc | CAR nucleotide |
| | taccatacatcaagattacactcaggagtcccatcaaggttcagtggcagtgggtctggaacagattatt | sequence |
| | ctctcaccattagcaacctggagcaagaagatattgccacttacttttgccaacagggtaatacgcttcc | |
| | gtacacgttcggaggggggactaagttggaaataacaggctccacctctggatccggcaagcccggatct | |
| | ggcgagggatccaccaagggcgaggtgaaactgcaggagtcaggacctggcctggtggcgccctcacaga | |
| | gcctgtccgtcacatgcactgtctcaggggtctcattacccgactatggtgtaagctggattcgccagcc | |
| | tccacgaaagggtctggagtggctgggagtaatatggggtagtgaaaccacatactataattcagctctc | |
| | aaatccagactgaccatcatcaaggacaactccaagagccaagttttcttaaaaatgaacagtctgcaaa | |
| | ctgatgacacagccatttactactgtgccaaacattattactacggtggtagctatgctatggactactg | |
| | gggtcaaggaacctcagtcaccgtctcctcagcggccgcaattgaagttatgtatcctcctccttaccta | |
| | gacaatgagaagagcaatggaaccattatccatgtgaaagggaaacacctttgtccaagtcccctatttc | |
| | ccggaccttctaagcccttttgggtgctggtggtggttgggggagtcctggcttgctatagcttgctagt | |
| | aacagtggcctttattattttctgggtgaggagtaagaggagcaggctcctgcacagtgactacatgaac | |
| | atgactccccgccgccccgggcccacccgcaagcattaccagccctatgccccaccacgcgacttcgcag | |
| | cctatcgctccagagtgaagttcagcaggagcgcagacgcccccgcgtaccagcagggccagaaccagct | |
| | ctataacgagctcaatctaggacgaagagaggagtacgatgttttggacaagagacgtggccgggaccct | |
| | gagatggggggaaagccgagaaggaagaaccctcaggaaggcctgtacaatgaactgcagaaagataaga | |
| | tggcggaggcctacagtgagattgggatgaaaggcgagcgccggaggggcaaggggcacgatggccttta | |
| | ccagggtctcagtacagccaccaaggacacctacgacgcccttcacatgcaggccctgccccctcgc | |
| 81 | MLLLVTSLLLCELPHPAFLLIPDIQMTQTTSSLSASLGDRVTISCRASQ | Axicabtagene |
| | DISKYLNWYQQKPDGTVKLLIYHTSRLHSGVPSRFSGSGSGTDYSLTI | ciloleucel CD19 |
| | SNLEQEDIATYFCQQGNTLPYTFGGGTKLEITGSTSGSGKPGSGEGST | CAR amino |
| | KGEVKLQESGPGLVAPSQSLSVTCTVSGVSLPDYGVSWIRQPPRKGL | acid sequence |
| | EWLGVIWGSETTYYNSALKSRLTIIKDNSKSQVFLKMNSLQTDDTAI | |
| | YYCAKHYYYGGSYAMDYWGQGTSVTVSSAAAIEVMYPPPYLDNEK | |
| | SNGTIIHVKGKHLCPSPLFPGPSKPFWVLVVVGGVLACYSLLVTVAFI | |
| | IFWVRSKRSRLLHSDYMNMTPRRPGPTRKHYQPYAPPRDFAAYRSR | |
| | VKFSRSADAPAYQQGQNQLYNELNLGRREEYDVLDKRRGRDPEMG | |
| | GKPRRKNPQEGLYNELQKDKMAEAYSEIGMKGERRRGKGHDGLYQ | |
| | GLSTATKDTYDALHMQALPPR | |
| 82 | DIVLTQSPAILSASPGEKVTMTCRASSSVNYMDWYQKKPGSSPKPWI | Anti-CD20 |
| | YATSNLASGVPARFSGSGSGTSYSLTISRVEAEDAATYYCQQWSFNP | Leu16 scFv |
| | PTFGGGTKLEIKGSTSGSGKPGSGEGSTKGEVQLQQSGAELVKPGAS | entire sequence, |
| | VKMSCKASGYTFTSYNMHWVKQTPGQGLEWIGAIYPGNGDTSYNQ | with Whitlow |
| | KFKGKATLTADKSSSTAYMQLSSLTSEDSADYYCARSNYYGSSYWF | linker |
| | FDVWGAGTTVTVSS | |
| 83 | DIVLTQSPAILSASPGEKVTMTCRASSSVNYMDWYQKKPGSSPKPWI | Anti-CD20 |
| | YATSNLASGVPARFSGSGSGTSYSLTISRVEAEDAATYYCQQWSFNP | Leu16 scFv |
| | PTFGGGTKLEIK | light chain |
| | | variable region |
| 84 | RASSSVNYMD | Anti-CD20 |
| | | Leu16 scFv |
| | | light chain |
| | | CDR1 |
| 85 | ATSNLAS | Anti-CD20 |
| | | Leu16 scFv |
| | | light chain |
| | | CDR2 |
| 86 | QQWSFNPPT | Anti-CD20 |
| | | Leu16 scFv |
| | | light chain |
| | | CDR3 |
| 87 | EVQLQQSGAELVKPGASVKMSCKASGYTFTSYNMHWVKQTPGQGL | Anti-CD20 |
| | EWIGAIYPGNGDTSYNQKFKGKATLTADKSSSTAYMQLSSLTSEDSA | Leu16 scFv |
| | DYYCARSNYYGSSYWFFDVWGAGTTVTVSS | heavy chain |
| 88 | SYNMH | Anti-CD20 |
| | | Leu16 scFv |
| | | heavy chain |
| | | CDR1 |
| 89 | AIYPGNGDTSYNQKFKG | Anti-CD20 |
| | | Leu16 scFv |
| | | heavy chain |
| | | CDR2 |
| 90 | QVQLQQSGPGLVKPSQTLSLTCAISGDSVSSNSAAWNWIRQSPSRGL | Anti-CD22 |
| | EWLGRTYYRSKWYNDYAVSVKSRITINPDTSKNQFSLQLNSVTPEDT | m971 scFv |
| | AVYYCAREVTGDLEDAFDIWGQGTMVTVSSGGGGSGGGGSGGGGS | entire sequence, |
| | DIQMTQSPSSLSASVGDRVTITCRASQTIWSYLNWYQQRPGKAPNLLI | with 3xG4S |
| | YAASSLQSGVPSRFSGRGSGTDFTLTISSLQAEDFATYYCQQSYSIPQT | linker |
| | FGQGTKLEIK | |
| 91 | QVQLQQSGPGLVKPSQTLSLTCAISGDSVSSNSAAWNWIRQSPSRGL | Anti-CD22 |
| | EWLGRTYYRSKWYNDYAVSVKSRITINPDTSKNQFSLQLNSVTPEDT | m971 scFv |
| | AVYYCAREVTGDLEDAFDIWGQGTMVTVSS | heavy chain |
| | | variable region |
| 92 | GDSVSSNSAA | Anti-CD22 |
| | | m971 scFv |
| | | heavy chain |
| | | CDR1 |
| 93 | TYYRSKWYN | Anti-CD22 |
| | | m971 scFv |
| | | heavy chain |
| | | CDR2 |
| 94 | AREVTGDLEDAFDI | Anti-CD22 |
| | | m971 scFv |
| | | heavy chain |
| | | CDR3 |
| 95 | DIQMTQSPSSLSASVGDRVTITCRASQTIWSYLNWYQQRPGKAPNLLI | Anti-CD22 |
| | YAASSLQSGVPSRFSGRGSGTDFTLTISSLQAEDFATYYCQQSYSIPQT | m971 scFv light |
| | FGQGTKLEIK | chain |
| 96 | QTIWSY | Anti-CD22 |
| | | m971 scFv light |
| | | chain CDR1 |
| 97 | AAS | Anti-CD22 |
| | | m971 scFv light |
| | | chain CDR2 |
| 98 | QQSYSIPQT | Anti-CD22 |
| | | m971 scFv light |
| | | chain CDR3 |
| 99 | QVQLQQSGPGMVKPSQTLSLTCAISGDSVSSNSVAWNWIRQSPSRGL | Anti-CD22 |
| | EWLGRTYYRSTWYNDYAVSMKSRITINPDTNKNQFSLQLNSVTPED | m971-L7 scFv |
| | TAVYYCAREVTGDLEDAFDIWGQGTMVTVSSGGGGSGGGGSGGGG | entire sequence, |
| | SDIQMIQSPSSLSASVGDRVTITCRASQTIWSYLNWYRQRPGEAPNLL | with 3xG4S |
| | IYAASSLQSGVPSRFSGRGSGTDFTLTISSLQAEDFATYYCQQSYSIPQ | linker |
| | TFGQGTKLEIK | |
| 100 | QVQLQQSGPGMVKPSQTLSLTCAISGDSVSSNSVAWNWIRQSPSRGL | Anti-CD22 |
| | EWLGRTYYRSTWYNDYAVSMKSRITINPDTNKNQFSLQLNSVTPED | m971-L7 scFv |
| | TAVYYCAREVTGDLEDAFDIWGQGTMVTVSS | heavy chain |
| | | variable region |
| 101 | GDSVSSNSVA | Anti-CD22 |
| | | m971-L7 scFv |
| | | heavy chain |
| | | CDR1 |
| 102 | TYYRSTWYN | Anti-CD22 |
| | | m971-L7 scFv |
| | | heavy chain |
| | | CDR2 |
| 103 | AREVTGDLEDAFDI | Anti-CD22 |
| | | m971-L7 scFv |
| | | heavy chain |
| | | CDR3 |
| 104 | DIQMIQSPSSLSASVGDRVTITCRASQTIWSYLNWYRQRPGEAPNLLI | Anti-CD22 |
| | YAASSLQSGVPSRFSGRGSGTDFTLTISSLQAEDFATYYCQQSYSIPQT | m971-L7 scFv |
| | FGQGTKLEIK | light chain |
| | | variable region |
| 105 | QTIWSY | Anti-CD22 |
| | | m971-L7 scFv |
| | | light chain |
| | | CDR1 |
| 106 | AAS | Anti-CD22 |
| | | m971-L7 scFv |
| | | light chain |
| | | CDR2 |
| 107 | QQSYSIPQT | Anti-CD22 |
| | | m971-L7 scFv |
| | | light chain |
| | | CDR3 |
| 108 | DIVLTQSPASLAMSLGKRATISCRASESVSVIGAHLIHWYQQKPGQPP | Anti-BCMA |
| | KLLIYLASNLETGVPARFSGSGSGTDFTLTIDPVEEDDVAIYSCLQSRI | C11D5.3 scFv |
| | FPRTFGGGTKLEIKGSTSGSGKPGSGEGSTKGQIQLVQSGPELKKPGE | entire sequence, |
| | TVKISCKASGYTFTDYSINWVKRAPGKGLKWMGWINTETREPAYAY | with Whitlow |
| | DFRGRFAFSLETSASTAYLQINNLKYEDTATYFCALDYSYAMDYWG | linker |
| | QGTSVTVSS | |
| 109 | DIVLTQSPASLAMSLGKRATISCRASESVSVIGAHLIHWYQQKPGQPP | Anti-BCMA |
| | KLLIYLASNLETGVPARFSGSGSGTDFTLTIDPVEEDDVAIYSCLQSRI | C11D5.3 scFv |
| | FPRTFGGGTKLEIK | light chain |
| | | variable region |
| 110 | RASESVSVIGAHLIH | Anti-BCMA |
| | | C11D5.3 scFv |
| | | light chain |
| | | CDR1 |
| 111 | LASNLET | Anti-BCMA |
| | | C11D5.3 scFv |
| | | light chain |
| | | CDR2 |
| 112 | LQSRIFPRT | Anti-BCMA |
| | | C11D5.3 scFv |
| | | light chain |
| | | CDR3 |
| 113 | QIQLVQSGPELKKPGETVKISCKASGYTFTDYSINWVKRAPGKGLKW | Anti-BCMA |
| | MGWINTETREPAYAYDFRGRFAFSLETSASTAYLQINNLKYEDTATY | C11D5.3 scFv |
| | FCALDYSYAMDYWGQGTSVTVSS | heavy chain |
| | | variable region |
| 114 | DYSIN | Anti-BCMA |
| | | C11D5.3 scFv |
| | | heavy chain |
| | | CDR1 |
| 115 | WINTETREPAYAYDFRG | Anti-BCMA |
| | | C11D5.3 scFv |
| | | heavy chain |
| | | CDR2 |
| 116 | DYSYAMDY | Anti-BCMA |
| | | C11D5.3 scFv |
| | | heavy chain |
| | | CDR3 |
| 117 | DIVLTQSPPSLAMSLGKRATISCRASESVTILGSHLIYWYQQKPGQPPT | Anti-BCMA |
| | LLIQLASNVQTGVPARFSGSGSRTDFTLTIDPVEEDDVAVYYCLQSRT | C12A3.2 scFv |
| | IPRTFGGGTKLEIKGSTSGSGKPGSGEGSTKGQIQLVQSGPELKKPGE | entire sequence, |
| | TVKISCKASGYTFRHYSMNWVKQAPGKGLKWMGRINTESGVPIYAD | with Whitlow |
| | DFKGRFAFSVETSASTAYLVINNLKDEDTASYFCSNDYLYSLDFWGQ | linker |
| | GTALTVSS | |
| 118 | DIVLTQSPPSLAMSLGKRATISCRASESVTILGSHLIYWYQQKPGQPPT | Anti-BCMA |
| | LLIQLASNVQTGVPARFSGSGSRTDFTLTIDPVEEDDVAVYYCLQSRT | C12A3.2 scFv |
| | IPRTFGGGTKLEIK | light chain |
| | | variable region |
| 119 | RASESVTILGSHLIY | Anti-BCMA |
| | | C12A3.2 scFv |
| | | light chain |
| | | CDR1 |
| 120 | LASNVQT | Anti-BCMA |
| | | C12A3.2 scFv |
| | | light chain |
| | | CDR2 |
| 121 | LQSRTIPRT | Anti-BCMA |
| | | C12A3.2 scFv |
| | | light chain |
| | | CDR3 |
| 122 | QIQLVQSGPELKKPGETVKISCKASGYTFRHYSMNWVKQAPGKGLK | Anti-BCMA |
| | WMGRINTESGVPIYADDFKGRFAFSVETSASTAYLVINNLKDEDTAS | C12A3.2 scFv |
| | YFCSNDYLYSLDFWGQGTALTVSS | heavy chain |
| | | variable region |
| 123 | HYSMN | Anti-BCMA |
| | | C12A3.2 scFv |
| | | heavy chain |
| | | CDR1 |
| 124 | RINTESGVPIYADDFKG | Anti-BCMA |
| | | C12A3.2 scFv |
| | | heavy chain |
| | | CDR2 |
| 125 | DYLYSLDF | Anti-BCMA |
| | | C12A3.2 scFv |
| | | heavy chain |
| | | CDR3 |
| 126 | EVQLLESGGGLVQPGGSLRLSCAASGFTFSSYAMSWVRQAPGKGLE | Anti-BCMA |
| | WVSSISGSGDYIYYADSVKGRFTISRDISKNTLYLQMNSLRAEDTAV | FHVH33 entire |
| | YYCAKEGTGANSSLADYRGQGTLVTVSS | sequence |
| 127 | GFTFSSYA | Anti-BCMA |
| | | FHVH33 CDR1 |
| 128 | ISGSGDYI | Anti-BCMA |
| | | FHVH33 CDR2 |
| 129 | AKEGTGANSSLADY | Anti-BCMA |
| | | FHVH33 CDR3 |
| 130 | DIQMTQSPSSLSASVGDRVTITCRASQSISSYLNWYQQKPGKAPKLLI | Anti-BCMA |
| | YAASSLQSGVPSRFSGSGSGTDFTLTISSLQPEDFATYYCQQKYDLLT | CT103A scFv |
| | FGGGTKVEIKGSTSGSGKPGSGEGSTKGQLQLQESGPGLVKPSETLSL | entire sequence, |
| | TCTVSGGSISSSSYYWGWIRQPPGKGLEWIGSISYSGSTYYNPSLKSR | with Whitlow |
| | VTISVDTSKNQFSLKLSSVTAADTAVYYCARDRGDTILDVWGQGTM | linker |
| | VTVSS | |
| 131 | DIQMTQSPSSLSASVGDRVTITCRASQSISSYLNWYQQKPGKAPKLLI | Anti-BCMA |
| | YAASSLQSGVPSRFSGSGSGTDFTLTISSLQPEDFATYYCQQKYDLLT | CT103A scFv |
| | FGGGTKVEIK | light chain |
| | | variable region |
| 132 | QSISSY | Anti-BCMA |
| | | CT103A scFv |
| | | light chain |
| | | CDR1 |
| 133 | AAS | Anti-BCMA |
| | | CT103A scFv |
| | | light chain |
| | | CDR2 |
| 134 | QQKYDLLT | Anti-BCMA |
| | | CT103A scFv |
| | | light chain |
| | | CDR3 |
| 135 | QLQLQESGPGLVKPSETLSLTCTVSGGSISSSSYYWGWIRQPPGKGLE | Anti-BCMA |
| | WIGSISYSGSTYYNPSLKSRVTISVDTSKNQFSLKLSSVTAADTAVYY | CT103A scFv |
| | CARDRGDTILDVWGQGTMVTVSS | heavy chain |
| | | variable region |
| 136 | GGSISSSSYY | Anti-BCMA |
| | | CT103A scFv |
| | | heavy chain |
| | | CDR1 |
| 137 | ISYSGST | Anti-BCMA |
| | | CT103A scFv |
| | | heavy chain |
| | | CDR2 |
| 138 | ARDRGDTILDV | Anti-BCMA |
| | | CT103A scFv |
| | | heavy chain |
| | | CDR3 |
| 139 | atggccttaccagtgaccgccttgctcctgccgctggccttgctgctccacgccgccaggccggacatcc | Exemplary |
| | agatgacccagtctccatcctccctgtctgcatctgtaggagacagagtcaccatcacttgccgggcaag | BCMA CAR |
| | tcagagcattagcagctatttaaattggtatcagcagaaaccagggaaagcccctaagctcctgatctat | nucleotide |
| | gctgcatccagtttgcaaagtggggtcccatcaaggttcagtggcagtggatctgggacagatttcactc | sequence |
| | tcaccatcagcagtctgcaacctgaagattttgcaacttactactgtcagcaaaaatacgacctcctcac | |
| | ttttggcggagggaccaaggttgagatcaaaggcagcaccagcggctccggcaagcctggctctggcgag | |
| | ggcagcacaaagggacagctgcagctgcaggagtcgggcccaggactggtgaagccttcggagaccctg | |
| | tccctcacctgcactgtctctggtggctccatcagcagtagtagttactactggggctggatccgccag | |
| | cccccagggaaggggctggagtggattgggagtatctcctatagtgggagcacctactacaacccgtccc | |
| | tcaagagtcgagtcaccatatccgtagacacgtccaagaaccagttctccctgaagctgagttctgtgac | |
| | cgccgcagacacggcggtgtactactgcgccagagatcgtggagacaccatactagacgtatggggtcag | |
| | ggtacaatggtcaccgtcagctcattcgtgcccgtgttcctgcccgccaaacctaccaccacccctgccc | |
| | ctagacctcccaccccagccccaacaatcgccagccagcctctgtctctgcggcccgaagcctgtagacc | |
| | tgctgccggcggagccgtgcacaccagaggcctggacttcgcctgcgacatctacatctgggcccctctg | |
| | gccggcacctgtggcgtgctgctgctgagcctggtgatcaccctgtactgcaaccaccggaacaaacggg | |
| | gcagaaagaaactcctgtatatattcaaacaaccatttatgagaccagtacaaactactcaagaggaaga | |
| | tggctgtagctgccgatttccagaagaagaagaaggaggatgtgaactgagagtgaagttcagcagatcc | |
| | gccgacgcccctgcctaccagcagggacagaaccagctgtacaacgagctgaacctgggcagacgggaag | |
| | agtacgacgtgctggacaagcggagaggccgggaccccgagatgggcggaaagcccagacggaagaaccc | |
| | ccaggaaggcctgtataacgaactgcagaaagacaagatggccgaggcctacagcgagatcggcatgaag | |
| | ggcgagcggaggcgcggcaagggccacgatggcctgtaccagggcctgagcaccgccaccaaggacacct | |
| | acgacgccctgcacatgcaggccctgccccccaga | |
| 140 | MALPVTALLLPLALLLHAARPDIQMTQSPSSLSASVGDRVTITCRASQ | Exemplary |
| | SISSYLNWYQQKPGKAPKLLIYAASSLQSGVPSRFSGSGSGTDFTLTIS | BCMA CAR |
| | SLQPEDFATYYCQQKYDLLTFGGGTKVEIKGSTSGSGKPGSGEGSTK | amino acid |
| | GQLQLQESGPGLVKPSETLSLTCTVSGGSISSSSYYWGWIRQPPGKGL | sequence |
| | EWIGSISYSGSTYYNPSLKSRVTISVDTSKNQFSLKLSSVTAADTAVY | |
| | YCARDRGDTILDVWGQGTMVTVSSFVPVFLPAKPTTTPAPRPPTPAP | |
| | TIASQPLSLRPEACRPAAGGAVHTRGLDFACDIYIWAPLAGTCGVLL | |
| | LSLVITLYCNHRNKRGRKKLLYIFKQPFMRPVQTTQEEDGCSCRFPEE | |
| | EEGGCELRVKFSRSADAPAYQQGQNQLYNELNLGRREEYDVLDKRR | |
| | GRDPEMGGKPRRKNPQEGLYNELQKDKMAEAYSEIGMKGERRRGK | |
| | GHDGLYQGLSTATKDTYDALHMQALPPR | |
| 141 | ACRPAAGGAVHTRGLDFACDIYIWAPLAGTCGVLLLSLVITLYC | CD8 |
| | | transmembrane |
| 142 | TTTPAPRPPTPAPTIASQPLSLRPE | CD8 Hinge |
| 143 | GGGGSGGGGSGGGGS | linker |
| 144 | SNYYGSSYWFFDV | Anti-CD20 |
| | | Leu16 scFv |
| | | heavy chain |
| | | CDR3 |
| 145 | GFTFSGYW | CDR-H1 |
| 146 | ISPGGGST | CDR-H2 |
| 147 | ASSLTATHTYEYDY | CDR-H3 |
| 148 | EVQLVESGGGLVQSGGSLRLSCAASGFTFSGYWMYWVRQAPGKGL | VHH |
| | EWVSAISPGGGSTYYPDSVKGRFTISRDNAKNTLYLQMNSLEPEDTA | |
| | LYYCASSLTATHTYEYDYWGQGTQVTVSS | |
| 149 | GYTFSNYW | CDR-H1 |
| 150 | ILPGSGST | CDR-H2 |
| 151 | ARRGYGYDEGFDY | CDR-H3 |
| 152 | QDINSY | CDR-L1 |
| 153 | RAN | CDR-L2 |
| 154 | LQYDEFPPT | CDR-L3 |
| 155 | QVQLQQSGAELMKPGASVKMSCKATGYTFSNYWIEWVKQRPGHGL | VH |
| | EWIGEILPGSGSTSYNEKFKGKATFTADTSSSTAYMQLSSLTSEDSAV | |
| | YYCARRGYGYDEGFDYWGQGSTLTVSS | |
| 156 | DIKMTQSPSSMYASLGERVTITCKASQDINSYLSWFQQKPGKSPKTLI | VL |
| | YRANRLVDGVPSRFSGSGSGQDYSLTISSLEYEDMGIYYCLQYDEFPP | |
| | TFGAGTKLELKR | |
| 157 | QVQLQQSGAELMKPGASVKMSCKATGYTFSNYWIEWVKQRPGHGL | scFv |
| | EWIGEILPGSGSTSYNEKFKGKATFTADTSSSTAYMQLSSLTSEDSAV | |
| | YYCARRGYGYDEGFDYWGQGSTLTVSSGGGGSGGGGSGGGGSDIK | |
| | MTQSPSSMYASLGERVTITCKASQDINSYLSWFQQKPGKSPKTLIYR | |
| | ANRLVDGVPSRFSGSGSGQDYSLTISSLEYEDMGIYYCLQYDEFPPTF | |
| | GAGTKLELKR | |
| 158 | GYTFTDYV | CDR-H1 |
| 159 | IYPGSGSS | CDR-H2 |
| 160 | ARPGDLGFAY | CDR-H3 |
| 161 | QSVDYDGDSY | CDR-L1 |
| 162 | AAS | CDR-L2 |
| 163 | QQSNKDPFT | CDR-L3 |
| 164 | QVQLQQSGPELVKPGASVKMSCKASGYTFTDYVISWVRQAPGQGLE | VH |
| | WIGEIYPGSGSSYYNEKFKGRATLTADKSSNTAYMQLSSLRSEDSAV | |
| | YFCARPGDLGFAYWGQGTLVTVSS | |
| 165 | DIVLTQSPSSLAVSLGQRATISCKASQSVDYDGDSYMNWYQQKPGQ | VL |
| | PPKLLIYAASNLESGIPARFSGSGSGTDFTLTIHPVEEEDAATYYCQQS | |
| | NKDPFTFGGGTKLELKR | |
| 166 | QVQLQQSGPELVKPGASVKMSCKASGYTFTDYVISWVRQAPGQGLE | scFv |
| | WIGEIYPGSGSSYYNEKFKGRATLTADKSSNTAYMQLSSLRSEDSAV | |
| | YFCARPGDLGFAYWGQGTLVTVSSGGGGSGGGGSGGGGSDIVLTQS | |
| | PSSLAVSLGQRATISCKASQSVDYDGDSYMNWYQQKPGQPPKLLIY | |
| | AASNLESGIPARFSGSGSGTDFTLTIHPVEEEDAATYYCQQSNKDPFT | |
| | FGGGTKLELKR | |
| 167 | GGTFSSYA | CDR-H1 |
| 168 | INPNSGGT | CDR-H2 |
| 169 | ARDGYSGSYSD | CDR-H3 |
| 170 | QSVLSSSYNKNY | CDR-L1 |
| 171 | WAS | CDR-L2 |
| 172 | QQYYSTPWT | CDR-L3 |
| 173 | QVQLVQSGAEVKKPGASVKVSCKASGGTFSSYAISWVRQAPGQGLE | VH |
| | WMGWINPNSGGTNYAQKFQGRVTMTRDTSTSTVYMELSSLRSEDT | |
| | AVYYCARDGYSGSYSDWGQGTLVTVSS | |
| 174 | DIVMTQSPDSLAVSLGERATINCKSSQSVLSSSYNKNYLAWYQQKPG | VL |
| | QPPKLLIYWASTRESGVPDRFSGSGSGTDFTLTISSLQAEDVAVYYCQ | |
| | QYYSTPWTFGQGTKVEIK | |
| 175 | QVQLVQSGAEVKKPGASVKVSCKASGGTFSSYAISWVRQAPGQGLE | scFv |
| | WMGWINPNSGGTNYAQKFQGRVTMTRDTSTSTVYMELSSLRSEDT | |
| | AVYYCARDGYSGSYSDWGQGTLVTVSSGGGGSGGGGSGGGGSDIV | |
| | MTQSPDSLAVSLGERATINCKSSQSVLSSSYNKNYLAWYQQKPGQPP | |
| | KLLIYWASTRESGVPDRFSGSGSGTDFTLTISSLQAEDVAVYYCQQY | |
| | YSTPWTFGQGTKVEIK | |
| 176 | GYTFTSYD | CDR-H1 |
| 177 | IIPLSGAP | CDR-H2 |
| 178 | ARGALYNWNDGWFDP | CDR-H3 |
| 179 | QDIGDY | CDR-L1 |
| 180 | DAS | CDR-L2 |
| 181 | QQANSFPLT | CDR-L3 |
| 182 | QVQLVQSGAEVKKPGSSVKVSCKASGYTFTSYDINWVRQAPGQGLE | VH |
| | WMGGIIPLSGAPNYAHKFQGRVTITADESTSTAYMELSSLRSEDTAV | |
| | YYCARGALYNWNDGWFDPWGQGTLVTVSS | |
| 183 | DIQMTQSPSSLSASVGDRVTITCRASQDIGDYLAWYQQKPGKAPKLL | VL |
| | IYDASSLQSGVPSRFSGSGSGTDFTLTISSLQPEDFATYYCQQANSFPL | |
| | TFGGGTKVEIK | |
| 184 | QVQLVQSGAEVKKPGSSVKVSCKASGYTFTSYDINWVRQAPGQGLE | scFv |
| | WMGGIIPLSGAPNYAHKFQGRVTITADESTSTAYMELSSLRSEDTAV | |
| | YYCARGALYNWNDGWFDPWGQGTLVTVSSGGGGSGGGGSGGGGS | |
| | DIQMTQSPSSLSASVGDRVTITCRASQDIGDYLAWYQQKPGKAPKLL | |
| | IYDASSLQSGVPSRFSGSGSGTDFTLTISSLQPEDFATYYCQQANSFPL | |
| | TFGGGTKVEIK | |
| 185 | GDTFSDHA | CDR-H1 |
| 186 | MNPKIGNT | CDR-H2 |
| 187 | VYDSSGYDAFDI | CDR-H3 |
| 188 | QSVLSTSYNRNF | CDR-L1 |
| 189 | QQYYSTPYT | CDR-L3 |
| 190 | QVQLVQSGAEVKKPGASVKVSCKASGDTFSDHAINWVRQAPGQGL | VH |
| | EWMGWMNPKIGNTGYAQKFQGRVTMTRDTSTSTVYMELSSLRSED | |
| | TAVYYCVYDSSGYDAFDIWGQGTTVTVSS | |
| 191 | DIVMTQSPDSLAVSLGERATINCKSSQSVLSTSYNRNFLAWYQQKPG | VL |
| | QPPKLLIYWASTRQSGVPDRFSGSGSGTDFTLTISSLQAEDVAVYYCQ | |
| | QYYSTPYTFGQGTKLEIK | |
| 192 | QVQLVQSGAEVKKPGASVKVSCKASGDTFSDHAINWVRQAPGQGL | scFv |
| | EWMGWMNPKIGNTGYAQKFQGRVTMTRDTSTSTVYMELSSLRSED | |
| | TAVYYCVYDSSGYDAFDIWGQGTTVTVSSGGGGSGGGGSGGGGSDI | |
| | VMTQSPDSLAVSLGERATINCKSSQSVLSTSYNRNFLAWYQQKPGQP | |
| | PKLLIYWASTRQSGVPDRFSGSGSGTDFTLTISSLQAEDVAVYYCQQ | |
| | YYSTPYTFGQGTKLEIK | |
| 193 | GYSLITHW | CDR-H1 |
| 194 | INPSDGVT | CDR-H2 |
| 195 | AREYYGEGFDY | CDR-H3 |
| 196 | QGISNY | CDR-L1 |
| 197 | SAS | CDR-L2 |
| 198 | QQSYSTPLT | CDR-L3 |
| 199 | QVQLVQSGAEVKKPGASVKVSCKASGYSLITHWMHWVRQAPGQGL | VH |
| | EWMGMINPSDGVTYYAQTFQGRVTMTRDTSTSTVYMELSSLRSEDT | |
| | AVYYCAREYYGEGFDYWGQGTLVTVSS | |
| 200 | DIQMTQSPSSLSASVGDRVTITCRASQGISNYLAWYQQKPGKAPKLLI | VL |
| | YSASNLQSGVPSRFSGSGSGTDFTLTISSLQPEDFATYYCQQSYSTPLT | |
| | FGGGTKVEIKR | |
| 201 | QVQLVQSGAEVKKPGASVKVSCKASGYSLITHWMHWVRQAPGQGL | scFv |
| | EWMGMINPSDGVTYYAQTFQGRVTMTRDTSTSTVYMELSSLRSEDT | |
| | AVYYCAREYYGEGFDYWGQGTLVTVSSGGGGSGGGGSGGGGSDIQ | |
| | MTQSPSSLSASVGDRVTITCRASQGISNYLAWYQQKPGKAPKLLIYS | |
| | ASNLQSGVPSRFSGSGSGTDFTLTISSLQPEDFATYYCQQSYSTPLTFG | |
| | GGTKVEIKR | |
| 202 | GYWMY | CDR-H1 |
| 203 | AISPGGGSTYYPDSVKG | CDR-H2 |
| 204 | SLTATHTYEYDY | CDR-H3 |
| 205 | GFTFSGY | CDR-H1 |
| 206 | SPGGGS | CDR-H2 |
| 207 | NYWIE | CDR-H1 |
| 208 | EILPGSGSTSYNEKFKG | CDR-H2 |
| 209 | RGYGYDEGFDY | CDR-H3 |
| 210 | KASQDINSYLS | CDR-L1 |
| 211 | RANRLVD | CDR-L2 |
| 212 | GYTFSNY | CDR-H1 |
| 213 | LPGSGS | CDR-H2 |
| 214 | DYVIS | CDR-H1 |
| 215 | EIYPGSGSSYYNEKFKG | CDR-H2 |
| 216 | PGDLGFAY | CDR-H3 |
| 217 | KASQSVDYDGDSYMN | CDR-L1 |
| 218 | AASNLES | CDR-L2 |
| 219 | GYTFTDY | CDR-H1 |
| 220 | YPGSGS | CDR-H2 |
| 221 | SYAIS | CDR-H1 |
| 222 | WINPNSGGTNYAQKFQG | CDR-H2 |
| 223 | DGYSGSYSD | CDR-H3 |
| 224 | KSSQSVLSSSYNKNYLA | CDR-L1 |
| 225 | WASTRES | CDR-L2 |
| 226 | GGTFSSY | CDR-H1 |
| 227 | NPNSGG | CDR-H2 |
| 228 | SYDIN | CDR-H1 |
| 229 | GIIPLSGAPNYAHKFQG | CDR-H2 |
| 230 | GALYNWNDGWFDP | CDR-H3 |
| 231 | RASQDIGDYLA | CDR-L1 |
| 232 | DASSLQS | CDR-L2 |
| 233 | GYTFTSY | CDR-H1 |
| 234 | IPLSGA | CDR-H2 |
| 235 | DHAIN | CDR-H1 |
| 236 | WMNPKIGNTGYAQKFQG | CDR-H2 |
| 237 | DSSGYDAFDI | CDR-H3 |
| 238 | KSSQSVLSTSYNRNFLA | CDR-L1 |
| 239 | WASTRQS | CDR-L2 |
| 240 | GDTFSDH | CDR-H1 |
| 241 | NPKIGN | CDR-H2 |
| 242 | THWMH | CDR-H1 |
| 243 | MINPSDGVTYYAQTFQG | CDR-H2 |
| 244 | EYYGEGFDY | CDR-H3 |
| 245 | RASQGISNYLA | CDR-L1 |
| 246 | SASNLQS | CDR-L2 |
| 247 | GYSLITH | CDR-H1 |
| 248 | NPSDGV | CDR-H2 |

What is claimed:

1. A method of transducing T cells, the method comprising contacting a non-activated T cell with a lentiviral vector comprising a CD4 binding agent, wherein the lentiviral vector transduces the non-activated T cell.

2. The method of claim 1, wherein the T cell is a CD4+ T cell.

3. The method of claim 1 or claim 2, wherein the non-activated T cell is surface negative for one or more T cell activation markers selected from the group consisting of CD25, CD44 and CD69.

4. The method of any of claims 1-3, wherein the non-activated T cell has not been treated with an anti-CD3 antibody (e.g., OKT3).

5. The method of any of claims 1-4, wherein the non-activated T cell has not been treated with an anti-CD28 antibody (e.g., CD28.2).

6. The method of any of claims 1-5, wherein the non-activated T cell has not been treated with a bead coupled to an anti-CD3 antibody (e.g. OKT3) and an anti-CD28 antibody (e.g. CD28.2), optionally wherein the bead is a superparamagnetic bead.

7. The method of any of claims 1-6, wherein the non-activated T cell has not been treated with a T cell activating cytokine (e.g., recombinant IL-2, IL-7, IL-15, IL-21, or combinations thereof), optionally wherein the T cell activating cytokine is a human cytokine.

8. The method of any of claims 1-7, wherein the non-activated T cell has not been treated with a soluble T cell costimulatory molecule (e.g. anti-CD28 antibody or soluble CD80, soluble CD86, soluble CD137L or soluble ICOS-L).

9. The method of any of claims 1-8, wherein the lentiviral vector comprises a transgene encoding an engineered receptor that binds to or recognizes a protein or antigen expressed by or on cells associated with a disease or condition (e.g. tumor cells).

10. The method of claim 9, wherein the engineered receptor is a chimeric antigen receptor

11. The method of claim 10, wherein the CAR comprises an antigen-binding domain, a transmembrane domain, and an intracellular signaling domain comprising intracellular components of a CD3zeta signaling domain and a costimulatory signaling domain.

12. The method of claim 11, wherein the costimulatory signaling domain is a CD28 costimulatory domain, optionally wherein the CD28 costimulatory signaling domain comprises the amino acid sequence set forth in SEQ ID NO:60.

13. The method of claim 11 or claim 12, wherein the costimulatory signaling domain is a 4-1BB signaling domain, optionally wherein the 4-1BB signaling domain comprises the amino acid sequence set forth in SEQ ID NO:59.

14. The method of any of claims 11-13, wherein the CD3zeta signaling domain comprises the sequence set forth in SEQ ID NO:61 or SEQ ID NO:62.

15. The method of any of claims 11-14, wherein the transmembrane domain comprises the sequence set forth in any one of SEQ ID NOS:56, 57, and 58.

16. The method of any of claims 11-15, wherein the CAR comprises a hinge domain, optionally wherein the hinge domain comprises the sequence set forth in any one of SEQ ID NOS:50, 51, 52, 53, 54, 55, and 142.

17. The method of any of claims 11-16, wherein the antigen binding domain binds to an antigen selected from the group consisting of CD19, CD20, CD22, and BCMA.

18. The method of any of claims 11-17, wherein the antigen binding domain binds to CD19.

19. The method of any of claims 11-18, wherein the antigen binding domain comprises:
(a) a CDR-H1, a CDRH-2, and a CDR-H3 comprising the amino acid sequence set forth in SEQ ID NO: 70, 71, and 72, respectively, and a CDR-L1, a CDR-L2, and a CDR-L3 comprising the amino acid sequence set forth in SEQ ID NO: 65, 66, and 67, respectively;
(b) a VH region comprising the amino acid sequence set forth in SEQ ID NO:69, and a VL region comprising the amino acid sequence set forth in SEQ ID NO:64; and/or
(c) the amino acid sequence set forth in SEQ ID NO:63 or 73.

20. The method of any of claims 11-19, wherein the CAR comprises the amino acid sequence set forth in SEQ ID NO:75, 77, 79, or 81 and/or an amino acid sequence encoded by the polynucleotide sequence set forth in SEQ ID NO:74, 76, 78, or 80.

21. The method of any of claims 11-17, wherein the antigen binding domain binds to CD20.

22. The method of any of claims 11-17 and 21, wherein the antigen binding domain comprises:
(a) a CDR-H1, a CDRH-2, and a CDR-H3 comprising the amino acid sequence set forth in SEQ ID NO: 88, 89, and 144, respectively, and a CDR-L1, a CDR-L2, and a CDR-L3 comprising the amino acid sequence set forth in SEQ ID NO: 84, 85, and 86, respectively;
(b) a VH region comprising the amino acid sequence set forth in SEQ ID NO:87, and a VL region comprising the amino acid sequence set forth in SEQ ID NO:83; and/or
(c) the amino acid sequence set forth in SEQ ID NO:82.

23. The method of any of claims 11-17, wherein the antigen binding domain binds to CD22.

24. The method of any of claims 11-17 and 23, wherein the antigen binding domain comprises:
(a) a CDR-H1, a CDRH-2, and a CDR-H3 comprising the amino acid sequence set forth in SEQ ID NO: 92, 93, and 94, respectively, and a CDR-L1, a CDR-L2, and a CDR-L3 comprising the amino acid sequence set forth in SEQ ID NO: 96, 97, and 98, respectively; or
a CDR-H1, a CDRH-2, and a CDR-H3 comprising the amino acid sequence set forth in SEQ ID NO: 101, 102, and 103, respectively, and a CDR-L1, a CDR-L2, and a CDR-L3 comprising the amino acid sequence set forth in SEQ ID NO: 105, 106, and 107, respectively; and/or
(b) a VH region comprising the amino acid sequence set forth in SEQ ID NO:91, and a VL region comprising the amino acid sequence set forth in SEQ ID NO:95; or
a VH region comprising the amino acid sequence set forth in SEQ ID NO:100, and a VL region comprising the amino acid sequence set forth in SEQ ID NO:104; and/or
(c) the amino acid sequence set forth in SEQ ID NO:90 or 99.

25. The method of any of claims 11-17, wherein the antigen binding domain binds to BCMA.

26. The method of any of claims 11-17 and 25, wherein the antigen binding domain comprises:
(a) a CDR-H1, a CDRH-2, and a CDR-H3 comprising the amino acid sequence set forth in SEQ ID NO: 114, 115, and 116, respectively, and a CDR-L1, a CDR-L2, and a CDR-L3 comprising the amino acid sequence set forth in SEQ ID NO: 110, 111, and 112, respectively;
a CDR-H1, a CDRH-2, and a CDR-H3 comprising the amino acid sequence set forth in SEQ ID NO: 123, 124, and 125, respectively, and a CDR-L1, a CDR-L2, and a CDR-L3 comprising the amino acid sequence set forth in SEQ ID NO: 119, 120, and 121, respectively;
a CDR-H1, a CDRH-2, and a CDR-H3 comprising the amino acid sequence set forth in SEQ ID NO: 127, 128, and 129, respectively; or
a CDR-H1, a CDRH-2, and a CDR-H3 comprising the amino acid sequence set forth in SEQ ID NO: 136, 137, and 138, respectively, and a CDR-L1, a CDR-L2, and a CDR-L3 comprising the amino acid sequence set forth in SEQ ID NO:132, 133, and 134, respectively; and/or
(b) a VH region comprising the amino acid sequence set forth in SEQ ID NO:113, and a VL region comprising the amino acid sequence set forth in SEQ ID NO:109;
a VH region comprising the amino acid sequence set forth in SEQ ID NO:122, and a VL region comprising the amino acid sequence set forth in SEQ ID NO:118;
a VH region comprising the amino acid sequence set forth in SEQ ID NO:135, and a VL region comprising the amino acid sequence set forth in SEQ ID NO:131; or
a VH region comprising the amino acid sequence set forth in SEQ ID NO:126; and/or
(c) the amino acid sequence set forth in SEQ ID NO:108, 117, or 130.

27. The method of any of claims 11-17, 25, and 26, wherein the CAR comprises the amino acid sequence set forth in SEQ ID NO:140 and/or an amino acid sequence encoded by the polynucleotide sequence set forth in SEQ ID NO:139.

28. The method of claim 9, wherein the engineered receptor is an engineered T cell receptor (TCR).

29. The method of any of claims 1-28, wherein the non-activated T cell is a human T cell.

30. The method of any of claims 1-29, wherein the non-activated T cell is in a subject.

31. The method of any of claims 1-29, wherein the non-activated T cell is in vitro.

32. The method of any of claims 1-29, wherein the non-activated T cell is ex vivo from a subject.

33. The method of claim 30 or claim 32, wherein, prior to the contacting, the subject has not been administered a T cell activating treatment.

34. The method of any of claims 15, 17 and 18, wherein the subject has a disease or condition.

35. A method of transducing a population of T cells, the method comprising contacting a population of non-activated T cells with a composition comprising lentiviral vectors comprising a CD4 binding agent, wherein the population of non-activated T cells is transduced at an efficiency of at least 1%.

36. The method of claim 35, wherein the population of non-activated T cells is transduced at an efficiency of at least 5%.

37. The method of claim 35 or claim 36, wherein the population of non-activated T cells is transduced at an efficiency of at least 10%, at least 15%, at least 20%, at least 25%, at least 30%, at least 35%, or at least 40%.

38. The method of any of claims 35-37, wherein at least 75% of the T cells in the population of non-activated T cells are surface negative for one or more T cell activation marker(s) selected from the group consisting of CD25, CD44 and CD69 (e.g. at least 80%, at least 85%, at least 90%, at least 95% of the T cells in the population are surface negative for the one or more T cell activation marker(s)).

39. The method of any of claims 35-38, wherein the population of non-activated T cells comprises CD4+ T cells (e.g. at least 10%, at least 20%, at least 30%, at least 40%, at least 50%, at least 60%, at least 70%, at least 80%, at least 90% of the population of non-activated T cells are CD4+ T cells).

40. The method of claim 39, wherein at least 75% of the CD4+ T cells are surface negative for one or more T cell activation marker(s) selected from the group consisting of CD25, CD44 and CD69 (e.g. at least 80%, at least 85%, at least 90%, at least 95% of the CD4+ T cells are surface negative for the one or more T cell activation marker(s)).

41. The method of claim 39 or claim 40, wherein the CD4+ T cells are transduced at an efficiency of at least 1%, at least 5%, at least 10%, at least 15%, at least 20%, at least 25%, at least 30%, at least 35%, or at least 40%.

42. The method of any of claims 35-41, wherein the population of non-activated T cells has not been treated with an anti-CD3 antibody (e.g., OKT3).

43. The method of any of claims 35-42, wherein the population of non-activated T cell has not been treated with an anti-CD28 antibody (e.g., CD28.2).

44. The method of any of claims 35-43, wherein the population of non-activated T cells has not been treated with a bead coupled to an anti-CD3 antibody (e.g. OKT3) and an anti-CD28 antibody (e.g. CD28.2), optionally wherein the bead is a superparamagnetic bead.

45. The method of any of claims 35-44, wherein the population of non-activated T cell has not been treated with a T cell activating cytokine (e.g., recombinant IL-2, IL-7, IL-15, IL-21, or combinations thereof), optionally wherein the T cell activating cytokine is a human cytokine.

46. The method of any of claims 35-45, wherein the population of non-activated T cells has not been treated with a soluble T cell costimulatory molecule (e.g. anti-CD28 antibody or soluble CD80, soluble CD86, soluble CD137L or soluble ICOS-L).

47. The method of any of claims 35-46, wherein the population of non-activated T cells are human cells.

48. The method of any of claims 35-47, wherein the population of non-activated T cells is in a subject.

49. The method of claim 48, wherein, prior to the contacting, the subject has not been administered a T cell activating treatment.

50. The method of any of claims 35-47, wherein the population of non-activated T cells is in vitro.

51. The method of any of claims 35-47, wherein the population of non-activated T cells is ex vivo from a subject.

52. The method of any of claims 35-47, 50, and 51, wherein the population of non-activated T cells comprises peripheral blood mononuclear cells (PBMCs) or a subset thereof comprising CD4+ T cells.

53. The method of any of claims 35-47 and 50-52, wherein the population of non-activated cells is an enriched population of T cells selected from a biological sample from a subject, optionally wherein the T cells are selected for T cells surface positive for a T cell marker (e.g., CD3 or CD4).

54. The method of claim 53, wherein the biological sample is a whole blood sample, an apheresis sample, or a leukapheresis sample.

55. The method of claim 48, 49, and 51-54, wherein the subject has a disease or condition.

56. The method of any of claims 35-47 and 50-55, further comprising expanding the population of transduced T cells.

57. The method of claim 56, wherein the expanding comprises incubation of the transduced cells with one or more T cell activating cytokine(s) (e.g., recombinant IL-2, IL-7, IL-15, IL-21, or combinations thereof), optionally wherein the one or more T cell activating cytokine(s) is human.

58. The method of any of claims 35-47 and 50-56, further comprising incubating the transduced T cells with one or more T cell activating cytokine(s) (e.g., recombinant IL-2, IL-7, IL-15, IL-21, or combinations thereof), optionally wherein the one or more T cell activating cytokine(s) is human.

59. A method of in vivo transduction of T cells, the method comprising administering to a subject a composition comprising a lentiviral vector comprising a CD4 binding agent, wherein the lentiviral vector transduces T cells within the subject, and wherein the subject is not administered a T cell activating treatment (e.g. before, after or concurrently) with administration of the composition.

60. The method of claim 59, wherein the subject has a disease or condition.

61. Use of a composition comprising a lentiviral vector comprising a CD4 binding agent for treating a subject having a disease or condition, optionally a cancer.

62. A composition comprising a lentiviral vector comprising a CD4 binding agent for use in treating a subject having a disease or condition, optionally a cancer.

63. A method of treating a subject having a disease or condition, the method comprising administering to the subject a composition comprising a lentiviral vector comprising a CD4 binding agent, wherein the subject is not administered a T cell activating treatment (e.g. before, after or concurrently) with administration of the composition.

64. The method of any of claims 9-60 and 63, the use of claim 61, or the composition of claim 62, wherein the disease or condition is a cancer.

65. The method of any of claims 34, 55-58, 60, 63, and 64, the use of claim 61 or claim 64, or the composition of claim 62 or claim 64, wherein the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR).

66. A method for expanding T cells capable of recognizing and killing tumor cells in a subject in need thereof, the method comprising administering to the subject a composition comprising a lentiviral vector comprising a CD4 binding agent, wherein the subject is not administered a T cell activating treatment (e.g. before, after, or concurrently) with administration of the composition.

67. The method of claim 66, wherein the lentiviral vector comprises a transgene encoding an engineered receptor that binds to or recognizes a protein expressed on the tumor cells, optionally wherein the engineered receptor is a chimeric antigen receptor (CAR) or an engineered T cell receptor (TCR).

68. The method of any of claims 33-34, 49, 55-60, and 63-67, wherein the T cell activating treatment comprises administration of an anti-CD3 antibody (e.g., OKT3).

69. The method of any of claims 33-34, 49, 55-60, and 63-68, wherein the T cell activating treatment comprises administration of a soluble T cell costimulatory molecule (e.g., anti-CD28 antibody, or a recombinant CD80, CD86, CD137L, ICOS-L).

70. The method of any of claims 33-34, 49, 55-60, and 63-69, wherein the T cell activating treatment comprises administration of a T cell activating cytokine (e.g., recombinant IL-2, IL-7, IL-15, IL-21), optionally wherein the T cell activating cytokine is human.

71. The method of any of claims 33-34, 49, 55-60, and 63-70, wherein the T cell activating treatment comprises administration of recombinant IL-7, optionally human IL-7.

72. The method of any of claims 33-34, 49, 55-60, and 63-71, wherein the T cell activating treatment comprises administration of a lymphodepleting therapy, optionally administration of cyclophosphamide and/or fludarabine.

73. The method of any of claims 1-60 and 63-72, the use of any of claims 61, 64, and 65, or the composition of any of claims 62, 64, and 65, wherein the CD4 binding agent is an anti-CD4 antibody or an antigen-binding fragment.

74. The method, use, or composition of claim 73, wherein the anti-CD4 antibody or antigen-binding fragment is mouse, rabbit, human, or humanized.

75. The method, use, or composition of claim 73 or claim 74, wherein the anti-CD4 antibody or antigen-binding fragment is a single chain variable fragment (scFv).

76. The method, use, or composition of claim 73 or claim 74, wherein the anti-CD4 antibody or antigen-binding fragment is a single domain antibody.

77. The method, use, or composition of any of claims 73, 74, and 76, wherein the anti-CD4 antibody or antigen-binding fragment is a camelid (e.g. llama, alpaca, camel) anti-CD4 antibody or antigen-binding fragment (e.g. a VHH).

78. The method of any of claims 1-60 and 63-77, the use of any of claims 61, 64-65, and 73-77, or the composition of any of claims 62, 64, 65, and 73-77, wherein the CD4 binding agent is an anti-CD4 VHH.

79. The method of any of claims 1-60 and 63-78, the use of any of claims 61, 64-65, and 73-78, or the composition of any of claims 62, 64-65, and 73-78, wherein the CD4 binding agent is exposed on the surface of the lentiviral vector.

80. The method of any of claims 1-60 and 63-79, the use of any of claims 61, 64-65, and 73-79, or the composition of any of claims 62, 64-65, and 73-79, wherein the CD4 binding agent is fused to a transmembrane domain incorporated in the viral envelope.

81. The method of any of claims 1-60 and 63-80, the use of any of claims 61, 64-65, and 73-80, or the composition of any of claims 62, 64-65, and 73-80, wherein the lentiviral vector is pseudotyped with a viral fusion protein.

82. The method, use, or composition of claim 81, wherein the viral fusion protein is a VSV-G protein or a functional variant thereof.

83. The method, use, or composition of claim 81, wherein the virial fusion protein is a Cocal virus G protein or a functional variant thereof.

84. The method, use, or composition of claim 81, wherein the viral fusion protein is an Alphavirus fusion protein (e.g. Sindbis virus) or a functional variant thereof.

85. The method, use, or composition of claim 81, wherein the viral fusion protein is a Paramyxoviridae fusion protein (e.g., a Morbillivirus or a Henipavirus) or a functional variant thereof.

86. The method, use, or composition of claim 81 or claim 85, wherein the viral fusion protein is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.

87. The method, use, or composition of claim 81 or claim 85, wherein the viral fusion protein is a Henipavirus fusion protein (e.g., Nipah virus, Hendra virus, Cedar virus, Kumasi virus, Mójiāng virus) or a functional variant thereof.

88. The method, use, or composition of any of claims 81-87, wherein the viral fusion protein comprises one or modifications to reduce binding to its native receptor.

89. The method, use, or composition of any of claims 81-88, wherein the viral fusion protein is fused to the CD4 binding agent.

90. The method, use, or composition of any of claims 81, 85, and 87-89, wherein the viral fusion protein comprises a Nipah virus F glycoprotein (NiV-F) or a biologically active portion thereof and a Nipah virus G glycoprotein (NiV-G) or a biologically active portion thereof, and wherein the CD4 binding agent is fused to the NiV-G or the biologically active portion thereof.

91. The method, use, or composition of claim 90, wherein the CD4 binding agent is fused to the C-terminus of the Nipah virus G glycoprotein or the biologically active portion thereof.

92. The method, use, or composition of any of claims 89-91, wherein the CD4 binding protein is fused to the viral fusion proteindirectly or via a peptide linker.

93. The method, use, or composition of any of claims 89-92, wherein the NiV-G or the biologically active portion thereof is a wild-type NiV-G protein or a functionally active variant or biologically active portion thereof.

94. The method, use, or composition of any of claims 89-93, wherein the NiV-G protein or the biologically active portion is truncated and lacks up to 40 contiguous amino acid residues at or near the N-terminus of the wild-type NiV-G protein.

95. The method, use, or composition of any of claims 89-94, wherein the NiV-G protein or the biologically active portion has a 5 amino acid truncation at or near the N-terminus of the wild-type NiV-G protein, optionally wherein the NiV-G protein or the biologically active portion thereof has the amino acid sequence set forth in SEQ ID NO:12, or a sequence of amino acids that exhibits at least at or about 80%, 85%, 90% or 95% sequence identity to the sequence set forth in SEQ ID NO:12.

96. The method, use, or composition of any of claims 89-95, wherein the NiV-G protein or the biologically active portion has a 10 amino acid truncation at or near the N-terminus of the wild-type NiV-G protein, optionally wherein the NiV-G protein or the biologically active portion thereof has the amino acid sequence set forth in SEQ ID NO:44, or a sequence of amino acids that exhibits at least at or about 80%, 85%, 90% or 95% sequence identity to the sequence set forth in SEQ ID NO:44.

97. The method, use, or composition of any of claims 89-94, wherein the NiV-G protein or the biologically active portion has a 15 amino acid truncation at or near the N-terminus of the wild-type NiV-G protein, optionally wherein the NiV-G protein or the biologically active portion thereof has the amino acid sequence set forth in SEQ ID NO:45, or a sequence of amino acids that exhibits at least at or about 80%, 85%, 90% or 95% sequence identity to the sequence set forth in SEQ ID NO:45.

98. The method, use, or composition of any of claims 89-94, wherein the NiV-G protein or the biologically active portion has a 20 amino acid truncation at or near the N-terminus of the wild-type NiV-G protein, optionally wherein the NiV-G protein or the biologically active portion thereof has the amino acid sequence set forth in SEQ ID NO:13, or a sequence of amino acids that exhibits at least at or about 80%, 85%, 90% or 95% sequence identity to the sequence set forth in SEQ ID NO:13.

99. The method, use, or composition of any of claims 89-94, wherein the NiV-G protein or the biologically active portion has a 25 amino acid truncation at or near the N-terminus of the wild-type NiV-G protein, optionally wherein the NiV-G protein or the biologically active portion thereof has the amino acid sequence set forth in SEQ ID NO:14, or a sequence of amino acids that exhibits at least at or about 80%, 85%, 90% or 95% sequence identity to the sequence set forth in SEQ ID NO: 14.

100. The method, use, or composition of any of claims 89-94, wherein the NiV-G protein or the biologically active portion has a 30 amino acid truncation at or near the N-terminus of the wild-type NiV-G protein, optionally wherein the NiV-G protein or the biologically active portion thereof has the amino acid sequence set forth in SEQ ID NO:43, or a sequence of amino acids that exhibits at least at or about 80%, 85%, 90% or 95% sequence identity to the sequence set forth in SEQ ID NO:43.

101. The method, use, or composition of any of claims 89-94, wherein the NiV-G protein or the biologically active portion has a 34 amino acid truncation at or near the N-terminus of the wild-type NiV-G protein, optionally wherein the NiV-G protein or the biologically active portion thereof has the amino acid sequence set forth in SEQ ID NO:42, or a sequence of amino acids that exhibits at least at or about 80%, 85%, 90% or 95% sequence identity to the sequence set forth in SEQ ID NO:42.

102. The method, use, or composition of any of claims 89-101, wherein the NiV-G-protein or the biologically active portion thereof is a mutant NiV-G protein that exhibits reduced binding to Ephrin B2 or Ephrin B3.

103. The method, use, or composition of claim 102, wherein the mutant NiV-G protein or the biologically active portion comprises one or more amino acid substitutions corresponding to amino acid substitutions selected from the group consisting of E501A, W504A, Q530A and E533A with reference to numbering set forth in SEQ ID NO:4.

104. The method, use, or composition of claim 102 or claim 103, wherein the mutant NiV-G protein or the biologically active portion comprises the amino acid sequence set forth in SEQ ID NO: 17 or a sequence of amino acids that exhibits at least at or about 80%, 85%, 90% or 95% sequence identity to the sequence set forth in SEQ ID NO: 17.

105. The method, use, or composition of claim 102 or claim 103, wherein the NiV-G protein or the biologically active portion has the amino acid sequence set forth in SEQ ID NO: 18 or a sequence of amino acids that exhibits at least at or about 80%, 85%, 90% or 95% sequence identity to the sequence set forth in SEQ ID NO: 18.

106. The method, use, or composition of any of claims 89-105, wherein the NiV-F protein or the biologically active portion thereof is a wild-type NiV-F protein or is a functionally active variant or a biologically active portion thereof.

107. The method, use, or composition of any of claims 89-106, wherein the NiV-F protein or the biologically active portion thereof has a 20 amino acid truncation at or near the C-terminus of the wild-type NiV-F protein, optionally wherein the NiV-F protein or the biologically active portion thereof has the sequence set forth in SEQ ID NO: 20 or a sequence of amino acids that exhibits at least at or about 80%, 85%, 90% or 95% sequence identity to the sequence set forth in SEQ ID NO:
20.

108. The method, use, or composition of any of claims 89-107, wherein the NiV-F protein or the biologically active portion thereof comprises:
i) a 20 amino acid truncation at or near the C-terminus of the wild-type NiV-F protein; and
ii) a point mutation on an N-linked glycosylation site,
optionally wherein the NiV-F protein or the biologically active portion thereof has the sequence set forth in SEQ ID NO: 15, or a sequence of amino acids that exhibits at least at or about 80%, 85%, 90% or 95% sequence identity to the sequence set forth in SEQ ID NO: 15.

109. The method, use, or composition of any of claims 89-106, wherein the NiV-F protein or the biologically active portion thereof has a 22 amino acid truncation at or near the C-terminus of the wild-type NiV-F protein, optionally wherein the NiV-F protein or the biologically active portion thereof has the sequence set forth in SEQ ID NO: 16 or 21 or a sequence of amino acids that exhibits at least at or about 80%, 85%, 90% or 95% sequence identity to the sequence set forth in SEQ ID NO: 16 or 21.

110. The method, use, or composition of any of claims 89-106 and 109, wherein the NiV-F protein or the biologically active portion thereof comprises the amino acid sequence set forth in SEQ ID NO:21, or a sequence of amino acids that exhibits at least at or about 80%, 85%, 90% or 95% sequence identity to the sequence set forth in SEQ ID NO:21.

111. The method, use, or composition of any of claims 89-106, 109, and 110, wherein the Niv-G protein comprises the amino acid sequence set forth in SEQ ID NO: 17, and the Niv-F protein comprises the amino acid sequence set forth in SEQ ID NO:21.

112. The method, use, or composition of any of claims 1-111, wherein the lentiviral vector comprises a transgene.

113. The method, use, or composition of claim 112, wherein the transgene comprises a nucleic acid sequence encoding an RNA sequence capable of RNA interference (e.g. pre-miRNA, siRNA, or shRNA).

114. The method, use, or composition of claim 112, wherein the transgene is selected from the group consisting of a therapeutic gene, a reporter gene, a gene encoding an enzyme, a gene encoding a pro-drug enzyme, a gene encoding an apoptosis inducer, a gene encoding a fluorescent protein, a gene encoding a pro-drug-activating enzyme, a gene encoding an apoptotic protein, a gene encoding an apoptotic enzyme, a gene encoding a suicide protein, a gene encoding a cytokine, a gene encoding an anti-immunosuppressive protein, a gene encoding an epigenetic modulator, a gene encoding a T cell receptor (TCR), a gene encoding a chimeric antigen receptor (CAR), a gene encoding a protein that modifies the cell surface of transduced cells, a gene encoding a protein that modifies the expression of the endogenous TCR, and a gene encoding a switch receptor that converts pro-tumor into anti-tumor signals.

115. The method, use, or composition of claim 112, wherein the transgene encodes an engineered receptor that binds to or recognizes a protein or antigen expressed by cells or a lesion (e.g. tumor) associated with a disease or condition, optionally wherein the engineered receptor is a chimeric antigen receptor (CAR) or an engineered T cell receptor (TCR).

116. The method of any of claims 65 and 67-115, or the use or composition of any of claims 112-115, wherein the transgene encodes a chimeric antigen receptor (CAR).

117. The method, use or composition of claim 116, wherein the CAR comprises an antigen-binding domain, a transmembrane domain, and an intracellular signaling domain comprising intracellular components of CD3zeta signaling domain and a costimulatory signaling domain.

118. The method, use, or composition of claim 117, wherein the costimulatory signaling domain is a CD28 costimulatory domain, optionally wherein the CD28 costimulatory signaling domain comprises the amino acid sequence set forth in SEQ ID NO:60.

119. The method, use, or composition of claim 117 or claim 118, wherein the costimulatory signaling domain is a 4-1BB signaling domain, optionally wherein the 4-1BB signaling domain comprises the amino acid sequence set forth in SEQ ID NO:59.

120. The method, use, or composition of any of claims 117-119, wherein the CD3zeta signaling domain comprises the sequence set forth in SEQ ID NO:61 or SEQ ID NO:62.

121. The method, use, or composition of any of claims 117-120, wherein the transmembrane domain comprises the sequence set forth in any one of SEQ ID NOS:56, 57, and 58.

122. The method, use, or composition of any of claims 117-121, wherein the CAR comprises a hinge domain, optionally wherein the hinge domain comprises the sequence set forth in any one of SEQ ID NOS:50, 51, 52, 53, 54, 55, and 142.

123. The method, use, or composition of any of claims 117-122, wherein the antigen binding domain binds to an antigen selected from the group consisting of CD19, CD20, CD22, and BCMA.

124. The method, use, or composition of any of claims 117-123, wherein the antigen binding domain binds to CD19.

125. The method of any of claims method, use, or composition of any of claims 117-124, wherein the antigen binding domain comprises:
(a) a CDR-H1, a CDRH-2, and a CDR-H3 comprising the amino acid sequence set forth in SEQ ID NO: 70, 71, and 72, respectively, and a CDR-L1, a CDR-L2, and a CDR-L3 comprising the amino acid sequence set forth in SEQ ID NO: 65, 66, and 67, respectively;
(b) a VH region comprising the amino acid sequence set forth in SEQ ID NO:69, and a VL region comprising the amino acid sequence set forth in SEQ ID NO:64; and/or
(c) the amino acid sequence set forth in SEQ ID NO:63 or 73.

126. The method, use, or composition of any of claims 117-125, wherein the CAR comprises the amino acid sequence set forth in SEQ ID NO:75, 77, 79, or 81 and/or an amino acid sequence encoded by the polynucleotide sequence set forth in SEQ ID NO:74, 76, 78, or 80.

127. The method, use, or composition of any of claims 117-123, wherein the antigen binding domain binds to CD20.

128. The method, use, or composition of any of claims 117-123 and 127, wherein the antigen binding domain comprises:
(a) a CDR-H1, a CDRH-2, and a CDR-H3 comprising the amino acid sequence set forth in SEQ ID NO: 88, 89, and 144, respectively, and a CDR-L1, a CDR-L2, and a CDR-L3 comprising the amino acid sequence set forth in SEQ ID NO: 84, 85, and 86, respectively;
(b) a VH region comprising the amino acid sequence set forth in SEQ ID NO:87, and a VL region comprising the amino acid sequence set forth in SEQ ID NO:83; and/or
(c) the amino acid sequence set forth in SEQ ID NO:82.

129. The method, use, or composition of any of claims 117-123, wherein the antigen binding domain binds to CD22.

130. The method, use, or composition of any of claims 117-123 and 129, wherein the antigen binding domain comprises:
(a) a CDR-H1, a CDRH-2, and a CDR-H3 comprising the amino acid sequence set forth in SEQ ID NO: 92, 93, and 94, respectively, and a CDR-L1, a CDR-L2, and a CDR-L3 comprising the amino acid sequence set forth in SEQ ID NO: 96, 97, and 98, respectively; or
a CDR-H1, a CDRH-2, and a CDR-H3 comprising the amino acid sequence set forth in SEQ ID NO: 101, 102, and 103, respectively, and a CDR-L1, a CDR-L2, and a CDR-L3 comprising the amino acid sequence set forth in SEQ ID NO: 105, 106, and 107, respectively; and/or
(b) a VH region comprising the amino acid sequence set forth in SEQ ID NO:91, and a VL region comprising the amino acid sequence set forth in SEQ ID NO:95; or
a VH region comprising the amino acid sequence set forth in SEQ ID NO:100, and a VL region comprising the amino acid sequence set forth in SEQ ID NO:104; and/or
(c) the amino acid sequence set forth in SEQ ID NO:90 or 99.

131. The method, use, or composition of any of claims 117-123, wherein the antigen binding domain binds to BCMA.

132. The method, use, or composition of any of claims 117-123 and 131, wherein the antigen binding domain comprises:
(a) a CDR-H1, a CDRH-2, and a CDR-H3 comprising the amino acid sequence set forth in SEQ ID NO: 114, 115, and 116, respectively, and a CDR-L1, a CDR-L2, and a CDR-L3 comprising the amino acid sequence set forth in SEQ ID NO: 110, 111, and 112, respectively;
a CDR-H1, a CDRH-2, and a CDR-H3 comprising the amino acid sequence set forth in SEQ ID NO: 123, 124, and 125, respectively, and a CDR-L1, a CDR-L2, and a CDR-L3 comprising the amino acid sequence set forth in SEQ ID NO: 119, 120, and 121, respectively;
a CDR-H1, a CDRH-2, and a CDR-H3 comprising the amino acid sequence set forth in SEQ ID NO: 127, 128, and 129, respectively; or
a CDR-H1, a CDRH-2, and a CDR-H3 comprising the amino acid sequence set forth in SEQ ID NO: 136, 137, and 138, respectively, and a CDR-L1, a CDR-L2, and a CDR-L3 comprising the amino acid sequence set forth in SEQ ID NO:132, 133, and 134, respectively; and/or
(b) a VH region comprising the amino acid sequence set forth in SEQ ID NO:113, and a VL region comprising the amino acid sequence set forth in SEQ ID NO:109;
a VH region comprising the amino acid sequence set forth in SEQ ID NO:122, and a VL region comprising the amino acid sequence set forth in SEQ ID NO:118;
a VH region comprising the amino acid sequence set forth in SEQ ID NO:135, and a VL region comprising the amino acid sequence set forth in SEQ ID NO:131; or
a VH region comprising the amino acid sequence set forth in SEQ ID NO:126; and/or
(c) the amino acid sequence set forth in SEQ ID NO:108, 117, or 130.

133. The method of any of claims method, use, or composition of any of claims 117-123, 131, and 132, wherein the CAR comprises the amino acid sequence set forth in SEQ ID NO:140 and/or an amino acid sequence encoded by the polynucleotide sequence set forth in SEQ ID NO:139.

134. The method of any of claim 65 and claim 67-115, or the use or composition of any of claims 112-115, wherein the transgene encodes an engineered T cell receptor (TCR).

135. The method, use, or composition of any of claims 1-134, wherein the lentiviral vector does not comprise or encode a T cell activating agent, optionally wherein the T cell activating agent is a lymphoproliferative agent.

136. The method, use, or composition of claim 135, wherein the T cell activating agent is:
a polypeptide capable of binding CD3 and/or CD28;
a CD3 antibody (e.g. anti-CD3 scFv); a T cell activating cytokine (e.g. IL-2, IL-7, IL-15 or IL-21); or a T cell costimulatory molecule (e.g. anti-CD28 antibody, CD80, CD86, CD137L or ICOS-L);
a cytokine or a cytokine receptor or a signaling domain thereof that activates a STAT3 pathway, a STAT4 pathway, and/or a Jak/STAT5 pathway;
a T cell survival motif, optionally an IL-7 receptor, an IL-15 receptor, or CD28, or a functional portion thereof; and/or
a microRNA (miRNA) or short hairpin RNA (shRNA), wherein the miRNA or the shRNA stimulates the STAT5 pathway and/or inhibits the SOCS pathway.

137. The method, use, or composition of any of claims 1-136, wherein the lentiviral vector does not comprise or encode a T cell activating agent that is membrane bound and/or displayed on the surface, optionally wherein the T cell activating agent is a lymphoproliferative agent.

138. The method, use, or composition of any of claims 33-34, 49, and 55-137, wherein the subject is not administered a T cell activating treatment concurrently with the lentiviral vector.

139. The method, use, or composition of any of claims 33-34, 49, and 55-138, wherein the subject is not administered a T cell activating treatment within 1 month before the contacting with the lentiviral vector or before the administration of the composition comprising the lentiviral vectors.

140. The method, use, or composition of any of claims 33-34, 49, and 55-139, wherein the subject is not administered a T cell activating treatment within or at or about 1 week, 2 weeks, 3 weeks or 4 weeks, optionally at or about 1, 2, 3, 4, 5, 6 or 7 days, before the contacting with the lentiviral vector or before the administration of the composition comprising the lentiviral vectors.

141. The method, use, or composition of any of claims 33-34, 49, and 55-140, wherein the subject is not administered a T cell activating treatment within 1 month after the contacting with the lentiviral vector or after the administration of the composition comprising the lentiviral vectors.

142. The method, use, or composition of any of claims 33-34, 49, and 55-141, wherein the subject is not administered a T cell activating treatment within or at or about 1 week, 2 weeks, 3 weeks or 4 weeks, optionally at or about 1, 2, 3, 4, 5, 6 or 7 days, after the contacting with the lentiviral vector or after the administration of the composition comprising the lentiviral vectors.

143. The method of any one of claims 1-58, further comprising editing the T cell or the population of T cells to inactivate one or more of B2M, CIITA, TRAC, and TRB genes.

144. The method of claim 143, wherein the T cell or the population of T cells is edited to inactivate B2M, CIITA, and TRAC genes.

145. The method of claim 143, wherein the T cell or the population of T cells is edited to inactivate B2M, CIITA, and TRB genes.

146. The method of any one of claims 143-145, further comprising inserting a gene encoding CD47 into the T cell or the population of T cells at a defined locus.

147. The method of claim 146, wherein the defined locus is selected from the group consisting of a B2M locus, a CIITA locus, a TRAC locus, a TRB locus, or a safe harbor locus.

148. The method of claim 147, wherein the safe harbor locus is selected from the group consisting of an AAVS1 locus, a CCR5 locus, and a ROSA26 locus.

149. The method of any of claims 143-148, wherein the lentiviral vector comprises a transgene encoding an engineered receptor that binds to or recognizes a protein or antigen expressed by or on cells associated with the disease or condition (e.g. tumor cells), optionally wherein the engineered receptor is a chimeric antigen receptor (CAR) or an engineered T cell receptor (TCR).

150. The method of any of claims 1-60 or 63-149, wherein the contacting is carried out by ex vivo administration of the lentiviral vector to a subject.

151. The method of claim 150, wherein the extracorporeal administration comprises:
(a) obtaining whole blood from a subject;
(b) collecting the fraction of blood containing leukocyte components comprising T cells (e.g. CD4+ T cells);
(c) contacting the leukocyte components comprising T cells (e.g. CD4+ T cells) with a composition comprising the lentiviral vector; and
(d) reinfusing the contacted leukocyte components comprising T cells (e.g. CD4+ T cells) into the subject, wherein steps (a)-(d) are performed in-line in a closed fluid circuit.

152. The method of claim 151, wherein the contacting in step (c) is for no more than 24 hours, no more than 18 hours, no more than 12 hours, or no more than 6 hours.

153. A transduced T cell produced by the method of any of claims 1-29 and 31-60 and 68-152.

154. The transduced T cell of claim 153, wherein the T cell is inactivated at both alleles of the one or more genes.

155. A composition comprising the transduced T cell of claim 153 or claim 154, optionally wherein the composition is a pharmaceutical composition.

156. A population of transduced T cells produced by the method of any of claims 35-47, 50-58 and 68-152.

157. The population of transduced T cells of claim 156, wherein at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of the cells of the population of non-activated cells are inactivated at the one or more genes.

158. The population of transduced T cells of claim 156 or claim 157, wherein at least 1%, at least 5%, at least 10%, at least 15%, at least 20%, at least 25%, at least 30%, at least 35%, or at least 40% of the non-activated CD4+ T cells in the population are transduced and are inactivated at the one or more genes.

159. The population of transduced T cells of any of claims 156-158, wherein cells of the population are inactivated at both alleles of the one or more genes.

160. A composition comprising the population of transduced T cells of any of claims 156-159, optionally wherein the composition is a pharmaceutical composition.

161. A composition comprising a population of transduced T cells produced by the method of any of claims 35-47, 50-58 and 68-152, optionally wherein the composition is a pharmaceutical composition.

162. The composition of any of claims 62, 64, 65, 73-142, 155, 160, and 161, further comprising a cyropreservant, optionally wherein the cyropreservant is DMSO.

163. A method of treating a subject having a disease or condition, the method comprising administering to the subject a composition of any of claims 62, 64, 65, 73-142, 155, and 160-162, wherein the subject is not administered a T cell activating treatment (e.g. before, after or concurrently) with administration of the composition.

164. The method of claim 163, wherein the disease or condition is a cancer.

165. A method for expanding T cells capable of recognizing and killing tumor cells in a subject in need thereof, the method comprising administering to the subject a composition of any of claims 62, 64, 65, 73-142, 155, and 160-162, wherein the subject is not administered a T cell activating treatment (e.g. before, after, or concurrently) with administration of the composition.

166. The method of any of claims 138-142 and 163-165, wherein the T cell activating treatment comprises administration of an anti-CD3 antibody (e.g., OKT3).

167. The method of any of claims 138-142 and 163-166, wherein the T cell activating treatment comprises administration of a soluble T cell costimulatory molecule (e.g., anti-CD28 antibody, or a recombinant CD80, CD86, CD137L, ICOS-L).

168. The method of any of claims 138-142 and 163-167, wherein the T cell activating treatment comprises administration of a T cell activating cytokine (e.g., recombinant IL-2, IL-7, IL-15, IL-21), optionally wherein the T cell activating cytokine is a human cytokine.

169. The method of any of claims 138-142 and 163-168, wherein the T cell activating treatment comprises administration of recombinant IL-7, optionally human IL-7.

170. The method of any of claims 138-142 and 163-169, wherein the T cell activating treatment comprises administration of a lymphodepleting therapy, optionally administration of cyclophosphamide and/or fludarabine.

171. Use of a composition of any of claims 62, 64, 65, 73-142, 155, and 160-162 for formulation of a medicament for treating a subject having a disease or condition, optionally a cancer.

172. A composition of any of claims 62, 64, 65, 73-142, 155, and 160-162 for use in treating a subject having a disease or condition, optionally a cancer.

173. Use of a composition comprising a lentiviral vector comprising a CD4 binding agent for formulation of a medicament for expanding T cells capable of recognizing and killing tumor cells in a subject in need thereof.

174. Use of a composition of any of claims 62, 64, 65, 73-142, 155, and 160-162 for formulation of a medicament for expanding T cells capable of recognizing and killing tumor cells in a subject in need thereof.

175. A composition comprising a lentiviral vector comprising a CD4 binding agent for use in expanding T cells capable of recognizing and killing tumor cells in a subject in need thereof.

176. A composition of any of claims 62, 64, 65, 73-142, 155, and 160-162 for use in expanding T cells capable of recognizing and killing tumor cells in a subject in need thereof.

177. The use or the composition of any of claims 171-176 that is for use in a subject that is not administered or to be administered a T cell activating treatment (e.g. before, after or concurrently) with administration of the composition.

178. The method, use, or composition of any of claims 11-20 and 117-126, wherein the CAR comprises:
(a) an antigen binding domain comprising the VL region set forth in SEQ ID NO:64, a linker comprising the amino acid sequence set forth in SEQ ID NO:68, and the VH region set forth in SEQ ID NO:69; and/or the scFv set forth in SEQ ID NO:63;
(b) a hinge comprising the amino acid sequence set forth in SEQ ID NO:50;
(c) a transmembrane domain comprising the amino acid sequence set forth in SEQ ID NO:56;
(d) a 4-1BB signaling domain comprises the amino acid sequence set forth in SEQ ID NO:59; and/or
(e) a CD3zeta signaling domain comprising the amino acid sequence set forth in SEQ ID NO:61.

179. The method, use, or composition of any of claims 11-20, 117-126, and 178, wherein the CAR comprises the amino acid sequence set forth in SEQ ID NO:75 and/or is encoded by the nucleotide sequence set forth in SEQ ID NO:74.
